(19) United States
(12) Patent Application Publication
(10) Pub. No.: US 2026/0069726 A1
(43) Pub. Date: Mar. 12, 2026

(54) ZWITTERIONIC COMPOUNDS AND USES THEREOF

(71) Applicant: UNIVERSITY OF SASKATCHEWAN, Saskatoon (CA)

(72) Inventors: Eric William PRICE, Saskatoon (CA); Shvan Jassim RAHEEM, Saskatoon (CA)

(21) Appl. No.: 18/872,536

(22) PCT Filed: Jun. 9, 2023
(86) PCT No.: PCT/CA2023/050796

§ 371 Date: Dec. 6, 2024

Related U.S. Application Data
(60) Provisional application No. 63/350,598, filed on Jun. 9, 2022.

(51) Int. Cl. A61K 51/08; A61P 35/00; C07C 309/24; C07D 213/55; C07D 213/80

(52) U.S. Cl. CPC A61K 51/088; A61P 35/00; C07C 309/24; C07D 213/55; C07D 213/80; A61K 2121/00; A61K 2123/00; C07C 2603/18

(57) ABSTRACT

The present application relates to zwitterion compounds of Formula I, to processes of their preparations, to conjugates thereof, to compositions comprising them and to their use in diagnostics and/or therapy. wherein Q is selected from Q1, Q2, Q3, Q4 and Q5:

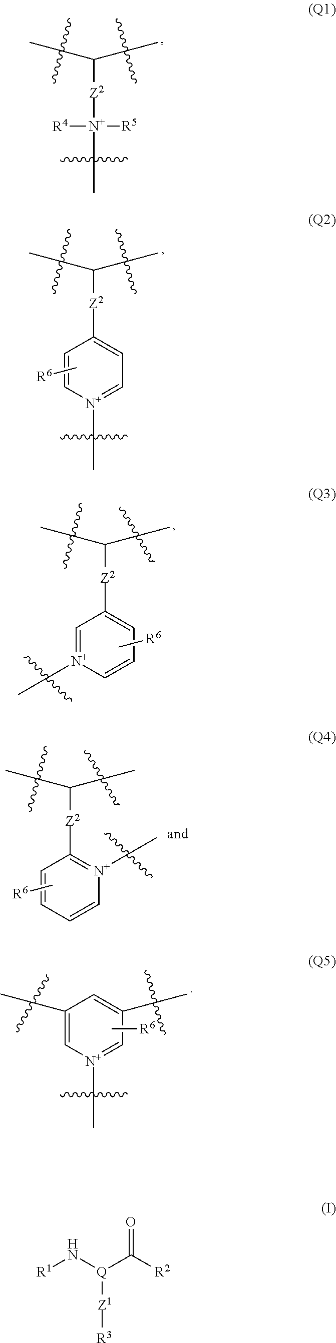

### RELATED APPLICATION

This application claims the benefit of priority to U.S. Provisional Application No. 63/350,598, filed Jun. 9, 2022, the contents of which are incorporated herein by reference in its entirety.

### FIELD

The present application relates to zwitterionic compounds and their use, for example, as linkers to form radiopharmaceuticals.

### BACKGROUND

Non-invasive molecular imaging (MI) and radionuclide therapy is revolutionizing the ability to apply the principles of personalized medicine in the diagnosis, treatment, and monitoring of diseases such as, for example, cancer [9]. The field of molecular imaging encompasses a number of imaging modalities, including single photon emission computed tomography (SPECT, nuclear), positron emission tomography (PET, nuclear), magnetic resonance imaging (MRI, non-nuclear), and optical imaging (visible and near-infrared dyes—non-nuclear). Molecular imaging, particularly when multiple modalities are integrated (e.g. PET/CT, PET/optical/CT, PET/MRI) enables specific, quantitative, real-time, and non-invasive investigation of biochemical activities at the cellular and sub-cellular level (effectively a limited full-body biopsy through imaging). Each radiopharmaceutical agent is tailored to specific receptors and disease states towards precision (personalized) medicine.

For example, cancer-targeting peptides can be transformed into molecular imaging or radiotherapeutic agents through attachment to a positron-emitting (β⁺) radionuclide (e.g. [⁶⁸Ga]Ga³⁺, [¹⁸F]AlF) for pre-therapy PET scans and patient selection, followed by high-energy particle-emitting radionuclides (e.g. [¹⁷⁷Lu]Lu³⁺) for peptide-receptor radionuclide-therapy (PRRT) [10-12]. The cancer-targeting peptide provides site-specific delivery and accumulation of the peptide and the attached radioactive “payload” to a molecular target such as a receptor, which is over-expressed on tumour cells (FIG. 1A) [13]. An exemplary application of this is the somatostatin (SSTR) receptor-targeting peptide—Tyr³-octreotate “TATE”—which is employed in the recently FDA-approved PRRT agent [¹⁷⁷Lu]Lu-DOTA-TATE. This PRRT agent only shows therapeutic efficacy if the patients cancer (e.g. neuroendocrine tumours) expresses high levels of the SSTR receptor.

PET is a technique able to visualize the tumour-receptor heterogeneity of all lesions simultaneously, unlike a biopsy which only takes a small sample of a single (and often heterogeneous) tumour. PET enables the clinical identification and stratification of patients based on the status of many therapeutically relevant receptors, depending on the cancer-targeting peptide used [14]. This method can identify tumours which lack sufficient expression of the target receptor and accurately predict response to follow-up PRRT [14]. PET is used to confirm receptor target accessibility in the personalized medicine development [15, 16].

A successful example of a peptide-agent is the PET radiopharmaceutical [⁶⁸Ga]Ga-DOTA-TATE (DOTA-Tyr³-octreotate, DOTA=radiometal chelator). It has been used for many years in Europe as an accurate method for detecting and staging neuroendocrine tumours/cancer, and this agent was FDA approved in 2016 in the USA under the name NetSpot® [17]. DOTA-TATE “gallium PET scans” replaced OctreoScan SPECT imaging as the standard of care due to its superior ability to detect cancerous lesions [18]. Therapeutic analogues of DOTA-TATE using the therapeutic radiometals lutetium-177 and yttrium-90 (high energy electron, β⁻ emitters) have been used in Germany in thousands of patients successfully [10, 19]. The PRRT agent [¹⁷⁷Lu]Lu-DOTA-TATE was FDA approved as “Lutathera” in January 2018. There is a knowledge-gap in the understanding of how changes in the chemical structure of peptide-based radiopharmaceuticals affect their biological behavior. These effects include unsuitable pharmacokinetics, poor tumour retention, and high retention in and subsequent irradiation of healthy tissues.

### Peptide-Linkers and Kidney Uptake

The effect that the chemical properties of “linkers” have on the overall behavior of radiopharmaceuticals is poorly understood, but it is generally known that: 1) increasing the space between radiometal chelator and peptide-agent with a linker can restore binding affinity in cases where it is lost due to steric interference, and 2) modulating polarity by adding in varying numbers of charged amino acids (e.g. aspartic acid) or short and polar polymers (e.g. polyethylene glycol (PEG)) can impart drastic changes on pharmacokinetics and tissue clearance [20-25].

As to why radiolabeled peptides “stick” in the kidneys, it has been suggested that after glomerular filtration in the kidneys, low molecular weight proteins (e.g. radioactive peptides) that remain in the ultrafiltrate, bind to endocytic receptors at the luminal surface of proximal tubular cells in the renal cortex (not the glomeruli or distal tubules) [26, 27]. This process is followed by internalization, transfer to lysosomes, proteolytic degradation, and finally retention of radiometal [26]. Normally, the broken-down peptide is transported back into the blood stream as amino acids, but radiometals are residualizing and remain trapped in the tubular cell lysosomes, causing them to become “stuck” where they deliver high radiation dose to the kidney tubules and glomeruli. Although the exact receptors and mechanisms responsible for tubular reabsorption of peptides are not fully understood, the receptor megalin (and associated receptor cubilin) has been identified as a scavenger receptor with the function of non-specific uptake of charged proteins and has high expression in the proximal tubules of the kidneys [28, 29]. This suggests that megalin-cubilin could be one of the causes of radiopeptide retention in the kidneys. It has been experimentally shown that comparing radiolabeled peptides (including several derivatives of octreotide and octreotate) in both healthy and megalin-deficient mice demonstrated a dramatic decrease in kidney uptake in megalin-deficient mice (˜2-3 fold) [26]. Peptide derivatives of [¹¹¹In]In-DTPA-octreotide with positive, neutral, and negative net charges have also confirmed high kidney retention, with positive charges showing more severe retention [30].

A method used clinically to reduce kidney uptake of radiolabeled peptides has been the co-infusion of amino acids such as lysine and glutamic acid [31, 32]. Specifically, lysine has been found to primarily block uptake of positively charged peptides [33], and glutamic acid to reduce the uptake of negatively charged peptides [34]. It has been demonstrated that kidney uptake was correlated with the number of charged amino acids, with positively charged amino acids appearing to play a role due to the negative surface charge of proximal tubular cells [34]. These co-infusions reduced kidney uptake of radiolabeled peptides by ˜10-50%, but also resulted in severe nausea in ˜50% of patients [33]. The ability to reduce kidney uptake of the radioactive peptides, rather than using a nausea-inducing cocktail of amino acids, would improve patient comfort. Amino acid charge interactions are clearly involved in kidney uptake and retention, and it appears that the charge-distribution over the surface of proteins also has an effect on uptake [34-36].

Although not for the purpose of blocking kidney uptake, permanent zwitterions (ZW) have been shown to minimize non-specific “sticking” of near-infrared dyes in vivo [1, 2]. Reduction of non-specific uptake and retention of radiolabeled peptides in excretory organs (e.g. kidneys) and other healthy tissues would reduce radioactive background, therefore improving PET imaging contrast and therapeutic index of radionuclide therapy agents. Reduced uptake and retention in healthy tissues would also reduce radiation toxicity and would be very desirable. Research and clinical translation of peptide-based radiopharmaceuticals has been rapidly expanding for both cancer and infectious disease, and a modular approach to reducing uptake and retention in kidney, spleen, and other healthy tissues would be of great utility [37, 38].

A zwitterion is a molecule possessing both a positive and a negative charge simultaneously at a certain pH. Most amino acid molecules form zwitterions at physiological pH, but not when incorporated into peptides and proteins. In recent years, a number of peer-reviewed publications have featured the concept of incorporating synthetic permanent zwitterions to increase polarity and reduce sticking to solid surfaces. These typically utilize quaternary amines for permanent positive charge, and often use sulfonate (R—SO3⁻) groups for negative charge at most physiologically relevant pH values. The conjugate acid of an alkyl sulfonate is the alkyl sulfonic acid, which have pKₐ values between ˜7 to 0, meaning they are deprotonated and negatively charged above pH 0 (physiological pH ˜5.5-7.5). Fluorescent dyes from Frangioni et al incorporate permanent zwitterions into their structure and promote improved pharmacokinetics and increased polarity [1-4]. Some nanoparticles have utilized permanent zwitterions on their surfaces to improve water solubility, reduce protein corona formation, and to reduce sticking to solid surfaces [5-8]. In 2020, a patent (U.S. Pat. No. 10,710,962, Zwitterionic Reagents) was issued to Siemens Healthcare Diagnostics for a range of permanent zwitterion structures that could be used to coat or incorporate into solid surfaces (e.g. test tubes, membranes, pipets tips) to reduce adherence and sticking of things such as proteins and other molecules.

### SUMMARY

The present application discloses zwitterionic (ZW) compounds, which can be used as modular linkers to form radiopharmaceuticals, for example, for diagnostics and/or therapy. The modularity of linkers enables fast incorporation into radiopharmaceuticals and improve pharmacokinetics and tissue clearance of the radiopharmaceuticals.

Accordingly the present application includes a zwitterion compound of Formula I, or a salt and/or solvate thereof:

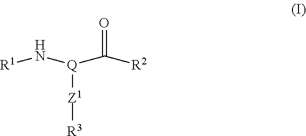

wherein
R¹ is H, OH or a protecting group;
R² is OH or a protecting group;
Q is selected from Q1, Q2, Q3, Q4 and Q5:

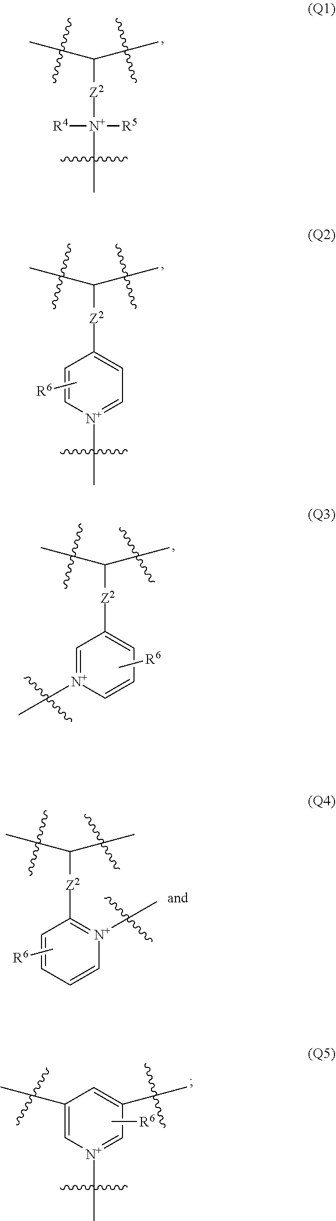

Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R⁴ and R⁵ are independently selected from C₁₋₁₀alkyl, C₂₋₁₀alkenyl and C₂₋₁₀alkynyl;
R⁶ is selected from one or more of H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R³ is an anion containing group selected from sulfonate, carboxylate, sulfate and phosphate.

The present application also includes a conjugate of Formula II, or a salt and/or solvate thereof:

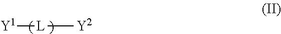

wherein
Y¹ is a complex comprising a chelating agent and one or more radionuclides;
Y² is a targeting ligand;
L is a zwitterionic (ZW) linker of the formula

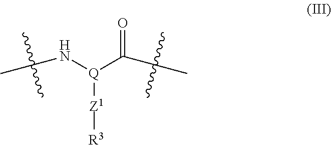

wherein
Q is selected from Q1, Q2, Q3, Q4 and Q5:

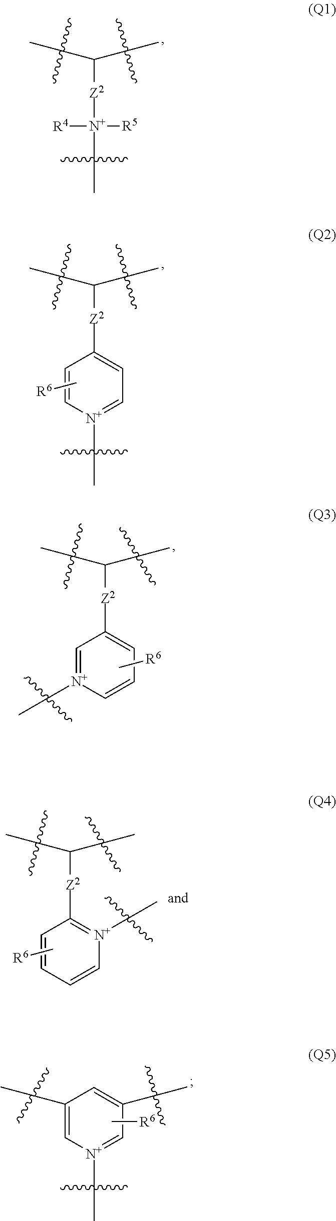

Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R³ is an anion containing group selected from sulfonate, carboxylate, sulfate and phosphate; and
n is an integer 0 to 5.

The present application also includes a conjugate of Formula IV, or a salt and/or solvate thereof:

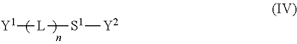

wherein
Y¹ is a complex comprising a chelating agent and one or more radionuclides, wherein the chelating agent is desferrioxamine (DFO) or a compound of the following formula;

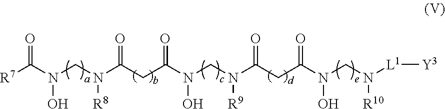

wherein
R⁷, R⁸, R⁹ and R¹⁰ are independently selected from H and C₁₋₄alkyl;
each a, c and e is an integer independently selected from 3, 4, 5, 6 and 7;
each b and d is an integer independently selected from 1, 2, 3 and 4;
L¹ is a linker group

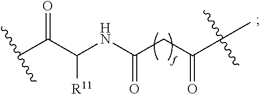

Y³ is

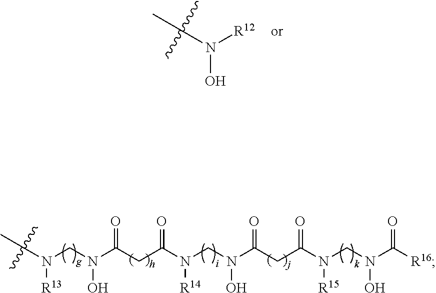

R¹¹ is selected from C₁₋₁₀alkyl and C₁₋₆alkylenephenyl, optionally substituted by one of NH₂, OH, SH, N₃, CO₂H and

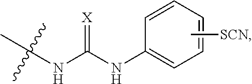

wherein X is O, S or NH;
R¹², R¹³, R¹⁴, R¹⁵ and R¹⁶ are independently selected from H and C₁₋₄alkyl;
f is an integer selected from 1, 2, 3 and 4;
each g, l and k is an integer independently selected from 3, 4, 5, 6 and 7;
each h and j is an integer independently selected from 1, 2, 3 and 4;
Y² is a targeting ligand;
S¹ is a biomolecule conjugating group;
L is a zwitterionic (ZW) linker of the formula

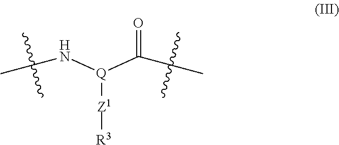

wherein
Q is selected from Q1, Q2, Q3, Q4 and Q5:

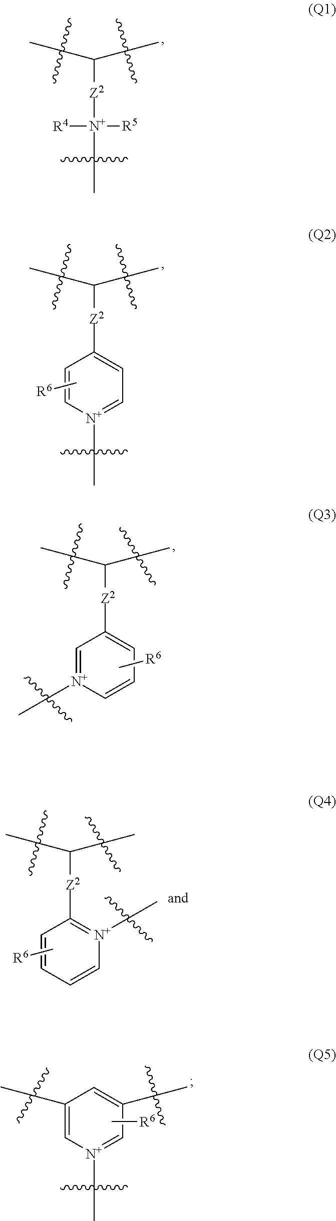

Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R³ is an anion containing group selected from sulfonate, carboxylate, sulfate and phosphate;
n is an integer 1 to 5;
wherein the chelating agent is a compound of Formula V, Y¹ is attached to L at R¹¹ of L¹ group

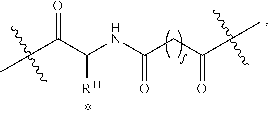

and the L is further optionally attached to one to three additional L groups, or the L is attached between the Y³

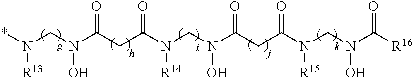

and L¹

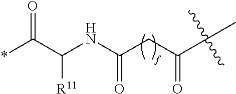

groups of Y¹, or one L is attached at R¹¹ of L¹ group and another L is attached between the Y³ and L¹ groups of Y¹, and each one of the L is further optionally attached to one to three additional L groups; and
wherein the chelating agent is DFO, the point of attachment of L is at the amine

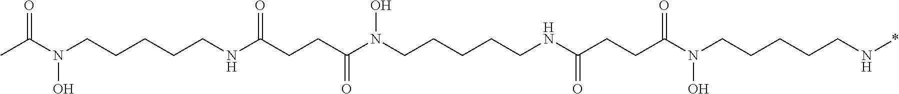 [SMILES: *NCCCCCN(O)C(=O)CCC(=O)NCCCCCN(O)C(=O)CCC(=O)NCCCCCN(O)C(C)=O]

and
wherein the * indicates the point of attachment.

The present application also includes a composition comprising a conjugate of the application and a carrier. The present application also includes a pharmaceutical composition comprising a conjugate of the application and a pharmaceutically acceptable carrier. In an embodiment, the composition of the application is a diagnostic or a theranostic composition.

The conjugates of the application are useful in medical diagnosis, diagnosing, treatment of one or more diseases, disorders or conditions, radionuclide therapy and/or theranostic treatments. Accordingly, the present application includes a method of diagnosing, treatment of one or more diseases, disorders or conditions, radionuclide treatment and/or theranostic treatments, comprising administering an effective amount of a conjugate of the application to a subject in need thereof.

The present application also includes a kit comprising a compound, a conjugate or a composition of the application.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DRAWINGS

The embodiments of the application will now be described in greater detail with reference to the attached drawings in which:

FIG. 1 shows the structural and conceptual introduction to A) peptide-based molecular imaging agents, peptide-receptor radionuclide-therapy (PRRT) agents, “linkers”, and B) the chemical structures of common “linkers”.

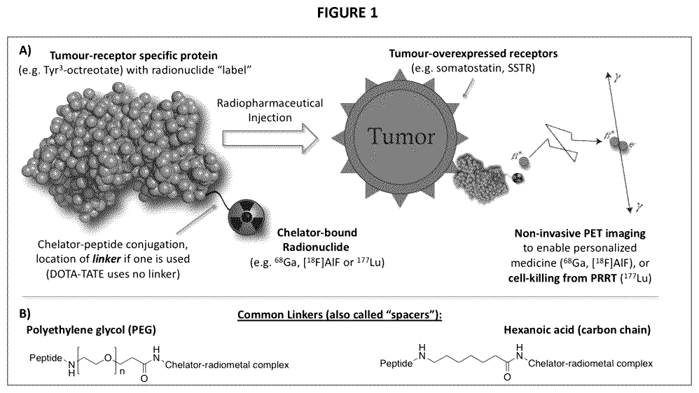

FIG. 2 shows the structure of FDA approved NetSpot radiotracer, unlabelled DOTA-Tyr³-octreotate (TATE) (Top), and labelled [⁶⁸Ga]Ga-DOTA-TATE (Bottom).

FIG. 3 shows the structure of unlabeled exemplary DOTA-Zwitterion(ZW)-PEG₄-TATE (Top), and labelled exemplary [⁶⁸Ga]Ga-DOTA-ZW-PEG₄-TATE (Bottom) with improved tumor-tissue ratios and faster pharmacokinetic of healthy-tissue clearance.

FIG. 4 shows the structure of two exemplary embedded ZW units in unlabelled DOTA-(ZW)₂-PEG₄-TATE (Top), and exemplary labelled [⁶⁸Ga]Ga-DOTA-(ZW)₂-PEG₄-TATE (Bottom) with improved tumor-tissue ratios and faster pharmacokinetic of healthy-tissue clearance.

FIG. 5 shows representative radio-HPLC chromatograms for human serum stability study of exemplary [⁶⁸Ga]Ga-DOTA-Lys(ZW)-PEG₄-TATE at gradient of MeCN:H₂O (both with 0.1% FA) with 0.5 mL/min flow rate.

FIG. 6 shows representative radio-HPLC chromatograms for mouse serum stability study of exemplary [⁶⁸Ga]Ga-DOTA-Lys(ZW)-PEG₄-TATE at gradient of MeCN:H₂O (both with 0.1% FA) with 0.65 mL/min flow rate.

FIG. 7 shows representative radio-HPLC chromatograms for human serum stability study of exemplary [⁶⁸Ga]Ga-DOTA-Lys(ZW)₂-PEG₄-TATE at gradient of MeCN:H₂O (both with 0.1% FA) with 0.5 mL/min flow rate.

FIG. 8 shows representative radio-HPLC chromatograms for mouse serum stability study of exemplary [⁶⁸Ga]Ga-DOTA-Lys(ZW)₂-PEG₄-TATE at gradient of MeCN:H₂O (both with 0.1% FA) with 0.65 mL/min flow rate.

FIG. 9 shows representative radio-HPLC chromatograms for human serum stability study of exemplary [⁶⁸Ga]Ga-DOTA-Pyr(ZW)-PEG₄-TATE at gradient of MeCN:H₂O (both with 0.1% FA) with 0.65 mL/min flow rate.

FIG. 10 shows representative radio-HPLC chromatograms for mouse serum stability study of exemplary [⁶⁸Ga]Ga-DOTA-Pyr(ZW)-PEG₄-TATE at gradient of MeCN:H₂O (both with 0.1% FA) with 0.65 mL/min flow rate.

FIG. 11 shows 90 minutes dynamic PET-CT imaging of 5-6 MBq of comparative [⁶⁸Ga]Ga-DOTA-TATE (Top), exemplary [⁶⁸Ga]Ga-DOTA-Lys(ZW)-PEG₄-TATE (Middle) and exemplary [⁶⁸Ga]Ga-DOTA-Lys(ZW)₂-PEG₄-TATE (Bottom) in healthy female CD1 mice (n=4) in 200 μL of sterile saline with less than 10% ethanol content injected intravenously via tail vein catheter, under isoflurane anesthesia (2.5%, 2-2.5 L/min).

FIG. 12 shows dynamic PET/CT imaging (0-90 minutes) of 5-6 MBq of exemplary [⁶⁸Ga]Ga-DOTA-Pyr(ZW)-PEG₄-TATE in healthy female (top) and male (bottom) CD1 mice (n=4) injected intravenously via tail vein catheter in 200 μL of sterile saline with less than 10% ethanol content, under isoflurane anesthesia (2.5%, 2-2.5 L/min).

FIG. 13 shows the time-activity curve (TAC) from 5-6 MBq of [⁶⁸Ga]Ga-comparative DOTA-TATE and exemplary ZW-bearing derivatives in healthy CD1 mice with the quantity of radioactivity in the kidneys displayed over time, with data extracted from dynamic PET/CT imaging (0-90 minutes) by contouring the organ of interest (OOI).

FIG. 14 shows the results of the ex vivo biodistribution study from the injection of 5-6 MBq (˜200 μL, sterile saline, <10% ethanol) of comparative [⁶⁸Ga]Ga-DOTA-TATE and exemplary ZW-bearing derivatives in healthy CD1 mice two hours post injection (top) and the same data displayed with expanded Y-axis revealing the uptake differences in other healthy organs (bottom). Significance was measured using Students one-tail and two-tail t-tests, *p<0.05, **p<0.01, ***p<0.001, ns (not significant).

FIG. 15 shows dynamic PET/CT imaging (0-90 minutes) following injection of 5-6 MBq of comparative [⁶⁸Ga]Ga-DOTA-TATE (top) and exemplary [⁶⁸Ga]Ga-DOTA-Lys(ZW)-PEG₄-TATE (bottom) in male NSG mice (n=3) in 200 μL of sterile saline with less than 10% ethanol content injected intravenously via tail vein catheter, under isoflurane anesthesia (2%, 2-2.5 L/min).

FIG. 16 shows time-activity curve (TAC) from 5-6 MBq of comparative [⁶⁸Ga]Ga-DOTA-TATE and exemplary [⁶⁸Ga]Ga-DOTA-Lys(ZW)-PEG₄-TATE, and [⁶⁸Ga]Ga-DOTA-Glu-PEG₄-TATE in NOD/SCID mice bearing subcutaneous AR42J xenografts with the quantity of radioactivity in the kidneys and tumors displayed overtime, with data extracted from dynamic PET/CT imaging (0-90 minutes) by contouring the organ of interest (OOI).

FIG. 17 shows the results of the biodistribution study from injection of 5-6 MBq of comparative [⁶⁸Ga]Ga-DOTA-TATE and exemplary [⁶⁸Ga]Ga-DOTA-Lys(ZW)-PEG₄-TATE, and [⁶⁸Ga]Ga-DOTA-Lys(ZW)₂-PEG₄-TATE in male NSG mice bearing subcutaneous xenografts (AR42J) two hours post injection (left) and the same data displayed with an expanded Y-axis revealing the uptake in other healthy organs (right). Significance was measured using Students t-tests with one tail and two tail, *P<0.05, **P<0.01, ***P<0.001, ns (not significant).

FIG. 18 shows structure of one exemplary embedded Pyr(ZW)-PZW units in unlabelled DOTA-PZW-PEG₄-TATE (Top), and exemplary labelled [⁶⁸Ga]Ga-DOTA-PZW-PEG₄-TATE (Bottom). PZW=Pyr(ZW).

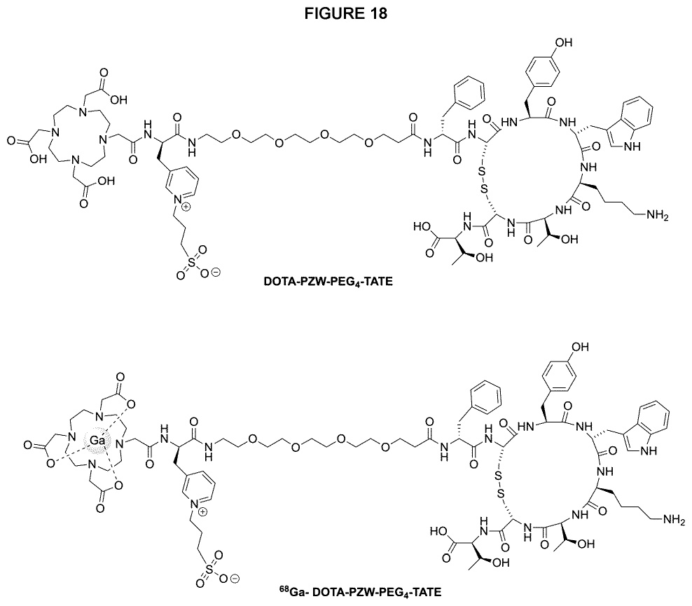

FIG. 19 shows representative radio-HPLC chromatograms for mouse serum stability study of exemplary [⁶⁸Ga]Ga-DOTA-Pyr(ZW)-PEG₄-TATE at gradient of MeCN:H₂O (both with 0.1% FA) with 0.65 mL/min flow rate

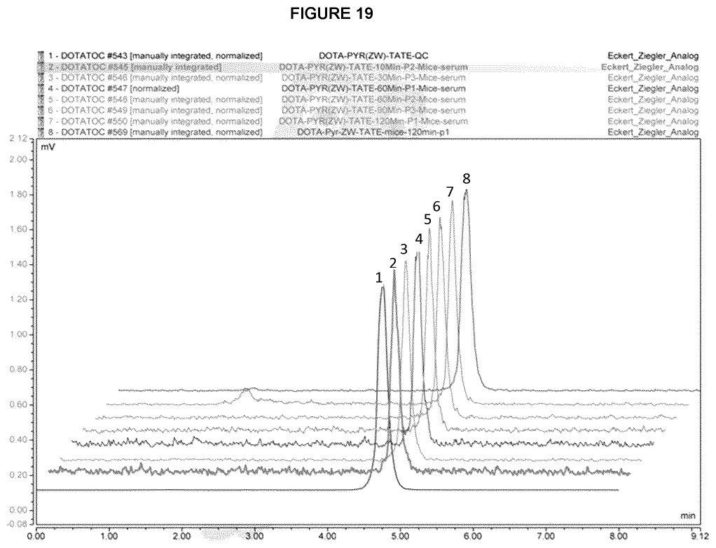

FIG. 20 shows representative radio-HPLC chromatograms for human serum stability study of exemplary [⁶⁸Ga]Ga-DOTA-Pyr(ZW)-PEG₄-TATE at gradient of MeCN:H₂O (both with 0.1% FA) with 0.65 mL/min flow rate.

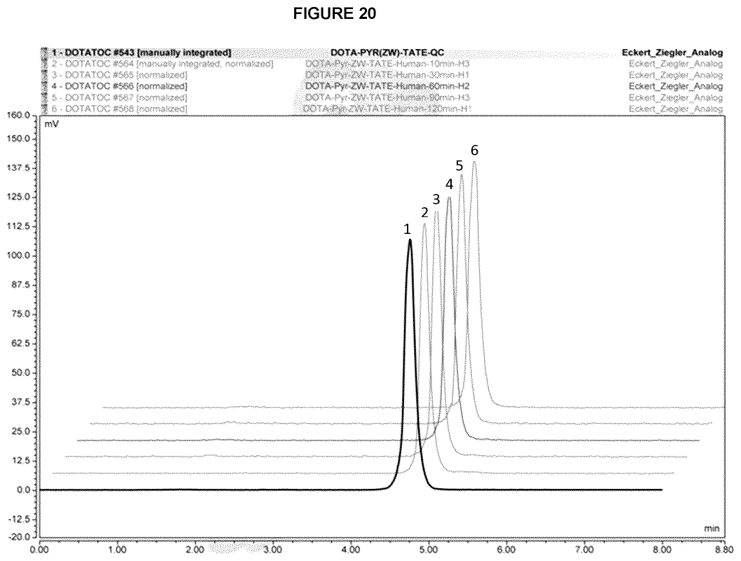

### DESCRIPTION OF VARIOUS EMBODIMENTS

### Definitions

Unless otherwise indicated, the definitions and embodiments described in this and other sections are intended to be applicable to all embodiments and aspects of the present application herein described for which they are suitable as would be understood by a person skilled in the art.

The term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this application, the singular forms “a”, “an” and “the” include plural references unless the content dearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The present application refers to a number of chemical terms and abbreviations used by those skilled in the art. Nevertheless, definitions of selected terms are provided for clarity and consistency.

The products of the processes of the application may be isolated according to known methods, for example, the compounds may be isolated by evaporation of the solvent, by filtration, centrifugation, chromatography or other suitable method.

One skilled in the art will recognize that where a reaction step of the present application is carried out in a variety of solvents or solvent systems, said reaction step may also be carried out in a mixture of the suitable solvents or solvent systems.

The term “chelating agent” as used herein includes molecules that form stable complexes with traceable metal atoms under physiological conditions such that the metal remains bound in vivo. For diagnostic imaging purposes, “chelating agent” or “bifunctional chelating agent” is a compound which has a reactive functional group to facilitate conjugation with a targeting ligand such as a peptide or antibody, and chelating moieties for labeling by a radionuclide and, on binding to a radionuclide metal, forms a complex that is stable under physiological conditions.

The term “targeting ligand” as used herein is a molecule or part of a molecule that binds with specificity to another molecule. The targeting ligand can be a tissue specific and/or organ and/or a receptor specific moiety. Thus, the targeting ligand can bind or attach to one or more tissues, organs and/or receptors. The targeting ligand can be any such molecule known to those of ordinary skill in the art. The binding may be by any mechanism of binding known to those of ordinary skill in the art.

The term “compound(s) of the application” or “compound(s) of the present application”, and the like, as used herein refers to a compound of Formula I or a salt and/or solvate thereof.

The term “conjugate(s) of the application” or “conjugate(s) of the present application”, and the like, as used herein refers to a chelator-linker-targeting peptide conjugate compound of Formula II or a salt and/or solvate thereof.

The term “composition(s) of the application” or “composition(s) of the present application, and the like, as used herein refers to a composition comprising one or more conjugates of the application and a carrier.

The term “alkyl” as used herein, whether it is used alone or as part of another group, means straight or branched chain, saturated alkyl groups. The number of carbon atoms that are possible in the referenced alkyl group are indicated by the prefix “Cn1-n2”. For example, the term C₁₋₁₀alkyl means an alkyl group having 1, 2, 3, 4, 5, 6, 7, 8, 9 or 10 carbon atoms. All alkyl groups are optionally fluoro-substituted unless otherwise indicated.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond. All alkenyl groups are optionally fluoro-substituted unless otherwise indicated.

The term “alkynyl” as used herein, whether it is used alone or as part of another group, means straight or branched chain, unsaturated alkynyl groups containing at least one triple bond. The number of carbon atoms that are possible in the referenced alkyl group are indicated by the prefix “Cₙ₁₋ₙ₂”. For example, the term C₂₋₆alkynyl means an alkynyl group having 2, 3, 4, 5 or 6 carbon atoms.

The term “alkylene”, whether it is used alone or as part of another group, means straight or branched chain, saturated alkylene group, that is, a saturated carbon chain that contains substituents on two of its ends. The number of carbon atoms that are possible in the referenced alkylene group are indicated by the prefix “Cn1-n2”. For example, the term C₁₋₁₀alkylene means an alkylene group having 1, 2, 3, 4, 5, 6, 7, 8, 9 or 10 carbon atoms. All alkyl groups are optionally fluorosubstituted unless otherwise indicated.

The term “alkenylene” as used herein, whether it is used alone or as part of another group, means straight or branched chain, unsaturated alkenylene groups containing at least one double bond and substituents on two of its ends. The number of carbon atoms that are possible in the referenced alkenylene group are indicated by the prefix “Cₙ₁₋ₙ₂”. For example, the term C₂₋₆alkenylene means an alkenylene group having 2, 3, 4, 5 or 6 carbon atoms and at least one double bond. All alkenylene groups are optionally fluoro-substituted unless otherwise indicated.

The term “alkynylene” as used herein, whether it is used alone or as part of another group, means straight or branched chain, unsaturated alkynylene groups containing at least one triple bond and substituents on two of its ends. The number of carbon atoms that are possible in the referenced alkynylene group are indicated by the prefix “Cₙ₁₋ₙ₂”. For example, the term C₂₋₆alkynylene means an alkynylene group having 2, 3, 4, 5 or 6 carbon atoms.

The term “halogen” (or “halo”) whether it is used alone or as part of another group, refers to a halogen atom and includes fluoro, chloro, bromo and iodo.

The term “heteromoiety” refers to heteroatoms and includes O, S and NH.

The term “substituted” refers to the addition of a substituent group to a parent compound.

The term “interrupted” refers to the insertion of a moiety between two carbon atoms. Non-limiting examples of such atoms and moieties include —O—, —S—, —N(H)—.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “anion containing group” as used herein refers to a group with negatively charged ion.

The term “zwitterion” or “zwitterionic” refers to a molecule possessing both a positive and negative charge simultaneously at a certain pH, and isolation. The total net charge of the chemical compound is zero (electrically neutral). A zwitterionic moiety which in itself may possess a net charge of zero may be incorporated into a larger molecule such as a chelator-linker-peptide conjugate which as a whole may possess a net charge other than zero.

The symbol “ ” when drawn perpendicularly across a bond indicates a point of attachment of the group.

The term “subject” as used herein includes all members of the animal kingdom, while members of the animal kingdom include mammals, and suitably refer to humans. Thus, the methods and uses of the present application are applicable to human therapy and veterinary therapy.

The term “cell” as used herein refers to a single cell or a plurality of cells and includes a cell either in a cell culture or in a subject.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations.

The term “theranostic application” as used herein means using one radioactive drug to diagnose and treat a condition of choice, typically with the radioactive drug remaining identical while the radionuclide being changed (e.g. gallium-68 for PET imaging, lutetium-177 for therapy).

The term “therapeutic treatment” as used herein can include a treatment administered to a subject in need of imaging. The subject can be in need of imaging to aid in diagnosis; to locate a position for a therapeutic intervention; to assess the functioning of a body part; and/or to assess the presence or absence of a condition. The effectiveness of a therapeutic imaging treatment can be confirmed based on the capture of an image sufficient for its intended purpose.

The term “administered” as used herein means administration of a therapeutically effective amount of a compound, or one or more compounds, or a composition of the application to a cell or a subject.

The term “radionuclide” as used herein means an atom with an unstable nucleus, which undergoes radioactive decay, resulting in the emission of gamma ray(s) or subatomic particles such as positrons, alpha or beta particles, or Auger electrons. These emissions constitute ionizing radiation. Radionuclides occur naturally, or can be produced artificially.

The term “spacer” as used herein means an entity which increases the space between the chelating agent complex and the targeting ligand. The spacer can restore binding affinity in cases where it is lost due to steric interference.

The term “biomolecule conjugating group” as used herein means a functional group that reacts with a functional group in a targeting ligand to form a covalent bond.

The term “Positron Emission Tomography (PET)” as used herein means a functional imaging technique applied in nuclear medicine, whereby a three-dimensional image (e.g. of functional processes) in the body is produced. The system detects pairs of gamma rays emitted indirectly by a positron-emitting radionuclide, which is introduced into the body in form of a pharmaceutical compound.

The term Single-Photon Emission Computed Tomography (SPECT)” as used herein means a three-dimensional diagnostic imaging technique using gamma rays emitted by radioisotopes. In contrast with PET, the tracer used in SPECT emits gamma radiation that is measured directly, whereas a PET tracer emits positrons that annihilate with near-by electrons, which are a few millimeters away, causing two gamma photons to be emitted in opposite directions.

The term “DMF” as used herein means dimethylformamide.

The term “Fmoc” as used herein means fluorenylmethoxycarbonyl protecting group.

The term “DCM” as used herein means dichloromethane.

The term “TIPS” as used herein means triisopropylsilane.

The term “TFA” as used herein means trifluoroacetic acid.

### Compounds and Compositions of the Application

Therefore, the present application includes a zwitterion compound of Formula I, or a salt and/or solvate thereof:

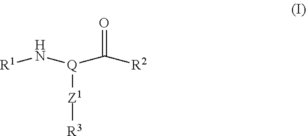

wherein
R¹ is H, OH or a protecting group;
R² is OH or a protecting group;
Q is selected from Q1, Q2, Q3, Q4 and Q5:

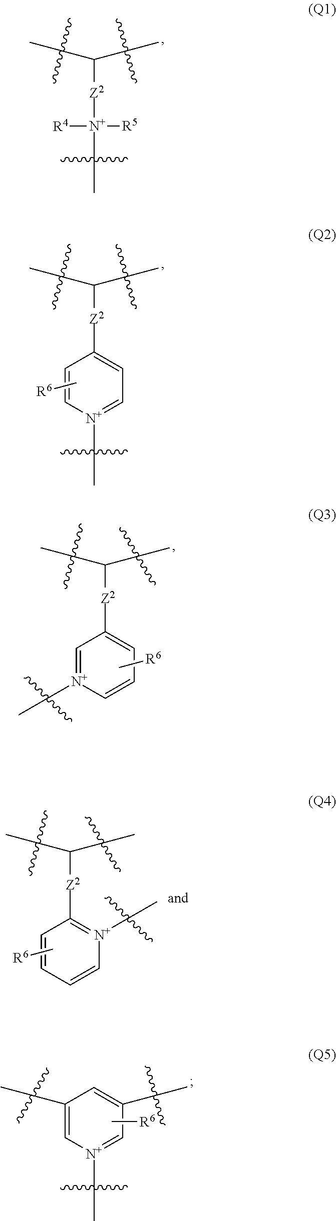

Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, N and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, N and S;
R³ is an anion containing group selected from sulfonate, carboxylate, sulfate and phosphate.

In some embodiments, R¹ is H or the protecting group.

In some embodiments, R² is OH or the protecting group.

Protecting group used in the present application can be any protecting group known in the art. In some embodiments, the protecting group is selected from 9-fluorenenylmethoxycarbonyl (Fmoc), tert-butyloxycarbonyl (Boc), tert-butyl (^{t}Bu), trityl (Trt), 2,4-dimethoxybenzyl (Dmb), 9-fluorenylmethyl (Fm) and benzyl (Bn). In some embodiments, the protecting group is selected from Fmoc and Boc.

In some embodiments, R¹ is the protecting group. In some embodiments, R¹ is Fmoc.

In some embodiments, R² is OH.

In some embodiments, R¹ is Fmoc, R² is OH and the compound of Formula I has the following structure:

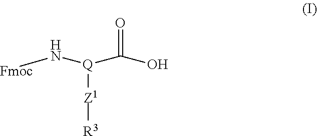

or a salt and/or solvate thereof,
wherein Q, Z¹ and R³ are as defined for Formula I.

In some embodiments, Q is Q1,

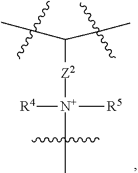

and the compound of Formula I has the following structure:

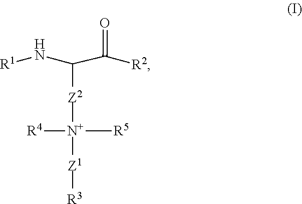

or a salt and/or solvate thereof,
wherein R¹, R², R³, R⁴, R⁵, Z¹ and Z² are as defined for Formula I.

In some embodiments, Q is Q2,

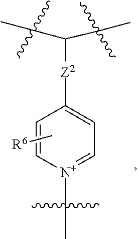

and the compound of Formula I has the following structure:

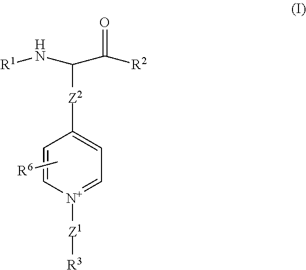

or a salt and/or solvate thereof,
wherein R¹, R², R³, R⁶, Z¹ and Z² are as defined for Formula I.

In some embodiments, Q is Q3,

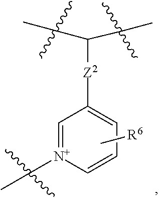

and the compound of Formula I has the following structure:

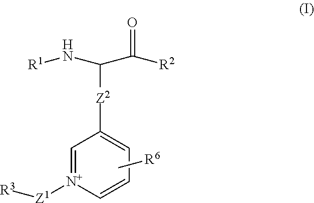

or a salt and/or solvate thereof,
wherein R¹, R², R³, R⁶, Z¹ and Z² are as defined for Formula I.

In some embodiments, Q is Q4,

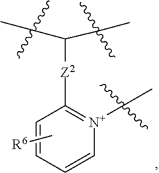

and the compound of Formula I has the following structure:

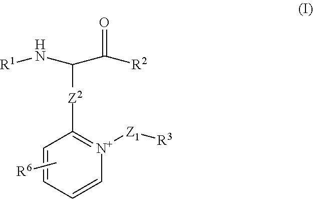

or a salt and/or solvate thereof,
wherein R¹, R², R³, R⁶, Z¹ and Z² are as defined for Formula I.

In some embodiments, Q is Q5,

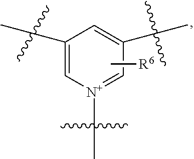

and the compound of Formula I has the following structure:

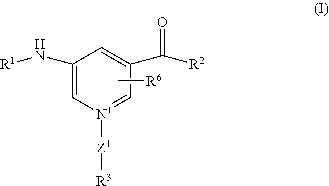

or a salt and/or solvate thereof,
wherein R¹, R², R³, R⁶ and Z¹ are as defined for Formula I.

In some embodiments, when Q is

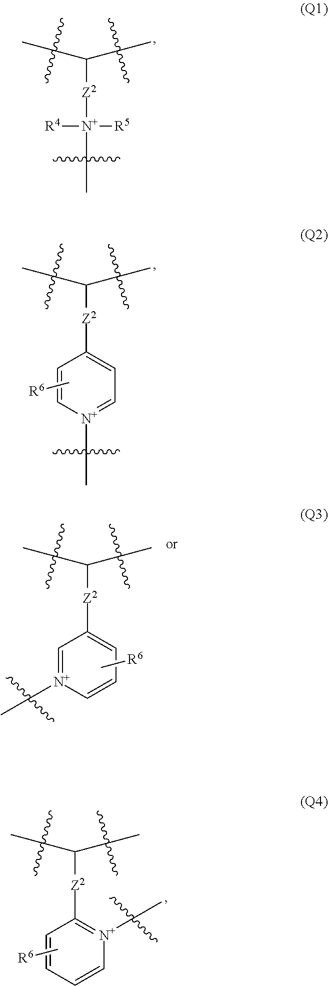

the stereochemistry at the carbon to which Z² is bonded is either R or S. Therefore, in some embodiments, Q1 is

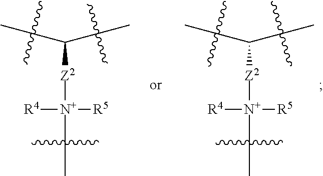

### Q2 is

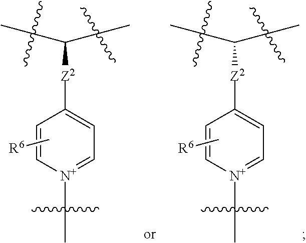

### Q3 is

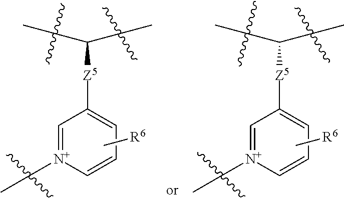

### and Q4 is

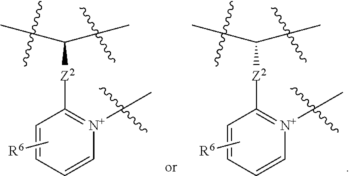

In some embodiments, the stereochemistry at the carbon to which Z² is attached is R. In some embodiments, the stereochemistry at the carbon to which Z² is attached is S.

In some embodiments, R⁴ and R⁵ are independently selected from C₁₋₆alkyl, C₂₋₆alkenyl and C₂₋₆alkynyl. In some embodiments, R⁴ and R⁵ are independently selected from C₁₋₄alkyl, C₂₋₄alkenyl and C₂₋₄alkynyl. In some embodiments, R⁴ and R⁵ are independently selected from CH₂CH₂CH₃, C(CH₃)₃, CH(CH₃)CH₂CH₃ and CH₂CH(CH₃)₂, CH(CH₃)₂, CH₂CH₃ and CH₃. In some embodiments, R⁴ and R⁵ are independently selected from CH₂CH₂CH₃. CH(CH₃)₂, CH₂CH₃ and CH₃. In some embodiments, at least one of R⁴ and R⁵ is CH₃. In some embodiments, each one of R⁴ and R⁵ is CH₃.

In some embodiments, R⁶ is H.

In some embodiments, Z² is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, N and S. In some embodiments, Z² is selected from C₁₋₆alkylene, C₂₋₆alkenylene and C₂₋₆alkynelene, unsubstituted or substituted with one or more of halo or OH and/or are optionally interrupted by one to three heteromoieties independently selected from O, N and S. In some embodiments, Z² is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, N and S. In some embodiments, Z² is C₁₋₄alkylene. In some embodiments, Z² is C₄alkylene. In some embodiments, Z² is C₁alkylene.

In some embodiments, Z¹ is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, N and S. In some embodiments, Z¹ is selected from C₁₋₆alkylene, C₂₋₆alkenylene and C₂₋₆alkynelene, unsubstituted or substituted with one or more of halo or OH and/or are optionally interrupted by one to three heteromoieties independently selected from O, N and S. In some embodiments, Z¹ is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, N and S. In some embodiments, Z¹ is C₃alkylene.

In some embodiments, R³ is sulfonate.

In some embodiments, the zwitterion compound of Formula I, or a salt and/or solvate thereof:

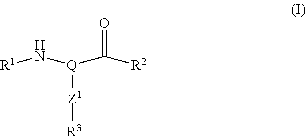

wherein
R¹ is a protecting group;
R² is OH;
Q is selected from Q1, Q2, Q3, Q4 and Q5:

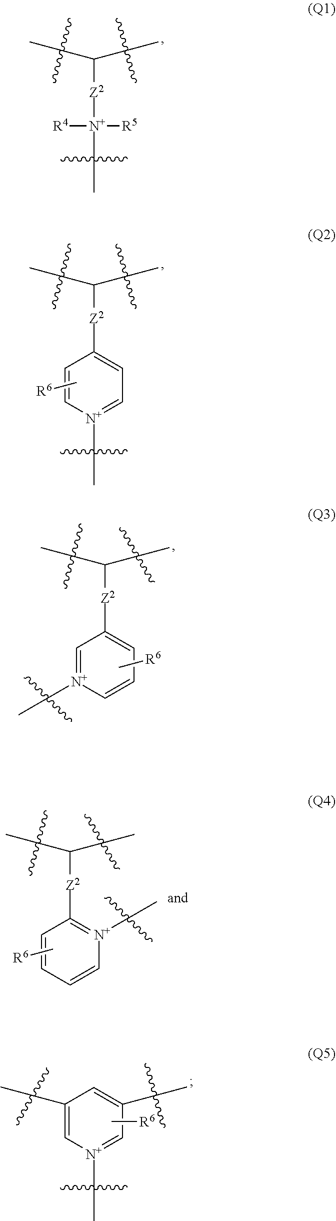

Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, N and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, N and S;
R³ is sulfonate;
provided that when Q is Q1, Q2, Q3 or Q4, then the compound of formula I is an (R)- or (S)-enantiomer of the carbon to which Z² is attached.

In some embodiments, the compound of Formula I is selected from

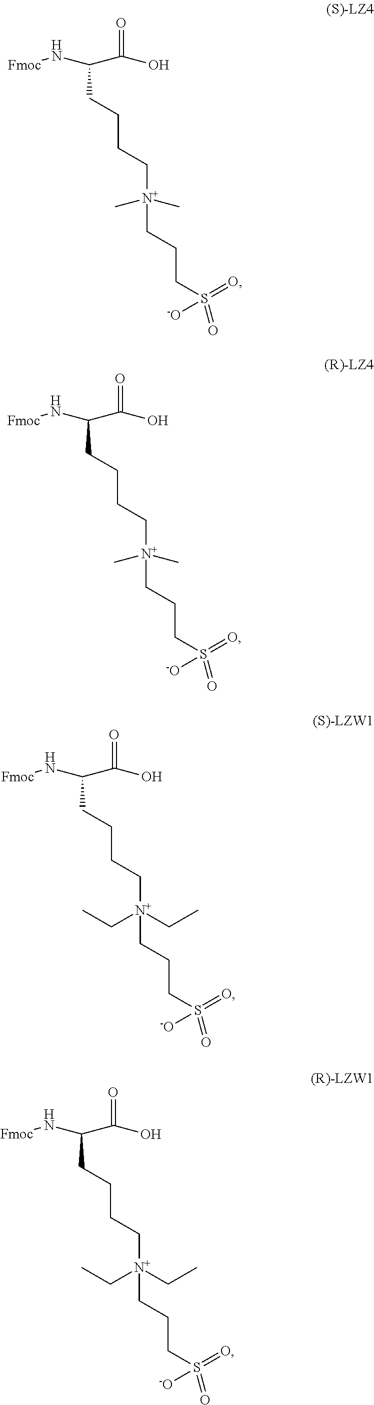

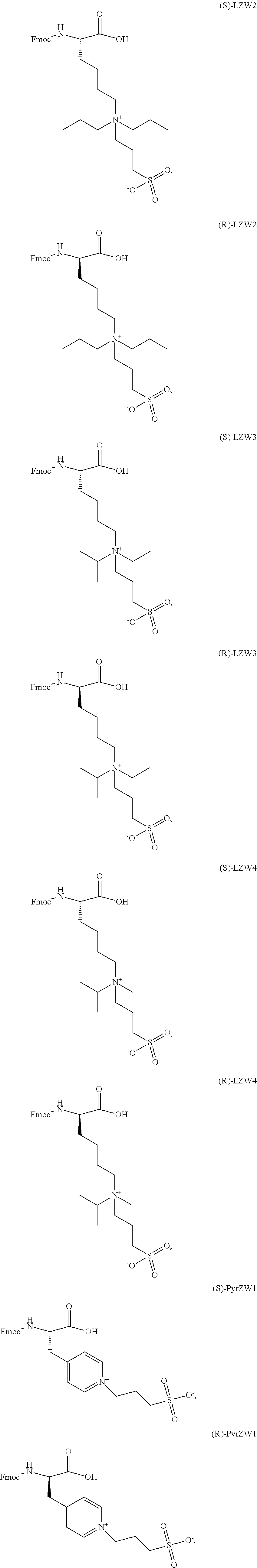

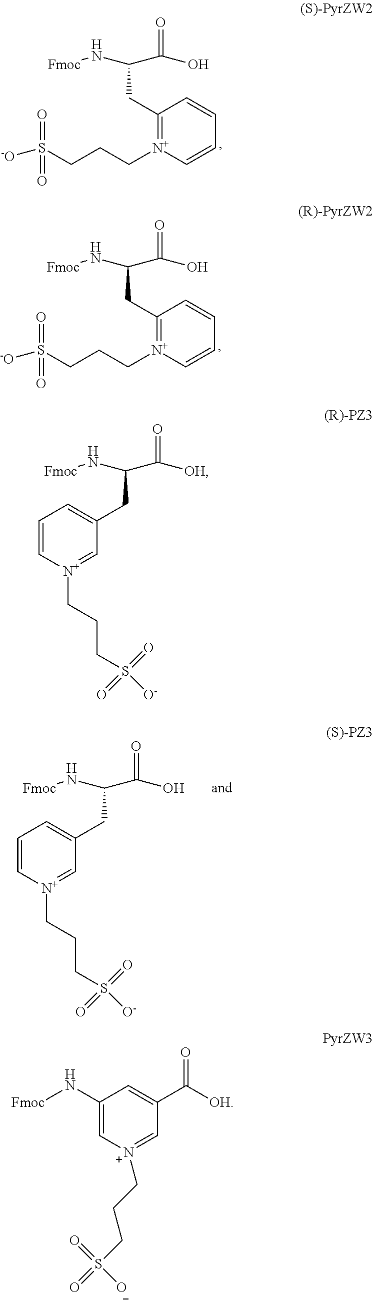

The present application further includes a conjugate of Formula II, or a salt and/or solvate thereof:

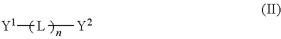

wherein
Y¹ is a complex comprising a chelating agent and one or more radionuclides;
Y² is a targeting ligand;
L is a zwitterion (ZW) linker of the formula

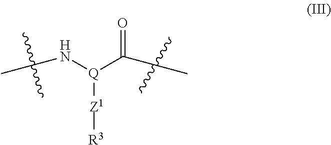

wherein
Q is selected from Q1, Q2, Q3, Q4 and Q5:

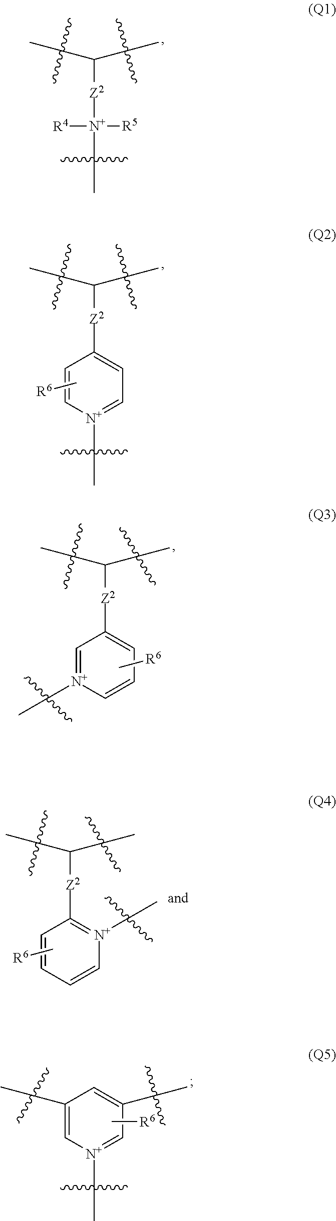

Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, N and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, N and S;
R³ is an anion containing group selected from sulfonate, carboxylate, sulfate and phosphate; and
n is an integer 0 to 5.

In some embodiments, Q is Q1,

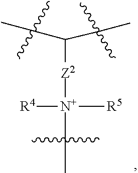

and the compound of Formula II has the following structure:

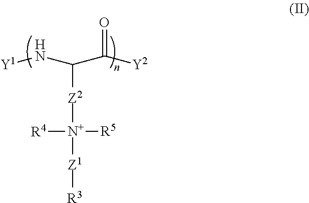

or a salt and/or solvate thereof,
wherein Y¹, Y², R³, R⁴, R⁵, Z¹, Z² and n are as defined for Formula II.

In some embodiments, Q is Q2,

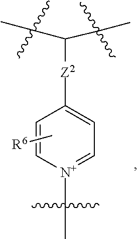

and the compound of Formula II has the following structure:

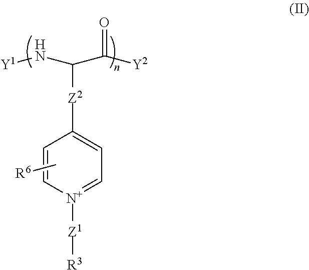

or a salt and/or solvate thereof,
wherein Y¹, Y², R³, R⁶, Z¹, Z² and n are as defined for Formula II.

In some embodiments, Q is Q3,

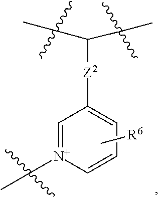

and the compound of Formula II has the following structure:

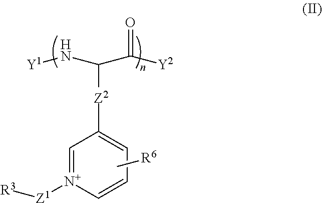

or a salt and/or solvate thereof,
wherein Y¹, Y², R³, R⁶, Z¹, Z² and n are as defined for Formula II.

In some embodiments, Q is Q4,

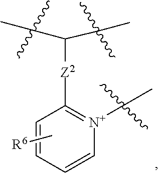

and the compound of Formula II has the following structure:

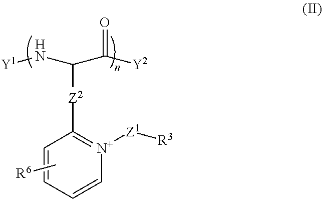

or a salt and/or solvate thereof,
wherein Y¹, Y², R³, R⁶, Z¹, Z² and n are as defined for Formula II.

In some embodiments, Q is Q5,

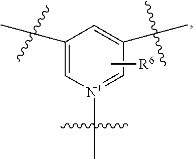

and the compound of Formula II has the following structure:

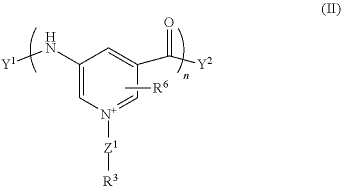

or a salt and/or solvate thereof,
wherein Y¹, Y², R³, R⁶, Z¹ and n are as defined for Formula II.

In some embodiments, when Q is

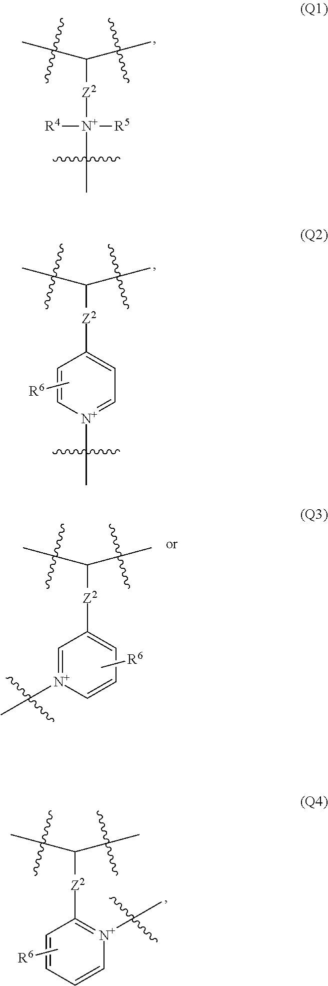

the stereochemistry at the carbon to which Z² is bonded is either R or S. In some embodiments, the stereochemistry at the carbon to which Z² is attached is R. In some embodiments, the stereochemistry at the carbon to which Z² is attached is S.

In some embodiments, R⁴ and R⁵ are independently selected from C₁₋₆alkyl, C₂₋₆alkenyl and C₂₋₆alkynyl. In some embodiments, R⁴ and R⁵ are independently selected from C₁₋₄alkyl, C₂₋₄alkenyl and C₂₋₄alkynyl. In some embodiments, R⁴ and R⁵ are independently selected from CH₂CH₂CH₃, C(CH₃)₃, CH(CH₃)CH₂CH₃ and CH₂CH(CH₃)₂, CH(CH₃)₂, CH₂CH₃ and CH₃. In some embodiments, R⁴ and R⁵ are independently selected from CH₂CH₂CH₃, CH(CH₃)₂, CH₂CH₃ and CH₃. In some embodiments, at least one of R⁴ and R⁵ is CH₃. In some embodiments, each one of R⁴ and R⁵ is CH₃.

In some embodiments, R⁶ is H.

In some embodiments, Z² is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, N and S. In some embodiments, Z² is selected from C₁₋₆alkylene, C₂₋₆alkenylene and C₂₋₆alkynelene, unsubstituted or substituted with one or more of halo or OH and/or are optionally interrupted by one to three heteromoieties independently selected from O, N and S. In some embodiments, Z² is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, N and S. In some embodiments, Z² is C₄alkylene. In some embodiments, Z² is C₁alkylene.

In some embodiments, Z¹ is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, N and S. In some embodiments, Z¹ is selected from C₁₋₆alkylene, C₂₋₆alkenylene and C₂₋₆alkynelene, unsubstituted or substituted with one or more of halo or OH and/or are optionally interrupted by one to three heteromoieties independently selected from O, N and S. In some embodiments, Z¹ is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, N and S. In some embodiments, Z¹ is C₃alkylene.

In some embodiments, R³ is sulfonate.

In some embodiments, the complex comprising a chelating agent and one or more radionuclides is a coordination complex, in which the one or more radionuclides are coordinatively bound to the chelating agent.

In some embodiments, the chelating agent is selected from DOTA, NOTA, DTPA, TETA, EDTA, NODAGA, NODASA, TRITA, CDTA, BAT, DFO and HYNIC. In some embodiments, the chelating agent is DOTA.

In some embodiments, the radionuclide is selected from a transition metal, rare-earth metal, lanthanide, actinide and metalloid.

Radionuclides that can be used in the present application include but are not limited to the following elements and their isotopes: ²²⁵Ac, ²²⁶Ac, ²²⁷Ac, ²²⁸Ac, ¹⁰⁵Ag, ^{106m}Ag, ^{110m}Ag, ¹¹¹Ag, ¹¹²Ag, ¹¹³Ag, ²³⁹Am, ²⁴⁰Am, ²⁴²Am, ²⁴⁴Am, ³⁷Ar, ⁷¹As, ⁷²As, ⁷³As, ⁷⁴As ⁷⁶As, ⁷⁷As, ²⁰⁹At, ²¹⁰At, ¹⁹¹Au, ¹⁹²Au, ¹⁹³Au, ¹⁹⁴Au, ¹⁹⁵Au, ¹⁹⁶Au, ^{196m2}Au, ¹⁹⁶Au, ^{198m}Au, ¹⁹⁹Au ^{200m}Au, ¹²⁸Ba, ¹³¹Ba, ^{133m}Ba, ^{135m}Ba, ¹⁴⁰Ba, ⁷Be, ²⁰³Bi, ²⁰⁴Bi, ²⁰⁵Bi, ²⁰⁶Bi, ²¹⁰Bi, ²¹²Bi, ²⁴³Bk, ²⁴⁴Bk ²⁴⁵Bk, ²⁴⁶Bk, ^{248m}Bk, ²⁵⁰Bk, ⁷⁶Br, ⁷⁷Br, ^{80m}Br, ⁸²Br, ¹¹C, ¹⁴C, ⁴⁵Ca, ⁴⁷Ca, ¹⁰⁷Cd, ¹¹⁵Cd, ¹¹⁵mCd ^{117m}Cd, ¹³²Ce, ^{133m}Ce, ¹³⁴Ce, ¹³⁵Ce, ¹³⁷Ce, ^{137m}Ce, ¹³⁹Ce, ¹⁴¹Ce, ¹⁴³Ce, ¹⁴⁴Ce, ²⁴⁶Cf, ²⁴⁷Cf, ²⁵³Cf, ²⁵⁴Cf, ²⁴⁰Cm, ²⁴¹Cm, ²⁴²Cm, ²⁵²Cm, ⁵⁵Co, ⁵⁶Co, ⁵⁷Co, ⁵⁸Co, ^{58m}Co, ⁶⁰Co, ⁴⁸Cr, ⁵¹Cr, ¹²⁷Cs, ¹²⁹Cs ¹³¹Cs, ¹³²Cs, ¹³⁶Cs, ¹³⁷Cs, ⁶¹Cu, ⁶²Cu, ⁶⁴Cu, ⁶⁷Cu, ¹⁵³Dy, ¹⁵⁵Dy, ¹⁵⁷Dy, ¹⁵⁹Dy, ¹⁶⁵Dy, ¹⁶⁶Dy, ¹⁶⁰Er ¹⁶¹Er, ¹⁶⁵Er, ¹⁶⁹Er, ¹⁷¹Er, ¹⁷²Er, ²⁵⁰Es, ²⁵¹Es, ²⁵³Es, ²⁵⁴Es, ^{254m}Es, ²⁵⁵Es, ^{256m}Es, ¹⁴⁵Eu, ¹⁴⁶Eu, ¹⁴⁷Eu ¹⁴⁸Eu, ¹⁴⁹Eu, ^{150m}Eu, ¹⁵²Eu, ¹⁵⁶Eu, ¹⁵⁷Eu, [¹⁸F]AlF, ⁵²Fe, ⁵⁹Fe, ²⁵¹Fm, ²⁵²Fm, ²⁵³Fm, ²⁵⁴Fm, ²⁵⁵Fm, ²⁵⁷Fm ⁶⁶Ga, ⁶⁷Ga, ⁶⁸Ga, ⁷²Ga, ⁷³Ga, ¹⁴⁶Gd, ¹⁴⁷Gd, ¹⁴⁹Gd, ¹⁵¹Gd, ¹⁵³Gd, ¹⁵⁹Gd, ⁶⁸Ge, ⁶⁹Ge, ⁷¹Ge, ⁷⁷Ge ¹⁷⁰Hf, ¹⁷¹Hf, ¹⁷³Hf, ¹⁷⁵Hf, ^{179m2}Hf, ^{180m}Hf, ¹⁸¹Hf, ¹⁸⁴Hf, ¹⁹²Hg, ¹⁹³Hg, ^{193m}Hg, ¹⁹⁵Hg, ^{195m}Hg, ¹⁹⁷Hg ^{197m}Hg, ²⁰³Hg, ^{160m}Ho, ¹⁶⁶Ho, ¹⁶⁷Ho, ¹²³I, ¹²⁴I, ¹²⁶I, ¹³⁰I, ¹³²I, ¹³³I, ¹³⁵I, ¹⁰⁹In, ¹¹⁰In, ¹¹¹In, ^{114m}In, ^{115m}In, ¹⁸⁴Ir, ¹⁸⁵Ir, ¹⁸⁶Ir, ¹⁸⁷Ir, ¹⁸⁸Ir, ¹⁸⁹Ir, ¹⁹⁰Ir, ^{190m2}Ir, ¹⁹²Ir, ^{193m}Ir, ¹⁹⁴Ir, ^{194m2}Ir, ^{195m}Ir, ⁴²K, ⁴³K, ⁷⁸Kr ⁷⁹Kr, ^{81m}Kr, ^{85m}Kr, ¹³²La, ¹³³La, ¹³⁵La, ¹⁴⁰La, ¹⁴¹La, ²⁶²Lr, ¹⁶⁹Lu, ¹⁷⁰Lu, ¹⁷¹Lu, ¹⁷²Lu, ^{174m}Lu, ^{176m}Lu ¹⁷⁷Lu, ^{177m}Lu, ¹⁷⁹Lu, ²⁵⁷Md, ²⁵⁸Md, ²⁶⁰Md, ²⁸Mg, ⁵²Mn, ⁹⁰Mo, ^{93m}Mo, ⁹⁹Mo, ¹⁰⁰Mo, ¹³N, ²⁴Na, ⁹⁰Nb, ^{91m}Nb ^{92m}Nb, ⁹⁵Nb, ^{95m}Nb, ⁹⁶Nb, ¹³⁸Nd, ^{139m}Nd, ¹⁴⁰Nd, ¹⁴⁷Nd, ⁵⁶Ni, ⁵⁷Ni, ⁶⁶Ni, ²³⁴Np, ²³⁶Np, ⁹⁹Mo, Np, ²³⁸Np ²³⁹Np, ¹⁵O, ¹⁸²Os, ¹⁸³Os, ^{183m}Os, ¹⁸⁵Os, ^{189m}Os, ¹⁹¹Os, ^{191m}Os, ¹⁹³Os, ³²P, ³³P, ²²⁸Pa, ²²⁹Pa, ²³⁰Pa, ²³²Pa, ²³³Pa, ²³⁴Pa, ²⁰⁰Pb, ²⁰¹Pb, ²⁰²mPb, ²⁰³Pb, ²⁰⁹Pb, ²¹²Pb, ¹⁰⁰Pd, ¹⁰¹Pd, ¹⁰³Pd, ¹⁰⁹Pd ^{111m}Pd, ¹¹²Pd, ¹⁴³Pm, ¹⁴⁸Pm, ^{148m}Pm, ¹⁴⁹Pm, ¹⁵¹Pm, ²⁰⁴Po, ²⁰⁶Po, ²⁰⁷Po, ²¹⁰Po, ¹³⁹Pr, ¹⁴²Pr, ¹⁴³Pr, ¹⁴⁵Pr, ¹⁸⁸Pt, ¹⁹¹Pt, ¹⁹¹Pt, ^{193m}Pt, ^{195m}Pt, ¹⁹⁷Pt, ²⁰⁰Pt, ²⁰²Pt, ²³⁴Pu, ²³⁷Pu, ²⁴³Pu, ²⁴⁵Pu, ²⁴⁶Pu, ²⁴⁷Pu ²²³Ra, ²²⁴Ra, ²²⁵Ra, ⁸¹Rb, ⁸²Rb, ⁸²mRb, ⁸³Rb, ⁸⁴Rb, ⁸⁶Rb, ¹⁸¹Re, ¹⁸²Re, ^{182m}Re, ¹⁸³Re, ¹⁸⁴Re, ^{184m}Re, ¹⁸⁶Re, ¹⁸⁸Re, ¹⁸⁹Re, ^{190m}Re, ⁹⁹Rh, ^{99m}Rh, ¹⁰⁰Rh, ^{101m}Rh, ¹⁰²Rh, ^{103m}Rh, ¹⁰⁵Rh, ²¹¹Rn, ²²²Rn, ⁹⁷Ru, ¹⁰³Ru, ¹⁰⁵Ru, ³⁵S, ^{118m}Sb, ¹¹⁹Sb, ¹²⁰Sb, ^{120m}Sb, ¹²²Sb, ¹²⁴Sb, ¹²⁶Sb, ¹²⁷Sb, ¹²⁸Sb, ¹²⁹Sb, ⁴³Sc, ⁴⁴Sc, ^{44m}Sc, ⁴⁶Sc, ⁴⁷Sc, ⁴⁸Sc, ⁷²Se, ⁷³Se, ⁷⁵Se, ¹⁵³Sm, ¹⁵⁶Sm, ¹¹⁰Sn, ¹¹³Sn, ^{117m}Sn ^{119m}Sn, ¹²¹Sn, ¹²³Sn, ¹²⁵Sn, ⁸²Sr, ⁸³Sr, ⁸⁵Sr, ⁸⁹Sr, ⁹¹Sr, ¹⁷³Ta, ¹⁷⁵Ta, ¹⁷⁶Ta, ¹⁷⁷Ta, ¹⁸⁰Ta, ¹⁸²Ta ¹⁸³Ta, ¹⁸⁷⁴Ta, ¹⁴⁹Tb, ¹⁵⁰Tb, ¹⁵¹Tb, ¹⁵²Tb, ¹⁵³Tb, ¹⁵⁴Tb, ^{154m}Tb, ^{154m2}Tb, ¹⁵⁵Tb, ¹⁵⁶Tb, ^{156m}Tb ^{156m2}Tb, ¹⁶⁰Tb, ¹⁶¹Tb, ⁹⁴Tc, ⁹⁵Tc, ^{95m}Tc, ⁹⁶Tc, ^{97m}Tc, ^{99m}Tc, ¹¹⁸Te, ¹¹⁹Te, ^{119m}Te, ¹²¹Te, ^{121m}Te, ^{123m}Te, ^{125m}Te, ¹²⁷Te, ^{127m}Te, ^{129m}Te, ^{131m}Te, ¹³²Te, ²²⁷Th, ²²⁹Th, ²³¹Th, ²³⁴Th, ⁴⁵Ti, ¹⁹⁸Tl, ¹⁹⁹Tl, ²⁰⁰Tl, ²⁰¹Tl, ²⁰²Tl, ²⁰⁴Tl, ¹⁶⁵Tm, ¹⁶⁶Tm, ¹⁶⁷Tm, ¹⁶⁸Tm, ¹⁷⁰Tm, ¹⁷²Tm, ¹⁷³Tm, ²³⁰U, ²³¹U, ²³⁷U, ²⁴⁰U, ⁴⁸V, ¹⁷⁸W, ¹⁸¹W, ¹⁸⁵W, ¹⁸⁷W, ¹⁸⁸W, ¹²²Xe, ¹²⁵Xe, ¹²⁷Xe, ^{129m}Xe, ^{131m}Xe, ¹³³Xe, ^{133m}Xe, ¹³⁵Xe, ^{85m}Y, ⁸⁶Y, ⁸⁷Y, ^{87m}Y, ⁸⁸Y, ⁹⁰Y, ^{90m}Y, ⁹¹Y, ⁹²Y, ⁹³Y, ₁₆₆Yb, ¹⁶⁹Yb, ¹⁷⁵Yb, ⁶²Zn, ⁶⁵Zn, ^{69m}Zn, ^{71m}Zn, ⁷²Zn, ⁸⁶Zr, ⁸⁸Zr, ⁸⁹Zr, ⁹⁵Zr, and ⁹⁷Zr and the like. The skilled person would understand that any other radionuclide known in the art can also be used in the conjugate of the present application.

In some embodiment, the radionuclide is selected from ⁶⁸Ga, ¹⁷⁷Lu, [¹⁸F]AlF and ⁹⁰Y.

In some embodiments, the conjugate of the present application comprises the chelating agent coordinatively bound to two radionuclides. In some embodiments, one radionuclide is diagnostically active and second radionuclide is therapeutically active. In some embodiments, the diagnostically active radionuclide is selected from ⁶⁸Ga and [¹⁸F]AlF. In some embodiments, the therapeutically active radionuclide is selected from ¹⁷⁷Lu and ⁹⁰Y.

In some embodiments, the targeting ligand is a receptor specific moiety. In some embodiments, the targeting ligand is selective of specific for a cell surface molecule expressed on the surface of a target cell. In some embodiments, the targeting ligand is selective or specific for the targeted organ or tissue. In some embodiments, the targeting ligand of the present application directing the conjugate of the present application to a targeted tissue, organ, receptor or other biologically expressed composition.

Examples of targeting ligands include, without limiting, proteins, peptides and peptidomimetics such as arginine-glycine-aspartic acid (RGD)-containing peptides and peptidomimetics, amino-acid sequences, antibodies or fragments thereof such as Fab fragments and antigen binding sites of antibodies, single-chain variable fragments of antibodies, nucleotide sequences, DNA sequences, RNA sequences, peptide nucleic acid (PNA) sequences, carbohydrates, and steroids. In some embodiments, the targeting ligand is selected from an oligonucleotide, an oligopeptide, a protein, a peptide and a small molecule compound.

Examples of targeting ligands include, without limiting, gemtuzumab, inotuzumab, trastuzumab (Herceptin), HD37, M195, LMB2, lym1, 8106, HMFG1, CC49, rituximab, epratuzumab, lorvotuzumab, 2C3, imgn388, SAR3419, BilB062, brentixumab, glembatumumab, SGN-75, PSMA ADC, ASG-5ME or mdx-1203. In some embodiments, the targeting antibody is a variant of gemtuzumab, inotuzumab, trastuzumab (Herceptin), HD37, M195, LMB2, lym1, 8106, HMFG1, CC49, rituximab, epratuzumab, lorvotuzumab, 2C3, imgn388, SAR3419, BilB062, brentixumab, glembatumumab, SGN-75, PSMA ADC, ASG-5ME, mdx-1203, TATE, TOC, PSMA, bombesin derivatives (e.g. RM2, BBN and the like), FAPI derivatives, and the like. A person skilled in the art would understand that any other targeting ligands known in the art can also be used in the conjugate of the present application.

In some embodiments, the targeting ligand is a tumor-targeting ligand. In some embodiments, the tumor-targeting ligand is selective or specific for a molecular target such as a receptor.

In some embodiments, the targeting ligand is selected from TATE, TOC and PSMA.

In some embodiments, n is an integer 1 to 3. In some embodiments, n is 1. In some embodiments, n is 2. As such, the conjugate of the present application comprises at least one ZW linker. In some embodiments, the conjugate of the present application comprises two ZW linkers.

In some embodiments, the targeting ligand and the complex comprising the chelating agent form a covalent bond with the ZW linker to form the conjugate. The ZW linker is a permanent zwitterionic group which possesses both a positive and a negative charge simultaneously. In some embodiments, the permanent zwitterionic group is formed at physiological pH of 5.5-7.5.

In some embodiments, the ZW linker is a natural or unnatural amino acid with a primary amine. In some embodiments, the natural amino acid is lysine. In some embodiments, the unnatural amino acid is pyridyl-alanine.

In some embodiments, the conjugate of the present application further comprises a spacer. The spacer used in the conjugate of the application can be any spacer known in the art. Examples include, but are not limited to, poly(ethylene)glycol (PEG) and hexanoic acid. In some embodiments, the spacer is poly(ethylene)glycol. Therefore, when the conjugate of the present application comprises a spacer, the conjugate has the formula IIa:

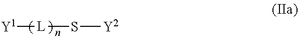

wherein,
S is a spacer.

The length of the spacer of the present application is defined by the number of the repeating PEG units. In some embodiments, PEG spacer contains 2 to 15 polyethylene glycol units. In some embodiments, PEG contains four units.

In some embodiments, the conjugate of the present application is a radiopharmaceutical. In some embodiments, the radiopharmaceutical is a protein-based radiopharmaceutical. In some embodiments, the radiopharmaceutical is a peptide-based radiopharmaceutical.

In some embodiments, the conjugate of the present application has a greater solubility and/or higher stability in vivo than [⁶⁸Ga]Ga-DOTA-TATE.

In some embodiments, the conjugate of the present application has lower kidney and other healthy organ uptake and/or retention than [⁶⁸Ga]Ga-DOTA-TATE.

The present application further includes a conjugate of Formula IV, or a salt and/or solvate thereof:

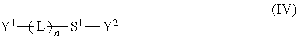

wherein
Y¹ is a complex comprising a chelating agent and one or more radionuclides, wherein the chelating agent is desferrioxamine (DFO) or a compound of the following formula;

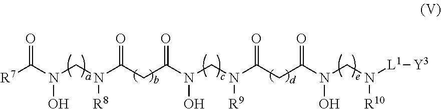

wherein
R⁷, R⁸, R⁹ and R¹⁰ are independently selected from H and C₁₋₄alkyl;
each a, c and e is an integer independently selected from 3, 4, 5, 6 and 7;
each b and d is an integer independently selected from 1, 2, 3 and 4;
L¹ is a linker group

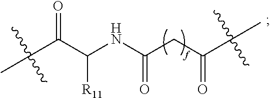

Y³ is

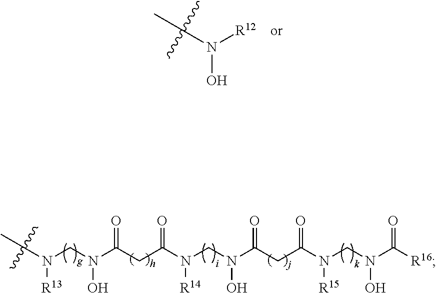

R¹¹ is selected from C₁₋₁₀alkyl and C₁₋₆alkylenephenyl, optionally substituted by one of NH₂, OH, SH, N₃, CO₂H and

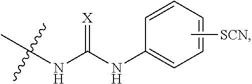

wherein X is O, S or NH;
R¹², R¹³, R¹⁴, R¹⁵ and R¹⁶ are independently selected from H and C₁₋₄alkyl;
f is an integer selected from 1, 2, 3 and 4;
each g, l and k is an integer independently selected from 3, 4, 5, 6 and 7;
each h and j is an integer independently selected from 1, 2, 3 and 4;
Y² is a targeting ligand;
S¹ is a biomolecule conjugating group;
L is a zwitterionic (ZW) linker of the formula

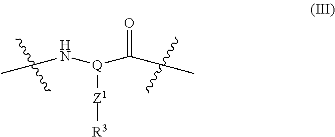

wherein
Q is selected from Q1, Q2, Q3, Q4 and Q5:

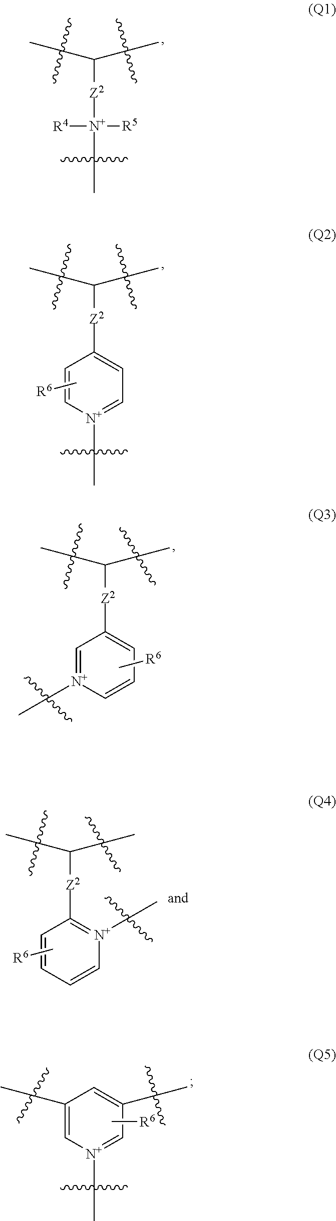

Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R³ is an anion containing group selected from sulfonate, carboxylate, sulfate and phosphate; and
n is an integer 1 to 5;
wherein the chelating agent is a compound of Formula V, Y¹ is attached to L at R¹¹ of L¹ group

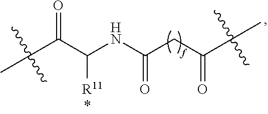

and the L is further optionally attached to one to three additional L groups, or the L is attached between the Y³

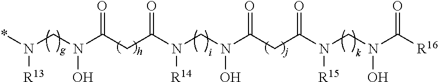

and L¹

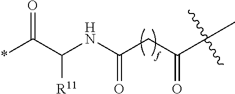

groups of Y¹, or one L is attached at R¹¹ of L¹ group and another L is attached between the Y³ and L¹ groups of Y¹, and each one of the L is further optionally attached to one to three additional L groups; and
wherein the chelating agent is DFO, the point of attachment of L is at the amine

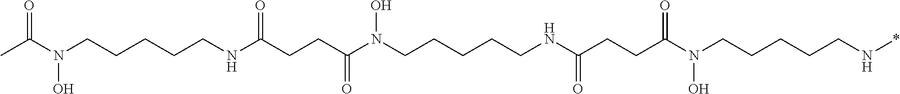 [SMILES: *NCCCCCN(O)C(=O)CCC(=O)NCCCCCN(O)C(=O)CCC(=O)NCCCCCN(O)C(C)=O]

and wherein the * indicates the point of attachment.

In some embodiments, Q is Q1,

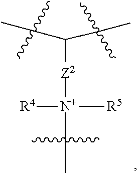

and the compound of Formula IV has the following structure:

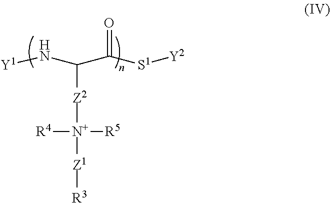

or a salt and/or solvate thereof,
wherein Y¹, Y², R³, R⁴, R⁵, Z¹, Z², S¹ and n are as defined for Formula IV.

In some embodiments, Q is Q2,

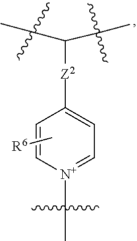

and the compound of Formula IV has the following structure:

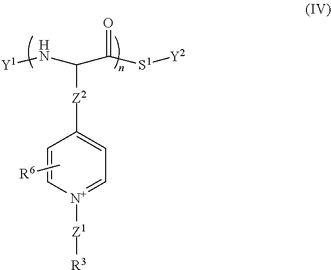

or a salt and/or solvate thereof,
wherein Y¹, Y², R³, R⁶, Z¹, Z², S¹ and n are as defined for Formula IV.

In some embodiments, Q is Q3,

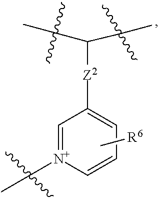

and the compound of Formula IV has the following structure:

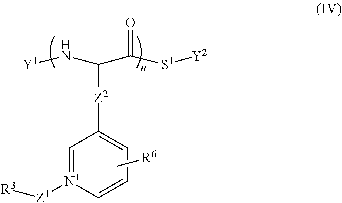

or a salt and/or solvate thereof,
wherein Y¹, Y², R³, R⁶, Z¹, Z², S¹ and n are as defined for Formula IV.

In some embodiments, Q is Q4,

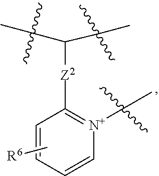

and the compound of Formula IV has the following structure:

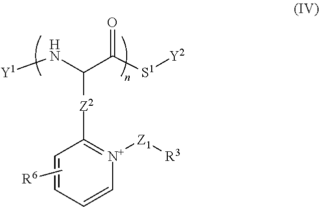

or a salt and/or solvate thereof,
wherein Y¹, Y², R³, R⁶, Z¹, Z², S¹ and n are as defined for Formula IV.

In some embodiments, Q is Q5,

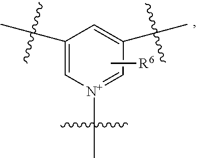

and the compound of Formula IV has the following structure:

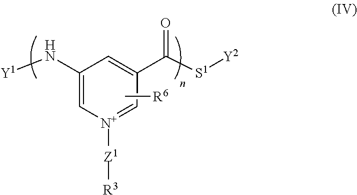

or a salt and/or solvate thereof,
wherein Y¹, Y², R³, R⁶, Z¹, S¹ and n are as defined for Formula IV.

In some embodiments, when Q is

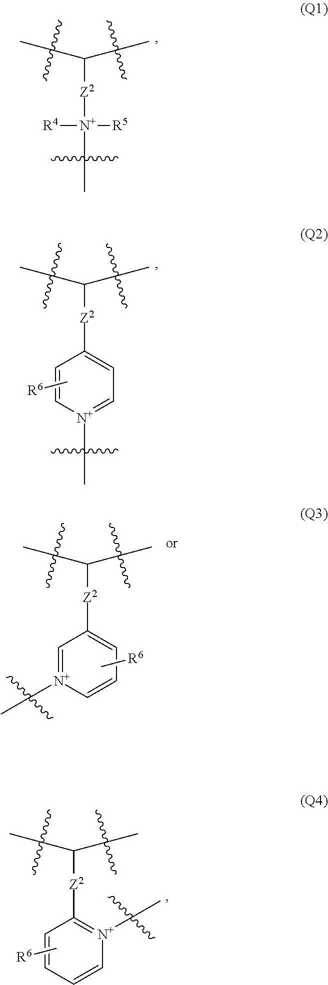

the stereochemistry at the carbon to which Z² is bonded is either R or S. In some embodiments, the stereochemistry at the carbon to which Z² is attached is R. In some embodiments, the stereochemistry at the carbon to which Z² is attached is S.

In some embodiments, R⁴ and R⁵ are independently selected from C₁₋₆alkyl, C₂₋₆alkenyl and C₂₋₆alkynyl. In some embodiments, R⁴ and R⁵ are independently selected from C₁₋₄alkyl, C₂₋₄alkenyl and C₂₋₄alkynyl. In some embodiments, R⁴ and R⁵ are independently selected from CH₂CH₂CH₃, C(CH₃)₃, CH(CH₃)CH₂CH₃ and CH₂CH(CH₃)₂, CH(CH₃)₂, CH₂CH₃ and CH₃. In some embodiments, R⁴ and R⁵ are independently selected from CH₂CH₂CH₃, CH(CH₃)₂, CH₂CH₃ and CH₃. In some embodiments, at least one of R⁴ and R⁵ is CH₃. In some embodiments, each one of R⁴ and R⁵ is CH₃.

In some embodiments, R⁶ is H.

In some embodiments, Z² is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, N and S. In some embodiments, Z² is selected from C₁₋₆alkylene, C₂₋₆alkenylene and C₂₋₆alkynelene, unsubstituted or substituted with one or more of halo or OH and/or are optionally interrupted by one to three heteromoieties independently selected from O, N and S. In some embodiments, Z² is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, N and S. In some embodiments, Z² is C₄alkylene. In some embodiments, Z² is C₁alkylene.

In some embodiments, Z¹ is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, N and S. In some embodiments, Z¹ is selected from C₁₋₆alkylene, C₂₋₆alkenylene and C₂₋₆alkynelene, unsubstituted or substituted with one or more of halo or OH and/or are optionally interrupted by one to three heteromoieties independently selected from O, N and S. In some embodiments, Z¹ is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, N and S. In some embodiments, Z¹ is C₃alkylene.

In some embodiments, R³ is sulfonate.

In some embodiments, L is selected from

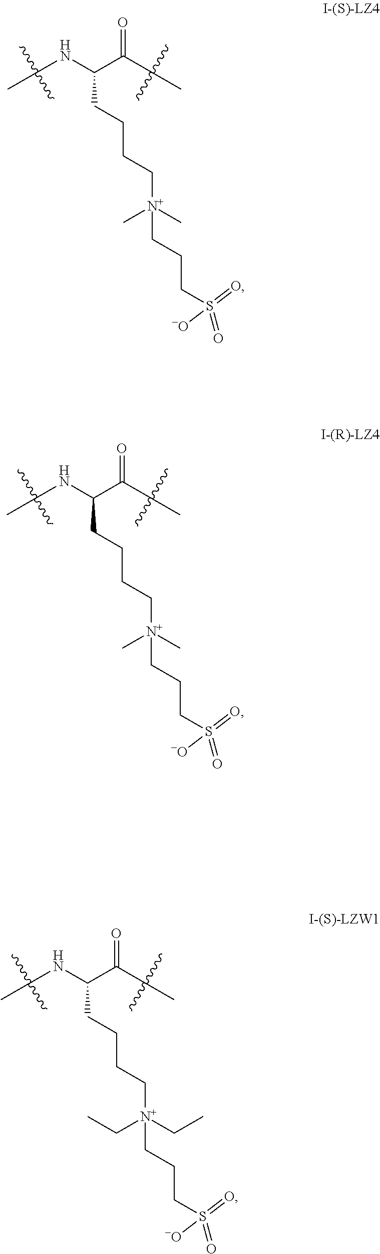

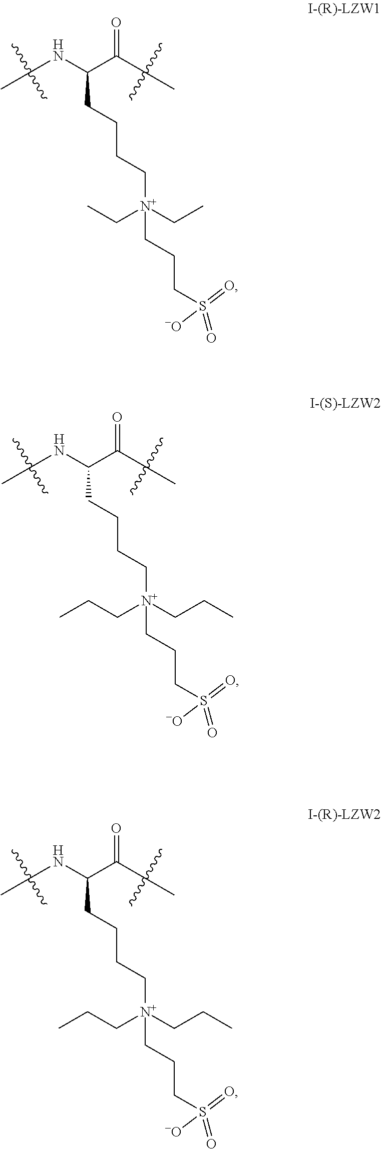

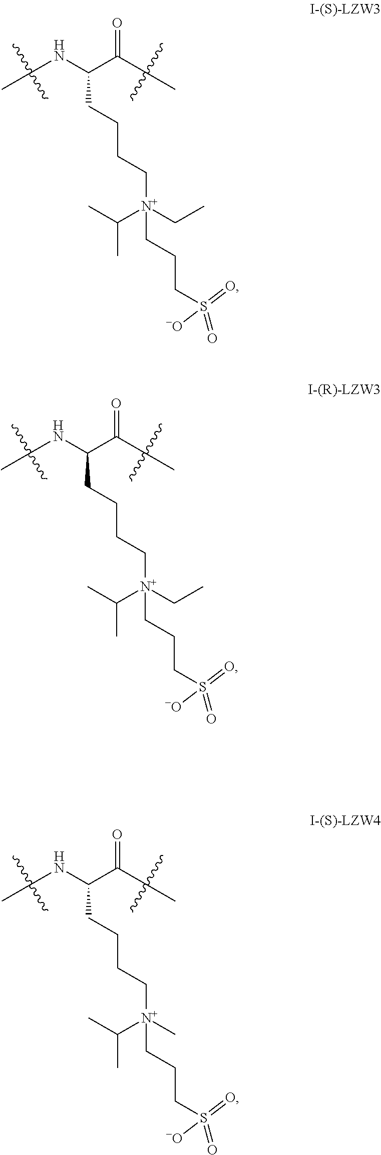

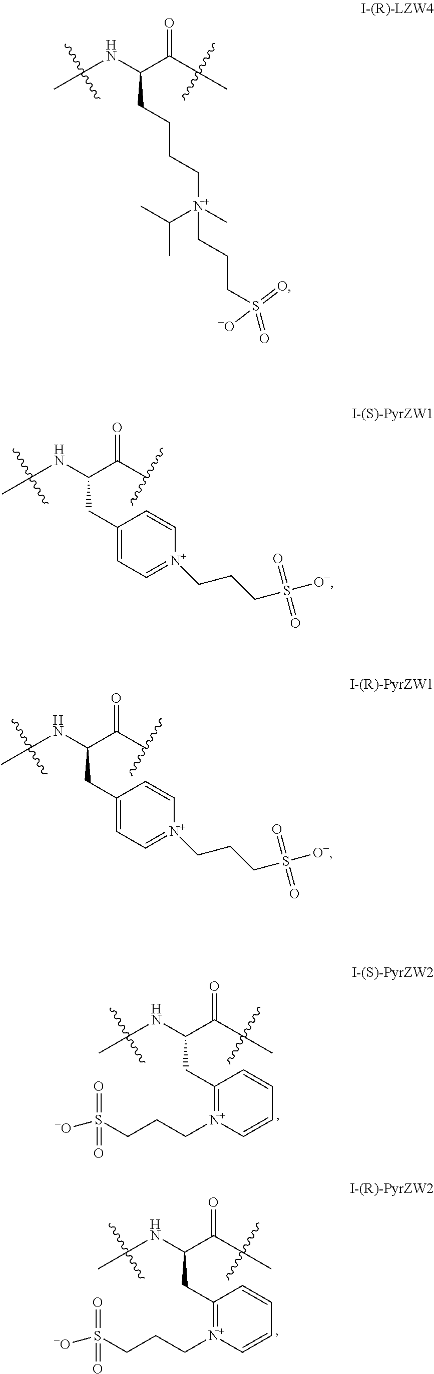

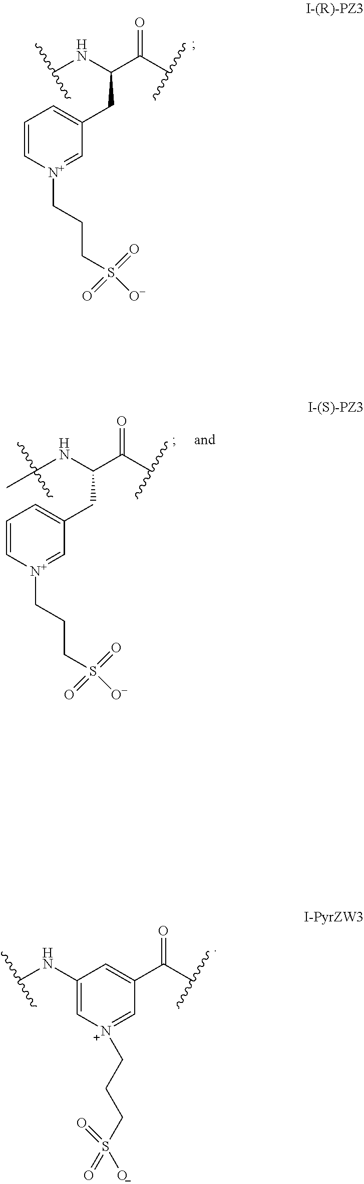 [SMILES: *N[C@H](Cc1ccc[n+](CCCS(=O)(=O)[O-])c1)C(*)=O ; *N[C@@H](Cc1ccc[n+](CCCS(=O)(=O)[O-])c1)C(*)=O ; *Nc1cc(C(*)=O)c[n+](CCCS(=O)(=O)[O-])c1]

In some embodiments, L is

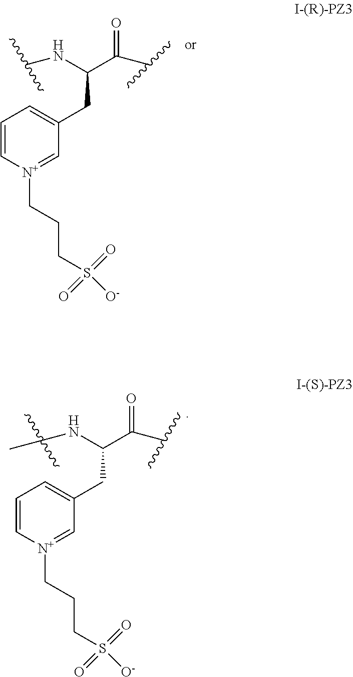 [SMILES: *N[C@H](Cc1ccc[n+](CCCS(=O)(=O)[O-])c1)C(*)=O ; "or" ; *N[C@@H](Cc1ccc[n+](CCCS(=O)(=O)[O-])c1)C(*)=O ; "."]

The ZW linker is a permanent zwitterionic group which possesses both a positive and a negative charge simultaneously. In some embodiments, the permanent zwitterionic group is formed at physiological pH of 5.5-7.5.

In some embodiments, the ZW linker is a natural or unnatural amino acid with a primary amine. In some embodiments, the natural amino acid is lysine. In some embodiments, the unnatural amino acid is pyridyl-alanine.

In some embodiments, Y¹ is a complex comprising a chelating agent and one or more radionuclides, wherein the chelating agent is DFO.

In some embodiments, Y¹ is a complex comprising a chelating agent and one or more radionuclides, wherein the chelating agent is a compound of Formula V:

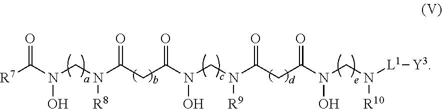

In some embodiments, when the chelating agent is a compound of Formula V, R⁷, R⁸, R⁹ and R¹⁰ are independently selected from H and C₁₋₃alkyl. In some embodiments, R⁷, R⁸, R⁹ and R¹⁰ are independently selected from H and C₁₋₂alkyl. In some embodiments, at least one of R⁷, R⁸, R⁹ and R¹⁰ is C₁₋₂alkyl. In some embodiments, R⁷ is CH₃. In some embodiments, each one of R⁸, R⁹ and R¹⁰ is H.

In some embodiments, each a, c and e is an integer independently selected from 4, 5 and 6. In some embodiments, a, c and e are different. In some embodiments, a, c and e are 5.

In some embodiments, each b and d is an integer independently selected from 1, 2 and 3. In some embodiments, b and d are different. In some embodiments, b and d are 2.

In some embodiments, f is selected from the integer 1, 2 and 3. In some embodiments, f is 2.

In some embodiments, when Y is

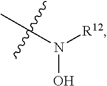

R¹² is selected from H and C₁₋₂alkyl. In some embodiments, R¹² is selected from H and CH₃. In some embodiments, R¹² is CH₃.

In some embodiments, when Y is

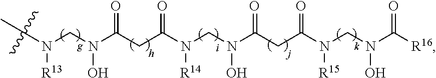

R¹³, R¹⁴, R¹⁵ and R¹⁶ are independently selected from H and C₁₋₃alkyl. In some embodiments, R¹³, R¹⁴, R¹⁵ and R¹⁶ are independently selected from H and C₁₋₂alkyl. In some embodiments, at least one of R¹³, R¹⁴, R¹⁵ and R¹⁶ is C₁₋₂alkyl. In some embodiments, R¹⁶ is CH₃. In some embodiments, each one of R¹³, R¹⁴ and R¹⁵ is H.

In some embodiments, each g, l and k is an integer independently selected from 4, 5 and 6. In some embodiments, g, l and k are different. In some embodiments, g, l and k are 5.

In some embodiments, each h and j is an integer independently selected from 1, 2 and 3. In some embodiments, h and j are different. In some embodiments, h and j are 2.

In some embodiments, a, c and e are 5 and b and d are 2 and the compound of Formula V has the following structure:

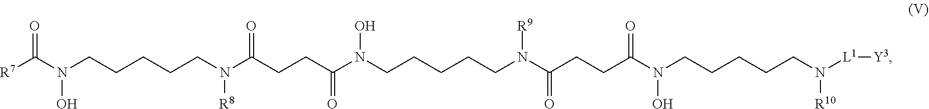

wherein R⁷, R⁸, R⁹, R¹⁰, L¹ and Y³ are as defined above.

In some embodiments, when Y³ is

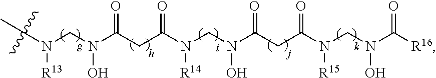

g, i and k are 5 and h and j are 2 and the compound of Formula V has the following structure:

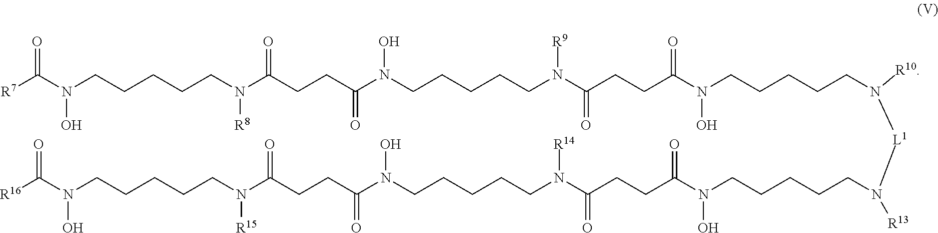

In some embodiments, the compound of Formula V is selected from

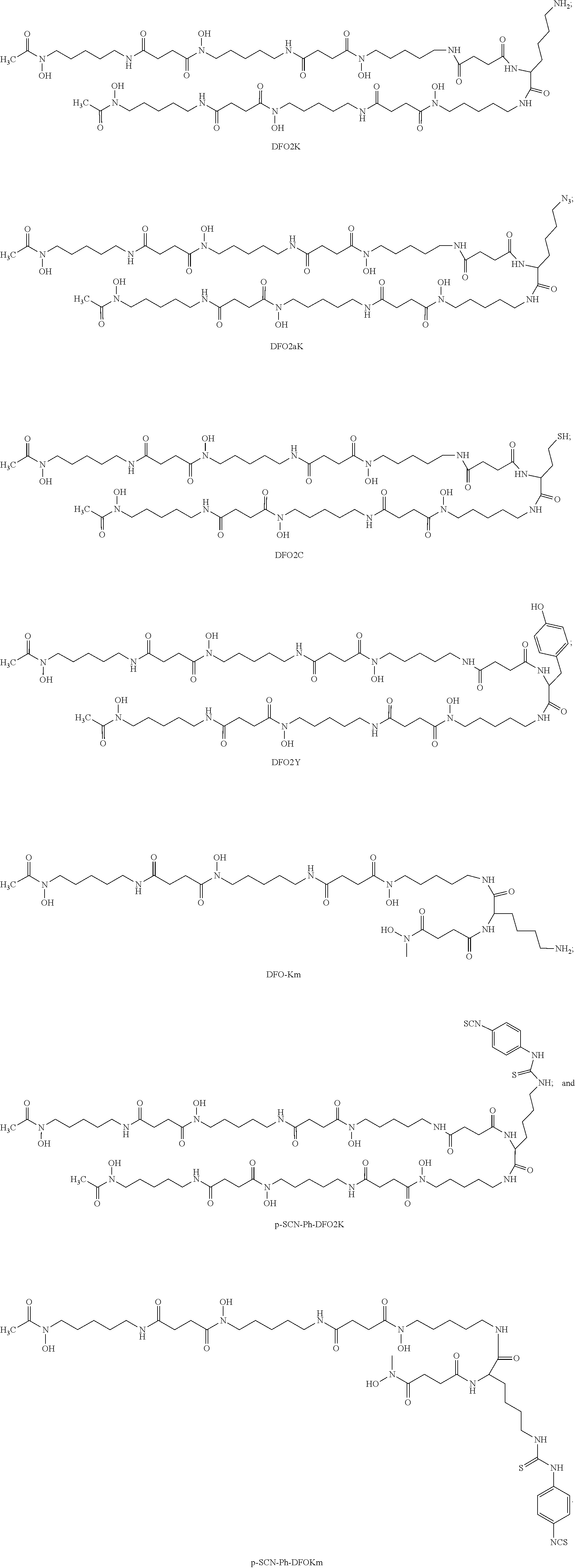

In some embodiments, n is an integer 1 to 3. In some embodiments, n is an integer 1. In some embodiments, n is an integer 2. As such, the conjugate of the present application comprises at least one ZW linker. In some embodiments, the conjugate of the present application comprises two ZW linkers.

In some embodiments, the chelating agent is a compound of Formula V and Y¹ is attached to L at R¹¹ of L¹ group

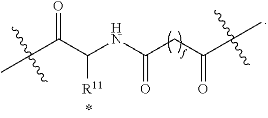

In some embodiments, the L is further optionally attached to one or two additional L groups. In some embodiments, the L is further optionally attached to one L group.

In some embodiments, the chelating agent is a compound of Formula V and L is attached between the Y³

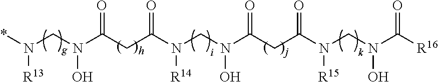

### and L¹

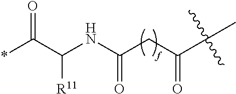

groups of Y¹. In some embodiments, L is further optionally attached to one or two additional L groups.

In some embodiments, the chelating agent is a compound of Formula V and one L is attached at R¹¹ of L¹ group

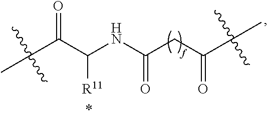

and another L is attached between the Y³

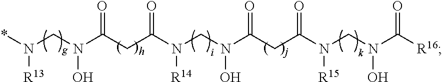

### and L¹

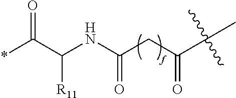

groups of Y¹. In some embodiments, L group attached at R¹¹ of L¹ group is further attached to one to three additional L groups. In some embodiments, L group attached between the Y³ and L¹ groups of Y¹ is further attached to one additional L group.

In some embodiments, when the chelating agent is DFO, the point of attachment of L is at the amine

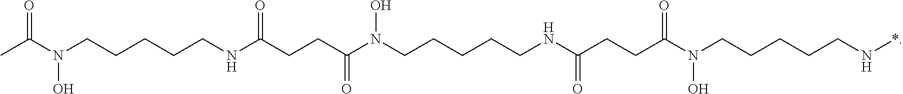

In some embodiments, Y¹-L is selected from

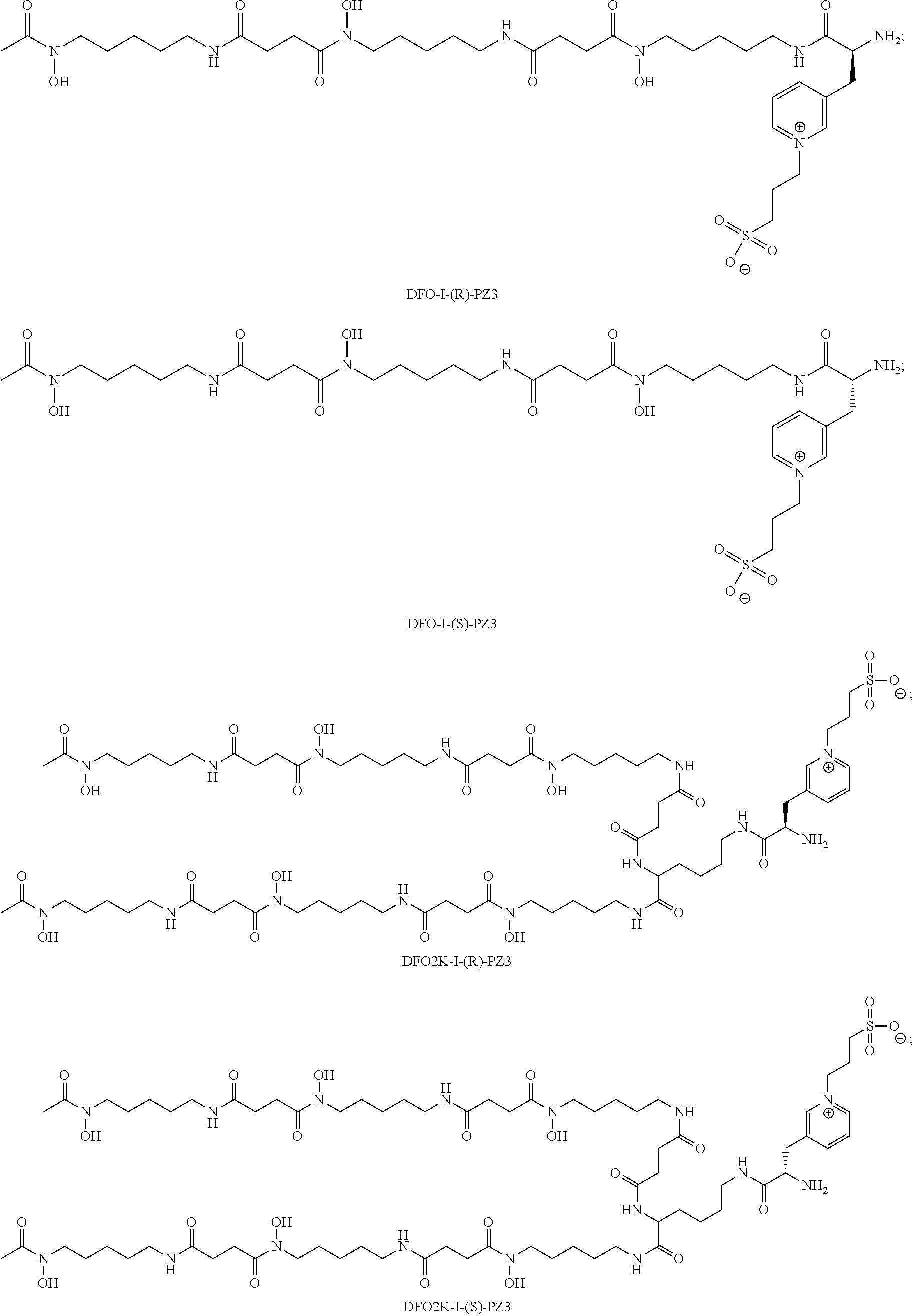

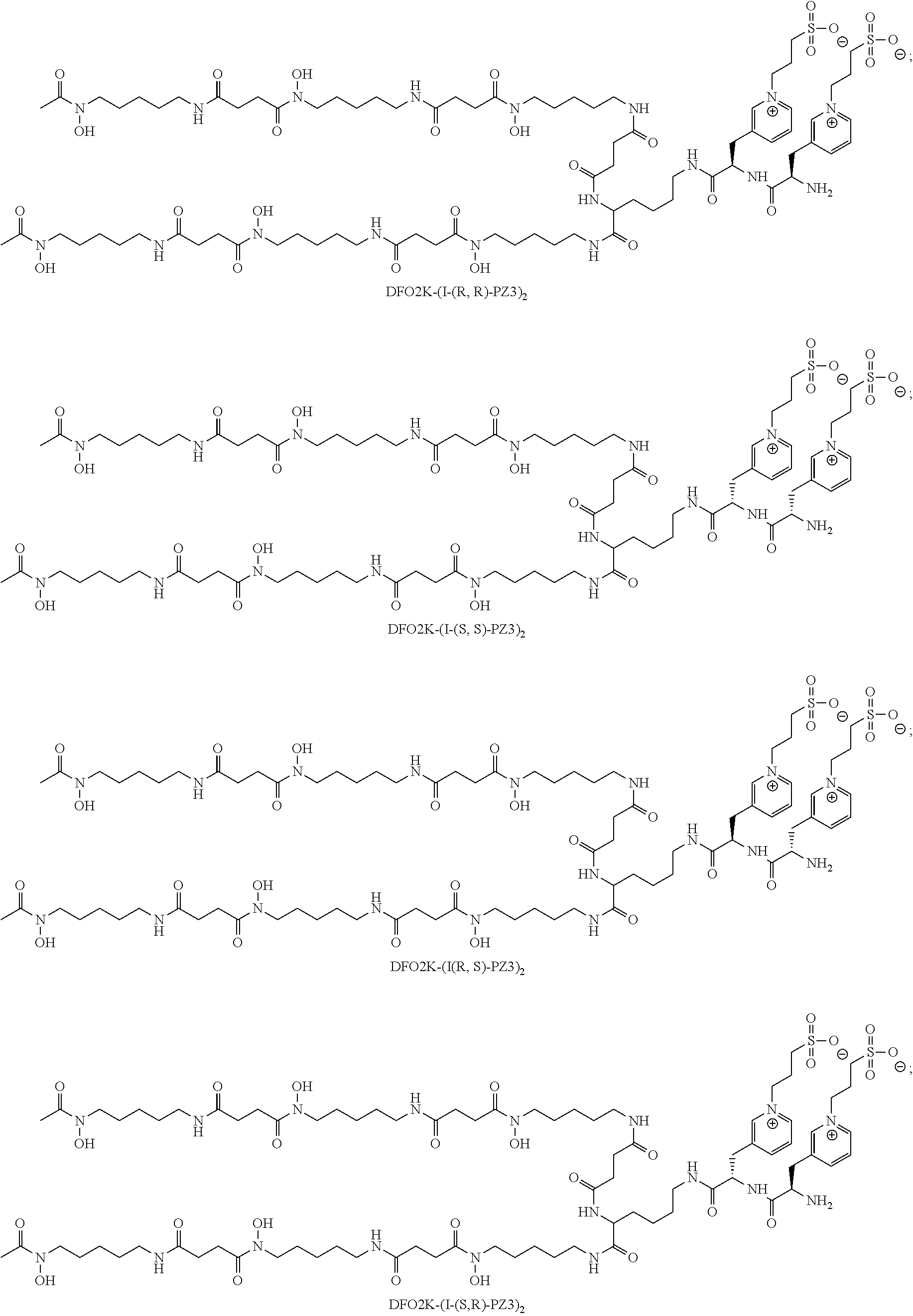

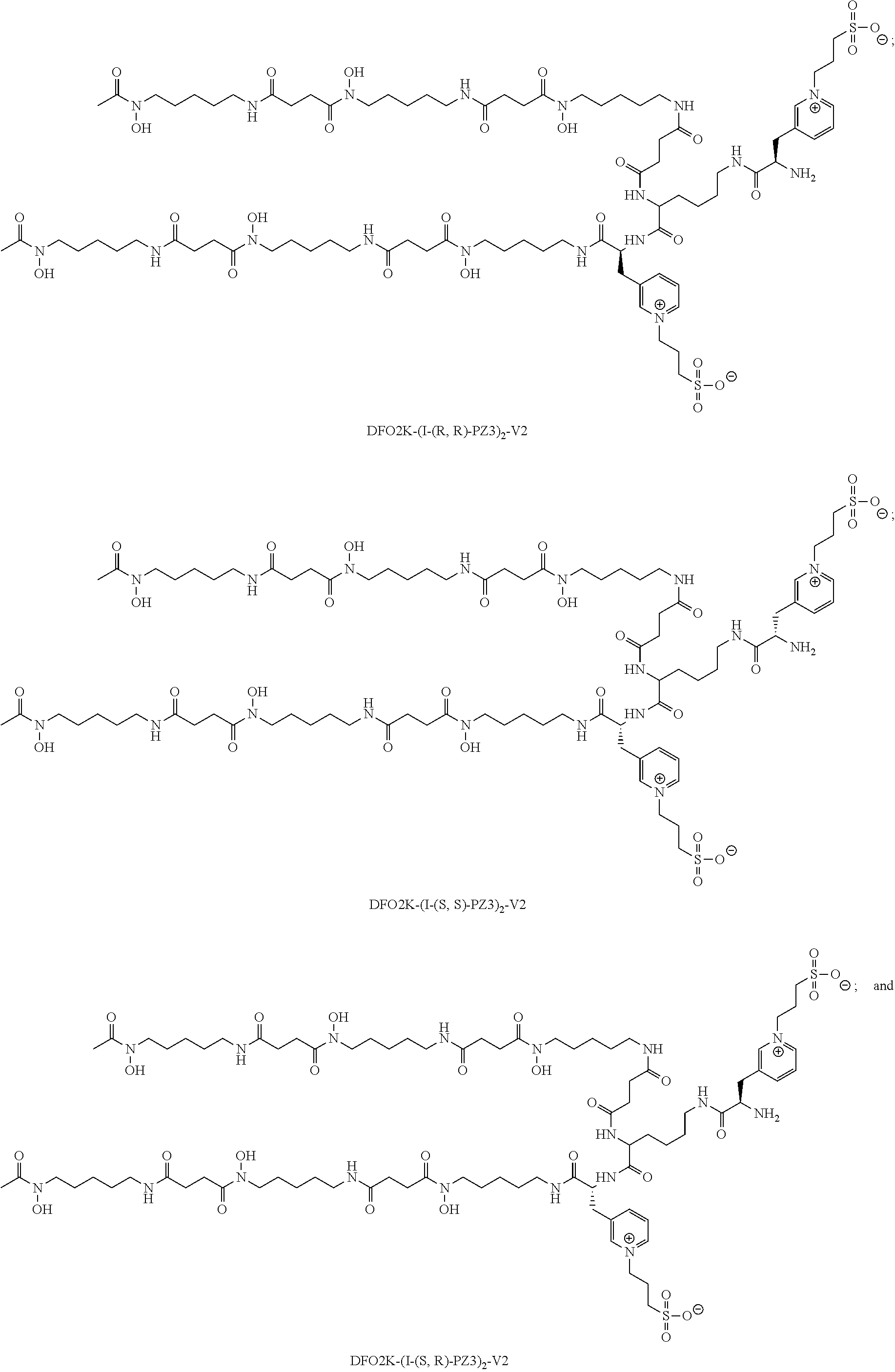

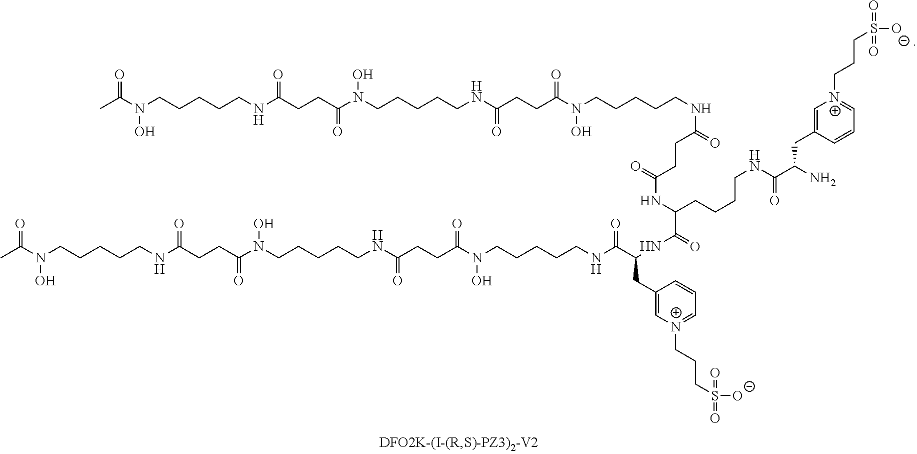 [SMILES: CC(=O)N(O)CCCCCNC(=O)CCC(=O)N(O)CCCCCNC(=O)CCC(=O)N(O)CCCCCNC(=O)CCC(=O)NC(CCCCNC(=O)[C@@H](N)Cc1ccc[n+](CCCS(=O)(=O)[O-])c1)C(=O)N[C@@H](Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O]

In some embodiments, S¹ is a biomolecule conjugating group selected from NH₂, OH, SH, N₃, CO₂H, ONH₂, NHNH₂, C(O)C₁₋₄alkyl, C≡CH, SCN, C(O)NH₂, NHC(X)NH₂, C₁₋₄alkyleneN₃, NHC(O)C₁₋₄alkyleneN₃, NHC(O)C₁₋₄alkyleneONH₂, NHC(O)C₁₋₄alkyleneNHNH₂, NHC(O)OC₁₋₄alkyleneN₃, C(O)NHC₁₋₄alkylene-phenylene-tetrazine,

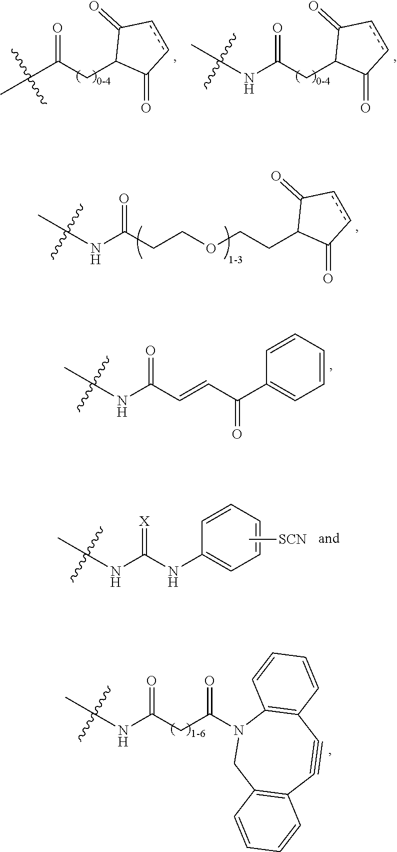

wherein [symbol] represents a single or a double bond and X is O, S or NH. A person skilled in the art would understand that other functional groups known in the art can also be used as a biomolecule conjugating group. This includes any functional group comprising, for example, a nucleophilic group, an electrophilic group, a Michael acceptor, a group that participates in a Click reaction, a group that participates in a cross-coupling reaction, a group that is activated to participate in a nucleophilic displacement reaction, and the like.

In some embodiments, the biomolecule conjugating group is

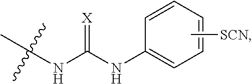

wherein X is O, S or NH.

In some embodiments, the biomolecule conjugating group is

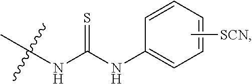

In some embodiments, the radionuclide is selected from a transition metal, rare-earth metal, lanthanide, actinide and metalloid.

Radionucleotides that can be used in the present application include but are not limited to the following elements and their isotopes: ²²⁵Ac, ²²⁶Ac, ²²⁷Ac, ²²⁸Ac, ¹⁰⁵Ag, ^{106m}Ag, ^{110m}Ag, ¹¹¹Ag, ¹¹²Ag, ¹¹³Ag, ²³⁹Am, ²⁴⁰Am, ²⁴²Am, ²⁴⁴Am, ³⁷Ar, ⁷¹As, ⁷²As, ⁷³As, ⁷⁴As ⁷⁶As, ⁷⁷As, ²⁰⁹At, ²¹⁰At, ¹⁹¹Au, ¹⁹²Au, ¹⁹³Au, ¹⁹⁴Au, ¹⁹⁵Au, ¹⁹⁶Au, ^{196m2}Au, ¹⁹⁶Au, ^{198m}Au, ¹⁹⁹Au ^{200m}Au, ¹²⁸Ba, ¹³¹Ba, ^{133m}Ba, ^{135m}Ba, ¹⁴⁰Ba, ⁷Be, ²⁰³Bi, ²⁰⁴Bi, ²⁰⁵Bi, ²⁰⁶Bi, ²¹⁰Bi, ²¹²Bi, ²⁴³Bk, ²⁴⁴Bk ²⁴⁵Bk, ²⁴⁶Bk, ^{248m}Bk, ²⁵⁰Bk, ⁷⁶Br, ⁷⁷Br, ^{80m}Br, ⁸²Br, ¹¹C, ¹⁴C, ⁴⁵Ca, ⁴⁷Ca, ¹⁰⁷Cd, ¹¹⁵Cd, ¹¹⁵mCd ^{117m}Cd, ¹³²Ce, ^{133m}Ce, ¹³⁴Ce, ¹³⁵Ce, ¹³⁷Ce, ^{137m}Ce, ¹³⁹Ce, ¹⁴¹Ce, ¹⁴³Ce, ¹⁴⁴Ce, ²⁴⁶Cf, ²⁴⁷Cf, ²⁵³Cf, ²⁵⁴Cf, ²⁴⁰Cm, ²⁴¹Cm, ²⁴²Cm, ²⁵²Cm, ⁵⁵Co, ⁵⁶Co, ⁵⁷Co, ⁵⁸Co, ^{58m}Co, ⁶⁰Co, ⁴⁸Cr, ⁵¹Cr, ¹²⁷Cs, ¹²⁹Cs ¹³¹Cs, ¹³²Cs, ¹³⁶Cs, ¹³⁷Cs, ⁶¹Cu, ⁶²Cu, ⁶⁴Cu, ⁶⁷Cu, ¹⁵³Dy, ¹⁵⁵Dy, ¹⁵⁷Dy, ¹⁵⁹Dy, ¹⁶⁵Dy, ¹⁶⁶Dy, ¹⁶⁰Er ¹⁶¹Er, ¹⁶⁵Er, ¹⁶⁹Er, ¹⁷¹Er, ¹⁷²Er, ²⁵⁰Es, ²⁵¹Es, ²⁵³Es, ²⁵⁴Es, ^{254m}Es, ²⁵⁵Es, ^{256m}Es, ¹⁴⁵Eu, ¹⁴⁶Eu, ¹⁴⁷Eu ¹⁴⁸Eu, ¹⁴⁹Eu, ^{150m}Eu, ¹⁵²Eu, ¹⁵⁶Eu, ¹⁵⁷Eu, [¹⁸F]AlF, ⁵²Fe, ⁵⁹Fe, ²⁵¹Fm, ²⁵²Fm, ²⁵³Fm, ²⁵⁴Fm, ²⁵⁵Fm, ²⁵⁷Fm ⁶⁶Ga, ⁶⁷Ga, ⁶⁸Ga, ⁷²Ga, ⁷³Ga, ¹⁴⁶Gd, ¹⁴⁷Gd, ¹⁴⁹Gd, ¹⁵¹Gd, ¹⁵³Gd, ¹⁵⁹Gd, ⁶⁸Ge, ⁶⁹Ge, ⁷¹Ge, ⁷⁷Ge ¹⁷⁰Hf, ¹⁷¹Hf, ¹⁷³Hf, ¹⁷⁵Hf, ^{179m2}Hf, ^{180m}Hf, ¹⁸¹Hf, ¹⁸⁴Hf, ¹⁹²Hg, ¹⁹³Hg, ^{193m}Hg, ¹⁹⁵Hg, ^{195m}Hg, ¹⁹⁷Hg ^{197m}Hg, ²⁰³Hg, ^{160m}Ho, ¹⁶⁶Ho, ¹⁶⁷Ho, ¹²³I, ¹²⁴I, ¹²⁶I, ¹³⁰I, ¹³²I, ¹³³I, ¹³⁵I, ¹⁰⁹In, ¹¹⁰In, ¹¹¹In, ^{114m}In, ^{115m}In, ¹⁸⁴Ir, ¹⁸⁵Ir, ¹⁸⁶Ir, ¹⁸⁷Ir, ¹⁸⁸Ir, ¹⁸⁹Ir, ¹⁹⁰Ir, ^{190m2}Ir, ¹⁹²Ir, ^{193m}Ir, ¹⁹⁴Ir, ^{194m2}Ir, ^{195m}Ir, ⁴²K, ⁴³K, ⁷⁸Kr ⁷⁹Kr, ^{81m}Kr, ^{85m}Kr, ¹³²La, ¹³³La, ¹³⁵La, ¹⁴⁰La, ¹⁴¹La, ²⁶²Lr, ¹⁶⁹Lu, ¹⁷⁰Lu, ¹⁷¹Lu, ¹⁷²Lu, ^{174m}Lu, ^{176m}Lu ¹⁷⁷Lu, ^{177m}Lu, ¹⁷⁹Lu, ²⁵⁷Md, ²⁵⁸Md, ²⁶⁰Md, ²⁸Mg, ⁵²Mn, ⁹⁰Mo, ^{93m}Mo, ⁹⁹Mo, ¹⁰⁰Mo, ¹³N, ²⁴Na, ⁹⁰Nb, ^{91m}Nb ^{92m}Nb, ⁹⁵Nb, ^{95m}Nb, ⁹⁶Nb, ¹³⁸Nd, ^{139m}Nd, ¹⁴⁰Nd, ¹⁴⁷Nd, ⁵⁶Ni, ⁵⁷Ni, ⁶⁶Ni, ²³⁴Np, ²³⁶Np, ⁹⁹Mo, Np, ²³⁸Np ²³⁹Np, ¹⁵O, ¹⁸²Os, ¹⁸³Os, ^{183m}Os, ¹⁸⁵Os, ^{189m}Os, ¹⁹¹Os, ^{191m}Os, ¹⁹³Os, ³²P, ³³P, ²²⁸Pa, ²²⁹Pa, ²³⁰Pa, ²³²Pa, ²³³Pa, ²³⁴Pa, ²⁰⁰Pb, ²⁰¹Pb, ²⁰²mPb, ²⁰³Pb, ²⁰⁹Pb, ²¹²Pb, ¹⁰⁰Pd, ¹⁰¹Pd, ¹⁰³Pd, ¹⁰⁹Pd ^{111m}Pd, ¹¹²Pd, ¹⁴³Pm, ¹⁴⁸Pm, ^{148m}Pm, ¹⁴⁹Pm, ¹⁵¹Pm, ²⁰⁴Po, ²⁰⁶Po, ²⁰⁷Po, ²¹⁰Po, ¹³⁹Pr, ¹⁴²Pr, ¹⁴³Pr, ¹⁴⁵Pr, ¹⁸⁸Pt, ¹⁹¹Pt, ¹⁹¹Pt, ^{193m}Pt, ^{195m}Pt, ¹⁹⁷Pt, ²⁰⁰Pt, ²⁰²Pt, ²³⁴Pu, ²³⁷Pu, ²⁴³Pu, ²⁴⁵Pu, ²⁴⁶Pu, ²⁴⁷Pu ²²³Ra, ²²⁴Ra, ²²⁵Ra, ⁸¹Rb, ⁸²Rb, ⁸²mRb, ⁸³Rb, ⁸⁴Rb, ⁸⁶Rb, ¹⁸¹Re, ¹⁸²Re, ^{182m}Re, ¹⁸³Re, ¹⁸⁴Re, ^{184m}Re, ¹⁸⁶Re, ¹⁸⁸Re, ¹⁸⁹Re, ^{190m}Re, ⁹⁹Rh, ^{99m}Rh, ¹⁰⁰Rh, ^{101m}Rh, ¹⁰²Rh, ^{103m}Rh, ¹⁰⁵Rh, ²¹¹Rn, ²²²Rn, ⁹⁷Ru, ¹⁰³Ru, ¹⁰⁵Ru, ³⁵S, ^{118m}Sb, ¹¹⁹Sb, ¹²⁰Sb, ^{120m}Sb, ¹²²Sb, ¹²⁴Sb, ¹²⁶Sb, ¹²⁷Sb, ¹²⁸Sb, ¹²⁹Sb, ⁴³Sc, ⁴⁴Sc, ^{44m}Sc, ⁴⁶Sc, ⁴⁷Sc, ⁴⁸Sc, ⁷²Se, ⁷³Se, ⁷⁵Se, ¹⁵³Sm, ¹⁵⁶Sm, ¹¹⁰Sn, ¹¹³Sn, ^{117m}Sn ^{119m}Sn, ¹²¹Sn, ¹²³Sn, ¹²⁵Sn, ⁸²Sr, ⁸³Sr, ⁸⁵Sr, ⁸⁹Sr, ⁹¹Sr, ¹⁷³Ta, ¹⁷⁵Ta, ¹⁷⁶Ta, ¹⁷⁷Ta, ¹⁸⁰Ta, ¹⁸²Ta ¹⁸³Ta, ¹⁸⁷⁴Ta, ¹⁴⁹Tb, ¹⁵⁰Tb, ¹⁵¹Tb, ¹⁵²Tb, ¹⁵³Tb, ¹⁵⁴Tb, ^{154m}Tb, ^{154m2}Tb, ¹⁵⁵Tb, ¹⁵⁶Tb, ^{156m}Tb ^{156m2}Tb, ¹⁶⁰Tb, ¹⁶¹Tb, ⁹⁴Tc, ⁹⁵Tc, ^{95m}Tc, ⁹⁶Tc, ^{97m}Tc, ^{99m}Tc, ¹¹⁸Te, ¹¹⁹Te, ^{119m}Te, ¹²¹Te, ^{121m}Te, ^{123m}Te, ^{125m}Te, ¹²⁷Te, ^{127m}Te, ^{129m}Te, ^{131m}Te, ¹³²Te, ²²⁷Th, ²²⁹Th, ²³¹Th, ²³⁴Th, ⁴⁵Ti, ¹⁹⁸Tl, ¹⁹⁹Tl, ²⁰⁰Tl, ²⁰¹Tl, ²⁰²Tl, ²⁰⁴Tl, ¹⁶⁵Tm, ¹⁶⁶Tm, ¹⁶⁷Tm, ¹⁶⁸Tm, ¹⁷⁰Tm, ¹⁷²Tm, ¹⁷³Tm, ²³⁰U, ²³¹U, ²³⁷U, ²⁴⁰U, ⁴⁸V, ¹⁷⁸W, ¹⁸¹W, ¹⁸⁵W, ¹⁸⁷W, ¹⁸⁸W, ¹²²Xe, ¹²⁵Xe, ¹²⁷Xe, ^{129m}Xe, ^{131m}Xe, ¹³³Xe, ^{133m}Xe, ¹³⁵Xe, ^{85m}Y, ⁸⁶Y, ⁸⁷Y, ^{87m}Y, ⁸⁸Y, ⁹⁰Y, ^{90m}Y, ⁹¹Y, ⁹²Y, ⁹³Y, ₁₆₆Yb, ¹⁶⁹Yb, ¹⁷⁵Yb, ⁶²Zn, ⁶⁵Zn, ^{69m}Zn, ^{71m}Zn, ⁷²Zn, ⁸⁶Zr, ⁸⁸Zr, ⁸⁹Zr, ⁹⁵Zr, and ⁹⁷Zr. The skilled person would understand that any other radionuclide known in the art can also be used in the conjugate of the present application.

In some embodiments, the targeting ligand is a receptor specific moiety. In some embodiments, the targeting ligand is selective of specific for a cell surface molecule expressed on the surface of a target cell. In some embodiments, the targeting ligand is selective or specific for the targeted organ or tissue. In some embodiments, the targeting ligand of the present application directing the conjugate of the present application to a targeted tissue, organ, receptor or other biologically expressed composition.

Examples of targeting ligands include, without limiting, proteins, peptides and peptidomimetics such as arginine-glycine-aspartic acid (RGD)-containing peptides and peptidomimetics, amino-acid sequences, antibodies or fragments thereof such as Fab fragments and antigen binding sites of antibodies, single-chain variable fragments of antibodies, nucleotide sequences, DNA sequences, RNA sequences, peptide nucleic acid (PNA) sequences, carbohydrates, and steroids. In some embodiments, the targeting ligand is selected from an oligonucleotide, an oligopeptide, a protein, a peptide and a small molecule compound.

Examples of targeting ligands include, without limiting, gemtuzumab, inotuzumab, trastuzumab (Herceptin), HD37, M195, LMB2, lym1, 8106, HMFG1, CC49, rituximab, epratuzumab, lorvotuzumab, 2C3, imgn388, SAR3419, BilB062, brentixumab, glembatumumab, SGN-75, PSMA ADC, ASG-5ME or mdx-1203. In some embodiments, the targeting antibody is a variant of gemtuzumab, inotuzumab, trastuzumab (Herceptin), HD37, M195, LMB2, lym1, 8106, HMFG1, CC49, rituximab, epratuzumab, lorvotuzumab, 2C3, imgn388, SAR3419, BilB062, brentixumab, glembatumumab, SGN-75, PSMA ADC, ASG-5ME, mdx-1203, TATE, TOC, PSMA, bombesin derivatives (e.g. RM2, BBN and the like), FAPI derivatives, and the like. A person skilled in the art would understand that any other targeting ligands known in the art can also be used in the conjugate of the present application.

The present application further includes a conjugate of Formula VI, or a salt and/or solvate thereof:

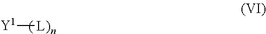

wherein
Y¹ is a complex comprising a chelating agent, wherein the chelating agent is desferrioxamine (DFO) or a compound of the following formula;

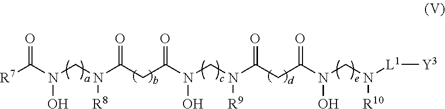

wherein
R⁷, R⁸, R⁹ and R¹⁰ are independently selected from H and C₁₋₄alky;
each a, c and e is an integer independently selected from 3, 4, 5, 6 and 7;
each b and d is an integer independently selected from 1, 2, 3 and 4;
L¹ is a linker group

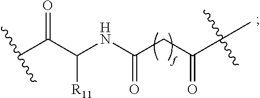

Y³ is

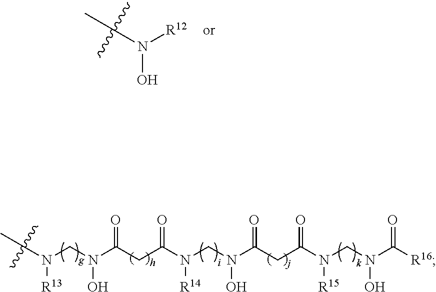

R¹¹ is selected from C₁₋₁₀alkyl and C₁₋₆alkylenephenyl, optionally substituted by one of NH₂, OH, SH, N₃, CO₂H and

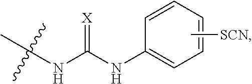

wherein X is O, S or NH;
R¹², R¹³, R¹⁴, R¹⁵ and R¹⁰ are independently selected from H and C₁₋₄alkyl;
f is an integer selected from 1, 2, 3 and 4;
each g, l and k is an integer independently selected from 3, 4, 5, 6 and 7;
each h and j is an integer independently selected from 1, 2, 3 and 4;
L is a zwitterionic (ZW) compound of the formula

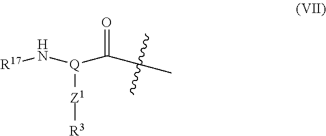

wherein
Q is selected from Q1, Q2, Q3, Q4 and Q5:

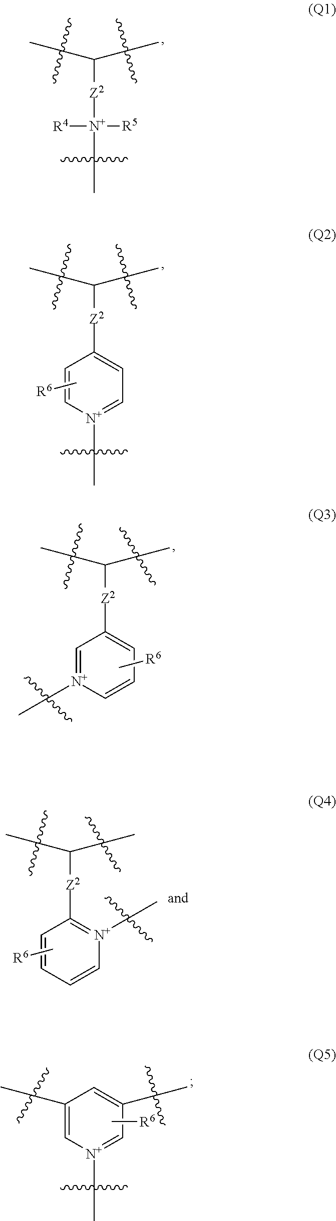

Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R³ is an anion containing group selected from sulfonate, carboxylate, sulfate and phosphate;
R¹⁷ is absent or selected from H or OH; and
n is an integer 1 to 5;
provided that wherein the chelating agent is a compound of Formula V, Y¹ is attached to L at R¹¹ of L¹ group

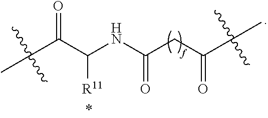

then R¹⁷ is H and the L is further optionally attached to one to three additional L groups, or the L is attached between the Y³

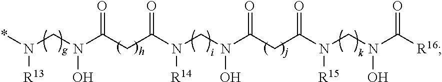

and L¹

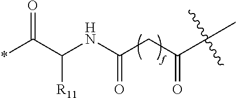

groups of Y¹ and then R¹⁷ is absent, or one L is attached at R¹¹ of L group and another L is attached between the Y³ and L¹ groups of Y¹, and each one of the L is further optionally attached to one to three additional L groups; and
wherein the chelating agent is DFO, the point of attachment of L is at the amine

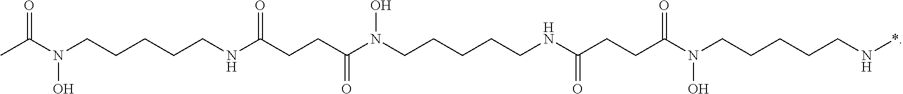

and then R¹⁷ is H, and wherein the * indicates the point of attachment.

In some embodiments, Q is Q1,

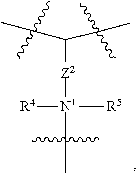

and the compound of Formula VI has the following structure:

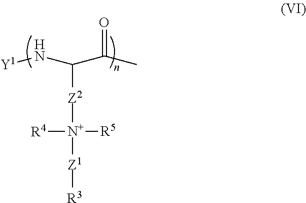

or a salt and/or solvate thereof,
wherein Y¹, R³, R⁴, R⁵, Z¹, Z² and n are as defined for Formula VI.

In some embodiments, Q is Q2,

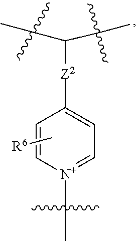

and the compound of Formula VI has the following structure:

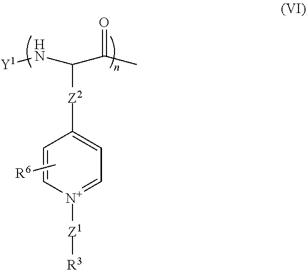

or a salt and/or solvate thereof,
wherein Y¹, R³, R⁶, Z¹, Z², and n are as defined for Formula VI.

In some embodiments, Q is Q3,

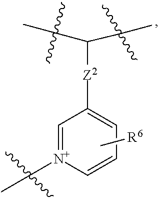

and the compound of Formula VI has the following structure:

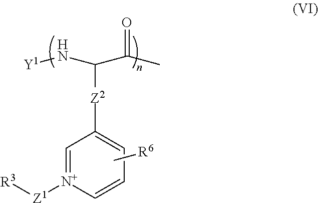

or a salt and/or solvate thereof,
wherein Y¹, R³, R⁶, Z¹, Z² and n are as defined for Formula VI.

In some embodiments, Q is Q4,

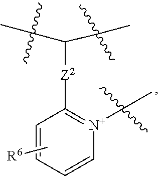

and the compound of Formula VI has the following structure:

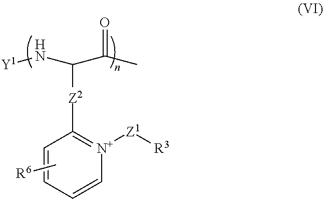

or a salt and/or solvate thereof,
wherein Y¹, R³, R⁶, Z¹, Z² and n are as defined for Formula VI.

In some embodiments, Q is Q5,

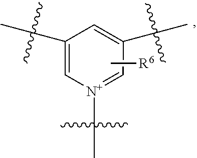

and the compound of Formula VI has the following structure:

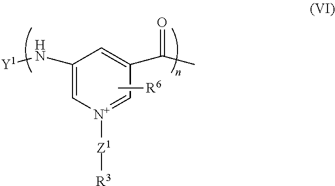

or a salt and/or solvate thereof,
wherein Y¹, R³, R⁶, Z¹ and n are as defined for Formula VI.

In some embodiments, when Q is

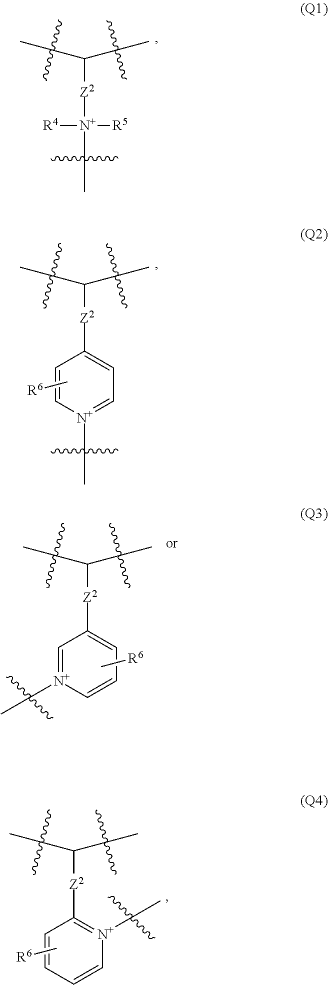

the stereochemistry at the carbon to which Z² is bonded is either R or S. In some embodiments, the stereochemistry at the carbon to which Z² is attached is R. In some embodiments, the stereochemistry at the carbon to which Z² is attached is S.

In some embodiments, R⁴ and R⁵ are independently selected from C₁₋₆alkyl, C₂₋₆alkenyl and C₂₋₆alkynyl. In some embodiments, R⁴ and R⁵ are independently selected from C₁₋₄alkyl, C₂₋₄alkenyl and C₂₋₄alkynyl. In some embodiments, R⁴ and R⁵ are independently selected from CH₂CH₂CH₃, C(CH₃)₃, CH(CH₃)CH₂CH₃ and CH₂CH(CH₃)₂, CH(CH₃)₂, CH₂CH₃ and CH₃. In some embodiments, R⁴ and R⁵ are independently selected from CH₂CH₂CH₃, CH(CH₃)₂, CH₂CH₃ and CH₃. In some embodiments, at least one of R⁴ and R⁵ is CH₃. In some embodiments, each one of R⁴ and R⁵ is CH₃.

In some embodiments, R⁶ is H.

In some embodiments, Z² is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, N and S. In some embodiments, Z² is selected from C₁₋₆alkylene, C₂₋₆alkenylene and C₂₋₆alkynelene, unsubstituted or substituted with one or more of halo or OH and/or are optionally interrupted by one to three heteromoieties independently selected from O, N and S. In some embodiments, Z² is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, N and S. In some embodiments, Z² is C₄alkylene. In some embodiments, Z² is C₁alkylene.

In some embodiments, Z¹ is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, N and S. In some embodiments, Z¹ is selected from C₁₋₆alkylene, C₂₋₆alkenylene and C₂₋₆alkynelene, unsubstituted or substituted with one or more of halo or OH and/or are optionally interrupted by one to three heteromoieties independently selected from O, N and S. In some embodiments, Z¹ is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, N and S. In some embodiments, Z¹ is C₃alkylene.

In some embodiments, R³ is sulfonate.

In some embodiments, L is selected from

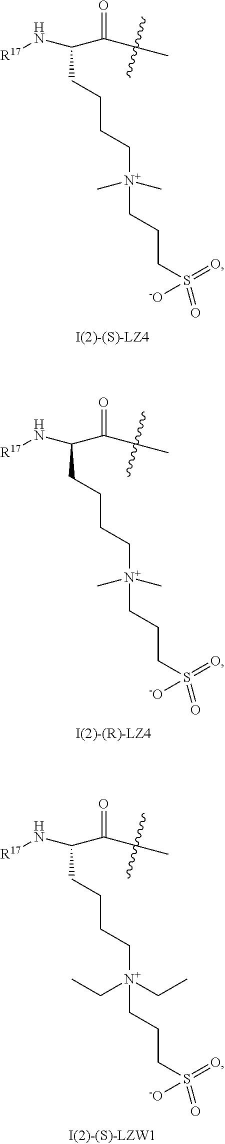

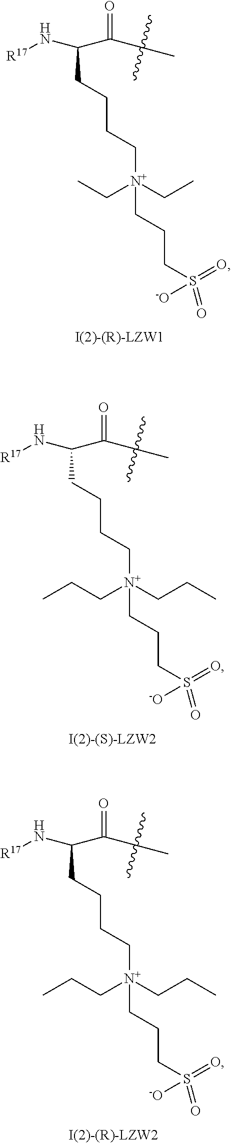

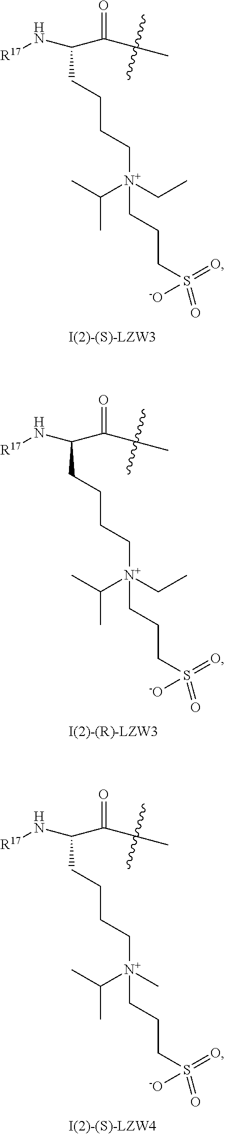

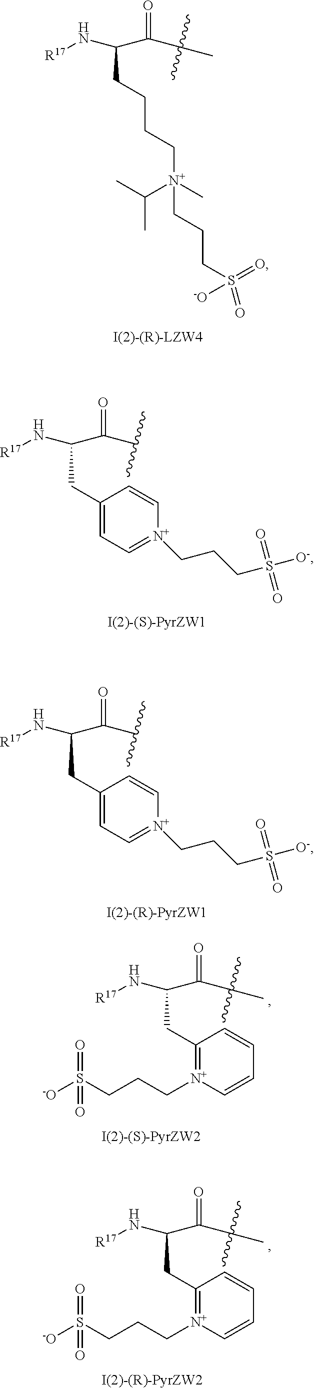

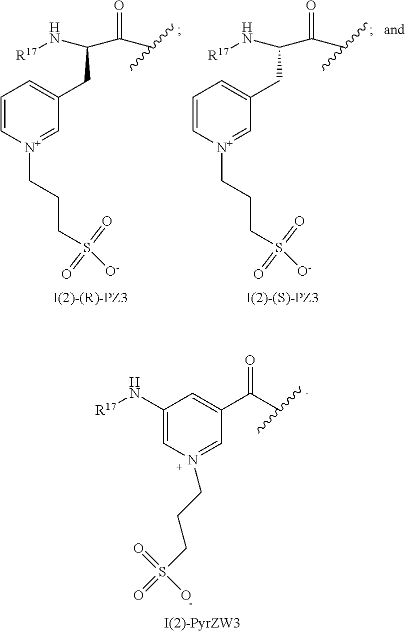

In some embodiments, L is

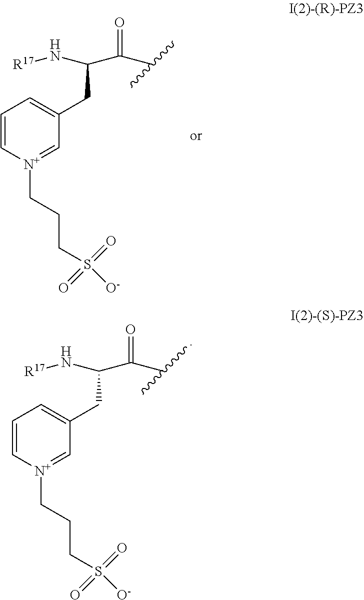

The ZW linker is a permanent zwitterionic group which possesses both a positive and a negative charge simultaneously. In some embodiments, the permanent zwitterionic group is formed at physiological pH of 5.5-7.5.

In some embodiments, the ZW linker is a natural or unnatural amino acid with a primary amine. In some embodiments, the natural amino acid is lysine. In some embodiments, the unnatural amino acid is pyridyl-alanine.

In some embodiments, Y¹ is a complex comprising a chelating agent, wherein the chelating agent is DFO.

In some embodiments, Y¹ is a complex comprising a chelating agent, wherein the chelating agent is a compound of Formula V:

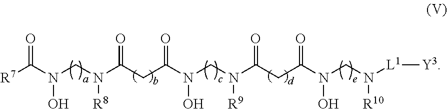

In some embodiments, when the chelating agent is a compound of Formula V, R⁷, R⁸, R⁹ and R¹⁰ are independently selected from H and C₁₋₃alkyl. In some embodiments, R⁷, R⁸, R⁹ and R¹⁰ are independently selected from H and C₁₋₂alkyl. In some embodiments, at least one of R⁷, R⁸, R⁹ and R¹⁰ is C₁₋₂alkyl. In some embodiments, R⁷ is CH₃. In some embodiments, each one of R⁸, R⁹ and R¹⁰ is H.

In some embodiments, each a, c and e is an integer independently selected from 4, 5 and 6. In some embodiments, a, c and e are different. In some embodiments, a, c and e are 5.

In some embodiments, each b and d is an integer independently selected from 1, 2 and 3. In some embodiments, b and d are different. In some embodiments, b and d are 2.

In some embodiments, f is selected from the integer 1, 2 and 3. In some embodiments, f is 2.

In some embodiments, when Y is

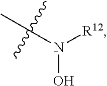

R¹² is selected from H and C₁₋₂alkyl. In some embodiments, R¹² is selected from H and CH₃. In some embodiments, R¹² is CH₃.

In some embodiments, when Y is

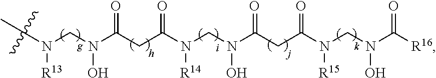

R¹³, R¹⁴, R¹⁵ and R¹⁶ are independently selected from H and C₁₋₃alkyl. In some embodiments, R¹³, R¹⁴, R¹⁵ and R¹⁶ are independently selected from H and C₁₋₂alkyl. In some embodiments, at least one of R¹³, R¹⁴, R¹⁵ and R¹⁶ is C₁₋₂alkyl. In some embodiments, R¹⁶ is CH₃. In some embodiments, each one of R¹³, R¹⁴ and R¹⁵ is H.

In some embodiments, each g, l and k is an integer independently selected from 4, 5 and 6. In some embodiments, g, l and k are different. In some embodiments, g, l and k are 5.

In some embodiments, each h and j is an integer independently selected from 1, 2 and 3. In some embodiments, h and j are different. In some embodiments, h and j are 2.

In some embodiments, a, c and e are 5 and b and d are 2 and the compound of Formula V has the following structure:

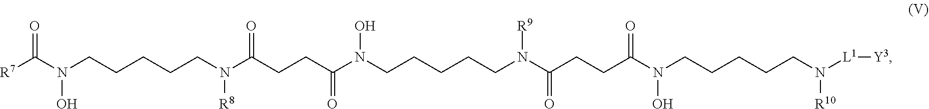

wherein R⁷, R⁸, R⁹, R¹⁰, L¹ and Y³ are as defined above.

In some embodiments, when Y³ is

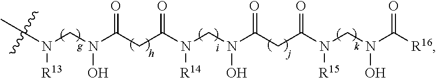

g, i and k are 5 and h and j are 2 and the compound of Formula V has the following structure:

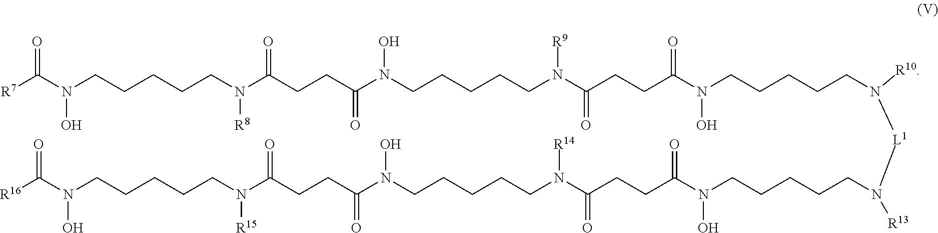

In some embodiments, the compound of Formula V is selected from

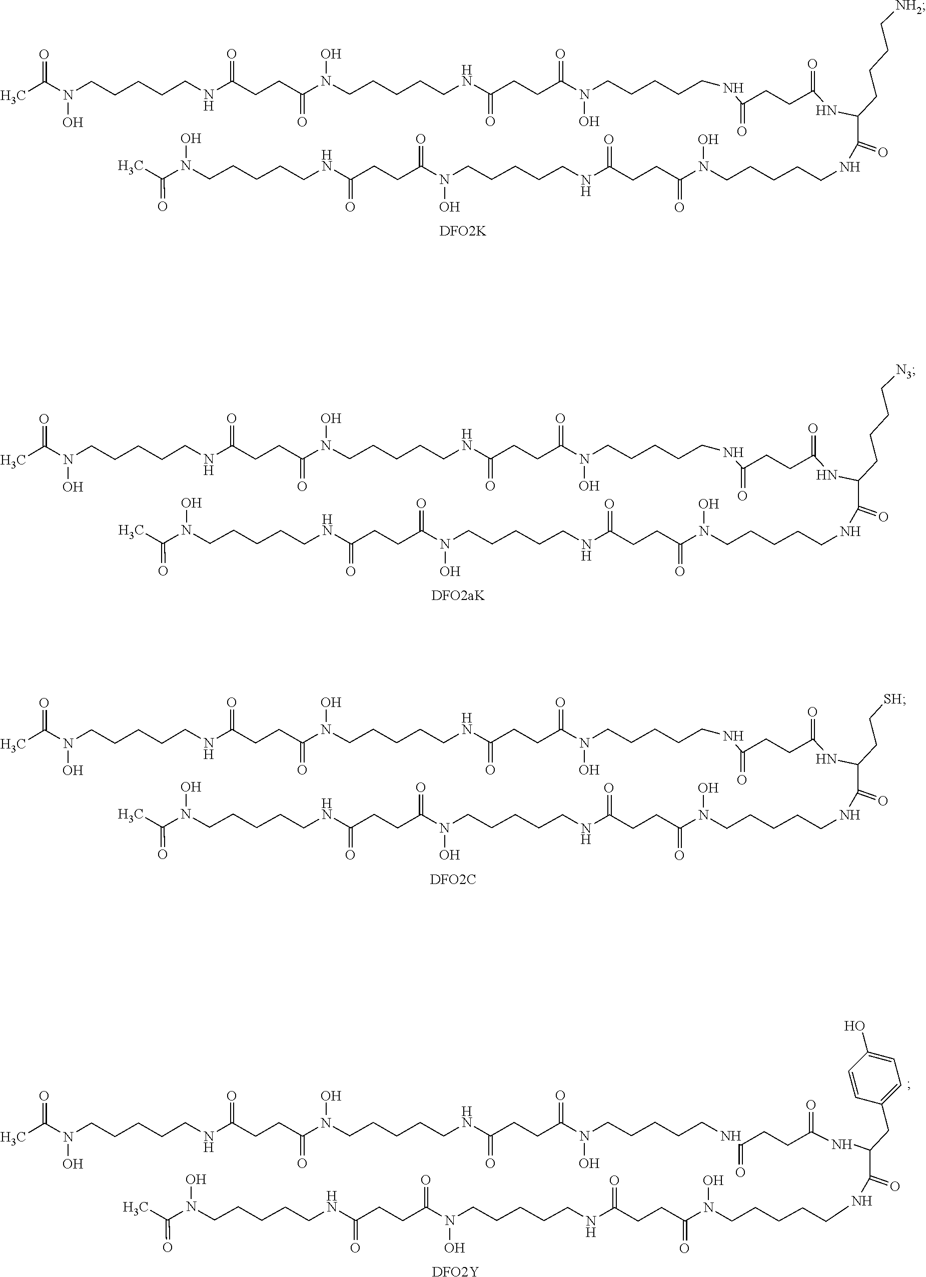

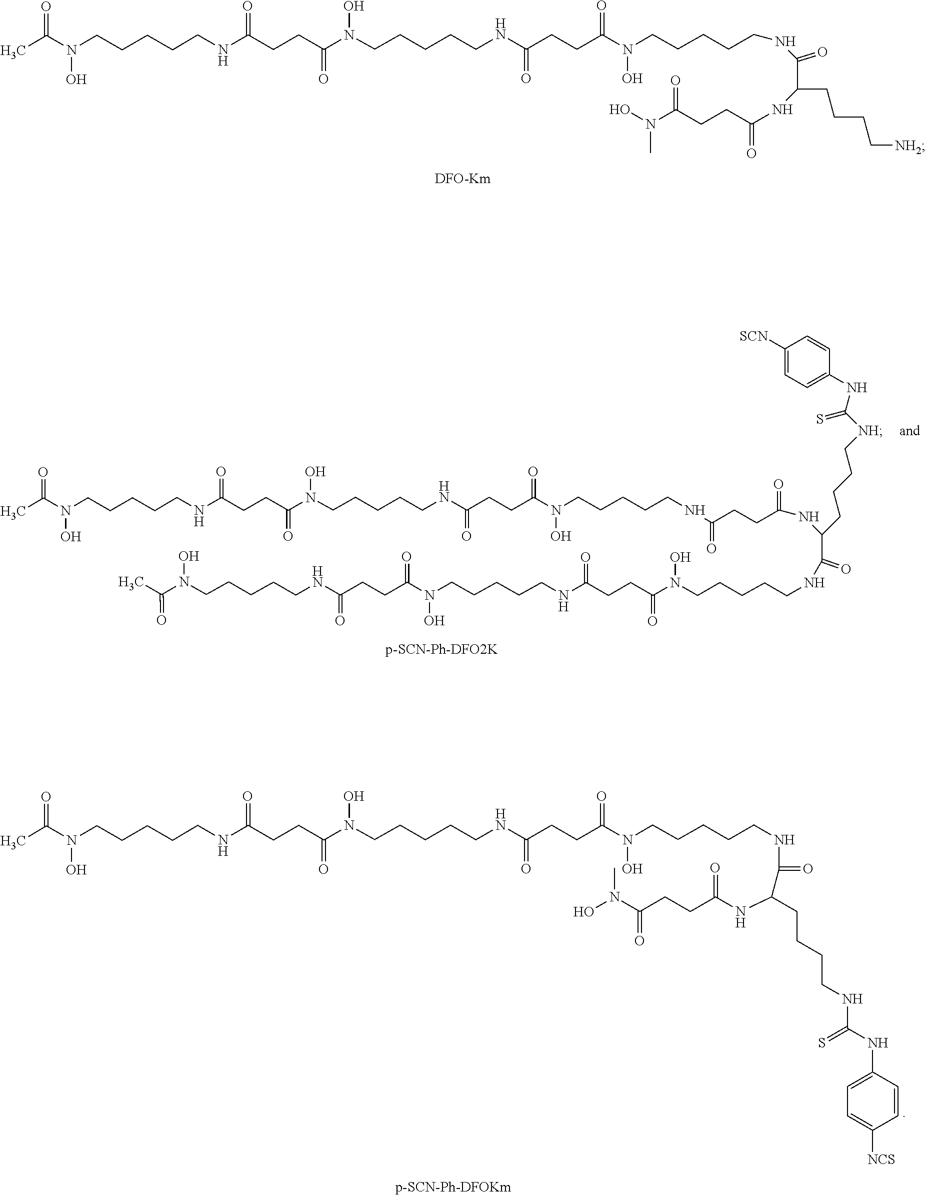

In some embodiments, n is an integer 1 to 3. In some embodiments, n is an integer 1. In some embodiments, n is an integer 2. As such, the conjugate of the present application comprises at least one ZW linker. In some embodiments, the conjugate of the present application comprises two ZW linkers.

In some embodiments, the chelating agent is a compound of Formula V and Y¹ is attached to L at R¹¹ of L¹ group

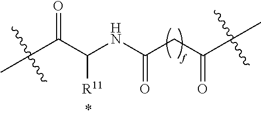

and then R¹⁷ is H. In some embodiments, the L is further optionally attached to one or two additional L groups. In some embodiments, the L is further optionally attached to one L group.

In some embodiments, the chelating agent is a compound of Formula V and L is attached between the Y³

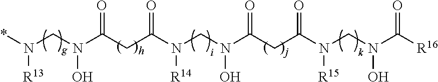

### and L¹

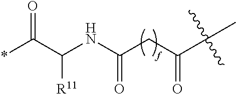

groups of Y¹ and then R¹⁷ is absent. In some embodiments, L is further optionally attached to one or two additional L groups.

In some embodiments, the chelating agent is a compound of Formula V and one L is attached at R¹¹ of L¹ group

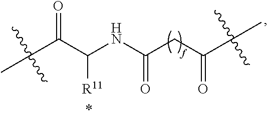

and another L is attached between the Y³

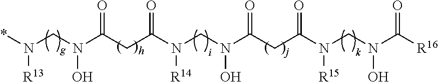

### and L¹

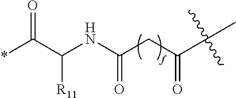

groups of Y¹. In some embodiments, L group attached at R¹¹ of L¹ group is further attached to one to three additional L groups. In some embodiments, L group attached between the Y³ and L¹ groups of Y¹ is further attached to one additional L group.

In some embodiments, when the chelating agent is DFO, the point of attachment of L is at the amine and then R¹⁷ is H

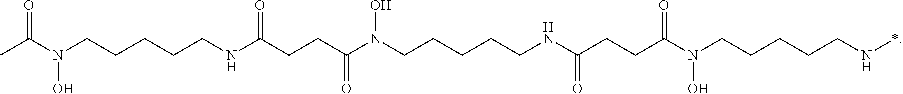

In some embodiments, the Y¹-L is selected from

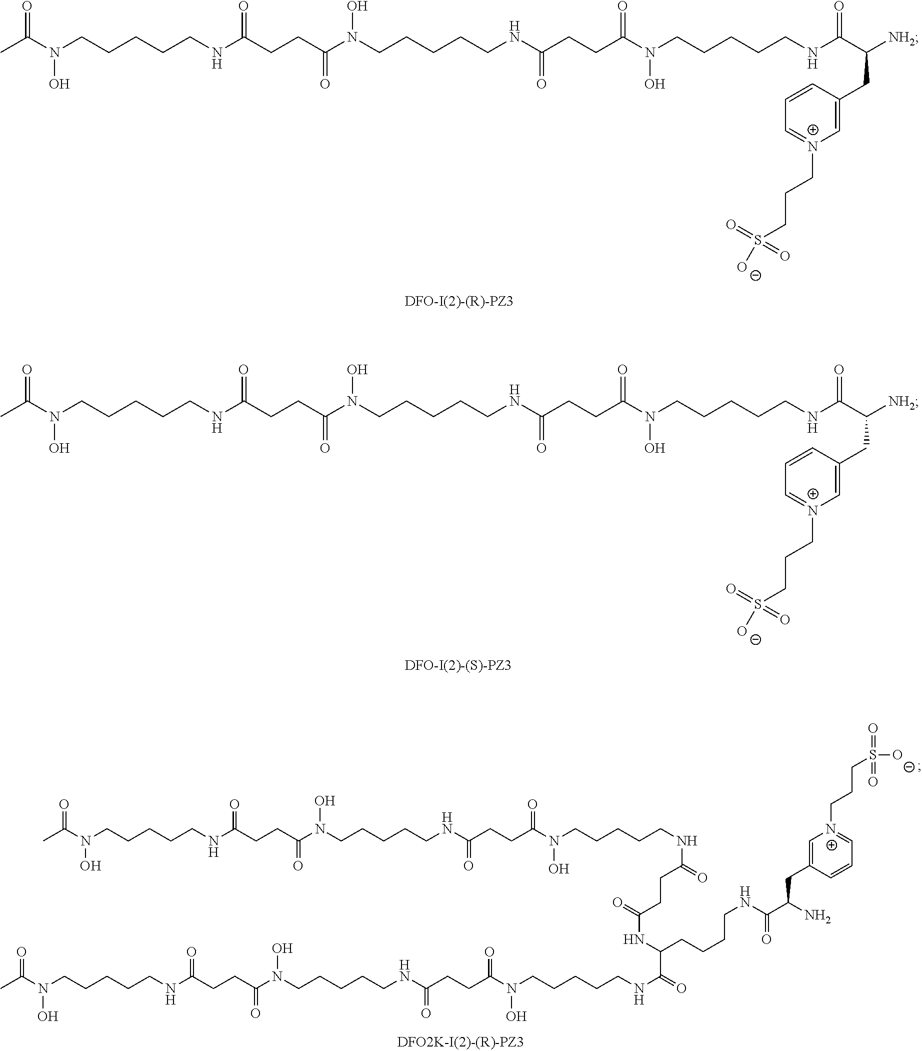 [SMILES: CC(=O)N(O)CCCCCNC(=O)CCC(=O)N(O)CCCCCNC(=O)CCC(=O)N(O)CCCCCNC(=O)[C@@H](N)Cc1ccc[n+](CCCS(=O)(=O)[O-])c1 ; CC(=O)N(O)CCCCCNC(=O)CCC(=O)N(O)CCCCCNC(=O)CCC(=O)N(O)CCCCCNC(=O)[C@H](N)Cc1ccc[n+](CCCS(=O)(=O)[O-])c1 ; CC(=O)N(O)CCCCCNC(=O)CCC(=O)N(O)CCCCCNC(=O)CCC(=O)N(O)CCCCCNC(=O)CCC(=O)NC(CCCCNC(=O)[C@H](N)Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O]

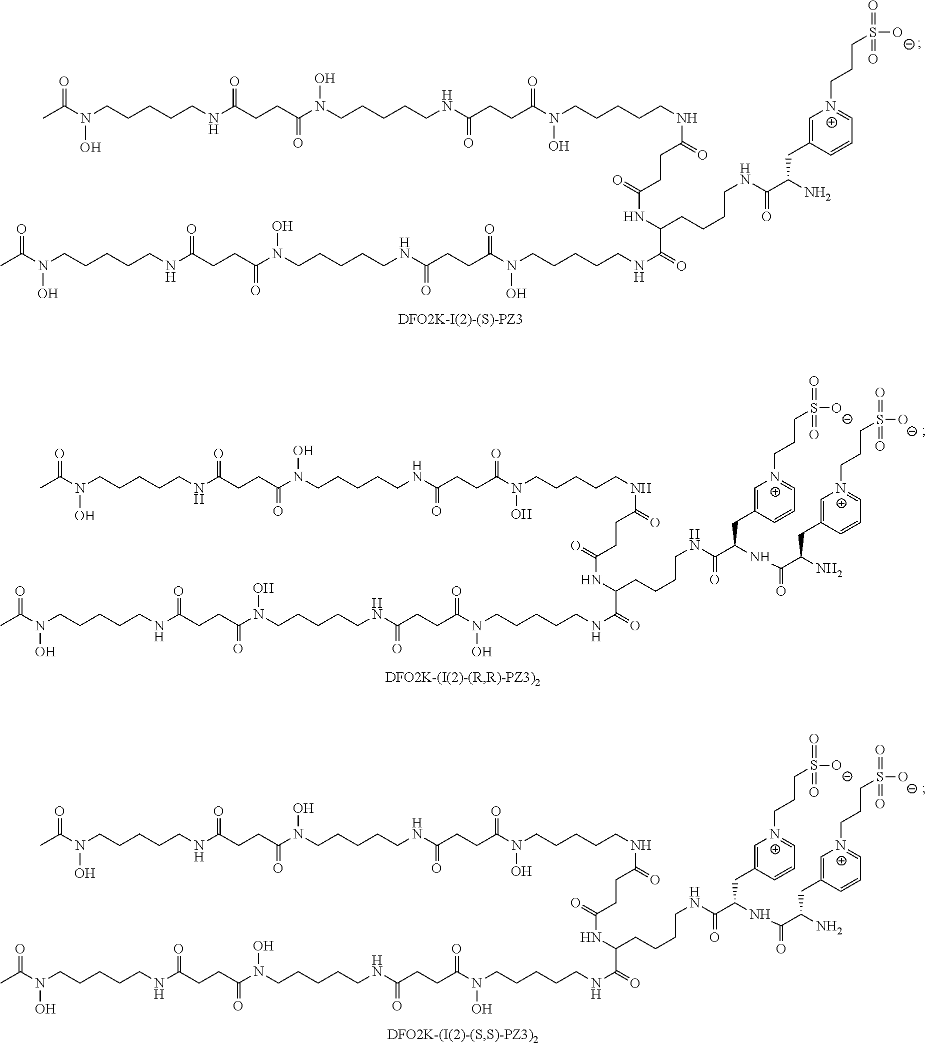

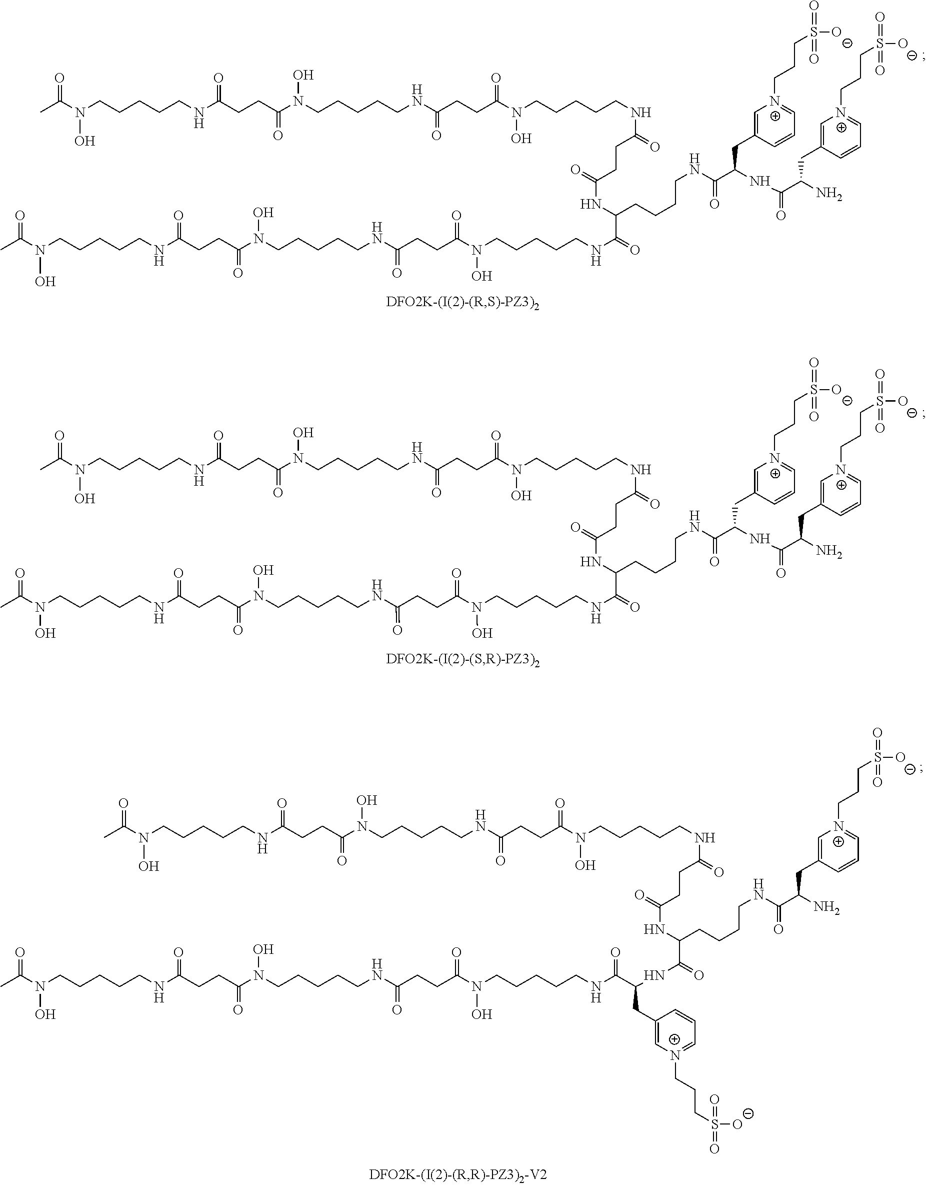

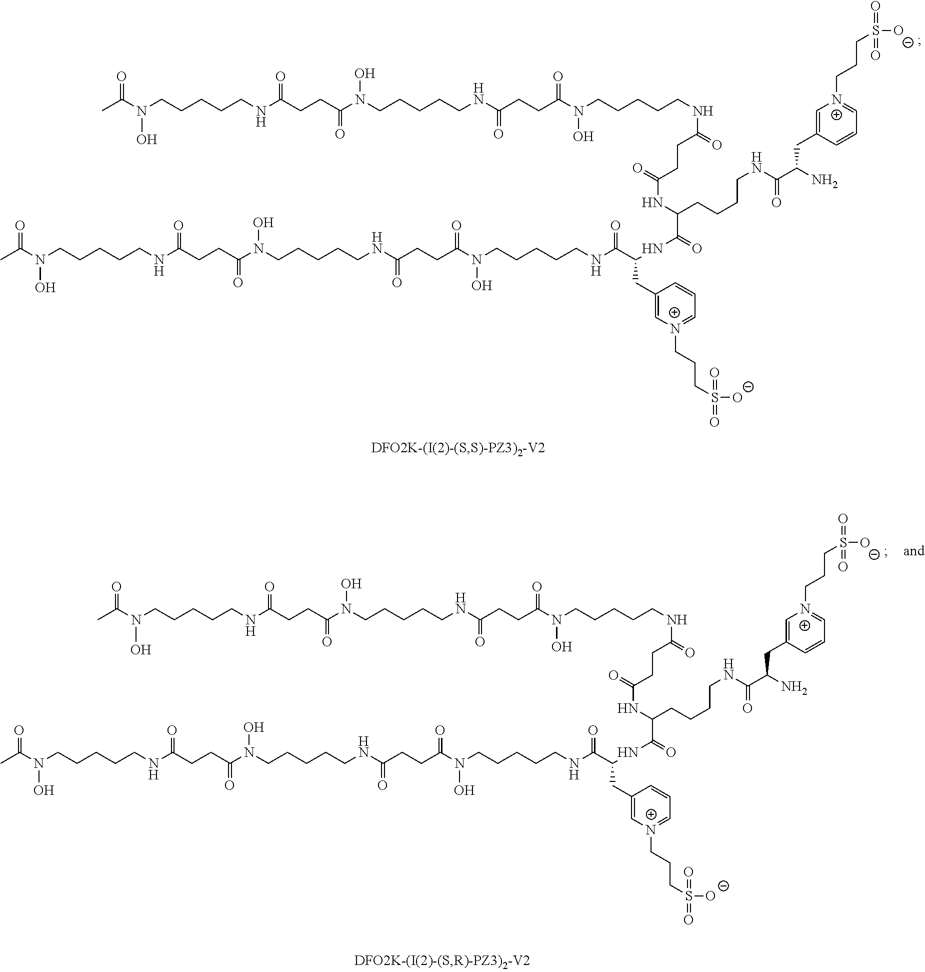 [SMILES: CC(=O)N(O)CCCCCNC(=O)CCC(=O)N(O)CCCCCNC(=O)CCC(=O)N(O)CCCCCNC(=O)CCC(=O)NC(CCCCNC(=O)[C@@H](N)Cc1ccc[n+](CCCS(=O)(=O)[O-])c1)C(=O)N[C@H](Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O ; CC(=O)N(O)CCCCCNC(=O)CCC(=O)N(O)CCCCCNC(=O)CCC(=O)N(O)CCCCCNC(=O)CCC(=O)NC(CCCCNC(=O)[C@H](N)Cc1ccc[n+](CCCS(=O)(=O)[O-])c1)C(=O)N[C@H](Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O]

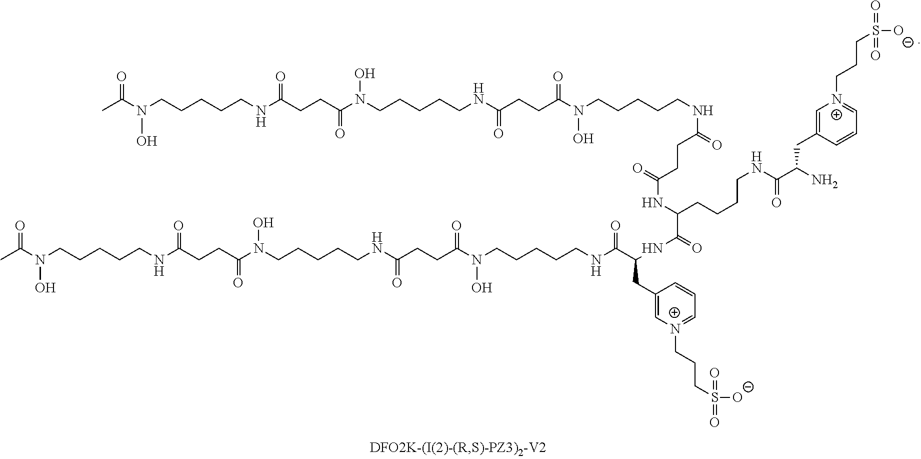 [SMILES: CC(=O)N(O)CCCCCNC(=O)CCC(=O)N(O)CCCCCNC(=O)CCC(=O)N(O)CCCCCNC(=O)CCC(=O)NC(CCCCNC(=O)[C@@H](N)Cc1ccc[n+](CCCS(=O)(=O)[O-])c1)C(=O)N[C@@H](Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O]

In some embodiments, the conjugate of Formula VI is further attached to a biomolecule conjugating group selected from NH₂, OH, SH, N₃, CO₂H, ONH₂, NHNH₂, C(O)C₁₋₄alkyl, C≡CH, SCN, C(O)NH₂, NHC(X)NH₂, C₁₋₄alkyleneN₃, NHC(O)C₁₋₄alkyleneN₃, NHC(O)C₁₋₄alkyleneONH₂, NHC(O)C₁₋₄alkyleneNHNH₂, NHC(O)OC₁₋₄alkyleneN₃, C(O)NHC₁₋₄alkylene-phenylene-tetrazine,

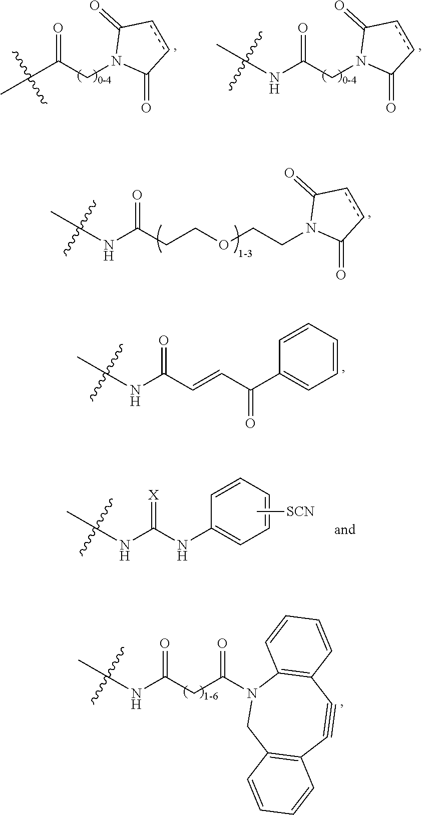

wherein [symbol] represents a single or a double bond and X is O, S or NH. A person skilled in the art would understand that other functional groups known in the art can also be used as a biomolecule conjugating group. This includes any functional group comprising, for example, a nucleophilic group, an electrophilic group, a Michael acceptor, a group that participates in a Click reaction, a group that participates in a cross-coupling reaction, a group that is activated to participate in a nucleophilic displacement reaction, and the like.

In some embodiments, the chelating agent further comprises one or more radionuclides selected from a transition metal, rare-earth metal, lanthanide, actinide and metalloid.

Radionucleotides that can be used in the present application include but are not limited to the following elements and their isotopes: ²²⁵Ac, ²²⁶Ac, ²²⁷Ac, ²²⁸Ac, ¹⁰⁵Ag, ^{106m}Ag, ^{110m}Ag, ¹¹¹Ag, ¹¹²Ag, ¹¹³Ag, ²³⁹Am, ²⁴⁰Am, ²⁴²Am, ²⁴⁴Am, ³⁷Ar, ⁷¹As, ⁷²As, ⁷³As, ⁷⁴As ⁷⁶As, ⁷⁷As, ²⁰⁹At, ²¹⁰At, ¹⁹¹Au, ¹⁹²Au, ¹⁹³Au, ¹⁹⁴Au, ¹⁹⁵Au, ¹⁹⁶Au, ^{196m2}Au, ¹⁹⁶Au, ^{198m}Au, ¹⁹⁹Au ^{200m}Au, ¹²⁸Ba, ¹³¹Ba, ^{133m}Ba, ^{135m}Ba, ¹⁴⁰Ba, ⁷Be, ²⁰³Bi, ²⁰⁴Bi, ²⁰⁵Bi, ²⁰⁶Bi, ²¹⁰Bi, ²¹²Bi, ²⁴³Bk, ²⁴⁴Bk ²⁴⁵Bk, ²⁴⁶Bk, ^{248m}Bk, ²⁵⁰Bk, ⁷⁶Br, ⁷⁷Br, ^{80m}Br, ⁸²Br, ¹¹C, ¹⁴C, ⁴⁵Ca, ⁴⁷Ca, ¹⁰⁷Cd, ¹¹⁵Cd, ¹¹⁵mCd ^{117m}Cd, ¹³²Ce, ^{133m}Ce, ¹³⁴Ce, ¹³⁵Ce, ¹³⁷Ce, ^{137m}Ce, ¹³⁹Ce, ¹⁴¹Ce, ¹⁴³Ce, ¹⁴⁴Ce, ²⁴⁶Cf, ²⁴⁷Cf, ²⁵³Cf, ²⁵⁴Cf, ²⁴⁰Cm, ²⁴¹Cm, ²⁴²Cm, ²⁵²Cm, ⁵⁵Co, ⁵⁶Co, ⁵⁷Co, ⁵⁸Co, ^{58m}Co, ⁶⁰Co, ⁴⁸Cr, ⁵¹Cr, ¹²⁷Cs, ¹²⁹Cs ¹³¹Cs, ¹³²Cs, ¹³⁶Cs, ¹³⁷Cs, ⁶¹Cu, ⁶²Cu, ⁶⁴Cu, ⁶⁷Cu, ¹⁵³Dy, ¹⁵⁵Dy, ¹⁵⁷Dy, ¹⁵⁹Dy, ¹⁶⁵Dy, ¹⁶⁶Dy, ¹⁶⁰Er ¹⁶¹Er, ¹⁶⁵Er, ¹⁶⁹Er, ¹⁷¹Er, ¹⁷²Er, ²⁵⁰Es, ²⁵¹Es, ²⁵³Es, ²⁵⁴Es, ^{254m}Es, ²⁵⁵Es, ^{256m}Es, ¹⁴⁵Eu, ¹⁴⁶Eu, ¹⁴⁷Eu ¹⁴⁸Eu, ¹⁴⁹Eu, ^{150m}Eu, ¹⁵²Eu, ¹⁵⁶Eu, ¹⁵⁷Eu, [¹⁸F]AlF, ⁵²Fe, ⁵⁹Fe, ²⁵¹Fm, ²⁵²Fm, ²⁵³Fm, ²⁵⁴Fm, ²⁵⁵Fm, ²⁵⁷Fm ⁶⁶Ga, ⁶⁷Ga, ⁶⁸Ga, ⁷²Ga, ⁷³Ga, ¹⁴⁶Gd, ¹⁴⁷Gd, ¹⁴⁹Gd, ¹⁵¹Gd, ¹⁵³Gd, ¹⁵⁹Gd, ⁶⁸Ge, ⁶⁹Ge, ⁷¹Ge, ⁷⁷Ge ¹⁷⁰Hf, ¹⁷¹Hf, ¹⁷³Hf, ¹⁷⁵Hf, ^{179m2}Hf, ^{180m}Hf, ¹⁸¹Hf, ¹⁸⁴Hf, ¹⁹²Hg, ¹⁹³Hg, ^{193m}Hg, ¹⁹⁵Hg, ^{195m}Hg, ¹⁹⁷Hg ^{197m}Hg, ²⁰³Hg, ^{160m}Ho, ¹⁶⁶Ho, ¹⁶⁷Ho, ¹²³I, ¹²⁴I, ¹²⁶I, ¹³⁰I, ¹³²I, ¹³³I, ¹³⁵I, ¹⁰⁹In, ¹¹⁰In, ¹¹¹In, ^{114m}In, ^{115m}In, ¹⁸⁴Ir, ¹⁸⁵Ir, ¹⁸⁶Ir, ¹⁸⁷Ir, ¹⁸⁸Ir, ¹⁸⁹Ir, ¹⁹⁰Ir, ^{190m2}Ir, ¹⁹²Ir, ^{193m}Ir, ¹⁹⁴Ir, ^{194m2}Ir, ^{195m}Ir, ⁴²K, ⁴³K, ⁷⁸Kr ⁷⁹Kr, ^{81m}Kr, ^{85m}Kr, ¹³²La, ¹³³La, ¹³⁵La, ¹⁴⁰La, ¹⁴¹La, ²⁶²Lr, ¹⁶⁹Lu, ¹⁷⁰Lu, ¹⁷¹Lu, ¹⁷²Lu, ^{174m}Lu, ^{176m}Lu ¹⁷⁷Lu, ^{177m}Lu, ¹⁷⁹Lu, ²⁵⁷Md, ²⁵⁸Md, ²⁶⁰Md, ²⁸Mg, ⁵²Mn, ⁹⁰Mo, ^{93m}Mo, ⁹⁹Mo, ¹⁰⁰Mo, ¹³N, ²⁴Na, ⁹⁰Nb, ^{91m}Nb ^{92m}Nb, ⁹⁵Nb, ^{95m}Nb, ⁹⁶Nb, ¹³⁸Nd, ^{139m}Nd, ¹⁴⁰Nd, ¹⁴⁷Nd, ⁵⁶Ni, ⁵⁷Ni, ⁶⁶Ni, ²³⁴Np, ²³⁶Np, ⁹⁹Mo, Np, ²³⁸Np ²³⁹Np, ¹⁵O, ¹⁸²Os, ¹⁸³Os, ^{183m}Os, ¹⁸⁵Os, ^{189m}Os, ¹⁹¹Os, ^{191m}Os, ¹⁹³Os, ³²P, ³³P, ²²⁸Pa, ²²⁹Pa, ²³⁰Pa, ²³²Pa, ²³³Pa, ²³⁴Pa, ²⁰⁰Pb, ²⁰¹Pb, ²⁰²mPb, ²⁰³Pb, ²⁰⁹Pb, ²¹²Pb, ¹⁰⁰Pd, ¹⁰¹Pd, ¹⁰³Pd, ¹⁰⁹Pd ^{111m}Pd, ¹¹²Pd, ¹⁴³Pm, ¹⁴⁸Pm, ^{148m}Pm, ¹⁴⁹Pm, ¹⁵¹Pm, ²⁰⁴Po, ²⁰⁶Po, ²⁰⁷Po, ²¹⁰Po, ¹³⁹Pr, ¹⁴²Pr, ¹⁴³Pr, ¹⁴⁵Pr, ¹⁸⁸Pt, ¹⁹¹Pt, ¹⁹¹Pt, ^{193m}Pt, ^{195m}Pt, ¹⁹⁷Pt, ²⁰⁰Pt, ²⁰²Pt, ²³⁴Pu, ²³⁷Pu, ²⁴³Pu, ²⁴⁵Pu, ²⁴⁶Pu, ²⁴⁷Pu ²²³Ra, ²²⁴Ra, ²²⁵Ra, ⁸¹Rb, ⁸²Rb, ⁸²mRb, ⁸³Rb, ⁸⁴Rb, ⁸⁶Rb, ¹⁸¹Re, ¹⁸²Re, ^{182m}Re, ¹⁸³Re, ¹⁸⁴Re, ^{184m}Re, ¹⁸⁶Re, ¹⁸⁸Re, ¹⁸⁹Re, ^{190m}Re, ⁹⁹Rh, ^{99m}Rh, ¹⁰⁰Rh, ^{101m}Rh, ¹⁰²Rh, ^{103m}Rh, ¹⁰⁵Rh, ²¹¹Rn, ²²²Rn, ⁹⁷Ru, ¹⁰³Ru, ¹⁰⁵Ru, ³⁵S, ^{118m}Sb, ¹¹⁹Sb, ¹²⁰Sb, ^{120m}Sb, ¹²²Sb, ¹²⁴Sb, ¹²⁶Sb, ¹²⁷Sb, ¹²⁸Sb, ¹²⁹Sb, ⁴³Sc, ⁴⁴Sc, ^{44m}Sc, ⁴⁶Sc, ⁴⁷Sc, ⁴⁸Sc, ⁷²Se, ⁷³Se, ⁷⁵Se, ¹⁵³Sm, ¹⁵⁶Sm, ¹¹⁰Sn, ¹¹³Sn, ^{117m}Sn ^{119m}Sn, ¹²¹Sn, ¹²³Sn, ¹²⁵Sn, ⁸²Sr, ⁸³Sr, ⁸⁵Sr, ⁸⁹Sr, ⁹¹Sr, ¹⁷³Ta, ¹⁷⁵Ta, ¹⁷⁶Ta, ¹⁷⁷Ta, ¹⁸⁰Ta, ¹⁸²Ta ¹⁸³Ta, ¹⁸⁷⁴Ta, ¹⁴⁹Tb, ¹⁵⁰Tb, ¹⁵¹Tb, ¹⁵²Tb, ¹⁵³Tb, ¹⁵⁴Tb, ^{154m}Tb, ^{154m2}Tb, ¹⁵⁵Tb, ¹⁵⁶Tb, ^{156m}Tb ^{156m2}Tb, ¹⁶⁰Tb, ¹⁶¹Tb, ⁹⁴Tc, ⁹⁵Tc, ^{95m}Tc, ⁹⁶Tc, ^{97m}Tc, ^{99m}Tc, ¹¹⁸Te, ¹¹⁹Te, ^{119m}Te, ¹²¹Te, ^{121m}Te, ^{123m}Te, ^{125m}Te, ¹²⁷Te, ^{127m}Te, ^{129m}Te, ^{131m}Te, ¹³²Te, ²²⁷Th, ²²⁹Th, ²³¹Th, ²³⁴Th, ⁴⁵Ti, ¹⁹⁸Tl, ¹⁹⁹Tl, ²⁰⁰Tl, ²⁰¹Tl, ²⁰²Tl, ²⁰⁴Tl, ¹⁶⁵Tm, ¹⁶⁶Tm, ¹⁶⁷Tm, ¹⁶⁸Tm, ¹⁷⁰Tm, ¹⁷²Tm, ¹⁷³Tm, ²³⁰U, ²³¹U, ²³⁷U, ²⁴⁰U, ⁴⁸V, ¹⁷⁸W, ¹⁸¹W, ¹⁸⁵W, ¹⁸⁷W, ¹⁸⁸W, ¹²²Xe, ¹²⁵Xe, ¹²⁷Xe, ^{129m}Xe, ^{131m}Xe, ¹³³Xe, ^{133m}Xe, ¹³⁵Xe, ^{85m}Y, ⁸⁶Y, ⁸⁷Y, ^{87m}Y, ⁸⁸Y, ⁹⁰Y, ^{90m}Y, ⁹¹Y, ⁹²Y, ⁹³Y, ₁₆₆Yb, ¹⁶⁹Yb, ¹⁷⁵Yb, ⁶²Zn, ⁶⁵Zn, ^{69m}Zn, ^{71m}Zn, ⁷²Zn, ⁸⁶Zr, ⁸⁸Zr, ⁸⁹Zr, ⁹⁵Zr, and ⁹⁷Zr and the like. The skilled person would understand that any other radionuclide known in the art can also be used in the conjugate of the present application.

In some embodiments, the biomolecule conjugating group is further attached to a targeting ligand.

In some embodiments, the targeting ligand is a receptor specific moiety. In some embodiments, the targeting ligand is selective of specific for a cell surface molecule expressed on the surface of a target cell. In some embodiments, the targeting ligand is selective or specific for the targeted organ or tissue. In some embodiments, the targeting ligand of the present application directing the conjugate of the present application to a targeted tissue, organ, receptor or other biologically expressed composition.

Examples of targeting ligands include, without limiting, proteins, peptides and peptidomimetics such as arginine-glycine-aspartic acid (RGD)-containing peptides and peptidomimetics, amino-acid sequences, antibodies or fragments thereof such as Fab fragments and antigen binding sites of antibodies, single-chain variable fragments of antibodies, nucleotide sequences, DNA sequences, RNA sequences, peptide nucleic acid (PNA) sequences, carbohydrates, and steroids. In some embodiments, the targeting ligand is selected from an oligonucleotide, an oligopeptide, a protein, a peptide and a small molecule compound.

Examples of targeting ligands include, without limiting, gemtuzumab, inotuzumab, trastuzumab (Herceptin), HD37, M195, LMB2, lym1, 8106, HMFG1, CC49, rituximab, epratuzumab, lorvotuzumab, 2C3, imgn388, SAR3419, BilB062, brentixumab, glembatumumab, SGN-75, PSMA ADC, ASG-5ME or mdx-1203. In some embodiments, the targeting antibody is a variant of gemtuzumab, inotuzumab, trastuzumab (Herceptin), HD37, M195, LMB2, lym1, 8106, HMFG1, CC49, rituximab, epratuzumab, lorvotuzumab, 2C3, imgn388, SAR3419, BilB062, brentixumab, glembatumumab, SGN-75, PSMA ADC, ASG-5ME, mdx-1203, TATE, TOC, PSMA, bombesin derivatives (e.g. RM2, BBN and the like), FAPI derivatives, and the like. A person skilled in the art would understand that any other targeting ligands known in the art can also be used in the conjugate of the present application.

In some embodiments, the salt and/or solvate of the compound and/or the conjugate of the application is a pharmaceutically acceptable salt and/or solvate. In some embodiments the pharmaceutically acceptable salt is an acid addition salt or a base addition salt. The selection of a suitable salt may be made by a person skilled in the art (see, for example, S. M. Berge, et al., “Pharmaceutical Salts,” J. Pharm. Sci. 1977, 66, 1-19).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In an embodiment, the mono- or di-acid salts are formed, and such salts exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. The selection of the appropriate salt may be useful, for example, so that an ester functionality, if any, elsewhere in a compound is not hydrolyzed. The selection criteria for the appropriate salt will be known to one skilled in the art.

Solvates of the compound and/or the conjugate of the application include, for example, those made with solvents that are pharmaceutically acceptable. Examples of such solvents include water (resulting solvate is called a hydrate) and ethanol and the like. Suitable solvents are physiologically tolerable at the dosage administered.

In some embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds of the present application may also exist in different tautomeric forms and it is intended that any tautomeric forms which the compounds form, as well as mixtures thereof, are included within the scope of the present application.

The compounds of the present application may further exist in varying polymorphic forms and it is contemplated that any polymorphs, or mixtures thereof, which form are included within the scope of the present application.

The present application also includes a composition comprising one or more conjugates of the application and a carrier. In some embodiments, the composition is a pharmaceutical composition. Therefore, the present application also includes a pharmaceutical composition comprising one or more conjugates of the application and a pharmaceutically acceptable carrier.

In some embodiments, the composition of the application is for medical diagnosis, therefore the present application also includes a diagnostic composition comprising one or more conjugates of the application and a carrier. In some embodiments, when the medical diagnosis is performed in vitro, the carrier needs not be pharmaceutically acceptable. Thus, if the medical diagnosis comprises administering the diagnostic composition to a subject then the carrier is a pharmaceutically acceptable carrier.

In some embodiments, the composition of the application is for theranostic applications, therefore the present application also includes a theranostic composition comprising one or more conjugates of the application and a pharmaceutically acceptable carrier.

In some embodiment, the conjugate of the present application comprises at least one ZW linker.

In some embodiments, the conjugate of the present application comprises two ZW linkers.

In some embodiments, the ZW linker is a natural or unnatural amino acid with a primary amine. In some embodiments, the natural amino acid is lysine. In some embodiments, the unnatural amino acid is pyridyl-alanine.

In some embodiments, the conjugate of the present application is a radiopharmaceutical. In some embodiments, the radiopharmaceutical is a protein-based radiopharmaceutical. In some embodiments, the radiopharmaceutical is a peptide-based radiopharmaceutical.

In some embodiments, the composition of the application is for medical diagnosis. In some embodiments, the composition of the application is for radionuclide therapy. In some embodiments, the composition of the application is for theranostic applications.

In some embodiments, the pharmaceutical acceptable carrier is a solvent or solution. In some embodiments, the pharmaceutical compositions comprise, for example, one or more of water, buffers (for example, neutral buffered saline, phosphate buffered saline, citrates and/or acetates), ethanol, oil, carbohydrates (for example, glucose, fructose, mannose, sucrose and/or mannitol), proteins, polypeptides and/or amino acids such as glycine, antioxidants (e.g. sodium bisulfite), tonicity adjusting agents (such as potassium and calcium chloride), chelating agents such as EDTA and/or glutathione, vitamins and/or preservatives.

The compounds and/or conjugates of the application are administered to a subject in a variety of forms depending on the selected route of administration, as will be understood by those skilled in the art. For example, a compound and/or conjugate of the application is administered by oral, inhalation, parenteral, buccal, sublingual, nasal, rectal, vaginal, patch, pump, minipump, topical or transdermal administration and the pharmaceutical compositions formulated accordingly. Conventional procedures and ingredients for the selection and preparation of suitable compositions are described, for example, in Remington's Pharmaceutical Sciences (2000—20th edition) and in The United States Pharmacopeia: The National Formulary (USP 24 NF19) published in 1999.

In some embodiments, the pharmaceutical composition is formulated for parenteral administration, including subcutaneous, intradermal, intravascular (for example, intravenous), intramuscular, spinal, intracranial, intrathecal, intraocular, periocular, intraorbital, intrasynovial or intraperitoneal injection, as well as any similar injection or infusion technique. Parenteral administration may be by continuous infusion over a selected period of time.

In some embodiments, a compound and/or conjugate of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound and/or conjugate is incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers or diluents include lactose and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds and/or conjugates of the application and use the lyophilizates obtained, for example, for the preparation of products for injection.

In some embodiments, the compound and/or the conjugate of the application is administered parenterally and for example, solutions of a compound and/or conjugate of the application are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. For parenteral administration, sterile solutions of the compounds and/or conjugates of the application are usually prepared, and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, the compound and/or the conjugate of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form is sterile and is fluid to the extent that easy syringability exists. Alternatively, the compounds and/or conjugates of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds and/or conjugates of the application are conveniently delivered in the form of a solution, dry powder formulation or suspension from a pump spray container that is squeezed or pumped by the subject or as an aerosol spray presentation from a pressurized container or a nebulizer. Aerosol formulations typically comprise a solution or fine suspension of the compound and/or conjugate in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges, and pastilles, wherein a compound and/or conjugate of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds and/or conjugates of the application are useful for vaginal, urethral and rectal administrations. Such suppositories will generally be constructed of a mixture of substances that is solid at room temperature but melts at body temperature. The substances commonly used to create such vehicles include but are not limited to Theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments a compound and/or conjugate of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound and/or conjugate of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In some embodiments, compounds and/or conjugates of the application may be coupled with viral, non-viral or other vectors. Viral vectors may include retrovirus, lentivirus, adenovirus, herpesvirus, poxvirus, alphavirus, vaccinia virus or adeno-associated viruses. Non-viral vectors may include nanoparticles, cationic lipids, cationic polymers, metallic nanoparticles, nanorods, liposomes, micelles, microbubbles, cell-penetrating peptides, or lipospheres. Nanoparticles may include silica, lipid, carbohydrate, or other pharmaceutically acceptable polymers.

A compound and/or conjugate of the application including pharmaceutically acceptable salts and/or solvates thereof is suitably used on their own but will generally be administered in the form of a pharmaceutical composition in which the one or more compounds and/or conjugates of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

The present application also includes kits. Any of the components disclosed herein may be combined in the form of a kit. In some embodiments, the kit comprises one or more compounds of the application, one or more conjugates of the application and/or one or more compositions of the application. In some embodiments, the kit further comprises instructions for use in a method or use of the application.

In some embodiments, the kit comprises a container or package and a label or package insert on or associated with the container. In some embodiments, the container or package will include at least one bottle, vial, syringe, blister pack and/or other container. The containers may be formed from a variety of materials such as glass or plastic. In some the containers hold a composition of the application which comprises an amount of one or more compounds and/or conjugates of the application that are effective for treating and/or imaging a condition. In some embodiments, the containers have a sterile access port (for example the containers may be an intravenous solution bag or a vial having a stopper pierceable by a hypodermic injection needle). In some embodiments, the label or package insert indicates that the compound, conjugate and/or composition of the application is used for treating and/or imaging a condition of choice.

Diagnostic and/or Therapeutic Methods and Uses of the Application

The present application includes a method of diagnosing and/or monitoring one or more diseases, disorders or conditions comprising administering an effective amount of one or more conjugates of the application to a subject in need thereof and performing a medical diagnostic method on the subject.

The present application also includes a use of one or more conjugates of the application in a medical diagnostic method or a use of one or more conjugates of the application for preparation of a composition for use in a medical diagnostic method.

In some embodiments, the medical diagnostic method is performed using single photon emission computed tomography (SPECT, nuclear), positron emission tomography (PET, nuclear), magnetic resonance imaging (MRI, non-nuclear), optical imaging (visible and near-infrared dyes—non-nuclear) or a combination thereof. Examples of combinations of multiple modalities are PET/CT, PET/optical/CT, PET/MRI and the like. Methods of performing such imaging techniques are well known to those skilled in the art.

In some embodiments, the medical diagnostic method is a peptide-targeted imaging.

The present application also includes a method of treatment of one or more diseases, disorders or conditions, comprising administering an effective amount of one or more conjugates of the application to a subject in need thereof.

The present application also includes a use of one or more conjugates of the application for treatment of one or more diseases, disorders or conditions or a use of one or more conjugates of the application for preparation of a medicament for treatment of one or more diseases, disorders or conditions.

The present application also includes a method of radionuclide therapy comprising administering an effective amount of one or more conjugates of the application to a subject in need thereof.

The present application also includes a use of one or more conjugates of the application for radionuclide therapy or a use of one or more conjugates of the application for preparation of a medicament for radionuclide therapy.

In some embodiments, the radionuclide therapy is a peptide-receptor radionuclide-therapy (PRRT).

The present application also includes a method of theranostic treatment comprising administering an effective amount of one or more conjugates of the application to a subject in need thereof and performing a medical diagnostic method and radionuclide therapy on the subject.

In some embodiments, the treatment and/or diagnostic methods are for one or more diseases, disorders or conditions associated with cellular proliferation. In some embodiments, conditions that are treated and/or diagnosed are various cancers, thyroid diseases (e.g., hyperthyroidism or thyrotoxicosis), blood disorders (e.g., Polycythemia vera, an excess of red blood cells produced in the bone marrow), and/or cellular proliferation in blood vessels following balloon angioplasty and/or stent placement (known as restenosis).

In some embodiments, the cellular proliferation is due to a cancer.

In some embodiments, the cancer is adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease cancer, intestinal cancer, kidney cancer, larynx cancer, leukemia, liver cancer, lymph node cancer, lymphoma, lung cancer, melanoma, mesothelioma, myeloma, nasopharynx cancer, neuroblastoma, non-Hodgkin's lymphoma, oral cancer, ovarian cancer, pancreatic cancer, penile cancer, pharynx cancer, prostate cancer, rectal cancer, sarcomcancer, seminomcancer, skin cancer, stomach cancer, teratomcancer, testicular cancer, thyroid cancer, uterine cancer, vaginal cancer, vascular tumor cancer, and/or cancer from metastases thereof.

Treatment methods comprise administering to a subject a therapeutically effective amount of one or more of the conjugates of the application and optionally consist of a single administration, or alternatively comprise a series of administrations, and optionally comprise concurrent administration or use of one or more other therapeutic agents. For example, in some embodiments, the conjugates of the application are administered at least once a week. In some embodiments, the conjugates are administered to the subject from about one time per two or three weeks, or about one time per week to about once daily for a given treatment. In another embodiment, the conjugates are administered 2, 3, 4, 5 or 6 times daily. The length of the treatment period depends on a variety of factors, such as the severity of the disease, disorder or condition, the age of the subject, the concentration and/or the activity of the conjugates of the application, and/or a combination thereof. It will also be appreciated that the effective dosage of the conjugate used for the treatment may increase or decrease over the course of a particular treatment regime. Changes in dosage may result and become apparent by standard diagnostic assays known in the art. In some instances, chronic administration may be required. For example, the conjugates are administered to the subject in an amount and for duration sufficient to treat the subject. In some embodiments treatment comprise prophylactic treatment. For example, a subject with early cancer can be treated to prevent progression, or alternatively a subject in remission can be treated with a conjugate of the application to prevent recurrence.

The dosage of conjugates of the application varies depending on many factors such as the pharmacodynamic properties of the conjugates, the mode of administration, the age, health and weight of the recipient, the nature and extent of the symptoms, the frequency of the treatment and the type of concurrent treatment, if any, and the clearance rate of the conjugate in the subject to be treated. One of skill in the art can determine the appropriate dosage based on the above factors. Conjugates of the application may be administered initially in a suitable dosage that may be adjusted as required, depending on the clinical response. Dosages will generally be selected to maintain a serum level of conjugates of the application from about 0.01 μg/cc to about 1000 μg/cc, or about 0.1 μg/cc to about 100 μg/cc. As a representative example, oral dosages of one or more conjugates of the application will range between about 0.01 mg per day to about 1000 mg per day for an adult, suitably about 0.1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg. Conjugates of the application may be administered in a single daily, weekly or monthly dose or the total daily dose may be divided into two, three or four daily doses.

In some embodiments, when administering one or more conjugates of the application the total dose of absorbed radiation may include about 3 grays (Gy) to about 20 Gy, about 2 Gy to about 10 Gy, about 1 Gy to about 10 Gy, about 1 Gy, about 5 Gy, about 10 Gy, about 25 Gy, about 50 Gy, about 75 Gy, about 100 Gy, about 200 Gy, about 300 Gy, about 400 Gy, about 500 Gy, about 600 Gy, about 700 Gy, about 800 Gy, about 900 Gy or about 1000 Gy.

In some embodiments doses of absorbed radiation are achieved by delivering an appropriate amount of one or more conjugates of the application. Exemplary amounts of conjugates include about 0.05 mg/kg to about 5.0 mg/kg administered to a subject per day in one or more doses. For certain indications, in some embodiments, the total daily dose is about 0.05 mg/kg to about 3.0 mg/kg administered intravenously to a subject one to three times a day, including administration of total daily doses of about 0.05 mg/kg/day to about 3.0 mg/kg/day, about 0.1 mg/kg/day to about 3.0 mg/kg/day, about 0.5 mg/kg/day to about 3.0 mg/kg/day, about 1.0 mg/kg/day to about 3.0 mg/kg/day, about 1.5 mg/kg/day to about 3.0 mg/kg/day, about 2.0 mg/kg/day to about 3.0 mg/kg/day, about 2.5 mg/kg/day to about 3.0 mg/kg/day or about 0.5 mg/kg/day to about 3.0 mg/kg/day of one or more conjugates of the application using 60-minute QD, BI D, or TI D intravenous infusion dosing. In some embodiments, additional doses range from about 0.1 μg/kg to about 5 μg/kg or from about 0.5 μg/kg to about 1 μg/kg. In some embodiments, a dose is about 1 μg/kg, about 20 μg/kg, about 40 μg/kg, about 60 μg/kg, about 80 μg/kg, about 100 μg/kg, about 200 μg/kg, about 350 μg/kg, about 500 μg/kg, about 700 μg/kg, about 0.1 mg/kg to 5 about mg/kg, or from about 0.5 μg/kg to about 1 mg/kg. In some embodiments, a dose is about 1 mg/kg, about 10 mg/kg, about 20 mg/kg, about 40 mg/kg, about 60 mg/kg, about 80 mg/kg, about 100 mg/kg, about 200 mg/kg, about 400 mg/kg, about 500 mg/kg, about 700 mg/kg, about 750 mg/kg, about 1000 mg/kg, or more.

In some embodiments, therapeutically effective amounts of radionuclides are achieved by administering single or multiple doses during the course of an imaging and/or treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

In some embodiments, effective amounts vary according to factors such as the disease state, age, sex and/or weight of the subject. In a further embodiment, the amount of a given conjugate that will correspond to an effective amount will vary depending upon factors, such as the given drug(s) or compound(s), the pharmaceutical formulation, the route of administration, the type of condition, disease or disorder, the identity of the subject being treated, and the like, but can nevertheless be routinely determined by one skilled in the art.

In the context of cancers, in some embodiments, the therapeutic methods of treatment decrease the number of cancer cells, decrease the number of metastases, decrease tumour volume, increase life expectancy, induce chemo- or radiosensitivity in cancer cells, inhibit angiogenesis near cancer cells, inhibit cancer cell proliferation, inhibit tumour growth, prevent or reduce metastases, reduce cancer-associated pain and/or reduce relapse or re-occurrence of cancer.

In the context of hyperthyroidism or thyrotoxicosis, in some embodiments, the therapeutic methods of treatment aid in the return of thyroid secreted hormones, such as T3 and/or T4, to closer to normal levels (e.g. to about 80 ng/dl to about 180 ng/dl and/or about 4.6 μg/dl to about 12 μg/dl, respectively). In the context of polycythemia vera, in some embodiments, the therapeutic methods of treatment aid in the return of red blood cell counts to closer to normal level (e.g. to about 4.7 million cells/μL to about 8.1 million cells/μL).

In the context of restenosis, in some embodiments, the therapeutic methods comprise placing radionuclides in the region of a vessel where a stent is located or balloon angioplasty was performed to inhibit the narrowing of the vessel fur to proliferation of blood vessel cells. In some embodiments, treatment for restenosis is deemed effective if normal blood flow through the affected blood vessel is restored.

In some embodiments, the subject in need of is a subject in need of imaging. In some embodiments, the subject in need of imaging is a subject in need of diagnosis, in need of locating a position for a therapeutic intervention, in need of assessment of the functioning of a body part, and/or in need of assessment of the presence of absence of a condition.

In some embodiments, for use in diagnostic or therapeutic methods, the one or more compounds and/or conjugates are comprised in a pharmaceutical composition as described above.

### Preparation Methods of the Application

The zwitterions of the present application can be prepared by various synthetic processes. The choice of particular structural features and/or substituents may influence the selection of one process over another. The selection of a particular process to prepare a given compound of the application is within the purview of the person of skill in the art. Some starting materials for preparing compounds of the present application are available from commercial chemical sources. Other starting materials, for example as described below, are readily prepared from available precursors using straightforward transformations that are well known in the art.

In the Schemes below showing some embodiments of methods of preparation of zwitterions of the application, all variables are as defined in Formula I, unless otherwise stated.

Accordingly, in some embodiments, a compound of Formula I, wherein Q is Q1

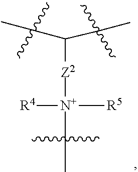

R¹ is a protecting group, R² is OH, Z¹ is C₃alkylene and R³ is sulfonate, is prepared as shown in Scheme 1.

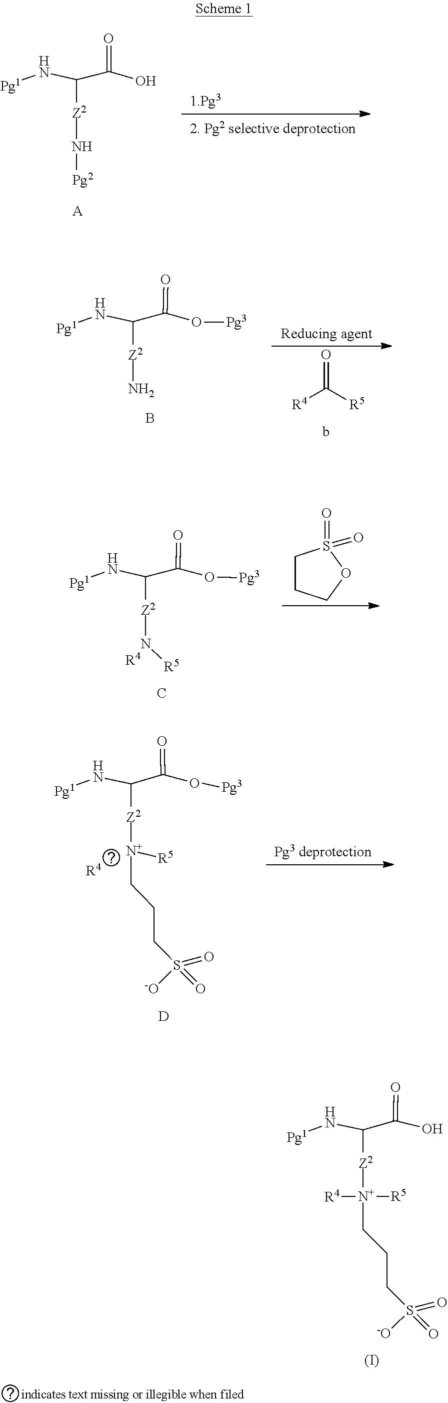

Accordingly, protection of group R² with Pg³ is followed by amine deprotection to form intermediate B, wherein Pg³ is any suitable protecting group and Z² is as defined by Formula I. The intermediate B undergoes reductive amination by adding compound b in a presence of reducing agent, such as sodium cyanoborohydride to form intermediate C, wherein R⁴ and R⁵ are as defined by Formula I. The resulting intermediate C is reacted with 1,3-propanesultone in a presence of a base, such as potassium carbonate to form intermediate compound of formula D, in which Z¹ is C₃alkylene and R³ is sulfonate. The resulting intermediate D undergoes hydrogenolysis in a presence of palladium catalyst to provide the compound of Formula I.

In some embodiments, a compound of Formula I, wherein Q is Q3

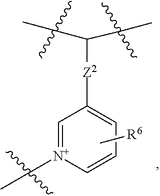

R¹ is a protecting group, R² is OH, Z¹ is C₃alkylene and R³ is sulfonate, is prepared as shown in Scheme 2.

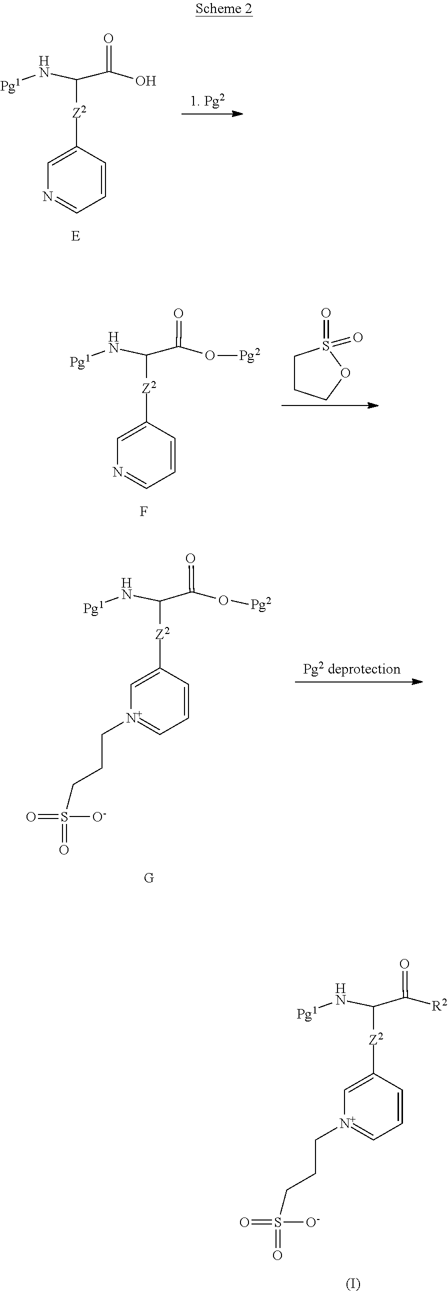

Accordingly, protection of group R² with Pg² to form indeterminate compound of formula F, wherein Pg¹ and Pg² are any suitable protecting groups and Z² is as defined by Formula I, is followed by reaction with -1,3-propanesultone in a presence of a base, such as potassium carbonate to form intermediate compound of formula G, in which Z¹ is C3alkylene and R³ is sulfonate. The resulting intermediate G undergoes hydrogenolysis in a presence of palladium catalyst to provide the compound of Formula I.

A person skilled in the art would appreciate that a similar reaction sequence can be used to prepared compounds Of formula I, wherein Q is Q2

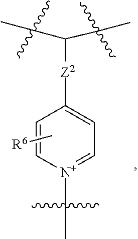

### or Q4

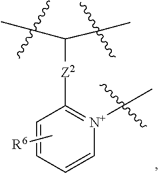

The chelating agents of Formula V of the present application can be prepared by various processes. The choice of particular structural features and/or substituents may influence the selection of one process over another. The selection of a particular process to prepare a given compound of the application is within the purview of the person of skill in the art. Some starting materials for preparing compounds of the present application are available from commercial chemical sources. Other starting materials, are readily prepared from available precursors using straightforward transformations that are well known in the art.

The compounds of the application generally can be prepared according to the processes illustrated in the Schemes below. In the structural formulae shown below the variables are as defined in Formula V unless otherwise stated.

Accordingly, in some embodiments, a compound of Formula V, wherein L¹ is

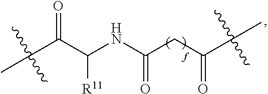

is prepared as shown in Scheme 3.

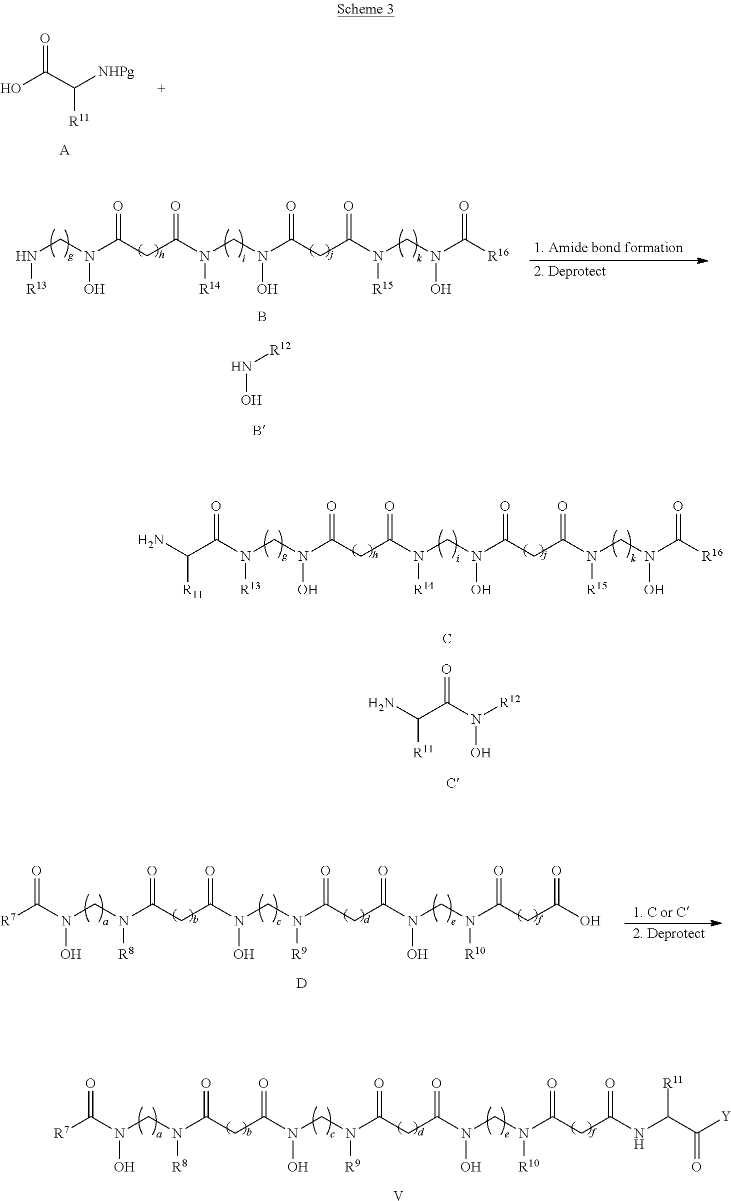

Therefore a compound of Formula A, wherein R¹¹ is as defined in Formula V or a protected version thereof and Pg is a suitable protecting group, is reacted with a compound of Formula B or B′, wherein R¹¹-R¹⁸, j, h, l, j and k are as defined in Formula V, under amide bond forming conditions to form, after removal of the protecting group, a compound of Formula C or C′ respectively. The compound of Formula C or C′ is then reacted with a compound of Formula D, wherein R⁷-R¹⁰, a, b, c, d, e and f are as defined in Formula V, under amide bond forming conditions, to provide after removal of any protecting groups if needed, the compound of Formula V, wherein L¹ is

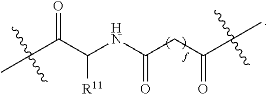

In some embodiments, a compound of Formula V, wherein L¹ is

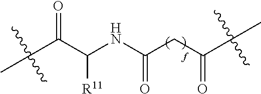

### and Y³ is

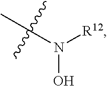

are prepared as shown in Scheme 4.

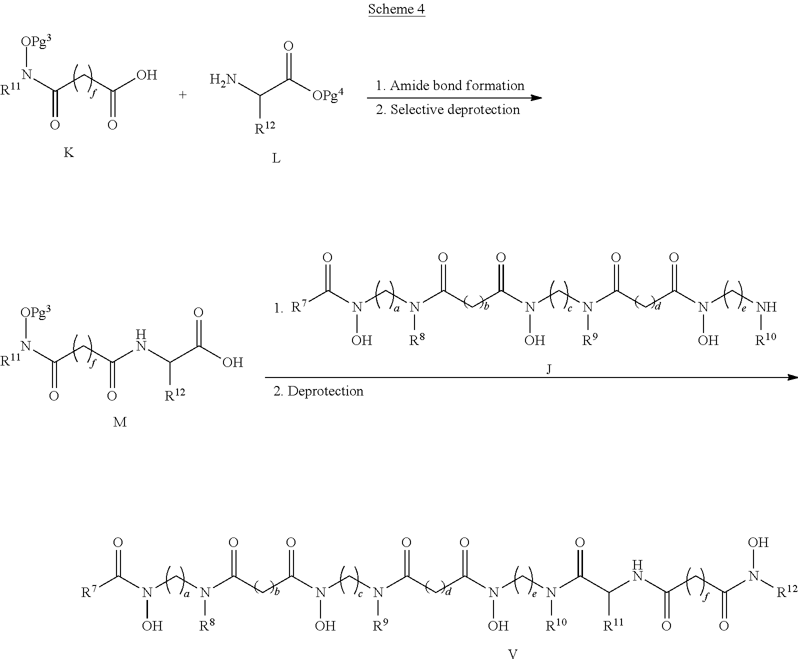

Therefore a compound of Formula K, wherein R¹¹ and f are as defined in Formula V and Pg³ is a suitable protecting group, is reacted with a compound of Formula L, wherein R¹² is as defined in Formula V or a protected version thereof and Pg⁴ is a suitable protecting group, under amide bond forming conditions followed by selective deprotection of Pg⁴, to provide a compound of Formula M. The compound of Formula M is then reacted with a compound of Formula J, wherein R⁷-R¹⁰, a, b, c, d, and e are as defined in Formula V, followed by removal of any protecting groups, to provide the compound of Formula V wherein L¹ is

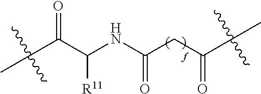

### and Y³ is

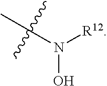

Amide bond forming conditions comprise any known method for the coupling of carboxylic acids and amines that is compatible with the intermediates and products shown in the above Schemes or that may be used to prepare a compound of the application. Known methods to prepare amides by the coupling of carboxylic acids and amines comprise use of either a coupling reagent or by prior conversion of the carboxylic acid into an activated derivative. Coupling reagents include, but are not limited to, any of the known peptide coupling reagents, such as 1-[bis(dimethylamino)methylene]-1H-1,2,3-triazolo[4,5-b]pyridinium 3-oxide hexafluorophosphate (HATU), 1-ethyl-3-(3-dimethylaminopropyl)carbodiimide (EDC), dicydohexylcarbodiimide (DCC) diisopropylcarbodiimide (DIC), hexafluorophosphate benzotriazole tetramethyl uronium (HBTU), 2-(1H-benzotriazole-1-yl)-1,1,3,3-tetramethylaminium tetrafluoroborate (TBTU), O-(1H-6-chlorobenzotriazole-1-yl)-1,1,3,3-tetramethyluronium hexafluorophosphate (HCTU), benzotriazol-1-yloxytripyrrolidinophosphonium hexafluorophosphate (PyBOP), (7-azabenzotriazol-1-yloxy)tripyrrolidinophosphonium hexafluorophosphate (PyAOP) and propanephosphonic acid anhydride.

In some embodiments, racemization of an enantiomer of a carboxylic acid occurs during amide bond formation using coupling reagents. In some embodiments, racemization is circumvented with ‘racemization suppressing’ additives such as the triazoles 1-hydroxy-benzotriazole (HOBt), 1-hydroxy-7-aza-benzotriazole (HOAt) and ethyl cyanohydroxyiminoacetate (Oxyma).

Nucleophilic displacement reaction conditions comprise any known method for the reaction of a nucleophile to displace a leaving group to form a bond that is compatible with the intermediates and products shown in the above Schemes or that may be used to prepare a compound of the application. In some embodiments, such conditions comprise combining reactants in the presence of a base in a suitable solvent.

Salts of the compounds of the application are generally formed by dissolving the neutral compound in an inert organic solvent and adding either the desired acid or base and isolating the resulting salt by either filtration or other known means.

The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Prodrugs of the compounds of the present application may be, for example, conventional esters formed with available hydroxy, thiol, amino or carboxyl groups. For example, available hydroxy or amino groups may be acylated using an activated acid in the presence of a base, and optionally, in inert solvent (e.g. an acid chloride in pyridine). Some common esters which have been utilized as prodrugs are phenyl esters, aliphatic (C1-C24) esters, acyloxymethyl esters, carbamates and amino acid esters.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

Techniques for purification of intermediates and final products include, for example, normal and reversed phase chromatography on column (manual, automated, and HPLC) or rotating plate, recrystallisation, distillation and liquid-liquid or solid-liquid extraction, which will be readily understood by one skilled in the art.

In some embodiments, a compound of the application is reacted with a complex comprising a chelating agent and one or more radionuclides under suitable conditions. In some embodiments, the complex comprising one radionuclide. In some embodiments, the complex comprising two radionuclides. In some embodiments, the compound of the application is reacted with a targeting ligand under suitable conditions. In some embodiments, the compound of the application is reacted with a biomolecule conjugating group under suitable conditions. In some embodiments, a conjugate of the application is formed using, for example, solid phase peptide synthesis under suitable conditions.

In some embodiments, the biomolecule conjugating group is reacted with the targeting ligand under suitable conditions. Targeting ligand can be conjugated to the biomolecule conjugating group of the application using any known methods for reacting, for example, but not limited to, a nucleophilic group, an electrophilic group, a Michael acceptor, a group that participates in a Click reaction, a group that participates in a cross-coupling reaction, a group that is activated to participate in a nucleophilic displacement reaction, to form a covalent bond. Such reactions are described in, for example “Comprehensive Organic Transformations—A Guide to Functional Group Preparations” R. C. Larock, VHC Publishers, Inc. (1989), “Advanced Organic Chemistry”, March, 4th ed. McGraw Hill (1992) or “Organic Synthesis”, Smith, McGraw Hill, (1994).

The following non-limiting examples are illustrative of the present application.

### EXAMPLES

The following non-limiting examples are illustrative of the present application.

### Materials and Methods

### Radiochemistry

Gallium-68 was procured from an in-house Eckert & Ziegler generator system (3131-0901 IGG100-30M), with a TiO₂-based stationary phase, eluting [⁶⁸Ge]Ge⁴⁺/[⁶⁸Ga]Ga³⁺, 1.11 GBq (30 mCi) with HCl (0.1 M). Activity was experimentally measured using a CRC-55tR dose calibrator (Capintec, Inc., NJ, USA) [44]. The activity was concentrated following generator elution using Bond Elut SCX Cartridge, 100 mg, particle size 40 pm (Agilent, cat. no. 12102013) and eluted with 500 μL of 5M NaCl and 0.05M HCl solution [45]. Reverse-phase radio-high-performance liquid chromatography (HPLC) was performed on a ThermoFisher Vanquish UHPLC (Dual λ Absorbance Detector, AcclaimPolar Advantage II, 120A, C18, 5.0 μm 4.6×250 mm column, Thermo Scientific) along with a flow count radiodetector F (Eckert & Ziegler, B-FC-3500 diode). The UV-detector was set at 254 and 280 nm and the solvent system was 0.1% trifluoroacetic acid (TFA) in water (A) and 0.1% TFA in acetonitrile (B) at a flow rate of 0.65 mL/min. Radio-iTLC were eluted using silica-gel impregnated glass-microfiber instant thin layer chromatography paper (iTLC-SG, iTLC-SA, Varian) using 1:1 methanol:ammonium acetate (1.0 M; pH˜4) as a developing solvent and measured using an Eckert & Ziegler AR2000 detector using P10 gas, with the included WinScan software.

### Chemical Characterization Methods

¹H and ¹³C nuclear magnetic resonance (NMR) spectra were recorded on a 500 MHz Bruker Avance NMR spectrometer at 25° C. in CDCl₃ or Methanol-d4 (MeOD). ¹H and ¹³C chemical shifts were referenced to the residual protons of the deuterated solvent (δ=7.26 and 3.31 ppm for CDCl3 and MeOD, respectively). Coupling constants are reported to the nearest 0.5 Hz (¹H NMR spectroscopy) or rounded to integer values in Hz (¹³C NMR spectroscopy). High resolution mass spectra were measured on a JEOL AccuTOF™ GCv 4G using field desorption ionization (FDI). For the isotopic pattern only, the mass peak of the isotopologue or isotope with the highest natural abundance is given. Low resolution mass spectrometry and liquid chromatography-mass spectrometry (LCMS) were performed using an Advion Expression-L system (mass range <2000 amu). HPLC purifications were performed using a Vanquish HPLC equipped with C18 reversed-phase column (Inspire Semipreparative DIKMA; 5 μm, 21.2×250 mm), a VF-D40-A UV detector, two VF-P10-A pumps, a Chromeleon 7 communication software, and a DIONEX UltiMate 3000 fraction collector, using a flow rate of 7 mL/min and a gradient of CH₃CN:H₂O (both with 0.1% TFA). LCMS was performed by coupling the above Thermofisher Vanquish with the above Advion Expression-L system.

### Reagents

All solvents and reagents were purchased from commercial suppliers (Sigma-Aldrich, St Louis, MO; TCI America, Portland, OR; Fisher Scientific, Waltham, MA; AK Scientific, Union City, USA) and were used as received unless otherwise indicated. Fmoc-L-Lys(Boc)-OH that used for synthesizing Fmoc-L-Lys(ZW)-OH ≥98.0% (cat. no. 47624); benzyl alcohol ≥99.0% (cat. no. 402834); formaldehyde solution 37 wt. % in H₂O (cat. no. 252549) and palladium on carbon 10 wt % loading (cat. no. 205699) were purchased from Sigma-Aldrich. Fmoc-β-(3-pyridyl)-D-Ala-OH was purchased from Sigma Aldrich. 1,3-propanesultone 99.92% (GC) (cat. no. 22005) was purchased from CHEM-IMPEX INT'L INC. All standard Nα-Fmoc amino acids used [Fmoc-L-Thr(tBu)-OH, Fmoc-L-Cys(Trt)-OH, Fmoc-L-Cys(Acm)-OH, Fmoc-L-Lys(Boc)-OH, Fmoc-D Trp(Boc)-OH, Fmoc-L-Tyr(tBu)-OH, and Fmoc-LPhe-OH]were purchased from AK Scientific, and coupling reagents such N,N,N′,N′-tetramethyl-O(1H-benzotriazol-1-yl)uronium hexafluorophosphate (HBTU) and 1-[bis(dimethylamino)methylene]-1H-1,2,3-triazolo[4,5-b]pyidinium3-oxide hexafluorophosphate (HATU) were purchased from Fischer Scientific. Fmoc-N-amido-PEG₄-acid was purchased from BroadPharma (California, USA). Fmoc-L-Thr(tBu)-Wang Resin was purchased from New England Peptide (capacity 0.8 mmol/g). 1,4,7,10-tetraazacyclododecane-1,4,7-tris (t-butyl acetate) (DO3A-tBu) was purchased from Macrocyclics, Inc. Water and buffers used for radiochemistry were millipure (18.2 mΩ) and further rendered metal-free by stirring overnight with 1.2 g Chelex-100 resin (200-400 mesh, Bio-Rad Laboratories, Inc.) per liter of solution and passing them through a 0.22 pm filter (Nalgene media filters).

### Example 1: Zwitterion (ZW) Preparation

### Synthesis of Fmoc-Lys(ZW)-COOH Amino Acid

The preparation of Fmoc-Lys(ZW)-COOH Amino Acid is shown in Scheme 5 and the synthetic details are provided below:

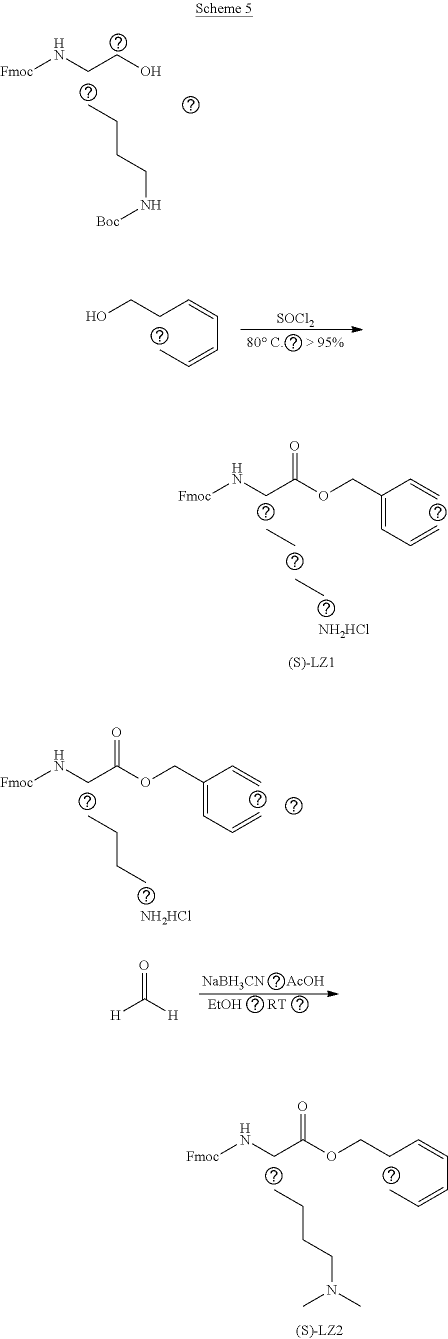

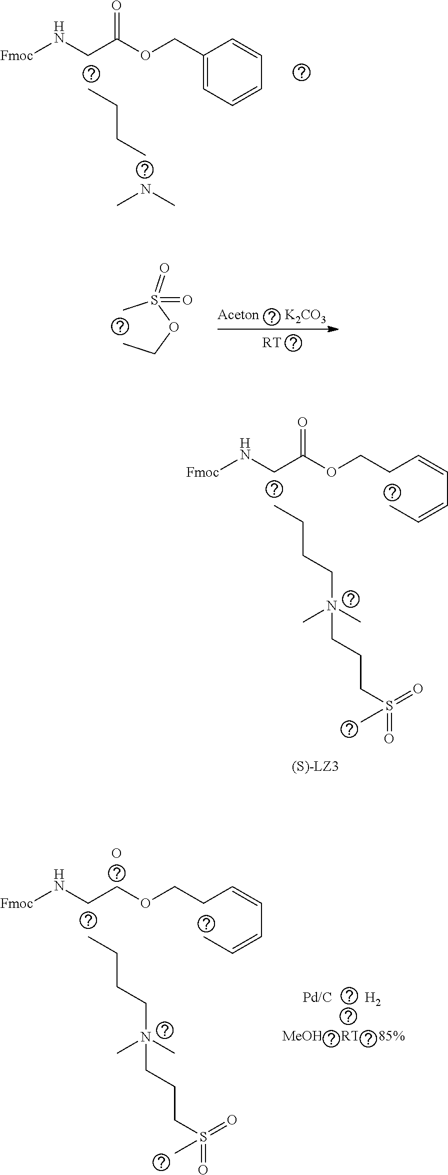

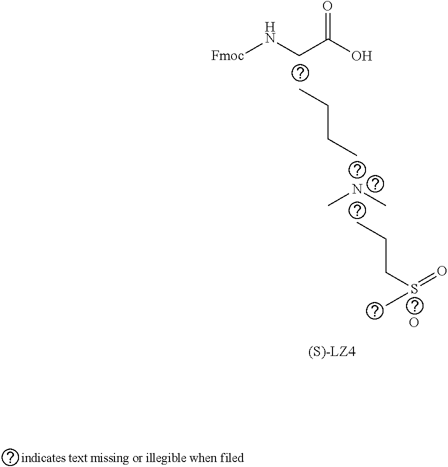

### Synthesis of (S)-benzyl 2-((((9H-fluoren-9-yl)methoxy)carbonyl)amino)-6-aminohexanoate ((S)-LZ1)

In a 100 mL round bottom flask, Fmoc-Lys(Boc)-OH (1.0 g, 2.1 mmol) and benzyl alcohol (10 mL) were mixed and stirred at room temperature. Then SOCl₂ (328 μL, 4.50 mmol) was added in a slow dropwise manner and the whole reaction was stirred at 80° C. for three hours. Cold diethyl ether (20 mL) was added and triturated and washed with more diethyl ether (3×20 mL) to give (S)-LZ1 (0.97 gm; 98%) as white solid. ¹H NMR (500 MHz, MeOD) δ (ppm): 1.44-1.88 (m, 6H), 2.88 (t, 2H, J=15.8 Hz), 4.17-4.23 (m, 2H), 4.28 (q, 1H, J=17.6 Hz), 4.39 (q, 1H, J=6.7 Hz), 5.15 (q, 2H, J=16.5 Hz), 7.27-7.80 (m, 13H). ¹³C NMR (MeOD) δ (ppm): 23.83, 27.97, 31.89, 40.47, 55.23, 67.93, 120.9, 126.1, 126.2, 128.1, 128.8, 129.3, 129.5, 137.2, 142.6, 145.1, 145.3, 158.7, 173.6. HRMS-ESI m/z calcd for [C₂₈H₃₀N₂O₄+H]⁺: 459.2278. found: 459.2287.

### Synthesis of (S)-benzyl 2-((((9H-fluoren-9-yl)methoxy)carbonyl)amino)-6-(dimethylamino)hexanoate ((S)-LZ2)

Each of Fmoc-Lys-OBn ((S)-LZ1) (0.970 g, 1.96 mmol), formaldehyde (1.95 mL, 19.6 mmol, 37% w/v solution in H₂O) and acetic acid (0.337 mL, 5.88 mmol) were dissolved in 50 mL ethanol and stirred at room temperature for five minutes. Sodium cyanoborohydride (0.493 g, 7.84 mmol) was added in portions. The reaction mixture was left to stir at room temperature for four hours. The solvent was removed and the residue was purified using reverse phase chromatography (80-20% H₂O: 20-80% CH₃CN gradient) and lyophilized to give (S)-LZ2 as a white powder (0.620 g, 60%). ¹H NMR (500 MHz, MeOD) δ (ppm): 1.37-1.48 (m, 6H), 2.72 (s, 6H), 2.90 (t, 2H, J=15.8 Hz), 4.19-4.23 (m, 2H), 4.30 (q, 1H, J=17.6 Hz), 4.43 (q, 1H, J=6.7 Hz), 5.16 (q, 2H, J=16.5 Hz), 7.28-7.81 (m, 13H). ¹³C NMR (MeOD) δ (ppm): 21.0, 22.4, 28.9, 40.7, 52.2, 55.9, 64.9, 65.0, 117.9, 123.1, 125.2, 125.8, 126.3, 126.6, 134.2, 139.6, 142.1, 155.7, 170.7. HRMS-ESI m/z calcd for [C₃₀H₃₄N₂O₄+H]⁺: 487.2597. found: 487.2619.

### Synthesis of (S)-3-((5-((((9H-fluoren-9-yl)methoxy)carbonyl)amino)-6-(benzyloxy)-6-oxohexyl)dimethyla-mmonio)propane-1-sulfonate ((S)-LZ3)

1,3-propanesultone (0.284 g, 2.33 mmol) was refluxed in acetone (10 mL) for 30 minutes. Separately, Fmoc-Lys(dimethyl)-OBn ((S)-LZ2) (0.610 g, 1.17 mmol) was stirred with potassium carbonate (0.323 g, 2.34 mmol) in acetone (5 mL) for 30 minutes at room temperature. After 30 minutes elapsed, Fmoc-Lys(dimethyl)-OBn solution was added to 1,3-propanesultone and the reaction was left for another hour to reflux and then stirred overnight at room temperature. The solvent was removed and the residue was purified by using reverse phase chromatography (80-20% H₂O: 20-80% CH₃CN gradient) and lyophilized to give (S)-LZ3 as a white powder (0.434 g, 40-61%). ¹H NMR (500 MHz, MeOD) δ (ppm): 1.41-1.79 (b, 6H), 2.15 (q, 2H, J=16.8 Hz), 2.85 (t, 2H, J=15.8 Hz), 3.05 (s, 6H), 3.23 (q, 2H, J=15.9 Hz), 3.48 (q, 2H, J=16.8 Hz), 4.19-4.42 (m, 4H), 5.18 (q, 2H, J=17.8 Hz), 7.29-7.81 (m, 13H). ¹³C NMR (MeOD) δ (ppm): 16.9, 19.9, 20.8, 28.8, 48.3, 52.2, 60.8, 62.0, 64.9, 65.0, 118.0, 123.2, 125.2, 125.3, 125.8, 126.4, 126.6, 134.2, 139.6, 142.1, 155.7, 170.6. HRMS-ESI m/z calcd for [C₃₃H₄₀N₂O₇S+Na]⁺: 541.1984. found: 541.1983.

### Synthesis of (S)-3-((5-((((9H-fluoren-9-yl)methoxy)carbonyl)amino)-5-carboxypentyl)dimethylammonio) propane-1-sulfonate ((S)-LZ4)

Both Fmoc-Lys(ZW)-OBn ((S)-LZ3) (0.430 g, 0.706 mmol) and palladium (0.0755 g; 0.071 mmol, 10% Pd/C) were dissolved in methanol (20 mL) in a Schlenk flask that was connected to water respiratory from side neck to create vacuum and from the top neck it was attached to a balloon filled with H₂ gas. Three cycles of vacuum and purging with H₂ gas were performed then filled with H₂ gas and left the reaction to stir at room temperature for four hours. The completion of the reaction was confirmed by thin-layer chromatography (TLC) and the catalyst was removed by filtration through celite powder. The solvent was removed under vacuo and the residue was purified using reverse phase chromatography (80-20% H₂O: 20-80% CH₃CN gradient) and lyophilized to give (S)-LZ4 as a white powder (0.293 gm, 80%). ¹H NMR (500 MHz, MeOD) δ (ppm): 1.48-2.21 (b, 10H), 2.89 (t, 2H, J=15.5 Hz), 3.09 (s, 6H), 3.52 (q, 2H, J=16.5 Hz), 4.19-4.44 (m, 4H), 7.34-7.83 (m, 8H). ¹³C NMR (MeOD) δ (ppm): 19.8, 22.9, 23.8, 32.1, 51.2, 55.0, 63.8, 65.0, 67.9, 120.9 126.2, 126.3, 128.1, 128.2, 128.8, 142.6, 145.1, 145.3, 158.7, 175.7. HRMS-ESI m/z calcd for [C₂₆H₃₄N₂O₇S+H]⁺: 519.2159. found: 519.2190.

### Synthesis of Fmoc-Ala-Pyr(ZW)-COOH Amino Acid

The preparation of Fmoc-Ala-Pyr(ZW)-COOH Amino Acid is shown in Scheme 6 and the synthetic details are provided below:

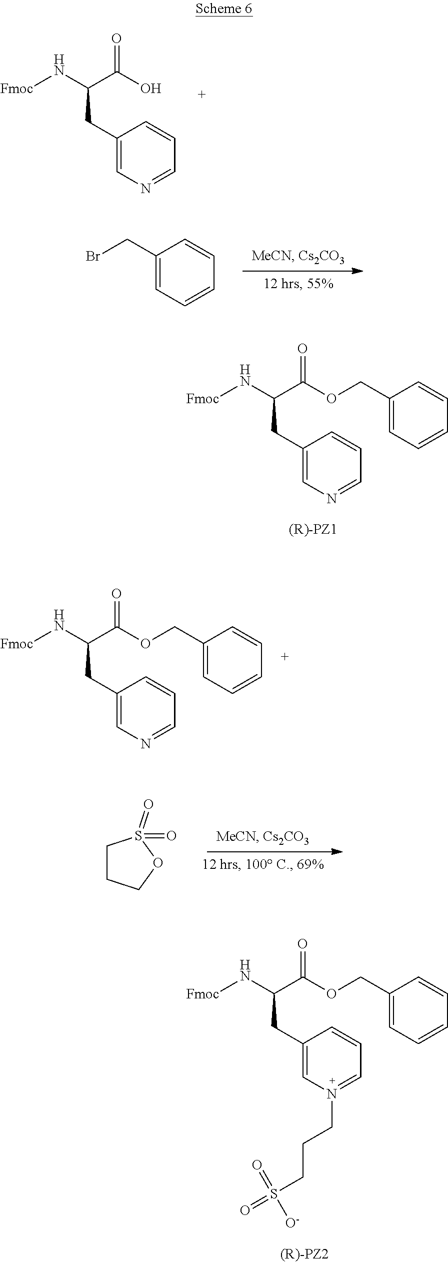

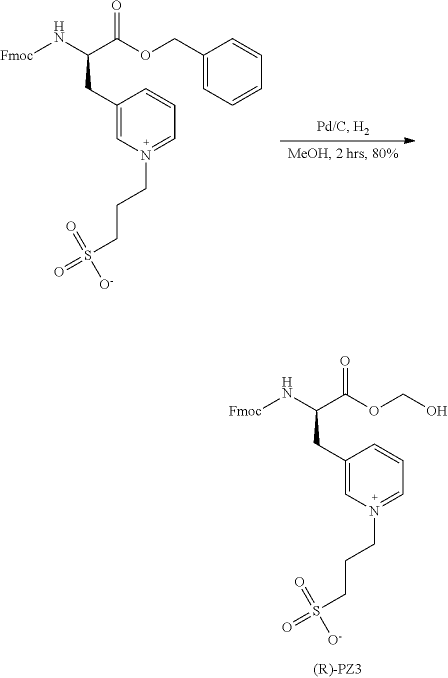

### Synthesis of (R)-benzyl 2-((((9H-fluoren-9-yl)methoxy)carbonyl)amino)-3-(pyridin-3-yl)propanoate ((R)-PZ1)

In a 100 mL round bottom flask, Fmoc-Ala-Pyr-OH (1.0 g, 2.6 mmol) and Cs₂CO₃ (0.85 g, 2.6 mmol) were stirred in 30 mL CH₃CN at room temperature for 15 minutes. This was followed by addition of benzyl bromide (0.29 mL, 2.5 mmol), then the reaction mixture was stirred at room temperature overnight. The solvent was removed, and 30 mL H₂O was added and extracted three times with ethyl acetate and dried over sodium sulfate. The residue was purified with normal phase chromatography (20-100% ethyl acetate:hexane gradient) to give (R)-PZ1 (0.65 g; 55%) as an off-white solid. ¹H NMR (500 MHz, DMSO-de) δ (ppm): 2.92-2.96 (dd, 1H, J=8.11 Hz), 3.10-3.14 (dd, 1H, J=6.32 Hz), 4.13-4.26 (m, 3H), 4.34-4.39 (dd, 1H, J=3.40 Hz), 5.12-5.13 (d, 2H, J=5.55 Hz), 7.26-7.42 (m, 10H), 7.60-7.68 (m, 3H), 7.87 (s, 1H), 7.89 (s, 1H), 8.01 (s, NH), 8.42-8.48 (m, 2H). ¹³C NMR (DMSO-de) δ (ppm): 33.92, 46.98, 55.54, 66.18, 66.60, 120.6, 123.7, 125.6, 127.5, 128.1, 128.2, 128.5, 128.8, 133.4, 136.2, 137.1, 141.1, 144.1, 148.2, 150.7, 156.4, 171.9. HRMS-ESI m/z calcd for [C₃₀H₂₆N₂O₄+H]⁺: 479.20. found: 479.197 with ppm Error 1.18.

### Synthesis of (R)-3-(3-(2-((((9H-fluoren-9-yl)methoxy)carbonyl)amino)-3-(benzyloxy)-3-oxopropyl)pyridin-1-ium-1-yl)propane-1-sulfonate ((R)-PZ2)

Fmoc-Ala-Pyr-OBn ((R)-PZ1) (0.75 g, 1.57 mmol) was stirred with potassium carbonate (0.65 g, 4.71 mmol) in 30 mL of CH₃CN for 15 minutes at room temperature then the solution was filtered to a new 100 mL round bottom flask to remove all insoluble potassium carbonate behind. 1,3-propanesultone (1.92 g, 15.7 mmol) was added and the reaction mixture refluxed at 100° C. overnight. The solvent was removed, and the residue was diluted with H₂O (30 mL), then extracted three times with 30 mL of ethyl acetate, and then dried over sodium sulfate. The crude mixture was purified using reverse phase chromatography (80-20% H₂O: 20-80% CH₃CN gradient) and lyophilized to give (R)-PZ2 as a white powder (0.65 g, 69%). ¹H NMR (500 MHz, DMSO-de) δ (ppm): 2.18-2.24 (m, 2H), 2.42 (t, 2H, J=8.11 Hz), 3.10-3.15 (dd, 1H, J=8.11 Hz), 4.17 (t, 1H, J=6.81 Hz), 4.23-4.32 (m, 2H), 4.54-4.58 (m, 1H), 4.65 (t, 2H, J=7.04 Hz), 5.15 (s, 2H), 7.29-7.42 (m, 10H), 7.62 (t, 2H, J=7.97 Hz), 7.88 (d, 2H, J=7.57 Hz), 7.98-8.03 (m, 2H), 8.42 (s, NH), 8.94-9.06 (m, 2H). ¹³C NMR (DMSO-de) δ (ppm): 27.79, 33.49, 46.98, 47.52, 54.38, 60.20, 66.18, 66.82, 120.6, 125.5, 127.5, 128.1, 128.3, 128.9, 136.1, 138.8, 141.1, 144.1, 145.6, 146.2, 156.4, 171.2. HRMS-ESI m/z calcd for [C₃₃H₃₂N₂O₇S+H]⁺: 601.20. found: 601.2005 with ppm Error 0.332.

### Synthesis of (R)-3-(3-(2-((((9H-fluoren-9-yl)methoxy)carbonyl)amino)-2-carboxyethyl)pyridin-1-ium-1-yl)propane-1-sulfonate ((R)-PZ3)

Both Fmoc-Ala-Pyr(ZW)-OBn ((R)-PZ2) (0.65 g, 1.08 mmol) and palladium on carbon (0.18 g; 10.8 mmol, 10% Pd/C) were dissolved in methanol (20 mL) in a Schlenk flask connected, with mild vacuum drawn using a water aspirator from the side neck, and a balloon filled with H₂ gas and fitted with a glass valve was attached to the top neck. Three cycles of vacuum and purging with H₂ gas were performed, then the reaction vessel was filled with H₂ gas and left to stir at room temperature for 2 hours. The completion of the reaction was confirmed by reversed-phase TLC and the catalyst was removed by filtration through celite powder. The solvent was removed under vacuo and the residue was purified using reverse phase chromatography (80-20% H₂O: 20-80% CH₃CN gradient) and lyophilized to give (R)-PZ3 as a white powder (0.44 g, 80%). ¹H NMR (500 MHz, DMSO-de) δ (ppm): 2.20-2.25 (m, 2H), 2.44 (t, 2H, J=3.29 Hz), 3.06-3.10 (dd, 1H, J=8.09 Hz), 4.17 (t, 1H, J=6.87 Hz), 4.21-4.28 (m, 2H), 4.34-4.38 (m, 1H), 4.67 (t, 2H, J=7.02 Hz), 7.31 (t, 2H, J=7.43 Hz), 7.40 (t, 2H, J=7.43 Hz), 7.64 (t, 2H, J=7.94 Hz), 7.79 (d, 1H, J=8.48 Hz), 7.88 (d, 2H, J=7.48 Hz), 8.04 (t, 1H, J=4.67 Hz), 8.44 (d, NH), 8.96-9.05 (m, 2H), 13.1 (b, OH). ¹³C NMR (DMSO-de) δ (ppm): 27.80, 33.73, 46.99, 47.53, 54.44, 60.170, 66.10, 120.6, 125.6, 127.5, 127.7, 128.1, 128.3, 139.3, 141.1, 143.5, 144.1, 145.5, 146.2, 156.4, 172.8. HRMS-ESI m/z calcd for [C₂₆H₂₆N₂O₇S+H]⁺: 511.15. found: 511.1518 with ppm Error −3.03.

### Example 2: DOTA-(ZW)n-TATE Preparation

### Synthesis and Purification of DOTA-TATE and DOTA-(ZW)ₙ-TATEs

For the synthesis and purification of each of DOTA-TATE, DOTA-PEG₄-TATE, DOTA-(ZW)₁-PEG₄-TATE (DOTA-(I-(S)-LZ₄)₁-PEG₄-TATE), DOTA-(ZW)₂-PEG₄-TATE (DOTA-(I-(S)-LZ₄)₂-PEG₄-TATE), and DOTA-(Pyr(ZW)-PEG₄-TATE (DOTA-(I-(R)-RZ3)-PEG₄-TATE) previously published ultrasonic agitation method was employed [44]. This bench-top solid phase peptide synthesis (SPPS) method began with 50 mg of Fmoc-Thr(tBu)-Wang resin (preloaded with first amino acid, loading capacity 0.8 mmol/g) was used as solid phase and was swelled in dimethylformamide (DMF) overnight before use. Amino acids were purchased or synthesized with Fmoc protecting groups on the N-terminus and a free carboxylic acid on the C-terminus. Peptides were built on solid phase in the C→N direction with the unprotected N-terminus coupling to each new amino acid. Each amino acid was coupled with 2 mol equiv. relative to the resin using ultrasonic agitation and with variable coupling times depending on the amino acid (determined by Kaiser test). The Fmoc deprotection steps following each coupling step was performed using 20% piperidine in DMF for 3 minutes with ultrasonic agitation. After each single step of Fmoc deprotection and peptide coupling, the resin was washed thoroughly with DMF (3×1 mL) and dichloromethane (DCM) (3×1 mL), and the procedure was repeated until the desired peptide was obtained. In the same manner as standard SPPS method, the Kaiser test was used to optimize the reaction times needed for each ultrasonic-assisted coupling step, as the time required to reach near quantitative yield varies between amino acids. Cleavage and drying of the peptide were performed traditionally using 95% TFA, 2.5% DCM, and 2.5% triisopropylsilane (TIPS). Semipreparative reverse-phase (C18) high-performance liquid chromatography was carried out on a Thermofisher Vanquish HPLC system with a gradient elution of 20-100% B in A (A=0.1% aqueous formic acid in H₂O; B=CH₃CN+0.1% formic acid) for 45 min (flow rate 7 mL/min). The desired peak was collected, and the organic solvent was removed in vacuo. The aqueous solution was frozen and lyophilized to give DOTA-TATE (Total Synthesis Time (TST)=2.2 hrs), DOTA-PEG₄-TATE (TST=2.6 hrs), DOTA-(ZW)₁-PEG₄-TATE (TST=3.3 hrs) and DOTA-(ZW)₂-PEG₄-TATE (TST=4 hrs) with yields of 29%, 29%, 29% and 28%, respectively.

Radiolabeling and Purification of DOTA-TATEs with [⁶⁸Ga]Ga³⁺

Gallium [⁶⁸Ga]Ga³⁺ activity was eluted from a commercial TiO₂-based [⁶⁸Ge]Ge⁴⁺/[⁶⁸Ga]Ga³⁺ generator (Eckert & Ziegler), according to the manufacturers instructions using 6 mL of 0.1 M HCl prepared from 30% HCl (TraceSELECT Ultra, Fluka) as eluent. If the desired activity (60-80 MBq) in 500 μL of abovementioned eluent was not obtained, then the concentration procedure of the activity was followed. In brief, the 6 mL of activity was passed through a preconditioned SCX column (Preconditioning: 1 mL 5.5 M HCl, 10 mL chelex treated H₂O, and 10 mL air) and the activity released with 500 μL of 5 M NaCl and 0.05 M HCl solution. The pH was adjusted to 3.8-4 using 1.0 M ammonium acetate buffer pH=4) and 14 nmol of the peptides were added and the reaction mixture was incubated in 95° C. on a shaker at 900 RPM for 12 minutes. The crude [⁶⁸Ga]Ga-DOTA-TATEs products were purified by Sep-Pak light (Waters) solid-phase extraction cartridge that was previously conditioned and equilibrated with 10 mL of deionized water, 10 mL pure ethanol and 10 mL of deionized water. The trapped labeled peptides were released in different fractions using 1 mL of EtOH:0.9% NaCl (1:2 ratio). The fraction with the highest activity was used for further in vitro and in vivo studies by taking in account that the alcohol percentage should be less than 10%.

### Determination of Partition Coefficient (log D_{7.4})

The lipophilicity of each one of the [⁶⁸Ga]Ga-DOTA-TATEs were assessed as described in the literature [46]. In brief, two phases of 1-octanol and phosphate buffer (PBS) at a pH of 7.4 were presaturated with each other. The presaturated 1-octanol (3.0 mL) was added to 3.0 mL of presaturated PBS that had about 0.3 MBq of labelled [⁶⁸Ga]Ga-DOTA-TATEs. The mixture was vortexed for 1.0 minute at room temperature then centrifuged for 1.0 minute at 3000 RPM. 1.0 mL of each phase was taken and measured its activity in automated gamma counter. Log D7.4 was determined for at least three experiments using Equation 1 below, where CPM (count per minute) is the reading from the gamma counter.

$$Log {D}_{7.4} = log ( {CPM}_{octanol} / {CPM}_{buffer} ) \qquad ( 1 )$$

### Serum Stability In Vitro

Serum stability was determined using a previously published method [47]. Aliquots of each one of the [⁶⁸Ga]Ga-DOTA-TATEs (5.0 MBq) were added to 0.4 mL human or mice serum and incubated at 37° C. Aliquots (20 μL) were taken at various time intervals (10, 30, 60, 90, 120 minutes) and quenched in ice-cold acetonitrile. The samples were centrifuged, and the supernatants were diluted further with deionized water and were analyzed by radio-HPLC (a flow rate 0.65 mL/min with a gradient of CH₃CN:H₂O with 0.1% TFA in both). 5.0 MBq of [⁶⁸Ga]Ga-DOTA-TATEs were prepared in 0.4 mL of PBS and used as a reference in the radio-HPLC measurement for the stability in serum study.

### Plasma-Protein Binding (PPB)

Serum binding assay for each one of [⁶⁸Ga]Ga-DOTA-TATEs were carried out as described in the literature [48]. Compendiously, labelled [⁶⁸Ga]Ga-DOTA-TATEs (4-3 MBq) were added to freshly prepared 0.1 mL of anticoagulated human or mice plasma and the mixtures were incubated for 120 minutes at 37° C. This was followed by addition of 1.0 mL ice-cold acetonitrile and centrifuged for 2 minutes at 3000 RPM. The addition of acetonitrile had been repeated two times and all supernatants (denoted as A) were combined and the total radioactivity was measured using automated gamma counter. The radioactivity of the precipitate (B) was measured as well. The plasma-protein binding rate was calculated using Equation 2 below (n=3).

$$PPB = B / ( A + B ) 100 \% \qquad ( 2 )$$

### PET/CT and Biodistribution in Healthy and Xenograft Mice

The experimental protocol was approved by the University of Saskatchewan Committee on Animal Care and procedures were performed in accordance with the guidelines of the Canadian Council on Animal Care and the Canadian Nuclear Safety Commission. Aliquots of about 200 μL of each one of [⁶⁸Ga]Ga-DOTA-TATEs (7-5 MBq with <10% EtOH) were loaded to 0.5 mL syringes and >96% Radiochemical yields (RCY) were confirmed by using radio-HPLC followed by injection to healthy CD1 female mice and xenograft bearing male NOD-SCID gamma mice (USASK colony F1 005557, strain NOD.Cg-PrkdcscidIl2rg) (n=4), via tail vein catheter while in the PET scanner with image acquisition started, under anesthesia (2.5% isoflurane in O₂, at a flow rate of 2 mL/min). 90-minute dynamic images of all four mice were collected simultaneously on the Sofie GNEXT PET/CT small animal imaging system using the included 4-mouse heated bed. The mice were euthanized (by cervical dislocation while they were under the effect of anesthesia) at 2 hrs post injection and blood and specific organs were harvested, weighed, and residual activity in the organs were measured by using an automated gamma counter. All images were reconstructed using VivoQuant® 2020 software (patch 1 64 bit, build vq-2020patch1-0-ga8255affc).

### Results and Discussion

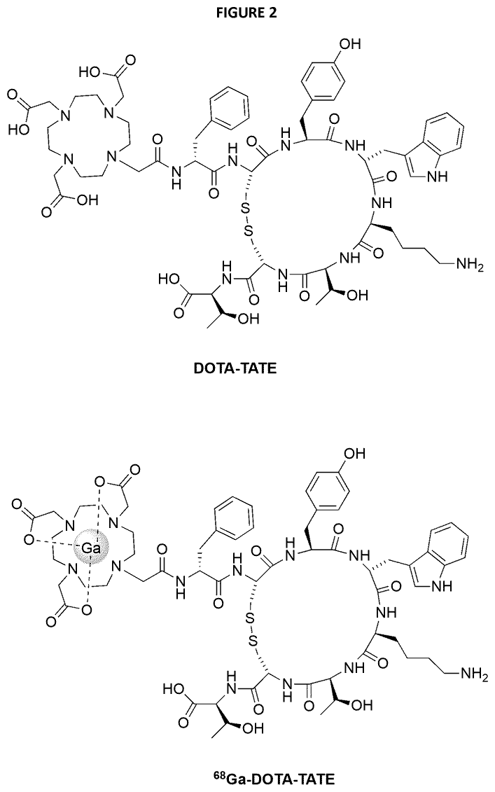

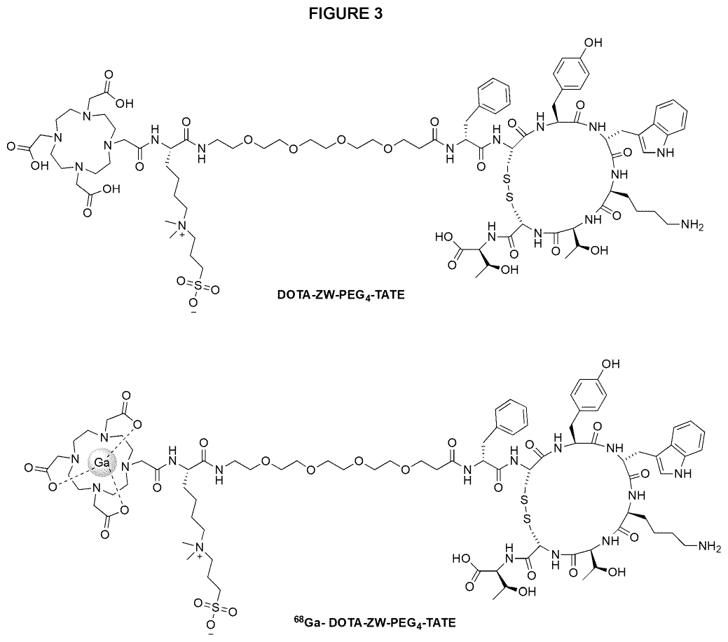

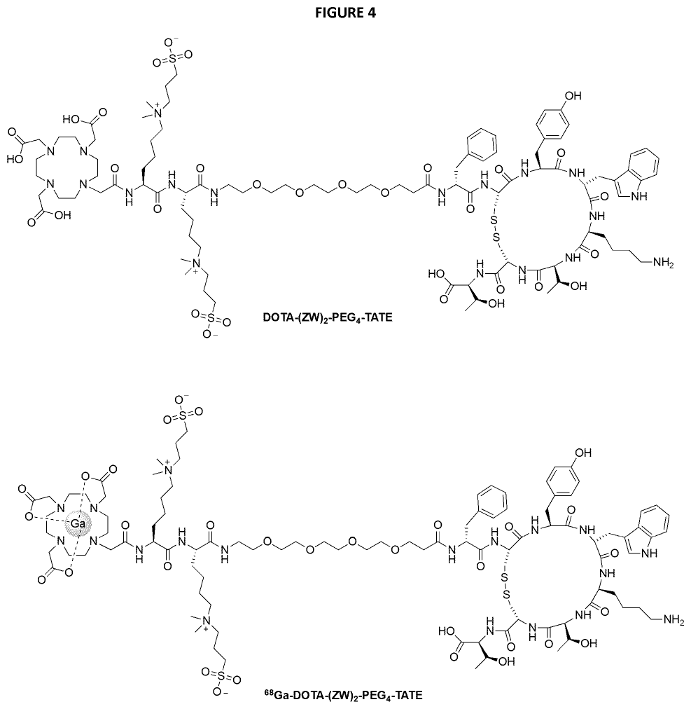

After synthesis, purification, and characterization of ZW amino acids, benchtop solid-phase peptide synthesis of DOTA-TATE and DOTA-(ZW)n-TATE derivatives (FIGS. 2-4 top) were performed. Then, gallium-68 radiolabeling and in vitro experiments to assess their behavior were conducted (FIG. 2-4 bottom). Instant thin layer chromatography (iTLC) was used to monitor the labelling process of [⁶⁸Ga]Ga-DOTA-TATE (the radiochemical conversions (RCC %) based on iTLC were 97.9%), and RCC % based on radio-HPLC were more than 95% for zwitterion TATE derivatives (Table 1 below).

Final apparent molar activity values for all peptide conjugates used for in vitro and in vivo studies were ˜5-5.8 GBq/μmol (135-157 mCi/μmol). The lipophilicity of each one of the [⁶⁸Ga]Ga-DOTA-TATEs were assessed by octanol-water partitioning as described in the literature [Maschauer, S.; Einsiedel, J.; Haubner, R.; Hocke, C.; Ocker, M.; Hubner, H.; Kuwert, T.; Gmeiner, P.; Prante, O., Labelling and glycosylation of peptides using click chemistry: a general approach to (18)F-glycopeptides as effective imaging probes for positron emission tomography. Angew. Chem. Int. Ed. Engl. 2010, 49 (5), 976-9], and there was a wide range of hydrophilicity among the tested compounds, with [⁶⁸Ga]Ga-DOTA-Pyr(ZW)-PEG₄-TATE showing the most negative value (most polar and water soluble) at Log D_{PBS·7.4}=−3.57 and the standard [⁶⁸Ga]Ga-DOTA-TATE showing the least at Log D_{PBS·7.4}=−2.81, while the other compounds varied between these two values (Table 1 below).

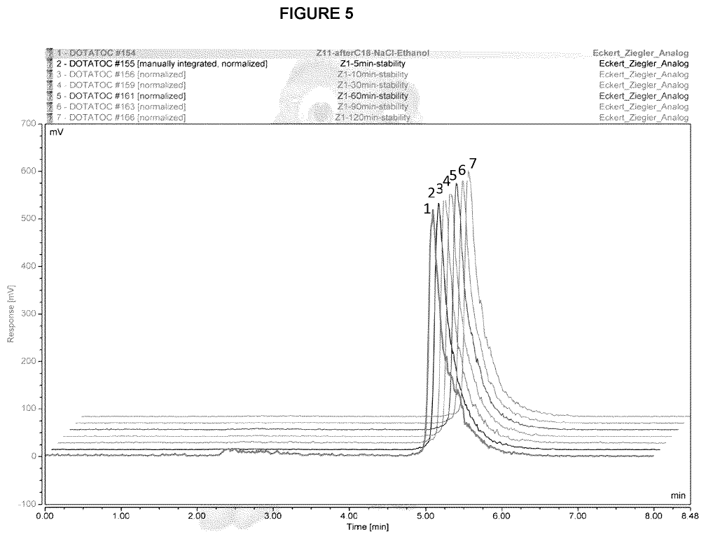

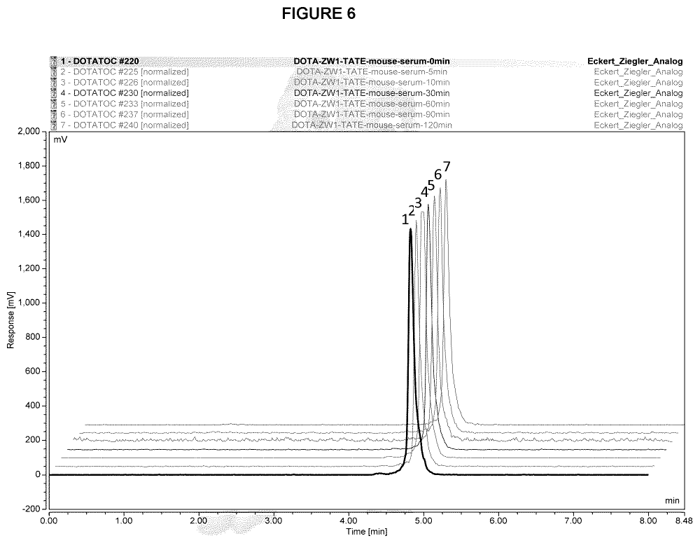

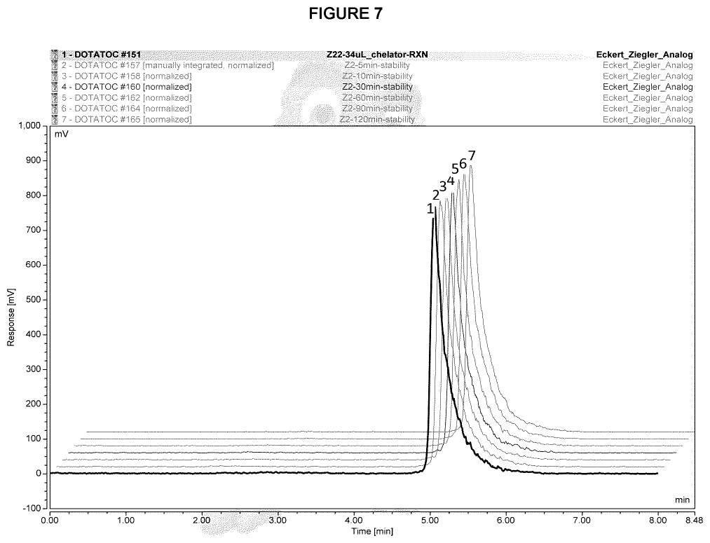

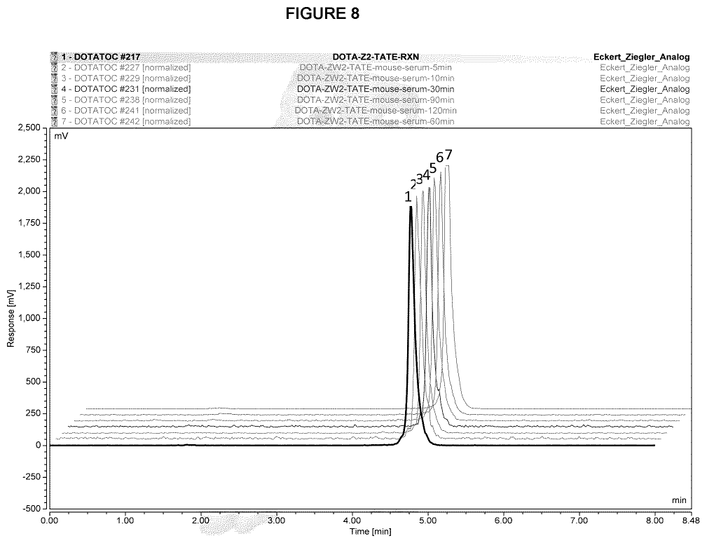

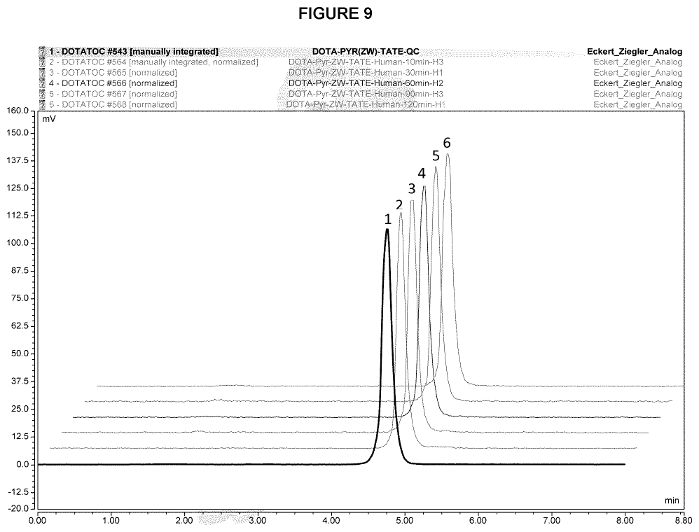

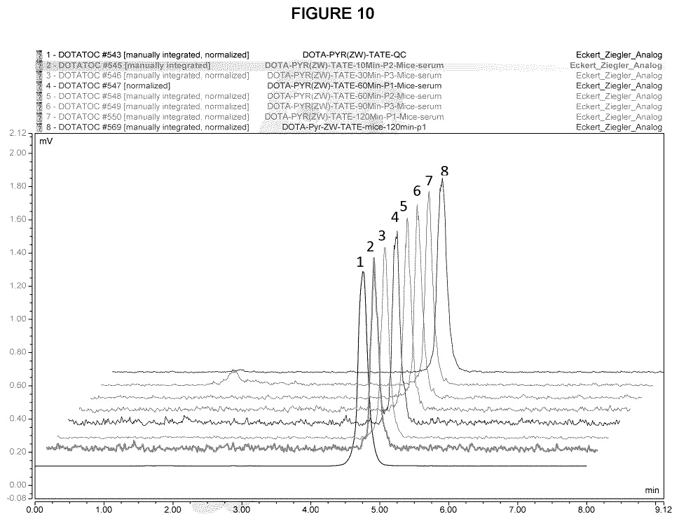

When the compounds' stability in mouse and human blood serum was examined (FIG. 5-10) [Xia, Y.; Zeng, C.; Zhao, Y.; Zhang, X.; Li, Z.; Chen, Y., Comparative evaluation of (68)Ga-labeled TATEs: the impact of chelators on imaging. EJNMMI Res. 2020, 10 (1), 36], DOTA-TATE and its Lys(ZW)/Lys(ZW)₂ derivatives remained completely intact even after 2 hours, whereas the Pyr(ZW) derivative exhibited minimal ˜0.4% instability in mouse serum but remained totally stable in human serum (Table 1 below). Without wishing to be limited by theory, the instability in mouse serum could be explained by the fact that murine blood serum contains more proteases than human blood serum [Puente, X. S.; Sanchez, L. M.; Overall, C. M.; Lopez-Otin, C., Human and mouse proteases: a comparative genomic approach. Nat. Rev. Genet. 2003, 4 (7), 544-58].

TABLE 1
In vitro data results

| | | RP-HPLC | MSS % | HSS % | TPB % |
|---|---|---|---|---|---|
| Peptides | LogD | RT (min) | (2 hrs.) | (2 hrs.) | (2 hrs.) |
| DOTA-TATE | −2.81 ± 0.11 | 4.82 | 100 | 100 | 43.3 ± 6.2 |
| DOTA-(ZW1)- | −3.39 ± 0.19 | 4.81 | 100 | 100 | 46.6 ± 8.9 |
| PEG4-TATE | | | | | |
| DOTA-(ZW2)- | −3.36 ± 0.18 | 4.77 | 100 | 100 | 48.5 ± 3.4 |
| PEG4-TATE | | | | | |
| DOTA-Pyr(ZW)- | −3.57 ± 0.03 | 4.78 | 99.6 ± 0.1 | 100 | 42.5 ± 0.5 |
| PEG4-TATE | | | | | |

MSS = Mouse Serum Stability; HSS = Human Serum Stability; TPB = Total Protein Binding ± SD, RP-HPLC RT is the reverse-phase C18 HPLC retention time, LogD is the octanol/phosphate buffered saline (pH 7.4) partition coefficient.

Experiments were performed in healthy female mice (CD1) to determine the behavior and distribution of these compounds (pharmacokinetics). Mice were catheterized via tail vein and injected while in the scanner, and dynamic PET-CT imaging was performed from time 0 until 1.5 hours post injection (n=4 each group, 4 mice in the scanner simultaneously).

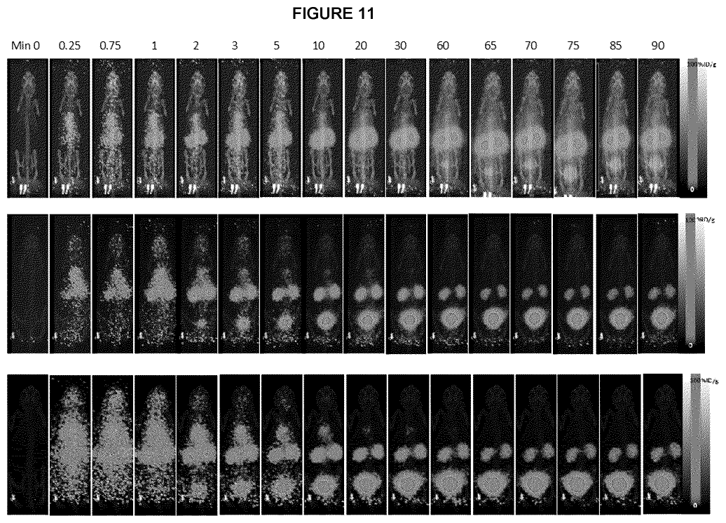

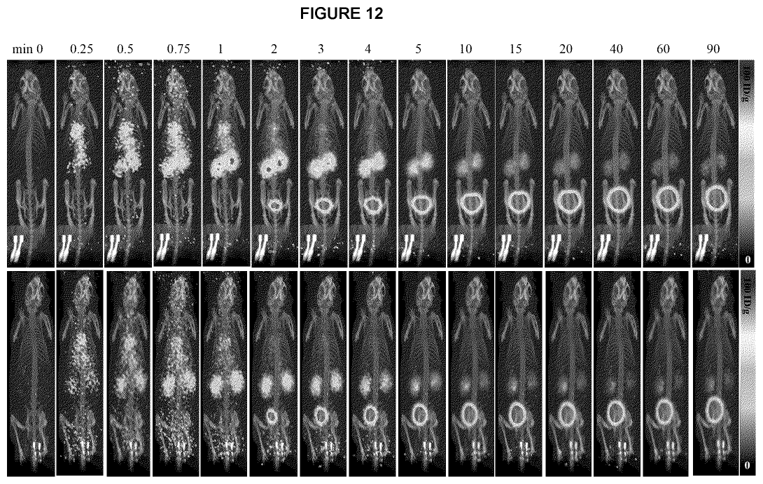

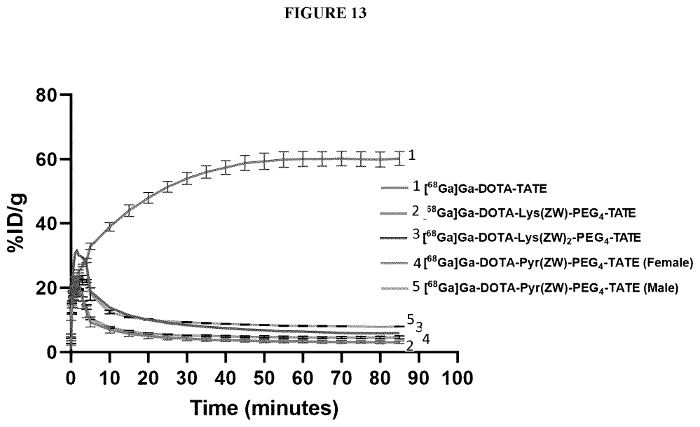

Rapid accumulation and prolonged retention of [⁶⁸Ga]Ga-DOTA-TATE in the kidneys were seen during dynamic PET/CT imaging, with late, sluggish excretion into the bladder (FIG. 11, top). In contrast, [⁶⁸Ga]Ga-DOTA-Lys(ZW)-PEG₄-TATE and [⁶⁸Ga]Ga-DOTA-Lys(ZW)₂-PEG₄-TATE demonstrated rapid clearance and minimal accumulation in the kidneys (FIG. 11, middle and bottom, respectively). When the [⁶⁸Ga]Ga-DOTA-Pyr(ZW)-PEG₄-TATE derivative was injected into both female and male CD1 healthy mice, a similar profile of fast clearance and minimal renal uptake was observed (FIG. 12, top and bottom, respectively). After 90 minutes following injections, time-activity curve (TAC) data were extracted from dynamic PET imaging, and they were in accordance with the PET images. At the end of imaging (90 min post injection) kidney uptake values were 60.2, 3.0, 5.8 and 4.5% ID/g for [⁶⁸Ga]Ga-DOTA-TATE, [⁶⁸Ga]Ga-DOTA-Lys(ZW)-PEG₄-TATE, [⁶⁸Ga]Ga-DOTA-Lys(ZW)₂-PEG₄-TATE, and [⁶⁸Ga]Ga-DOTA-Pyr(ZW)-PEG₄-TATE, respectively (FIG. 13).

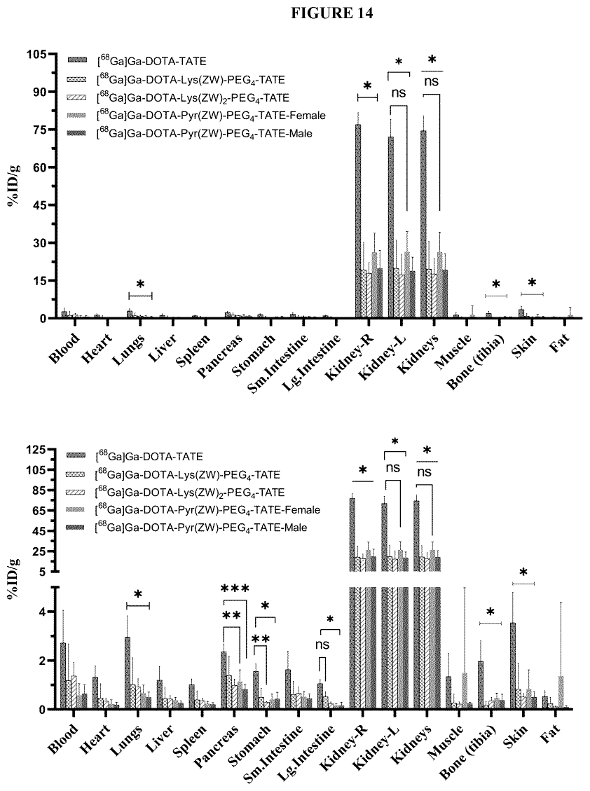

After imaging was complete, the mice were kept awake for an additional 30 minutes before being euthanized, bringing the total period of the injection to 2 hours. Biodistribution was performed by removing and weighing individual tissues and organs, and then measuring the activity of [⁶⁸Ga]Ga³⁺ inside each sample using an automated gamma counter. When comparing [⁶⁸Ga]Ga-DOTA-TATE to the three exemplary zwitterion-containing derivatives, biodistribution data revealed that the renal uptake was highest for [⁶⁸Ga]Ga-DOTA-TATE (˜74±51 D/g %) and lowest for the zwitterion derivatives (˜17-26±10 ID/g %) (FIG. 14).

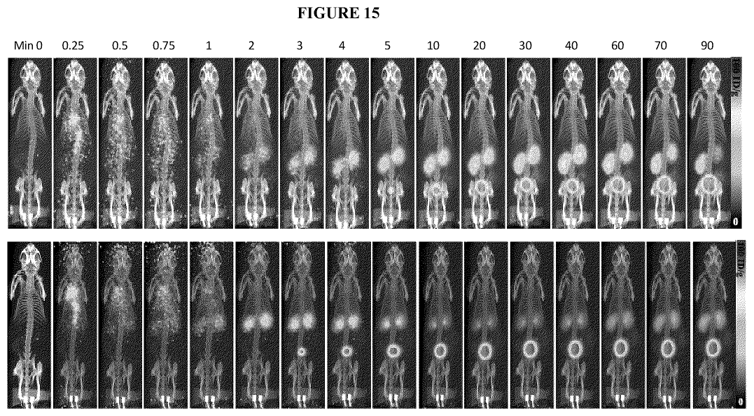

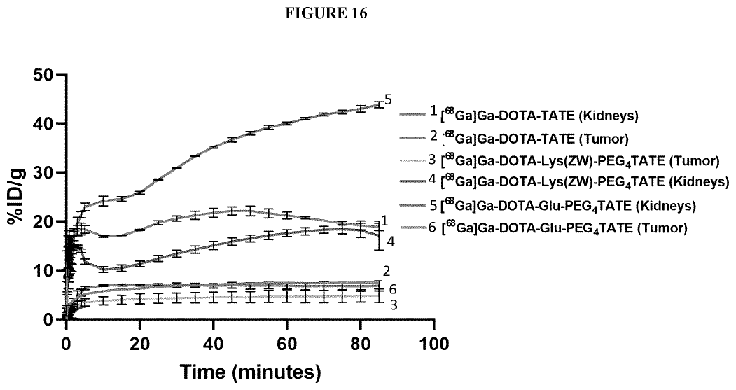

After success in healthy mice, murine xenograft models which had subcutaneous tumors over-expressing the target somatostatin receptor (SSTR2) for the TATE peptide of DOTA-TATE were tested. Each of [⁶⁸Ga]Ga-DOTA-TATE and its Lys(ZW) derivatives [⁶⁸Ga]Ga-DOTA-Lys(ZW)n-PEG₄-TATE were injected into male mice (NSG) bearing AR42J (SSTR2 over-expressing) subcutaneous tumors, and they were imaged from 0-90 minutes via dynamic PET/CT imaging (FIG. 15). Dynamic PET/CT imaging revealed rapid accumulation and protracted retention of [⁶⁸Ga]Ga-DOTA-TATE in the kidneys, with relatively slow outflow into the bladder. On the other hand, [⁶⁸Ga]Ga-DOTA-Lys(ZW)-PEG₄-TATE showed faster clearance into the kidneys and less retention in the kidneys, which is confirmed by TAC data as well (FIG. 16). The dynamic PET images and the TAC data reveal a rapid initial uptake into the kidneys between 0-5 minutes for both compounds, followed by a rapid decrease as activity was cleared into the bladder, and then a gradual increase in kidney uptake. This observation was most pronounced for the zwitterion containing compound, where less activity was retained in the kidneys than for [⁶⁸Ga]Ga-DOTA-TATE following the initial rapid uptake.

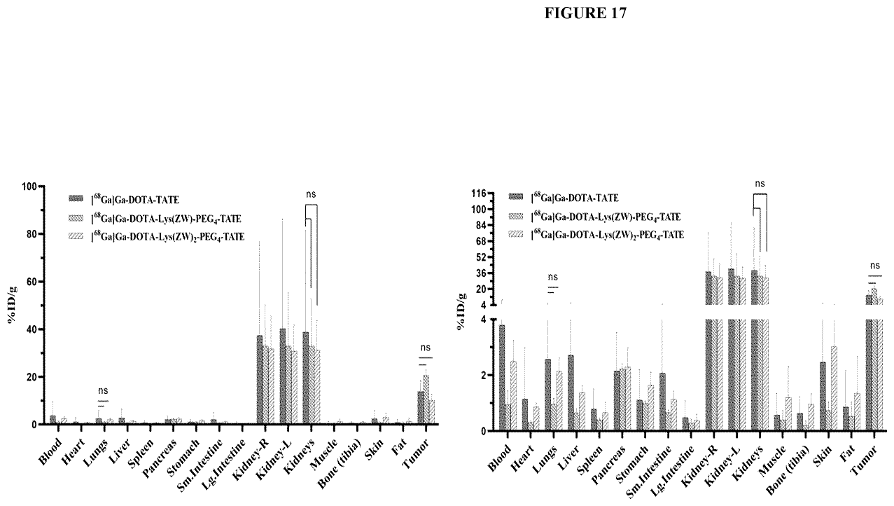

As done for healthy mouse studies, animals were awakened for 30 minutes following PET/CT and then euthanized at 2 hours post injection. Tissue/organ samples were collected, weighed, and the amount of activity present in the samples was measured using an automated gamma counter. According to biodistribution data, renal uptake was moderate for each of [⁶⁸Ga]Ga-DOTA-TATE and its Lys(ZW) derivatives (31-38% ID/g), while it was the highest for [⁶⁸Ga]Ga-DOTA-Lys(ZW)-PEG₄-TATE in tumor and lowest in other healthy organs (FIG. 17). Although the kidney uptake of ZW-containing derivatives trended lower than [⁶⁸Ga]Ga-DOTA-TATE, unlike in healthy mice there was not a statistically significant difference found. The ZW-containing derivatives trended lower in most healthy tissues with substantially improved profiles.

### Discussion

The application of permanent zwitterion moieties to molecules used in vivo have shown promising performance in both NIR and nanoparticle applications. In this study, the permanent zwitterion moieties were incorporated into peptide-based radiopharmaceuticals. Synthesizing amino acids that have a sulfobetaine as side chain group and are compatible with solid phase peptide synthesis would be a modular and versatile approach. Therefore, both commercially available lysine and pyridyl alanine were modified such that they contained zwitterionic side chains and possessed standard protection chemistry for SPPS. The Fmoc-Pyr(ZW)-OH amino acid was synthesized in three steps as compared to four steps for Fmoc-Lys(ZW)-OH. Also, Fmoc-Pyr(ZW)-OH synthesis was higher yielding and more reproducible. In addition, it has a UV-active pyridine group that is advantageous when linked to a chelator or peptide or other molecular of interest that lacks a UV-active moiety. The standard DOTA-TATE (NETSPOT/LUTATHERA®) and ZW-containing derivatives [⁶⁸Ga]Ga-DOTA-Ly(ZW)-PEG₄-TATE, [⁶⁸Ga]Ga-DOTA-Lys(ZW)₂-PEG₄-TATE, and [⁶⁸Ga]Ga-DOTA-Pyr(ZW)-PEG₄-TATE were synthesized using manual benchtop SPPS techniques with both US and mechanical agitation, with US providing slightly higher yields and much shorter coupling times.

Due to the unnatural structure and charge distribution, DOTA-TATE derivatives containing ZW amino acids were >99% stable in human and murine blood serum over 2 hours, which suggests they are not recognized by proteases in the blood.

Dynamic PET/CT images in healthy female mice clearly demonstrated that the standard [⁶⁸Ga]Ga-DOTA-TATE had rapid accumulation and protracted retention in the kidneys with slow excretion into the bladder that started ˜70 minutes post injection. However, the Lys(ZW) derivatives demonstrated rapid clearance from the kidneys, with minimal retention and faster renal elimination via bladder at ˜2-5 minutes post injection. In addition, the Pyr(ZW) derivative exhibited the same profile as the Lys(ZW) derivatives in both healthy female and male mice. The ZW-containing derivatives showed a faster clearance from the kidneys into the bladder from ˜0-5 minutes post injection, with a larger drop in kidney uptake and therefore less retention. At the end of dynamic PET/CT imaging (90 min p.i.) kidney uptake values were 60.2, 3.0, 5.8 and 4.5% ID/g for [⁶⁸Ga]Ga-DOTA-TATE, [⁶⁸Ga]Ga-DOTA-Lys(ZW)-PEG₄-TATE, [⁶⁸Ga]Ga-DOTA-Lys(ZW)₂-PEG₄-TATE, and [⁶⁸Ga]Ga-DOTA-Pyr(ZW)-PEG₄-TATE, which for the ZW derivatives correspond to decreases of ˜20-fold, ˜10-fold, and ˜13-fold, respectively (FIG. 13).

Biodistribution studies on healthy mice (both male and female) showed quantitatively that the ZW derivatives retained less activity in the kidneys than the standard [⁶⁸Ga]DOTA-TATE by more than 3-fold at 2 hours post injection. In addition to kidneys, the ZW derivatives exhibited a ˜2-fold reduction in uptake in many healthy organs relative to the standard. In tumor-bearing mice, the biodistribution profile of all compounds changed significantly with respect to kidney uptake and healthy tissue clearance. In male mice bearing AR42J tumors the profile of the standard [⁶⁸Ga]Ga-DOTA-TATE was close to that of healthy female mice, with the exception of decreased retention in the kidneys and quicker elimination via bladder. While not wishing to be limited by theory, this disparity may be explained by the mice's different sexes, types, immune systems, and the presence of large SSTR2-positive tumors which can bind but will also slowly release the injected peptides (washout). It is known that radiometallated-peptides undergo a process called “washout” where they quickly accumulate in tumors via receptor binding but are also released from the tumors over time, resulting in a gradual release of tracer from the tumor and much different rates of clearance from the blood pool and healthy tissues when compared with healthy mice. While the [⁶⁸Ga]Ga-DOTA-Lys(ZW)-PEG₄-TATE compound exhibited a profile comparable to that of healthy female mice, the kidney uptake was slightly higher presumable due to activity slowly being washed out of the tumor and back into the bloodstream. The [⁶⁸Ga]Ga-DOTA-Lys(ZW)₂-PEG₄-TATE compound is not represented in FIG. 15 due to a malfunction in the PET/CT equipment. Even though the Lys(ZW) derivatives had average healthy tissue uptake values in tumor-bearing mice that trended ˜2-3-fold lower than DOTA-TATE, the differences were not statistically significant. This is partially due to the large error values that are commonly encountered with ex vivo biodistribution experiments in mice, and from the limited sample size (n=3-4 per group), but the differences are clear as seen in the PET/CT images, TAC data, and biodistribution values. This contrasts with experiments in healthy mice, where the ZW derivatives were statistically significantly lower in many organs with a dramatically ˜3-fold lower kidney uptake. The Lys(ZW) derivative had greater tumor uptake (p=0.058,) and lower trending uptake in most healthy organs, showing an improved biodistribution profile. In comparison to the standard, the Lys(ZW)₂ derivative showed reduced tumor uptake. While not wishing to be limited by theory, this can be explained by its rapid elimination from the blood pool, which gives it less opportunity to adhere to tumor specific receptors (FIG. 17).

These results demonstrate that the DOTA-TATE derivatives that are integrated with ZW modules can provide better contrast PET images due to an increase in the target-to-background ratio and could provide improved therapeutic indexes when combined with therapeutic radionuclides.

### Conclusions

The SPPS compatible amino acids Fmoc-Lys(ZW)-OH and Fmoc-Pyr(ZW)-OH, which bear side chains with permanent zwitterions (ZW) were successfully synthesized. Using DOTA-TATE (NETSPOT/LUTATHERA®) as a “gold standard” radiopharmaceutical peptide, with synthesized 3 derivatives which incorporated these ZW amino acids. Peptides were successfully radiolabeled with [⁶⁸Ga]GaCl₃ (t_{1/2}=68 min) under standard conditions (12 min, 95° C.) with high radiochemical yields and purity. The ZW-containing derivatives showed higher hydrophilicity than the gold standard (more negative Log D values) and they showed high stability in both human and murine blood serum. Dynamic PET/CT of the ZW-containing derivatives in healthy mice exhibited rapid elimination and up to ˜10-20-fold reduction in kidney uptake compared to substantial accumulation and lengthy retention of [⁶⁸Ga]Ga-DOTA-TATE. In addition, quantitative ex vivo biodistribution revealed decreases in kidney uptake for both of our ZW derivatives by more than 3-fold and by ˜2-fold in other healthy organs compared to [⁶⁸Ga]Ga-DOTA-TATE. Interestingly, kidney uptake was not significantly decreased in male AR42J tumor bearing mice for the ZW compound [⁶⁸Ga]Ga-DOTA-Lys(ZW)-PEG₄-TATE, but it had the highest tumor uptake (20.6±2.5% ID/g vs 13.8±4.6% ID/g) and average uptake values in the kidneys (32.9±19.8% ID/g vs 38.8±42.7% ID/g) and other healthy tissues that clearly trended lower than [⁶⁸Ga]Ga-DOTA-TATE and was visually impactful from PET/CT images. Also, the tumor-to-kidney ratio for [⁶⁸Ga]Ga-DOTA-Lys(ZW)-PEG₄-TATE was 0.63. and 0.35 for the gold standard, demonstrating that the ZW-containing tracer outperforms the gold standard in terms of tumor and kidney uptake. This data suggests that amino acids bearing “permanent” zwitterions as side chain functional groups improve the biodistribution profiles of peptide-based radiopharmaceuticals. The substantially higher tumor uptake, lower kidney uptake, lower uptake in many healthy organs, resistance to proteases due to its unnatural structure and charge distribution, lack of reactivity with common bioconjugate chemistries and compatibility with standard SPPS, and high polarity with a new charge of 0 position these ZW-bearing amino acids as a valuable and highly modular approach to improving the biological distribution of peptide-based radiopharmaceuticals.

### Example 3: Preparation of Chelating Agents

Desferrioxamine (DFO) mesylate salt was purchased from Abcam and used as received.

### Desferrioxamine Derivative DFO2K

### Materials and Methods

### Materials

All reagents and solvents were purchased from commercial suppliers (Sigma-Aldrich, St. Louis, MO; TCI America, Portland, OR; Fisher Scientific, Waltham, MA) and were used without further purification unless otherwise indicated. Oxyma Pure and N,N-diisopropylethylamine (DIPEA), were purchased from Sigma-Aldrich. DFO mesylate was purchased from Abcam. 1-[Bis(dimethylamino)methylene]-1H-1,2,3-triazolo[4,5-b]pyridinium 3-oxide hexafluorophosphate (HATU) and Boo-Lys(Z)-OH were purchased from AK Scientific. ZrCl₄, EDTA, disodium salt, dihydrate (Molecular biology grade), ammonium acetate (Honeywell ≥99.99% trace metal basis), 1-ethyl-3-(3-dimethylaminopropyl)carbodiimide hydrochloride (EDC·HCl) and potassium carbonate anhydrous were purchased from Fisher Scientific.

### Chemical Characterization Methods

¹H and ¹³C NMR spectra were recorded on a 500 MHz Bruker Avance Ill HD NMR spectrometer at the Saskatchewan Structural Science Centre at the University of Saskatchewan, at 25° C. in D₂O, CDCl₃ or (CD₃)₂SO. Chemical shifts were referenced to the residual protons of the deuterated solvents for ¹H NMR (δ=4.79 ppm for D₂O; δ=7.26 ppm for CDCl₃ and δ=2.50 ppm for (CD₃)₂SO. Similarly, ¹³C chemical shifts were referenced to the CDCl₃ signal at 77.16 ppm and (CD₃)₂SO signal at δ=39.52 ppm. Coupling constants are reported to the nearest 0.5 Hz (¹H NMR spectroscopy) and for ¹³C NMR spectroscopy they are rounded to integer values in Hz. Field desorption ionization (FDI) was used on JEOL AccuTOF GCv 4G mass spectrometer to measure high resolution mass spectra and only the mass peak of the isotope with the highest natural abundance is recorded for the isotopic patterns. HPLC purifications were performed on Thermofisher Vanquish HPLC using C18 reversed-phase column (Inspire Semipreparative DIKMA; 5 μm, 21.2×250 mm), a VF-D40-A UV detector, two VF-P10-A pumps with Chromeleon 7 communication software. A flow rate of 6-8 mL/minute was used with a gradient of MeCN:H₂O (with 0.1% formic acid (FA) in both solvents). Low resolution mass spectrometry was performed on Advion Expression-L system (mass range <2000 amu). The Advion Expression-L was coupled with the Thermofisher Vanquish system to perform liquid chromatography-mass spectrometry (LCMS).

### Preparation of DFO2K

The preparation of DFO2K is shown in Scheme 7 and the synthetic details are provided below:

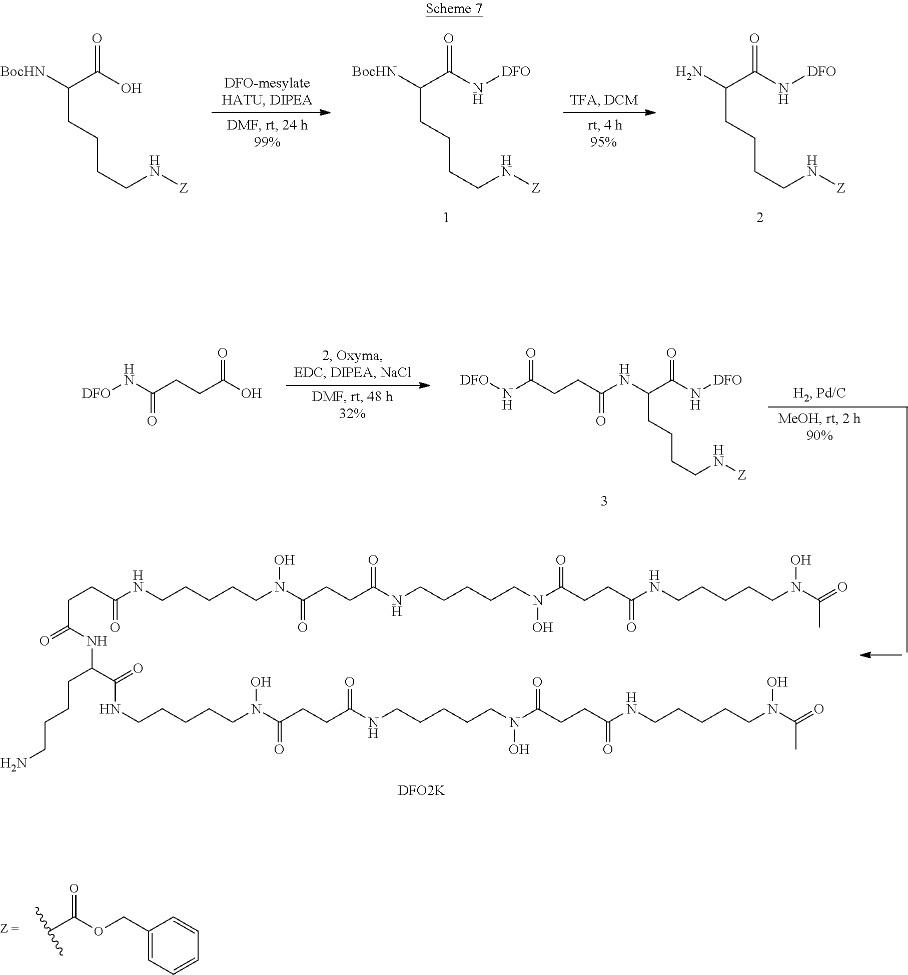

### Synthesis of DFO-Lys(Z)-Boc (1)

Compound 1 was prepared according to a published procedure with modification [Sarbisheh, E. K.; Salih, A. K.; Raheem, S. J.; Lewis, J. S.; Price, E. W., A High-Denticity Chelator Based on Desferrioxamine for Enhanced Coordination of Zirconium-89. Inorg. Chem. 2020, 59 (16), 11715-11727]. A solution of Boo-Lys(Z)-OH (264 mg, 0.69 mmol) in DMF (15 mL) was added to a stirring mixture of HATU (306.1 gm, 0.81 mmol) and DIPEA (0.14 mL, 0.81 mmol) in DMF (15 mL). DFO mesylate (501 mg, 0.76 mmol) was dissolved in DMF (17 mL) and DMSO (8 mL) at 65° C. The clear solution of DFO was cooled to around 40° C. and then it was added to the above activated ester solution. The reaction was stirred at ambient temperature for 24 h and then the solvent was reduced to ˜2 mL under reduced pressure. The residue was added dropwise to ethyl acetate and left in a spark proof −20° C. freezer for 30 minutes, to maximize precipitate formation. The precipitate was separated by centrifugation, washed with cold ethyl acetate 2 times, flash frozen in liquid nitrogen and then dried by freeze dryer. Compound 1 (632 mg, 99%) was used in the next step without further purification. ¹H NMR [(CD₃)₂SO, 500 MHz]: δ 1.21 (br s, 8H, CH₂), 1.34-1.39 (m, 17H, tBu and CH₂), 1.96 (s, 3H, CH₃), 2.26 (t, J=7.3, 4H, CH₂), 2.57 (t, J=7.2, 4H, CH₂), 2.94-3.01 (m, 8H, CH₂), 3.43-3.45 (m, 6H, CH₂), 3.80-3.81 (m, 1H, CH), 4.99 (s, 2H, CH₂), 7.23 (t, J=5.5, 1H, NH), 7.29-7.37 (m, 5H, CH Ar), 7.75 (t, J=5.4, 1H, NH), 7.79 (t, J=5.0, 2H, NH). ¹³C NMR [(CD₃)₂SO, 126 MHz]: δ 20.4, 22.9, 23.4, 23.5, 26.1, 27.6, 28.1, 28.2, 28.8, 28.9, 29.1, 29.9, 31.8, 38.3, 38.4, 46.8, 47.1, 54.4, 59.8, 65.1, 77.9, 127.8, 128.4, 137.3, 155.3, 156.1, 170.2, 171.3 and 172.0. HRMS: m/z Calcd for [C₄₄H₇₄NaO₁₃Na+Na]⁺: 945.5267. found: 945.5276.

### Synthesis of DFO-Lys(Z)-NH₂ (2)

This Boc-deprotection procedure was modified from a published procedure [Gudmundsdottir A V, Paul C E, Nitz M. Stability studies of hydrazide and hydroxylamine-based glycoconjugates in aqueous solution. Carbohydr Res. 2009; 344: 278-84]. A mixture of trifluoroacetic acid (TFA) (0.5 mL, 6.26 mmol) and dichloromethane (DCM) (0.5 mL) was added to compound 1 (385.1 mg, 0.42 mmol). The reaction mixture was stirred for 4 hours at ambient temperature. Then the solvent was evaporated via rotary evaporator and the crude product was purified with reverse phase column chromatography (10-50% MeCN in water) to isolate compound 2 (327.1 mg, 95%) as a white solid. 1H NMR [(CD₃)₂SO, 500 MHz]: δ 1.21-1.63 (m, 26H, CH₂), 1.96 (s, 3H, CH₃), 2.26 (t, J=7.2, 4H, CH₂), 2.57 (t, J=7.1, 4H, CH₂), 2.95-3.03 (m, 6H, CH₂), 3.05-3.11 (m, 2H, CH₂), 3.44-3.46 (m, 7H), 5.00 (s, 2H, CH₂), 7.23 (t, J=5.6 1H, NH), 7.29 (m, 5H, CH), 7.79 (t, J=5.3, 2H, NH), 8.20 (t, J=5.3, 1H, NH), 9.65 (br s, 3H, OH). ¹³C NMR [(CD₃)₂SO, 126 MHz]: δ 20.4, 21.6, 23.4, 23.5, 26.0, 26.1, 27.6, 28.5, 28.9, 29.9, 30.8, 38.4, 38.7, 46.8, 52.3, 65.2, 168.3, 170.2, 171.3 and 172.0. HRMS: m/z Calcd for [C₃₉H₆₆N₈O₁₃+Na]⁺: 845.4740. found: 845.4741.

### Synthesis of DFO2-Lys-Z (3)

This procedure was adapted from a published literature procedure [Figueras, E.; Martins, A.; Borbely, A.; Le Joncour, V.; Cordella, P.; Perego, R.; Modena, D.; Pagani, P.; Esposito, S.; Auciello, G.; Frese, M.; Gallinari, P.; Laakkonen, P.; Steinkühler, C.; Sewald, N. Octreotide Conjugates for Tumor Targeting and Imaging. Pharmaceutics 2019, 11 (5), 220]. DFO-COOH (470 mg, 0.71 mmol) was dissolved in a mixture of DMF (10 mL) and DMSO (4 mL) with help of heating up to 75° C. After the solution became clear, a mixture of ethyl cyanohydroxyiminoacetate (Oxyma) (74.3 mg, 0.523 mmol) and EDC (103.3 mg, 0.665 mmol) in DMF (10 mL) was added. The mixture was stirred for 30 minutes at ambient temperature. Then a solution of 2 (391 mg, 0.48 mmol) and NaCl (27.8 mg, 0.475 mmol) in DMF (10 mL) was added, and the mixture was sonicated for 5 minutes to ensure complete dissolution. The reaction was stirred for 48 h at ambient temperature then the solvent was reduced to around 1 mL. The residue was added dropwise to ethyl acetate and left in a spark proof −20° C. freezer for 30 minutes. The precipitate was separated with help of centrifugation and washed two times with cold ethyl acetate. The crude product was purified with reverse phase chromatography (10-50% MeCN in water) and the product (336 mg, 32%) was collected as a white powder. ¹H NMR [(CD₃)₂SO, 500 MHz]: δ 1.21-1.1.49 (m, 46H, CH₂), 1.96 (s, 3H, CH₆), 2.24-2.36 (m, 12H, CH₂), 2.56-2.28 (m, 8H, CH₂), 2.94-3.00 (m, 14H, CH₂), 3.43-3.46 (m, 12H, CH₂), 4.06-4.11 (m, 1H, CH), 4.99 (s, 2H, CH₂), 7.22-7.37 (m, 7H, CH—Ar and NH), 7.80-7.99 (m, 8H, NH and OH). ¹³C NMR [(CD₃)₂SO, 126 MHz]: δ 20.4, 22.86, 23.5, 26.1, 27.6, 28.7, 28.8, 29.2, 29.9, 30.7, 30.8, 31.5, 38.4, 46.8, 47.1, 52.7, 65.1, 127.7, 137.3, 156.1, 170.1, 171.4, 171.6, 171.7 and 171.9. HRMS: m/z Calcd for [C₆₈H₁₂₀N₁₆O₁₉+Na]⁺:1487.8331. found: 1487.8366.

### Synthesis of DFO2K

DFO2K was prepared according to a published procedure with modification [Summa, V.; Petrocchi, A.; Bonelli, F.; Crescenzi, B.; Donghi, M.; Ferrara, M.; Fiore, F.; Gardelli, C.; Gonzalez Paz, O.; Hazuda, D. J.; Jones, P.; Kinzel, O.; Laufer, R.; Monteagudo, E.; Muraglia, E.; Nizi, E.; Orvieto, F.; Pace, P.; Pescatore, G.; Scarpelli, R.; Stillmock, K.; Witmer, M. V.; Rowley, M. Discovery of Raltegravir, a Potent, Selective Orally Bioavailable HIV-Integrase Inhibitor for the Treatment of HIV-AIDS Infection. J Med Chem 2008, 51 (18), 5843-5855]. In a Shlenk flask, compound 3 (47.5 mg, 30 μmol) was dissolved in ethanol (30 mL) and 10% Pd/C (6.0 mg, 5 μmol) was added. The flask was sealed with a rubber septa and connected to an H₂ gas balloon fitted with a glass stopcock and a needle, and the flask was additionally connected to vacuum via Schlenk line. The flask was put under vacuum for 15 seconds and then the vacuum was closed and the flask was purged with H₂ gas by slowly opening the glass stopcock valve. The vacuum and purging were repeated 4 times and finally the flask was left stirring under H₂ for 2 hours. After reaction completion was confirmed with low resolution mass spectrometry, the reaction mixture was filtered through celite to remove the Pd/C for safe disposal. The solvent was evaporated, and the crude product mixture was purified by semipreparative HPLC. HPLC conditions: C18 Inspire Semipreparative DIKMA; 5 μm, 21.2×250 mm; (20-30% acetonitrile in water (0.1% formic acid); flow rate, 4 mL/min, t_{R}=18 min). Compound DFO2K (39.4 mg, 90%) was obtained as an off-white solid following rotary evaporation and then freeze drying of pooled HPLC fractions. ¹H NMR [D₂O, 500 MHz]: δ 1.24-1.81 (m, 44H, CH₂), 2.10 (s, 3H, CH₃), 2.44-2.63 (m, 12H, CH₂), 2.62-2.63 (m, 2H, CH₂), 2.76 (t, J=7.0, 6H, CH₂), 2.96 (t, J=7.7, 2H, CH₂), 3.12-3.15 (m, 12H, CH₂), 3.56-3.62 (m, 12H, CH₂), 4.14-4.17 (m, 1H, CH). ¹³C NMR D₂O, 126 MHz]: δ 19.2, 22.2, 23.0, 25.4, 26.2, 17.6, 27.9, 30.4, 30.8, 38.7, 39.1, 47.7, 47.8, 53.9, 173.5, 173.8, 174.3, 174.8, and 174.9. HRMS: m/z Calcd for [C₆₀H₁₁₀N₁₄O₁₉+2H]²⁺: 666.4109. found: 666.4123.

### Preparation of DFO-Km

### Materials and Methods

### Materials

All reagents and solvents were purchased from commercial suppliers (Sigma-Aldrich, St. Louis, MO; TCI America, Portland, OR; Fisher Scientific, Waltham, MA) and were used without further purification unless otherwise indicated. O-benzylhydroxylamine hydrochloride, Oxyma Pure and N,N-diisopropylethylamine (DIPEA), were purchased from Sigma-Aldrich. DFO mesylate was purchased from Abcam. 1-[Bis(dimethylamino)methylene]-1H-1,2,3-triazolo[4,5-b]pyridinium 3-oxide hexafluoroph-osphate (HATU) and H-Lys(Z)-OtBu were purchased from AK Scientific. ZrCl₄, EDTA, disodium salt, dihydrate (Molecular biology grade), ammonium acetate, succinic anhydride and 1-ethyl-3-(3-dimethylaminopropyl)carbodiimide hydrochloride (EDC. HCl) were purchased from Fisher Scientific.

### Chemical Characterization Methods

¹H and ¹³C NMR spectra were recorded on a 500 MHz Bruker Avance III HD NMR spectrometer at the Saskatchewan Structural Science Centre at the University of Saskatchewan, at 25° C. in D₂O, CDCl₃ or (CD₃)₂SO. Chemical shifts were referenced to the residual protons of the deuterated solvents for ¹H NMR (δ=4.79 ppm for D₂O; δ=7.26 ppm for CDCl₃ and δ=2.50 ppm for (CD₃)₂SO. Similarly, ¹³C chemical shifts were referenced to the CDCl₃ signal at 77.16 ppm and (CD₃)₂SO signal at δ=39.52 ppm. Coupling constants are reported to the nearest 0.5 Hz (¹H NMR spectroscopy) and for ¹³C NMR spectroscopy they are rounded to integer values in Hz. Field desorption ionization (FDI) was used on JEOL AccuTOF GCv 4G mass spectrometer to measure high resolution mass spectra and only the mass peak of the isotope with the highest natural abundance is recorded for the isotopic patterns. Antibody conjugates were analyzed on MALDI-TOF MS/MS using Bruker Ultraflex MALDI-TOF/TOF (Bruker Daltonic GmbH) (Alberta Proteomics and Mass spectrometry Facility, University of Alberta, Canada). HPLC purifications were performed on Thermofisher Vanquish HPLC using C18 reversed-phase column (Inspire Semipreparative DIKMA; 5 μm, 21.2×250 mm), a VF-D40-A UV detector, two VF-P10-A pumps and Chromeleon 7 communication software. A flow rate of 4 mL/minute was used with a gradient of MeCN:H₂O (with 0.1% formic acid (FA) in both solvents). Low resolution mass spectrometry was performed on Advion Expression-L system (mass range <2000 amu). The Advion Expression-L was coupled with the Thermofisher Vanquish system to perform LCMS. Concentration of antibody mixtures was determined using a Thermofisher NanoDrop UV/Vis instrument.

The synthesis is shown in Scheme 8 and the synthetic details are provided below:

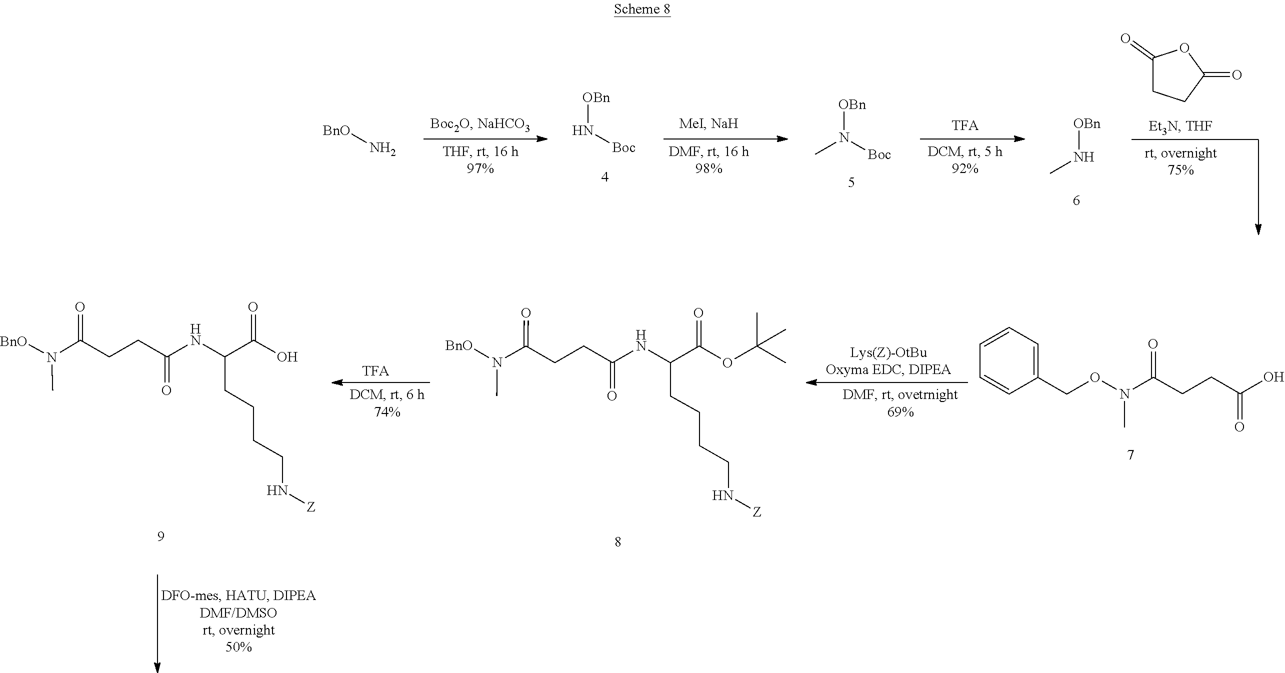

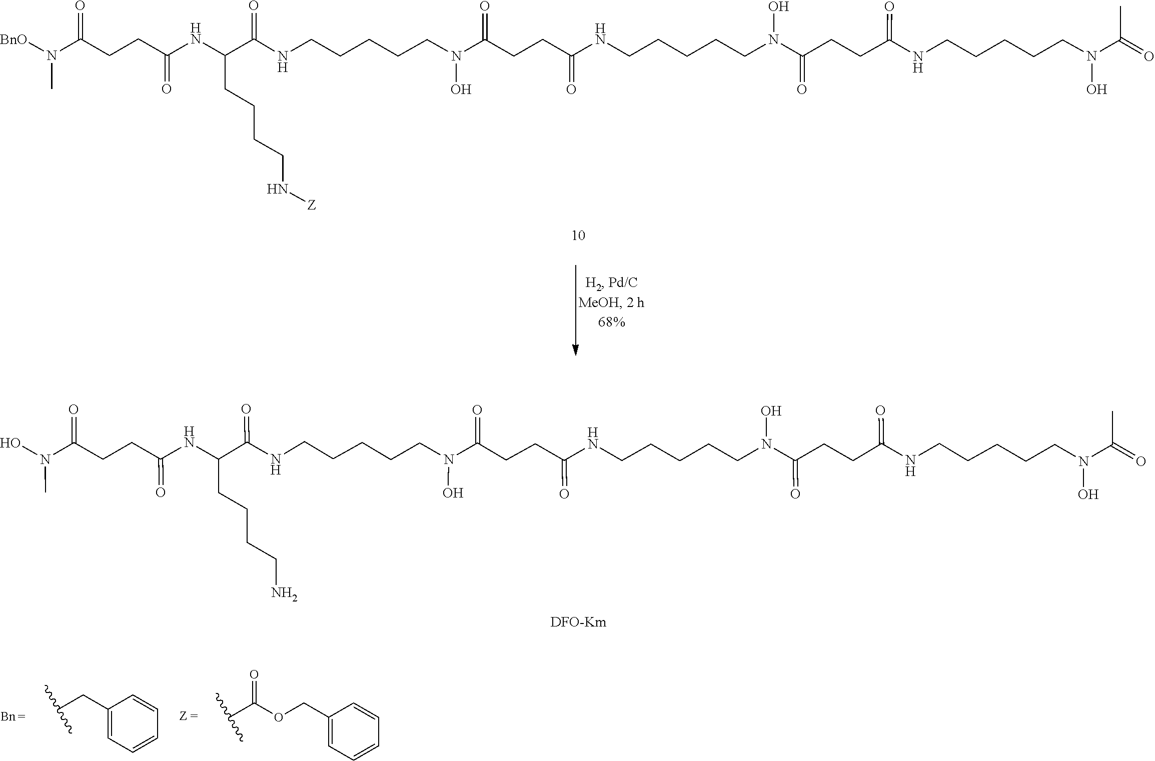

### Synthesis of Tert-butyl N-(benzyloxy)carbamate (4)

Compound 4 was prepared according to a published procedure with modification [Bieliauskas A V, Weerasinghe S V W, Negmeldin A T, Pflum M K H. Structural Requirements of Histone Deacetylase Inhibitors: SAHA Analogs Modified on the Hydroxamic Acid. Arch Pharm (Weinheim). 2016; 349: 373-82]. O-benzylhydroxylamine hydrochloride (1.00 gm, 6.27 mmol) and di-tert-butyl dicarbonate (1.33 mL, 6.23 mmol) were dissolved in THF (7 mL). Then an aqueous solution of sodium hydrogen carbonate (1 M, 7 mL) was added slowly. The reaction was stirred at ambient temperature overnight. The organic solvent was removed by rotary evaporator, then the residue was diluted with water (20 mL) and extracted with dichloromethane (20 mL, 3 times). The organic layers were collected, dried over anhydrous sodium sulfate and then the solvent was removed by rotary evaporator to give the product as a white solid (1.27 gm, 97%), no further purification was required. ¹H NMR [CDCl₃, 500 MHz]: δ 1.48 (s, 9H, CH₃), 4.86 (s, 2H, CH₂), 7.1 (s, 1H, NH), 7.3-7.4 (m, 5H, CH). ¹³C NMR [CDCl₃, 126 MHz]: δ 28.3, 78.5, 81.8, 128.6, 129.2, 135.82, 156.8. HRMS (TOF): m/z Calcd for [C₁₂H₁₇NO₃+Na]⁺:246.1100. found: 246.1103.

### Synthesis of Tert-butyl N-(benzyloxy)-N-methylcarbamate (5)

This procedure was adapted from published literature [Olshvang, E.; Szebesczyk, A.; Kozlowski, H.; Hadar, Y.; Gumienna-Kontecka, E.; Shanzer, A. Biomimetic Ferrichrome: Structural Motifs for Switching between Narrow- and Broad-Spectrum Activities in P. putida and E. coli. Dalton Trans. 2015, 44 (48)]. Sodium hydride (90%, 197.07 mg, 7.39 mmol) was weighed carefully in a Schlenk flask under nitrogen gas and washed with hexane (4 mL) 3 times. Then anhydrous DMF (15 mL) was added and followed by slow addition of compound 4 (1.50 g, 6.72 mmol). After stirring the mixture for 30 minutes at ambient temperature, iodomethane (0.46 mL, 7.39 mmol) was added and the reaction was left to stir at ambient temperature overnight. The reaction was then quenched by careful addition of water (5 mL) and stirred for 10 minutes. The mixture was further diluted with 20 mL water and extracted with hexane (25 mL, 3 times). The organic layers were pooled, dried over anhydrous sodium sulfate and the solvent was removed by rotary evaporator to give compound 5 as a clear oil (1.54 g, 98%). ¹H NMR [CDCl₃, 500 MHz]: δ 1.49 (s, 9H, CH₃), 3.04 (s, 3H, CH₃), 4.82 (s, 2H, CH₂), 7.31-7.41 (m, 5H, CH). ¹³C NMR [CDCl₃, 126 MHz]: δ 28.3, 36.9, 76.6, 81.3, 128.5, 128.6, 129.5, 136.7 and 157.1. HRMS (TOF): m/z Calcd for [C₁₃H₁₉NO₃+H]⁺: 238.1437. found: 238.1432.

### Synthesis of (benzyloxy)(methyl)amine (6)

This Boc-deprotection procedure was modified from published procedure [Gudmundsdottir A V, Paul C E, Nitz M. Stability studies of hydrazide and hydroxylamine-based glycoconjugates in aqueous solution. Carbohydr Res. 2009; 344: 278-84]. To a solution of compound 5 (1.49 g, 6.28 mmol) in dichloromethane (7.5 mL), trifluoroacetic acid (7.5 mL, 97.32 mmol) was added and stirred for 5 h at ambient temperature. A saturated sodium hydrogen carbonate solution (20 mL) was added and extracted with dichloromethane (20 mL, 3 times). The organic layers were collected and dried over anhydrous sodium sulfate. The solvent was evaporated via rotary evaporator and the crude product mixture was purified with column chromatography (30% ethyl acetate in hexane). Compound 6 was obtained (0.79 g, 92%) as a clear oil. ¹H NMR [CDCl₃, 500 MHz]: δ 2.74 (s, 3H, CH₃), 4.73 (2H, CH₂), 5.56 (s, 1H, NH), 7.31-7.41 (m, 5H, CH). ¹³C NMR [CDCl₃, 126 MHz]: δ 39.3, 75.6, 127.8, 128.3, 128.4, 138.0. HRMS (TOF): m/z Calcd for [C₁₈H₁₁NO+H]⁺: 138.0913. found: 138.0908.

### Synthesis of Compound (7)

Compound 7 was synthesized based on a published procedure with modifications [Olshvang, E.; Szebesczyk, A.; Kozlowski, H.; Hadar, Y.; Gumienna-Kontecka, E.; Shanzer, A. Biomimetic Ferrichrome: Structural Motifs for Switching between Narrow- and Broad-Spectrum Activities in P. putida and E. coli. Dalton Trans. 2015, 44 (48)]. Succinic anhydride (2.19 g, 21.9 mmol) was added to a mixture of 6 (1.00 g, 7.29 mmol) and triethylamine (2.0 mL, 14.34 mmol) in THF (16 mL). The reaction mixture was stirred overnight at ambient temperature. After the reaction completion was confirmed with TLC (50% ethyl acetate in hexane, Rf=0.28), the solvent was evaporated, and the residue was dissolved in aqueous sodium hydroxide (30 mL, 0.5 M). The solution was washed with diethyl ether (20 mL×2). Then the solution was acidified to pH ˜2 with hydrochloric acid and the product was extracted with dichloromethane (40 mL×3). The organic layers were combined, dried over anhydrous sodium sulfate, and the solvent was removed by rotary evaporator to give compound 7 (1.29 g, 75%). ¹H NMR [CDCl₃, 500 MHz]: δ 2.62 (t, J=6.5 Hz, 2H, CH₂), 2.72 (t, J=6.4 Hz, 2H, CH₂), 3.19 (s, 3H, CH₃), 4.81 (s, 2H, CH₂), 7.32-737 (m, 5H, CH Ar), 10.93 (s, 1H, OH). ¹³C NMR [CDCl₃, 126 MHz]: δ 27.0, 28.6, 76.2, 128.7, 129.3, 134.2, 173.6, 177.6. HRMS: m/z Calcd for [C₁₂H₁₅NO₄+H]⁺: 238.1074. found: 238.1082.

### Synthesis of Compound (8)

To solution of 7 (500.20 mg, 2.11 mmol) in DMF (15 mL), Oxyma (331.1 mg, 2.33 mmol), EDC (448.1 mg, 2.32 mmol), and N,N-diisopropylethylamine (1.1 mL, 6.32 mmol) were added. The mixture was stirred for 20 minutes at ambient temperature before H-Lys(Z)-OtBu HCl (851.4 mg, 2.28 mmol) was added, and the reaction was stirred overnight at ambient temperature. Then the reaction mixture was added to water (50 mL) and extracted with dichloromethane (30 mL×4). The combined organic layers were collected and washed with water, dried with anhydrous sodium sulfate. The solvent was removed by using rotary evaporator and the obtained crude product mixture was purified with column chromatography (50% ethyl acetate in hexane, Rf=0.21) to give compound 8 (809.7 mg, 69%). ¹H NMR [CDCl₃, 500 MHz]: δ 1.34-1.41 (m, 2H, CH₂), 1.44 (s, 9H, CH₃), 1.50 (quint, J=7.0, 2H, CH₂), 1.58-1.86 (m, 2H, CH₂), 1.50 (quint, J=7.0 Hz, 2H, CH₂), 2.45-2.48 (m, 2H, CH₂), 2.67-2.84 (m, 2H, CH₂), 3.1 (s, 3H, CH₃), 3.14-3.22 (m, 2H, CH₂), 4.43, 4.48 (m, 1H, CH), 4.82 (s, 2H, CH₂), 5.06 (q, J=11.4 Hz, 2H, CH₂), 5.31 (br s, 1H, NH), 6.45 (d, J=7.8 Hz, 1H, NH), 7.27-7.32 (m, 5H, CH), 7.36-7.38 (m, 5H, CH). ¹³C NMR [CDCl₃, 126 MHz]: δ 22.3, 27.7, 28.0, 29.2, 30.5, 32.1, 40.5, 66.5, 76.3, 81.9, 128.0, 128.1, 128.5, 128.7, 129.0, 129.3, 156.6, 171.6, 171.9. HRMS: m/z Calcd for [C₃₀H₄₁N₃O₇+H]⁺: 556.3017. found: 556.2992.

### Synthesis of Compound (9)

This procedure was modified from a published procedure [Gudmundsdottir A V, Paul C E, Nitz M. Stability studies of hydrazide and hydroxylamine-based glycoconjugates in aqueous solution. Carbohydr Res. 2009; 344: 278-84]. Compound 8 (1.11 g, 2.00 mmol) was dissolved in a mixture of TFA (2.5 mL, 32.7 mmol) and dichloromethane (5 mL). The reaction was stirred for 6 hours at RT. Then the volatiles were removed by using rotary evaporator and the crude mixture was purified through column chromatography (10% methanol in ethyl acetate, Rf=0.4) to give compound 9 (0.73 g, 74%). ¹H NMR [CDCl₃, 500 MHz]: δ 1.36-1.1.50 (m, 4H, CH₂), 1.66-1.92 (m, 2H, CH₂), 2.51 (br s, 2H, CH₂), 2.77 (br s, 2H, CH₂), 3.11 (s, 3H, CH₃), 3.15-3.19 (m, 2H, CH₂), 4.54 (q, J=6.7 Hz, 1H, CH), 4.82 (s, 2H, CH₂), 5.03-5.12 (m, 2H, CH₂). 7.03 (br s, 1H, NH), 7.28-7.36 (m, 10H, CH), 10.17 (br s, 1H, OH). ¹³C NMR [CDCl₃, 126 MHz]: δ 22.3, 27.7, 29.3, 30.4, 31.4, 33.7, 40.6, 52.3, 66.7, 76.4, 128.1, 128.2, 128.6, 128.8, 129.1, 129.4, 134.3, 136.7, 156.9, 173.1, 174.2, 174.8. HRMS: m/z Calcd for [C₂₆H₃₃N₃O₇+H]⁺: 500.2391. found: 500.2410.

### Synthesis of Compound (10)

A solution of HATU (0.27 g, 0.71 mmol) and DIPEA (0.35 mL, 0.70 mmol) in 14 mL DMF was added to a solution of 9 (0.30 g, 0.60 mmol) in 13 mL DMF and the mixture was stirred for 10 minutes at ambient temperature. DFO mesylate (0.44 g, 0.67 mmol) was dissolved in 10 mL DMF and 4 mL DMSO (at 80° C.). Once fully dissolved, the DFO mixture was allowed to cool for few minutes (˜50° C.), it was then added to the above solution of the activated ester. The reaction was left to stir overnight at ambient temperature. Then the reaction mixture was condensed to ˜2 mL by using rotary evaporator and the residue was added dropwise to ˜70 mL ethyl acetate spread into 2 falcon tubes (50 mL size tubes). The tubes were kept in a spark proof −20° C. freezer until maximum amount of precipitate was formed (overnight). The precipitate was separated by decantation after centrifugation (4000 rpm, 15 minutes) and washed two times with cold ethyl acetate. The crude was then purified by using reverse phase column chromatography (Biotage Isolera) (10-100% acetonitrile in water) to collect 10 (316 mg, 50%) as an off-white powder. ¹H NMR [(CD₃)₂SO, 500 MHz]: δ 1.19-1.67 (m, 28H, CH₂), 1.96 (s, 3H, CHs), 2.26 (t, J=7.3 Hz, 4H, CH₂), 2.31-2.38 (m, 2H, CH₂), 2.57 (t, J=6.9 Hz, 4H, CH₂), 2.65 (br s, 2H, CH₂), 2.9-3.00 (m, 8H, CH₂), 3.12 (s, 3H, CHs), 3.44 (q, J=6.6 Hz, 6H, CH₂), 4.08-4.13 (m, 1H, CH), 4.90 (s, 2H, CH₂), 4.99 (s, 2H, CH₂), 7.22 (t, J=5.6 Hz, 1H, NH), 7.30-7.45 (m, 1OH, CH), 7.76 (t, J=5.5 Hz, 1H, NH), 7.82 (br s, 2H, NH), 8.00 (br d, J=8, 1H, NH), 9.74 (br s, 3H, OH). ¹³C NMR [(CD₃)₂SO, 126 MHz]: δ 22.8, 23.1, 23.5, 26.0, 27.1, 27.6, 28.7, 28.8, 29.1, 29.6, 30.0, 31.6, 38.4, 46.8, 47.1, 65.1, 127.7, 128.4, 128.5, 128.7, 129.4, 137.3, 156.1, 171.3, 171.6, 171.9. HRMS: m/z Calcd for [C₅₁H₇₉N₉O₁₄+H]⁺: 1042.5825. found: 1042.5819.

### Synthesis of Compound DFO-Km

In a Schlenk flask, 10% Palladium on carbon (25.0 mg, 23.5 μmol) was added to a solution of compound 10 (120.0 mg, 115 μmol) in 20 mL methanol. The flask was sealed with a rubber septa and connected to an H₂ gas balloon fitted with a glass stopcock and a needle, and the flask was additionally connected to vacuum via Schlenk line. The flask was put under vacuum for 15 seconds and then the vacuum was closed, and the flask was purged with H₂ gas by slowly opening the glass stopcock valve. The vacuum and purging were repeated 4 times and finally the flask was left stirring under H₂ for 2 hours. Then the reaction solution was filtered through celite to remove the Pd/C, and the solvent was evaporated by using rotary evaporator. The crude product mixture was purified by using semipreparative RP-HPLC. HPLC conditions: C18 Inspire Semipreparative DIKMA; 5 μm, 21.2×250 mm; (20-32% acetonitrile in water (0.1% formic acid); flow rate, 4 mL/min, t_{R}=15 min) to give DFO-Km (1-7) (83.5 mg, 68%) as white powder. ¹H NMR [D₂O, 500 MHz]: δ 1.20-1.84 (m, 24H, CH₂), 2.10 (s, 3H, CH₃), 2.44-2.53 (m, 6H, CH₂), 2.62-2.81 (m, 6H, CH₂), 2.96 (t, J=7.6 Hz, 2H, CH₂), 3.12-3.16 (m, 6H, CH₂), 3.19 (s, 3H, CHs), 3.55-3.62 (m, 6H, CH₂), 4.16 (q, J=4.8, 1H, CH). ¹³C NMR [D₂O, 126 MHz]: δ 19.3, 22.1, 23.0, 25.4, 26.2, 27.1, 27.6, 27.8, 28.0, 29.8, 30.3, 30.4, 30.7, 35.8, 39.1, 47.6, 47.8, 51.0, 51.5, 53.8, 170.8, 173.5, 173.8, 173.9, 174.7, 175.4. HRMS: m/z Calcd for [C₃₆H₆₇N₉O₁₂+H]⁺: 818.4982. found: 818.4942.

### Example 4: Preparation of DFO-PyrZW-p-SCN-Ph

The preparation of DFO-PyrZW-p-SCN-Ph is shown in Scheme 9 and the synthetic details are provided below:

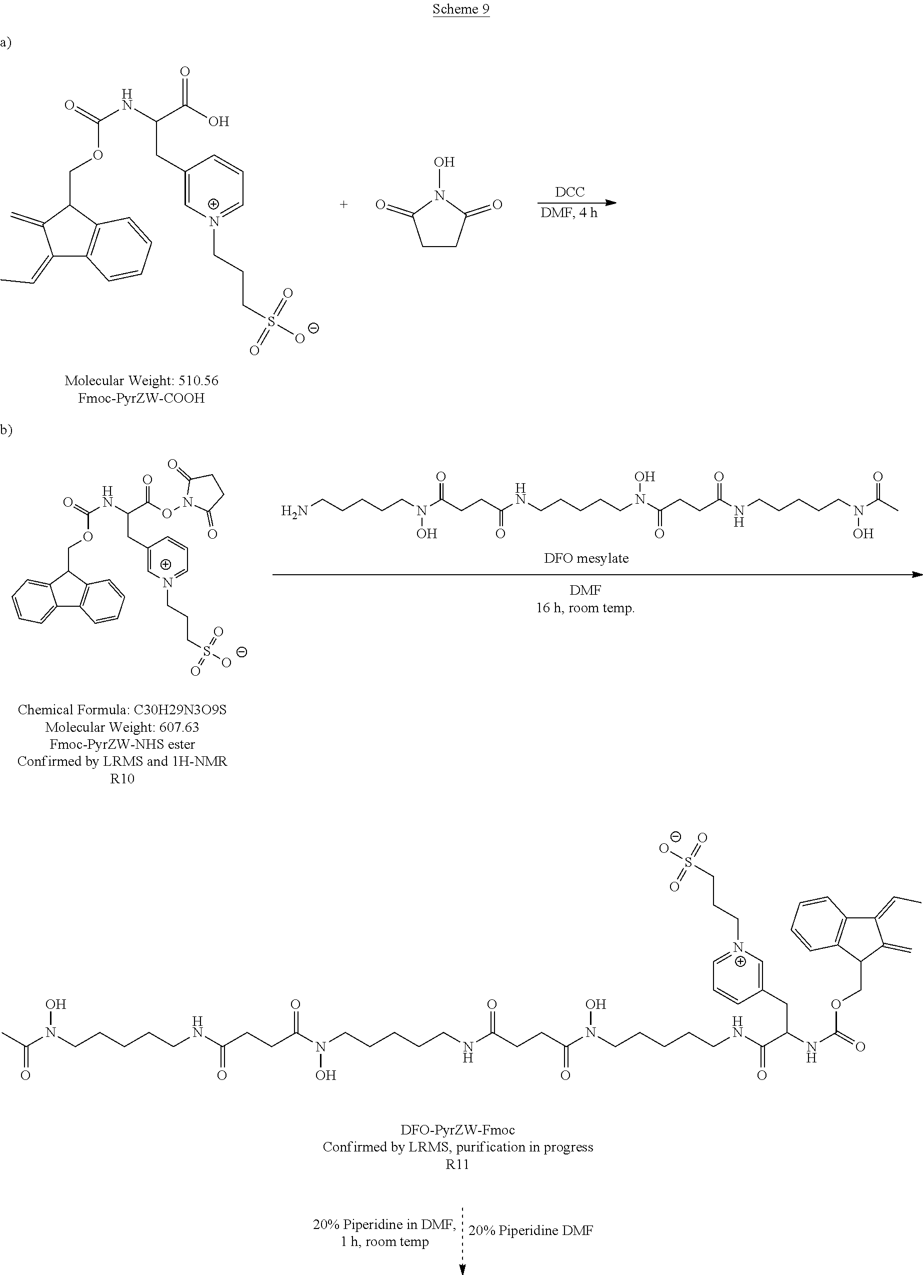

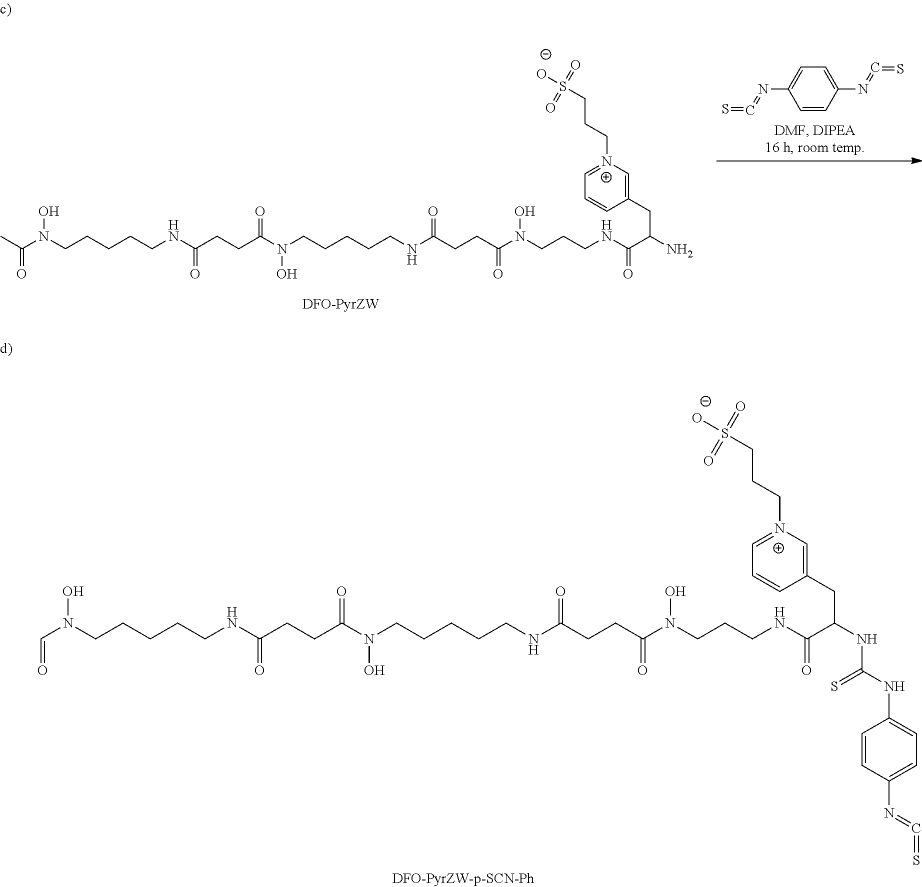 [SMILES: CC(=O)N(O)CCCCCNC(=O)CCC(=O)N(O)CCCCCNC(=O)CCC(=O)N(O)CCCNC(=O)C(N)Cc1ccc[n+](CCCS(=O)(=O)[O-])c1 ; S=C=Nc1ccc(N=C=S)cc1 ; O=CN(O)CCCCCNC(=O)CCC(=O)N(O)CCCCCNC(=O)CCC(=O)N(O)CCCNC(=O)C(Cc1ccc[n+](CCCS(=O)(=O)[O-])c1)NC(=S)Nc1ccc(N=C=S)cc1]

### Synthesis of R10 (Step a)

Fmoc-Ala-PyrZW-OH (100 mg, 0.2 mmol) was dissolved in DMF (2.0 ml). To this, 1 eq. of DCC (41.3 mg) and 1 eq. NHS (23.0 mg) were added and the reaction mixture was stirred for 4 hours at room temperature. The by-product precipitate was filtered out and the filtrate concentrated and left on high-vacuum overnight. The oily product was dried by dissolving it in CHCl₃ and hexane and removing solvent under partial vacuum with a rotary evaporator a 2-3 times until it gives a dry white powder (81.86 mg, 67.4% yield). Calculated mass for C30H29N3O9S was 607.63, found 608.3 m/z.

### Synthesis of R11 (Steps b and c):

Fmoc-PyrZW-NHS (20.0 mg, 0.033 mmol) was dissolved in dry DMF (0.5 ml) in a round bottom flask equipped with a stirring bar. DFO mesylate (18.5 mg, 0.033 mmol, 1 eq.) was dissolved in DMF (0.3 ml) and DMSO (0.2 ml) heating in an oil bath at 75 degree celsius until the solution was clear, and it was added to the round bottom flask containing PyrZW-NHS in one portion. The reaction mixture was left to react overnight at room temperature. The crude reaction mixture was then checked with LRMS. Calculated mass for C51H72N8014S was 1053.24, found 527.12 [M+2H]+, and 1053.4 m/z.

The crude product was concentrated to remove the solvent by cold-finger before its purification on a C18 column (20% CH₃CN:H₂O gradient).

DFO-PyrZW was obtained as shown in step c) on scheme 9 above and the final product DFO-PyrZW-p-SCN-Ph was obtained as shown in step d) on scheme 9 above.

### FULL CITATIONS FOR DOCUMENTS REFERRED TO IN THE APPLICATION

1. Choi, H. S.; Nasr, K.; Alyabyev, S.; Feith, D.; Lee, J. H.; Kim, S. H.; Ashitate, Y.; Hyun, H.; Patonay, G.; Strekowski, L.; Henary, M.; Frangioni, J. V., Synthesis and In Vivo Fate of Zwitterionic Near-Infrared Fluorophores( ). Angewandte Chemie (International ed. in English) 2011, 50 (28), 6258-6263.
2. Choi, H. S.; Gibbs, S. L.; Lee, J. H.; Kim, S. H.; Ashitate, Y.; Liu, F.; Hyun, H.; Park, G.; Xie, Y.; Bae, S.; Henary, M.; Frangioni, J. V., Targeted zwitterionic near-infrared fluorophores for improved optical imaging. Nat. Biotechnol. 2013, 31 (2), 10.1038/nbt.2468.
3. Hyun, H.; Henary, M.; Gao, T.; Narayana, L.; Owens, E. A.; Lee, J. H.; Park, G.; Wada, H.; Ashitate, Y.; Frangioni, J. V.; Choi, H. S., 700-nm Zwitterionic Near-Infrared Fluorophores for Dual-Channel Image-Guided Surgery. Mol. Imaging Biol. 2016, 18 (1), 52-61.
4. de Valk, K. S.; Handgraaf, H. J.; Deken, M. M.; Sibinga Mulder, B. G.; Valentijn, A. R.; Terwisscha van Scheltinga, A. G.; Kuil, J.; van Esdonk, M. J.; Vuijk, J.; Bevers, R. F.; Peeters, K. C.; Holman, F. A.; Frangioni, J. V.; Burggraaf, J.; Vahrmeijer, A. L., A zwitterionic near-infrared fluorophore for real-time ureter identification during laparoscopic abdominopelvic surgery. Nat. Commun. 2019, 10 (1), 3118.
5. Sponchioni, M.; Rodrigues Bassam, P.; Moscatelli, D.; Arosio, P.; Capasso Palmiero, U., Biodegradable zwitterionic nanoparticles with tunable UCST-type phase separation under physiological conditions. Nanoscale 2019, 11 (35), 16582-16591.
6. Debayle, M.; Balloul, E.; Dembele, F.; Xu, X.; Hanafi, M.; Ribot, F.; Monzel, C.; Coppey, M.; Fragola, A.; Dahan, M.; Pons, T.; Lequeux, N., Zwitterionic polymer ligands: an ideal surface coating to totally suppress protein-nanoparticle corona formation? Biomaterials 2019, 219, 119357.
7. Ibrahim, G. P. S.; Isloor, A. M.; Inamuddin; Asir, A. M.; Ismail, N.; Ismail, A. F.; Ashraf, G. M., Novel, one-step synthesis of zwitterionic polymer nanoparticles via distillation-precipitation polymerization and its application for dye removal membrane. Scientific Reports 2017, 7 (1), 15889.
8. Estephan, Z. G.; Jaber, J. A.; Schlenoff, J. B., Zwitterion-Stabilized Silica Nanoparticles: Toward Nonstick Nano. Langmuir 2010, 26 (22), 16884-16889.
9. Hillner, B. E.; Siegel, B. A.; Shields, A. F.; Liu, D.; Gareen, I. F.; Hunt, E.; Coleman, R. E., Relationship Between Cancer Type and Impact of PET and PET/CT on Intended Management Findings of the National Oncologic PET Registry. J. Nucl. Med. 2008, 49 (12), 1928-1935.
10. Baum, R. P.; Kulkami, H. R., THERANOSTICS: From Molecular Imaging Using Ga-68 Labeled Tracers and PET/CT to Personalized Radionuclide Therapy—The Bad Berka Experience. Theranostics 2012, 2 (5), 437-447.
11. Baum, R. P.; Kulkami, H. R.; Carreras, C., Peptides and Receptors in Image-Guided Therapy: Theranostics for Neuroendocrine Neoplasms. Semin. Nucl. Med. 2012, 42 (3), 190-207.
12. Kelkar, S. S.; Reineke, T. M., Theranostics: Combining Imaging and Therapy. Bioconjugate Chemistry 2011, 22 (10), 1879-1903.
13. Der, M. A.; Zeglis, B. M.; Francesconi, L. C.; Lewis, J. S., PET imaging with 89Zr: From radiochemistry to the clinic. Nucl. Med. Biol. 2013, 40 (1), 3-14.
14. Price, E. W.; Camazza, K. E.; Carlin, S. D.; Cho, A.; Edwards, K. J.; Sevak, K. K.; Glaser, J. M.; de Stanchina, E.; Janjigian, Y. Y.; Lewis, J. S., 89Zr-DFO-AMG102 Immuno-PET to Determine Local HGF Protein Levels in Tumors for Enhanced Patient Selection. J. Nucl. Med. 2017, 58 (9), 1386-1394.
15. Bailly, C.; Clery, P. F.; Faivre-Chauvet, A.; Bourgeois, M.; Guerard, F.; Haddad, F.; Barbet, J.; Cherel, M.; Kraeber-Bodere, F.; Carlier, T.; Bodet-Milin, C., Immuno-PET for Clinical Theranostic Approaches. Int J Mol Sci 2016, 18 (1).
16. van Dongen, G. A.; Visser, G. W.; Lub-de Hooge, M. N.; de Vries, E. G.; Perk, L. R., Immuno-PET: a navigator in monoclonal antibody development and applications. Oncologist 2007, 12 (12), 1379-89.
17. Kunikowska, J.; Pawlak, D.; Bak, M. I.; Kos-Kudfa, B.; Mikolajczak, R.; Królicki, L., Long-term results and tolerability of tandem peptide receptor radionuclide therapy with 90Y/177Lu-DOTATATE in neuroendocrine tumors with respect to the primary location: a 10-year study. Ann. Nucl. Med. 2017, 31 (5), 347-356.
18. Hope, T. A.; Bergsland, E.; Bozkurt, M. F.; Graham, M. M.; Heaney, A. P.; Herrmann, K.; Howe, J. R.; Kulke, M. H.; Kunz, P. L.; Mailman, J.; May, L.; Metz, D. C.; Millo, C.; O'Dorisio, S.; Reidy-Lagunes, D. L.; Soulen, M. C.; Strosberg, J. R., Appropriate Use Criteria for Somatostatin Receptor PET Imaging in Neuroendocrine Tumors. J. Nucl. Med. 2017.
19. Kulkami, H. R.; Baum, R. P., Patient Selection for Personalized Peptide Receptor Radionuclide Therapy Using Ga-68 Somatostatin Receptor PET/CT. PET Clinics 2014, 9 (1), 83-90.
20. Litau, S.; Niedermoser, S.; Vogler, N.; Roscher, M.; Schirrmacher, R.; Fricker, G.; Wängler, B.; Wängler, C., Next Generation of SiFAlin-Based TATE Derivatives for PET Imaging of SSTR-Positive Tumors: Influence of Molecular Design on In Vitro SSTR Binding and In Vivo Pharmacokinetics. Bioconjugate Chemistry 2015, 26 (12), 2350-2359.
21. Lears, K. A.; Ferdani, R.; Liang, K.; Zheleznyak, A.; Andrews, R.; Sherman, C. D.; Achilefu, S.; Anderson, C. J.; Rogers, B. E., In Vitro and In Vivo Evaluation of 64Cu-Labeled SarAr-Bombesin Analogs in Gastrin-Releasing Peptide Receptor-Expressing Prostate Cancer. J. Nucl. Med. 2011, 52 (3), 470-477.
22. Fragogeorgi, E. A.; Zikos, C.; Goumi, E.; Bouziotis, P.; Paravatou-Petsotas, M.; Loudos, G.; Mitsokapas, N.; Xanthopoulos, S.; Mavri-Vavayanni, M.; Livaniou, E.; Varvarigou, A. D.; Archimandritis, S. C., Spacer Site Modifications for the Improvement of the in Vitro and in Vivo Binding Properties of 99mT-N3S-X-Bombesin[2 #14]Derivatives. Bioconjugate Chemistry 2009, 20 (5), 856-867.
23. Wang, L.; Shi, J.; Kim, Y.-S.; Zhai, S.; Jia, B.; Zhao, H.; Liu, Z.; Wang, F.; Chen, X.; Liu, S., Improving Tumor-Targeting Capability and Pharmacokinetics of 99mTc-Labeled Cyclic RGD Dimers with PEG4 Linkers. Mol. Pharm. 2009, 6 (1), 231-245.
24. Wang, L.; Shi, J.; Kim, Y.-S.; Zhai, S.; Jia, B.; Zhao, H.; Liu, Z.; Wang, F.; Chen, X.; Liu, S., Improving Tumor-Targeting Capability and Pharmacokinetics of 99mTc-Labeled Cyclic RGD Dimers with PEG4 Linkers. Mol. Pharm. 2008, 6 (1), 231-245.
25. Garcia Garayoa, E.; Schweinsberg, C.; Maes, V.; Brans, L.; Bläuenstein, P.; Tourwé, D. A.; Schibli, R.; Schubiger, P. A., Influence of the Molecular Charge on the Biodistribution of Bombesin Analogues Labeled with the [99mTc(CO)3]-Core. Bioconjugate Chemistry 2008, 19 (12), 2409-2416.
26. Vegt, E.; Melis, M.; Eek, A.; de Visser, M.; Brom, M.; Oyen, W. J. G.; Gotthardt, M.; de Jong, M.; Boerman, O. C., Renal uptake of different radiolabelled peptides is mediated by megalin: SPECT and biodistribution studies in megalin-deficient mice. Eur. J. Nucl. Med. Mol. Imaging 2011, 38 (4), 623-632.
27. de Jong, M.; Valkema, R.; van Gameren, A.; van Boven, H.; Bex, A.; van de Weyer, E. P.; Burggraaf, J. D.; Körner, M.; Reubi, J.-C.; Krenning, E. P., Inhomogeneous Localization of Radioactivity in the Human Kidney After Injection of [111In-DTPA]Octreotide. J. Nucl. Med. 2004, 45 (7), 1168-1171.
28. Christensen, E. I.; Bim, H., Megalin and cubilin: multifunctional endocytic receptors. Nat. Rev. Mol. Cell Biol. 2002, 3, 258.
29. Vegt, E.; de Jong, M.; Wetzels, J. F. M.; Masereeuw, R.; Melis, M.; Oyen, W. J. G.; Gotthardt, M.; Boerman, O. C., Renal Toxicity of Radiolabeled Peptides and Antibody Fragments: Mechanisms, Impact on Radionuclide Therapy, and Strategies for Prevention. J. Nucl. Med. 2010, 51 (7), 1049-1058.
30. Akizawa, H.; Arano, Y.; Mifune, M.; Iwado, A.; Saito, Y.; Mukai, T.; Uehara, T.; Ono, M.; Fujioka, Y.; Ogawa, K.; Kiso, Y.; Saji, H., Effect of molecular charges on renal uptake of 111In-DTPA-conjugated peptides. Nucl. Med. Biol. 2001, 28 (7), 761-768.
31. Sølling, K.; Mogensen, C. E., Studies on the mechanism of renal tubular protein reabsorption. Proceedings of the European Dialysis and Transplant Association. European Dialysis and Transplant Association 1977, 14, 543-549.
32. Arano, Y., Strategies to reduce renal radioactivity levels of antibody fragments. The quarterly journal of nuclear medicine: official publication of the Italian Association of Nuclear Medicine (AIMN) [and] the International Association of Radiopharmacology (IAR) 1998, 42 (4), 262-270.
33. Rolleman, E. J.; Valkema, R.; de Jong, M.; Kooij, P. P.; Krenning, E. P., Safe and effective inhibition of renal uptake of radiolabelled octreotide by a combination of lysine and arginine. Eur. J. Nucl. Med. Mol. Imaging 2003, 30 (1), 9-15.
34. Gotthardt, M.; van Eerd-Vismale, J.; Oyen, W. J. G.; de Jong, M.; Zhang, H.; Rolleman, E.; Maecke, H. R.; Béhé, M.; Boerman, O., Indication for Different Mechanisms of Kidney Uptake of Radiolabeled Peptides. J. Nucl. Med. 2007, 48 (4), 596-601.
35. Akizawa, H.; Imajima, M.; Hanaoka, H.; Uehara, T.; Satake, S.; Arano, Y., Renal Brush Border Enzyme-Cleavable Linkages for Low Renal Radioactivity Levels of Radiolabeled Antibody Fragments. Bioconjugate Chemistry 2013, 24 (2), 291-299.
36. Akizawa, H.; Uehara, T.; Arano, Y., Renal uptake and metabolism of radiopharmaceuticals derived from peptides and proteins. Advanced Drug Delivery Reviews 2008, 60 (12), 1319-1328.
37. Sun, X.; Li, Y.; Liu, T.; Li, Z.; Zhang, X.; Chen, X., Peptide-based imaging agents for cancer detection. Advanced Drug Delivery Reviews 2017, 110-111, 38-51.
38. Ebenhan, T.; Gheysens, O.; Kruger, H. G.; Zeevaart, J. R.; Sathekge, M. M., Antimicrobial peptides: their role as infection-selective tracers for molecular imaging. BioMed research international 2014, 2014.
39. Baum, R. P.; Kluge, A. W.; Kulkami, H.; Schorr-Neufing, U.; Niepsch, K.; Bitterlich, N.; van Echteld, C. J. A., [(177)Lu-DOTA](0)-D-Phe(1)-Tyr(3)-Octreotide ((177)Lu-DOTATOC) For Peptide Receptor Radiotherapy in Patients with Advanced Neuroendocrine Tumours: A Phase-II Study. Theranostics 2016, 6 (4), 501-510.
40. Läppchen, T.; Tönnesmann, R.; Eersels, J.; Meyer, P. T.; Maecke, H. R.; Rylova, S. N., Radioiodinated Exendin-4 Is Superior to the Radiometal-Labelled Glucagon-Like Peptide-1 Receptor Probes Overcoming Their High Kidney Uptake. PLoS One 2017, 12 (1), e0170435.
41. Breeman, W. A. P.; de Blois, E.; Sze Chan, H.; Konijnenberg, M.; Kwekkeboom, D. J.; Krenning, E. P., 68Ga-labeled DOTA-Peptides and 68Ga-labeled Radiopharmaceuticals for Positron Emission Tomography: Current Status of Research, Clinical Applications, and Future Perspectives. Semin. Nucl. Med. 2011, 41 (4), 314-321.
42. Correia, J. D. G.; Paulo, A.; Raposinho, P. D.; Santos, I., Radiometallated peptides for molecular imaging and targeted therapy. Dalton Trans. 2011, 40 (23), 6144-6167.
43. Bacher, L.; Fischer, G.; Litau, S.; Schirrmacher, R.; Wängler, B.; Baller, M.; Wängler, C., Improving the stability of peptidic radiotracers by the introduction of artificial scaffolds: which structure element is most useful? J. Label. Compd. Radiopharm. 2015, 58 (10). 395-402.
44. Raheem, S. J.; Schmidt, B. W.; Solomon, V. R.; Salih, A. K.; Price, E. W., Ultrasonic-Assisted Solid-Phase Peptide Synthesis of DOTA-TATE and DOTA-linker-TATE Derivatives as a Simple and Low-Cost Method for the Facile Synthesis of Chelator-Peptide Conjugates. Bioconjugate chemistry 2021, 32 (7), 1204-1213.
45. Mueller, D.; Breeman, W. A. P.; Klette, I.; Gottschaldt, M.; Odparlik, A.; Baehre, M.; Tworowska, I.; Schultz, M. K., Radiolabeling of DOTA-like conjugated peptides with generator-produced 68Ga and using NaCl-based cationic elution method. Nature Protocols 2016, 11, 1057.
46. Maschauer, S.; Einsiedel, J.; Haubner, R.; Hocke, C.; Ocker, M.; Hubner, H.; Kuwert, T.; Gmeiner, P.; Prante, O., Labeling and glycosylation of peptides using click chemistry: a general approach to (18)F-glycopeptides as effective imaging probes for positron emission tomography. Angew Chem Int Ed Engl 2010, 49 (5), 976-9.
47. Maschauer, S.; Heilmann, M.; Wängler, C.; Schirrmacher, R.; Prante, O., Radiosynthesis and Preclinical Evaluation of 18F-Fluoroglycosylated Octreotate for Somatostatin Receptor Imaging. Bioconjugate chemistry 2016, 27 (11), 2707-2714.
48. Xia, Y.; Zeng, C.; Zhao, Y.; Zhang, X.; Li, Z.; Chen, Y., Comparative evaluation of (68)Ga-labelled TATEs: the impact of chelators on imaging. EJNMMI Res 2020, 10 (1), 36.

1. A zwitterion compound of Formula I, or a salt and/or solvate thereof: 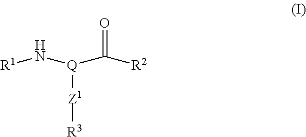
wherein
R¹ is H, OH or a protecting group;
R² is OH or a protecting group;
Q is selected from Q1, Q2, Q3, Q4 and Q5: 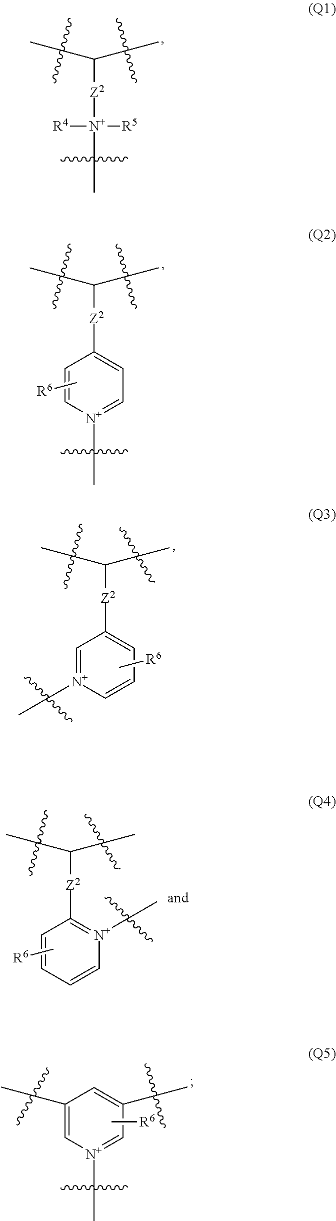
Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R³ is an anion containing group selected from sulfonate, carboxylate, sulfate and phosphate.

2. The compound of claim 1, wherein R¹ is H or the protecting group.

3. The compound of claim 1 or 2, wherein R² is OH or the protecting group.

4. The compound of any one of claims 1 to 3, wherein the protecting group is selected from 9-fluorenenylmethoxycarbonyl (Fmoc), tert-butyloxycarbonyl (Boc), tert-butyl (^{t}Bu), trityl (Trt), 2,4-dimethoxybenzyl (Dmb), 9-fluorenylmethyl (Fm) and benzyl (Bn).

5. The compound of claim 4, wherein the protecting group is selected from Fmoc and Boc.

6. The compound of any one of claims 1 to 5, wherein R¹ is the protecting group.

7. The compound of claim 6, wherein R¹ is Fmoc.

8. The compound of any one of claims 1 to 7, wherein R² is OH.

9. The compound of any one of claims 1 to 8, wherein the compound of Formula I, or a salt and/or solvate thereof, has the following structure: 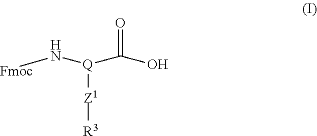
wherein Q, Z¹ and R³ are as defined in claim 1.

10. The compound of any one of claims 1 to 9, wherein R⁴ and R⁵ are independently selected from C₁₋₆alkyl, C₂₋₆alkenyl and C₂₋₆alkynyl.

11. The compound of claim 10, wherein R⁴ and R⁵ are independently selected from CH₂CH₂CH₃, CH(CHs)₂, CH₂CH₃ and CH₃.

12. The compound of claim 11, wherein each one of R⁴ and R⁵ is CH₃.

13. The compound of any one of claims 1 to 9, wherein R⁶ is H.

14. The compound of any one of claims 1 to 13, wherein Z² is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, NH and S.

15. The compound of claim 14, wherein Z² is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, NH and S.

16. The compound of claim 15, wherein Z² is C₄alkylene.

17. The compound of claim 15, wherein Z² is C₁alkylene.

18. The compound of any one of claims 1 to 17, wherein Z¹ is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, NH and S.

19. The compound of claim 18, wherein Z¹ is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, NH and S.

20. The compound of claim 19, wherein Z¹ is C₃alkylene.

21. The compound of any one of claims 1 to 20, wherein R³ is sulfonate.

22. A zwitterionic compound of Formula I, or a salt and/or solvate thereof: 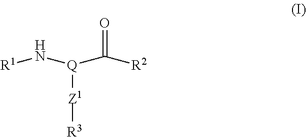
wherein
R¹ is a protecting group;
R² is OH;
Q is selected from Q1, Q2, Q3, Q4 and Q5: 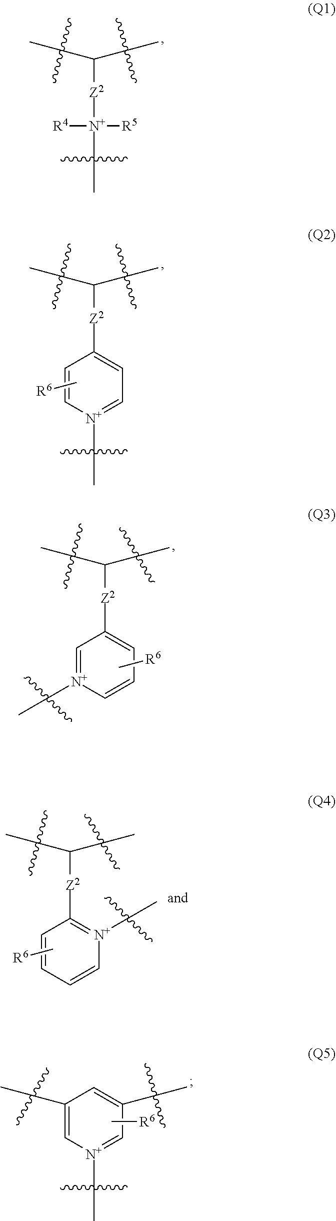
Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, N and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, N and S;
R³ is a sulfonate;
provided that when Q is Q1, Q2, Q3 or Q4, then the compound of Formula I is an (R)- or (S)-enantiomer of the carbon to which Z² is attached.

23. The compound of any one of claims 1 to 22 selected from:

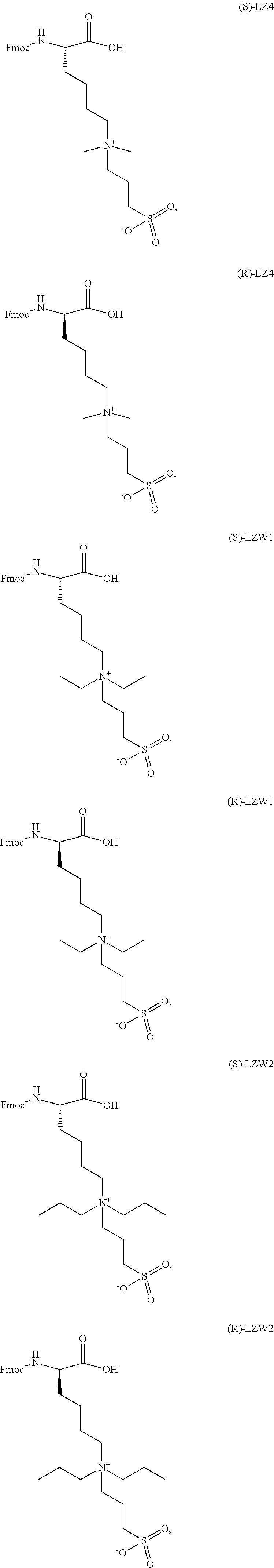

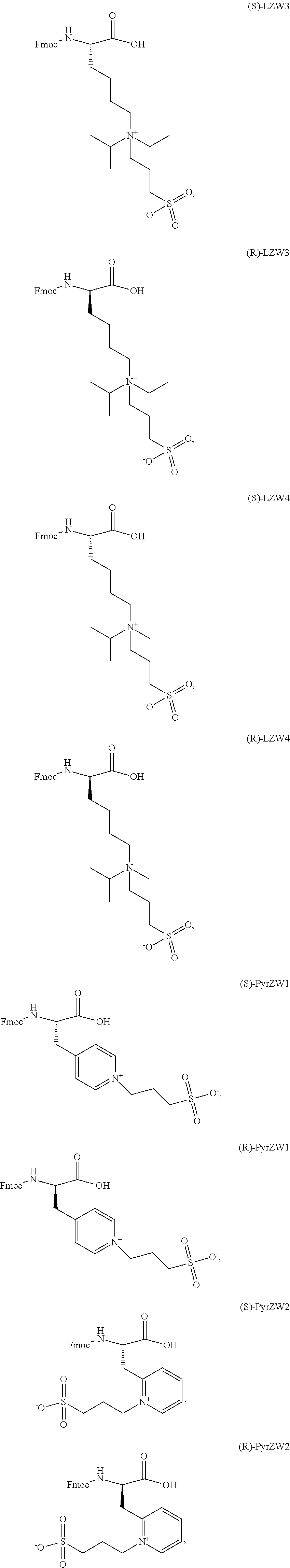

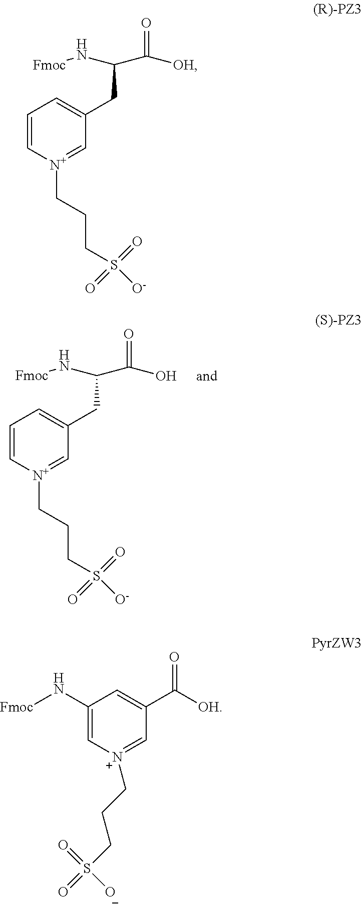

24. A conjugate of formula II, or a salt and/or solvate thereof: 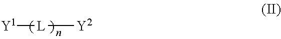
wherein
Y¹ is a complex comprising a chelating agent and one or more radionuclides;
Y² is a targeting ligand;
L is a zwitterionic (ZW) linker of the formula 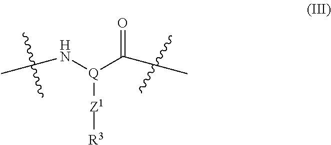
wherein
Q is selected from Q1, Q2, Q3, Q4 and Q5: 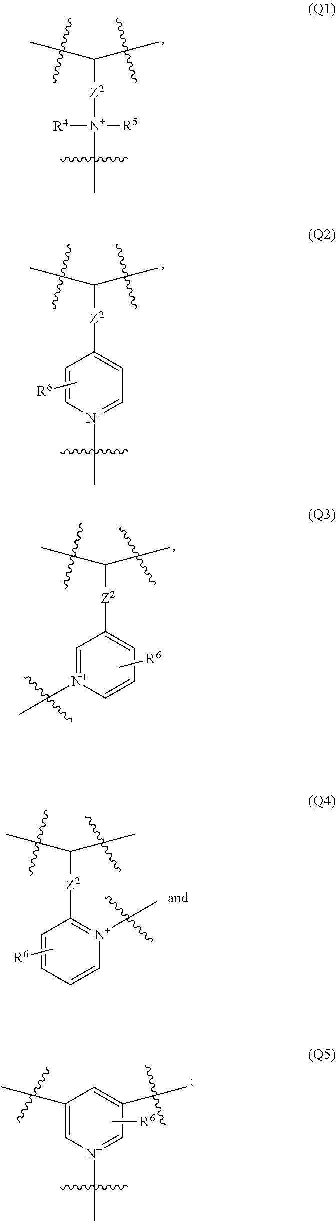
Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R³ is an anion containing group selected from sulfonate, carboxylate, sulfate and phosphate; and
n is an integer 0 to 5.

25. The conjugate of claim 24, wherein R⁴ and R⁵ are independently selected from C₁₋₆alkyl, C₂₋₆alkenyl and C₂₋₆alkynyl.

26. The conjugate of claim 25, wherein R⁴ and R⁵ are independently selected from CH₂CH₂CH₃, CH(CHs)₂, CH₂CH₃ and CH₃.

27. The conjugate of claim 26, wherein each one of R⁴ and R⁵ is CH₃.

28. The conjugate of claim 24, wherein R⁶ is H.

29. The conjugate of any one of claims 24 to 28, wherein Z² is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, NH and S.

30. The conjugate of claim 29, wherein Z² is C₄alkylene.

31. The conjugate of claim 30, wherein Z² is C₁alkylene.

32. The conjugate of any one of claims 24 to 31, wherein Z¹ is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, NH and S.

33. The conjugate of claim 32, wherein Z¹ is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, NH and S.

34. The conjugate of claim 33, wherein Z¹ is C₃alkylene.

35. The conjugate of any one of claims 24 to 34, wherein R³ is sulfonate.

36. The conjugate of any one of claims 24 to 35, wherein the chelating agent is selected from DOTA, NOTA, DTPA, TETA, EDTA, NODAGA, NODASA, TRITA, CDTA, BAT, DFO and HYNIC.

37. The conjugate of claim 36, wherein the chelating agent is DOTA.

38. The conjugate of any one of claims 24 to 37, wherein the one or more radionuclides is selected from ²²⁵Ac, ²²⁶Ac, ²²⁷Ac, ²²⁸Ac, ¹⁰⁵Ag, ^{106m}Ag, ^{110m}Ag, ¹¹¹Ag, ¹¹²Ag, ¹¹³Ag, ²³⁹Am, ²⁴⁰Am, ²⁴²Am, ²⁴⁴Am, ³⁷Ar, ⁷¹As, ⁷²As, ⁷³As, ⁷⁴As ⁷⁶As, ⁷⁷As, ²⁰⁹At, ²¹⁰At, ¹⁹¹Au, ¹⁹²Au, ¹⁹³Au, ¹⁹⁴Au, ¹⁹⁵Au, ¹⁹⁶Au, ^{196m2}Au, ¹⁹⁸Au, ^{198m}Au, ¹⁹⁹Au ^{200m}Au, ¹²⁸Ba, ¹³¹Ba, ^{133m}Ba, ^{135m}Ba, ¹⁴⁰Ba, ⁷Be, ²⁰³Bi, ²⁰⁴Bi, ²⁰⁵Bi, ²⁰⁶Bi, ²¹⁰Bi, ²¹²Bi, ²⁴³Bk, ²⁴⁴Bk ²⁴⁵Bk, ²⁴⁶Bk, ^{248m}Bk, ²⁵⁰Bk, ⁷⁶Br, ⁷⁷Br, ^{80m}Br, ⁸²Br, ¹¹C, ¹⁴C, ⁴⁵Ca, ⁴⁷Ca, ¹⁰⁷Cd, ¹¹⁵Cd, ¹¹⁵mCd ^{117m}d, ¹³²Ce, ^{133m}Ce, ¹³⁴Ce, ¹³⁵Ce, ¹³⁷Ce, ^{137m}Ce, ¹³⁹Ce, ¹⁴¹Ce, ¹⁴³Ce, ¹⁴⁴Ce, ²⁴⁶Cf, ²⁴⁷Cf, ²⁴⁷Cf, ²⁵³Cf, ²⁵⁴Cf, ²⁴⁰Cm, ²⁴¹Cm, ²⁴²Cm, ²⁵²Cm, ⁵⁵Co, ⁵⁶Co, ⁵⁷Co, ⁵⁸Co, ^{58m}Co, ⁶⁰Co, ⁴⁸Cr, ⁵¹Cr, ¹²⁷Cs, ¹²⁹Cs ¹³¹Cs, ¹³²Cs, ¹³⁶Cs, 137Cs, ⁶¹Cu, ⁶²Cu, ⁶⁴Cu, ⁶⁷Cu, ¹⁵³Dy, ¹⁵⁵Dy, ¹⁵⁷Dy, ¹⁵⁹Dy, ¹⁶⁵Dy, ¹⁶⁶Dy, ¹⁶⁰Er ¹⁶¹Er, ¹⁶⁵Er, ¹⁶⁹Er, ¹⁷¹Er, ¹⁷²Er, ²⁵⁰Es, 251Es, ²⁵³Es, ²⁵⁴Es, ^{254m}Es, ²⁵⁵Es, ^{256m}Es, ¹⁴⁵Eu, ¹⁴⁶Eu, ¹⁴⁷Eu ¹⁴⁸Eu, ¹⁴⁹Eu, ^{150m}Eu, ^{152m}Eu, ¹⁵⁶Eu, ¹⁵⁷Eu, [¹⁸F]AlF, ⁵²Fe, ⁵⁹Fe, ²⁵¹Fm, ²⁵²Fm, ²⁵³Fm, ²⁵⁴Fm, ²⁵⁵Fm, ²⁵⁷Fm ⁶⁶Ga, ⁶⁷Ga, ⁶⁸Ga, ⁷²Ga, ⁷³Ga, ¹⁴⁶Gd, ¹⁴⁷Gd, ¹⁴⁹Gd, ¹⁵¹Gd, ¹⁵³Gd, ¹⁵⁹Gd, ⁶⁸Ge, ⁶⁹Ge, ⁷¹Ge, ⁷⁷Ge ¹⁷⁰Hf, ¹⁷¹Hf, ¹⁷³Hf, ¹⁷⁵Hf, ^{179m2}Hf, ^{180m}Hf, ¹⁸¹Hf, ¹⁸⁴Hf, ¹⁹²Hg, ¹⁹³Hg, ^{193m}Hg, ¹⁹⁵Hg, ^{195m}Hg, ¹⁹⁷Hg ^{197m}Hg, ²⁰³Hg, ^{160m}Ho, ¹⁶⁶Ho, ¹⁶⁷Ho, ¹²³I, ¹²⁴I, ¹²⁶I, ¹³⁰I, ¹³²I, ¹³³I, ¹³⁵I, ¹⁰⁹In, ¹¹⁰In, ¹¹¹In, ^{114m}In, ^{115m}In, ¹⁸⁴Ir, ¹⁸⁵Ir, ¹⁸⁶Ir, ¹⁸⁷Ir, ¹⁸⁸Ir, ¹⁸⁹Ir, ¹⁹⁰Ir, ^{190m2}Ir, ¹⁹²Ir, ^{193m}Ir, ¹⁹⁴Ir, ^{194m2}Ir, ^{195m}Ir, ⁴²K, ⁴³K, ⁷⁸Kr ⁷⁹Kr, ^{81m}Kr, ^{85m}Kr, ¹³²La, ¹³³La, ¹³⁵La, ¹⁴⁰La, ¹⁴¹La, ²⁶²Lr, ¹⁶⁹Lu, ¹⁷⁰Lu, ¹⁷¹Lu, ¹⁷²Lu, ^{174m}Lu, ^{176m}Lu ¹⁷⁷Lu, ^{177m}Lu, ¹⁷⁹Lu, ²⁵⁷Md, ²⁵⁸Md, ²⁶⁰Md, ²⁸Mg, ⁵²Mn, ⁹⁰Mo, ^{93m}Mo, ⁹⁹Mo, ¹⁰⁰Mo, ¹³N, ²⁴Na, ⁹⁰Nb, ^{91m}Nb ^{92m}Nb, ⁹⁵Nb, ^{95m}Nb, ⁹⁶Nb, ¹³⁸Nd, ^{139m}Nd, ¹⁴⁰Nd, ¹⁴⁷Nd, ⁵⁶Ni, ⁵⁷Ni, ⁶⁶Ni, ²³⁴Np, ²³⁶Np, ⁹⁹Mo, Np, ²³⁸Np ²³⁹Np, ¹⁵O, ¹⁸²Os, ¹⁸³Os, ^{183m}Os, ¹⁸⁵Os, ^{189m}Os, ¹⁹¹Os, ^{191m}Os, ¹⁹³Os, ³²P, ³³P, ²²⁸Pa, ²²⁹Pa, ²³⁰Pa, ²³²Pa, ²³³Pa, ²³⁴Pa, ²⁰⁰Pb, ²⁰¹Pb, ²⁰²mPb, ²⁰³Pb, ²⁰⁹Pb, ²¹²Pb, ¹⁰⁰Pd, ¹⁰¹Pd, ¹⁰³Pd, ¹⁰⁹Pd ^{111m}Pd, ¹¹²Pd, ¹⁴³Pm, ¹⁴⁸Pm, ^{148m}Pm, ¹⁴⁹Pm, ¹⁵¹Pm, ²⁰⁴Po, ²⁰⁶Po, ²⁰⁷Po, ²¹⁰Po, ¹³⁹Pr, ¹⁴²Pr, ¹⁴³Pr, ¹⁴⁵Pr, ¹⁸⁸Pt, ¹⁹¹Pt, ¹⁹¹Pt, ^{193m}Pt, ^{195m}Pt, ¹⁹⁷Pt, ²⁰⁰Pt, ²⁰²Pt, ²³⁴Pu, ²³⁷Pu, ²⁴³Pu, ²⁴⁵Pu, ²⁴⁶Pu, ²⁴⁷Pu ²²³Ra, ²²⁴Ra, ²²⁵Ra, ⁸¹Rb, ⁸²Rb, ⁸²mRb, ⁸³Rb, ⁸⁴Rb, ⁸⁶Rb, ¹⁸¹Re, ¹⁸²Re, ^{182m}Re, ¹⁸³Re, ¹⁸⁴Re, ^{184m}Re, ¹⁸⁶Re, ¹⁸⁸Re, ¹⁸⁹Re, ^{190m}Re, ⁹⁹Rh, ^{99m}Rh, ¹⁰⁰Rh, ^{101m}Rh, ¹⁰²Rh, ^{103m}Rh, ¹⁰⁵Rh, ²¹¹Rn, ²²²Rn, ⁹⁷Ru, ¹⁰³Ru, ¹⁰⁵Ru, ³⁵S, ^{118m}Sb, ¹¹⁹Sb, ¹²⁰Sb, ^{120m}Sb, ¹²²Sb, ¹²⁴Sb, ¹²⁶Sb, ¹²⁷Sb, ¹²⁸Sb, ¹²⁹Sb, ⁴³Sc, ⁴⁴Sc, ^{44m}Sc, ⁴⁶Sc, ⁴⁷Sc, ⁴⁸Sc, ⁷²Se, ⁷³Se, ⁷⁵Se, ¹⁵³Sm, ¹⁵⁶Sm, ¹¹⁰Sn, ¹¹³Sn, ^{117m}Sn ^{119m}Sn, ¹²¹Sn, ¹²³Sn, ¹²⁵Sn, ⁸²Sr, ⁸³Sr, ⁸⁵Sr, ⁸⁹Sr, ⁹¹Sr, ¹⁷³Ta, ¹⁷⁵Ta, ¹⁷⁶Ta, ¹⁷⁷Ta, ¹⁸⁰Ta, ¹⁸²Ta ¹⁸³Ta, ¹⁸⁷⁴Ta, ¹⁴⁹Tb, ¹⁵⁰Tb, ¹⁵¹Tb, ¹⁵²Tb, ¹⁵³Tb, ¹⁵⁴Tb, ^{154m}Tb, ^{154m2}Tb, ¹⁵⁵Tb, ¹⁵⁶Tb, ^{156m}Tb ^{156m2}Tb, ¹⁶⁰Tb, ¹⁶¹Tb, ⁹⁴Tc, ⁹⁵Tc, ^{95m}Tc, ⁹⁶Tc, ^{97m}Tc, ^{99m}Tc, ¹¹⁸Te, ¹¹⁹Te, ^{119m}Te, ¹²¹Te, ^{121m}Te, ^{123m}Te, ^{125m}Te, ¹²⁷Te, ^{127m}Te, ^{129m}Te, ^{131m}Te, ¹³²Te, ²²⁷Th, ²²⁹Th, ²³¹Th, ²³⁴Th, ⁴⁵Ti, ¹⁹⁸Tl, ¹⁹⁹Tl, ²⁰⁰Tl, ²⁰¹Tl, ²⁰²Tl, ²⁰⁴Tl, ¹⁶⁵Tm, ¹⁶⁶Tm, ¹⁶⁷Tm, ¹⁶⁸Tm, ¹⁷⁰Tm, ¹⁷²Tm, ¹⁷³Tm, ²³⁰U, ²³¹U, ²³⁷U, ²⁴⁰U, ⁴⁸V, ¹⁷⁸W, ¹⁸¹W, ¹⁸⁵W, ¹⁸⁷W, ¹⁸⁸W, ¹²²Xe, ¹²⁵Xe, ¹²⁷Xe, ^{129m}Xe, ^{131m}Xe, ¹³³Xe, ^{133m}Xe, ¹³⁵Xe, ^{85m}Y, ⁸⁶Y, ⁸⁷Y, ^{87m}Y, ⁸⁸Y, ⁹⁰Y, ^{90m}Y, ⁹¹Y, ⁹²Y, ⁹³Y, ₁₆₆Yb, ¹⁶⁹Yb, ¹⁷⁵Yb, ⁶²Zn, ⁶⁵Zn, ^{69m}Zn, ^{71m}Zn, ⁷²Zn, ⁸⁶Zr, ⁸⁸Zr, ⁸⁹Zr, ⁹⁵Zr, and ⁹⁷Zr.

39. The conjugate of claim 38, wherein the radionuclide is selected from ⁶⁸Ga, ¹⁷⁷Lu, [¹⁸F]AlF and ⁹⁰Y.

40. The conjugate of any one of claims 24 to 39, wherein the chelating agent coordinatively bound to two radionuclides.

41. The conjugate of any one of claims 24 to 40, wherein the targeting ligand is selected from an oligonucleotide, an oligopeptide, a protein, a peptide and a small molecule compound.

42. The conjugate of claim 41, wherein the targeting ligand is a tumor-targeting ligand.

43. The conjugate of claim 42, wherein the targeting ligand is selected from TATE, TOC and PSMA.

44. The conjugate of any one of claims 24 to 43, wherein n is an integer 1 to 3.

45. The conjugate of claim 44, wherein n is 1.

46. The conjugate of claim 44, wherein n is 2.

47. The conjugate of any one of claims 24 to 46, wherein the conjugate comprises a spacer.

48. The conjugate of claim 47, wherein the conjugate has the formula IIa 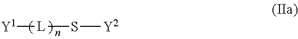
wherein,
S is a spacer.

49. The conjugate of claim 48, wherein the spacer is polyethylene glycol (PEG).

50. The conjugate of claim 49, wherein the PEG spacer contains four PEG units.

51. A conjugate of Formula IV, or a salt and/or solvate thereof: 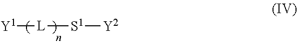
wherein
Y¹ is a complex comprising a chelating agent and one or more radionuclides, wherein the chelating agent is desferrioxamine (DFO) or a compound of the following formula; 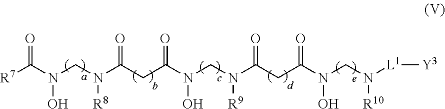
wherein
R⁷, R⁸, R⁹ and R¹⁰ are independently selected from H and C₁₋₄alkyl;
each a, c and e is an integer independently selected from 3, 4, 5, 6 and 7;
each b and d is an integer independently selected from 1, 2, 3 and 4;
L¹ is a linker group 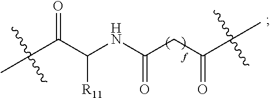
Y³ is 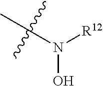
or 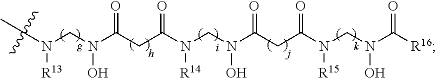
R¹¹ is selected from C₁₋₁₀alkyl and C₁₋₆alkylenephenyl, optionally substituted by one of NH₂, OH, SH, N₃, CO₂H and 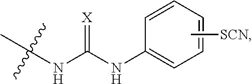
wherein X is O, S or NH;
R¹², R¹³, R¹⁴, R¹⁵ and R¹⁶ are independently selected from H and C₁₋₄alkyl;
f is an integer selected from 1, 2, 3 and 4;
each g, l and k is an integer independently selected from 3, 4, 5, 6 and 7;
each h and j is an integer independently selected from 1, 2, 3 and 4;
Y² is a targeting ligand;
S¹ is a biomolecule conjugating group;
L is a zwitterionic (ZW) linker of the formula 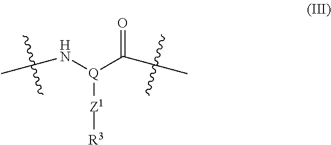
wherein
Q is selected from Q1, Q2, Q3, Q4 and Q5: 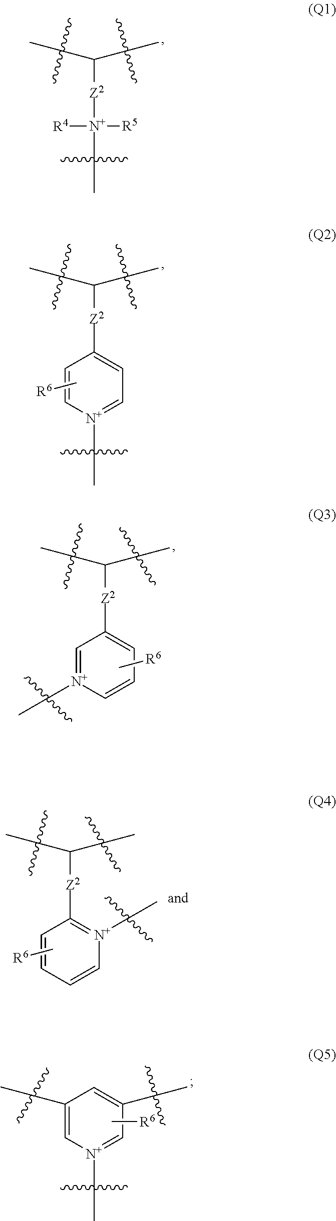
Z² is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R⁴ and R⁵ are independently selected from C₁₋₁₀alkyl, C₂₋₁₀alkenyl and C₂₋₁₀alkynyl;
R⁶ is selected from H, halo, NO₂, SO₂ and OH;
Z¹ is selected from C₁₋₁₅alkylene, C₂₋₁₅alkenylene and C₂₋₁₅alkynelene, unsubstituted or substituted with one or more of halo or OH and/or are optionally interrupted by one to three heteromoieties independently selected from O, NH and S;
R³ is an anion containing group selected from sulfonate, carboxylate, sulfate and phosphate;
n is an integer 1 to 5;
wherein the chelating agent is a compound of Formula V, Y¹ is attached to L at R¹¹ of L¹ group 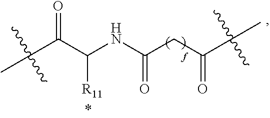
and the L is further optionally attached to one to three additional L groups, or the L is attached between the Y³ 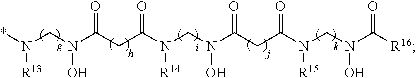
and L¹ 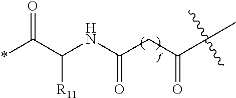
groups of Y¹, or one L is attached at R¹¹ of L¹ group and another L is attached between the Y³ and L¹ groups of Y¹, and each one of the L is further optionally attached to one to three additional L groups; and
wherein the chelating agent is DFO, the point of attachment of L is at the amine 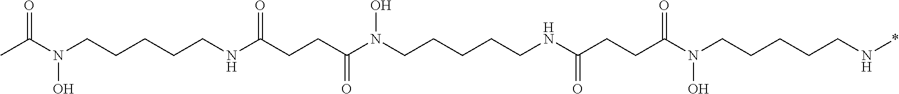 [SMILES: *NCCCCCN(O)C(=O)CCC(=O)NCCCCCN(O)C(=O)CCC(=O)NCCCCCN(O)C(C)=O]
and wherein the * indicates the point of attachment.

52. The conjugate of claim 51, wherein R⁴ and R⁵ are independently selected from C₁₋₆alkyl, C₂₋₆alkenyl and C₂₋₆alkynyl.

53. The conjugate of claim 52, wherein R⁴ and R⁵ are independently selected from CH₂CH₂CH₃, CH(CHs)₂, CH₂CH₃ and CH₃.

54. The conjugate of claim 53, wherein each one of R⁴ and R⁵ is CH₃.

55. The conjugate of claim 51, wherein R⁶ is H.

56. The conjugate of any one of claims 51 to 55, wherein Z² is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, NH and S.

57. The conjugate of claim 56, wherein Z² is C₄alkylene.

58. The conjugate of claim 56, wherein Z² is C₁alkylene.

59. The conjugate of any one of claims 51 to 58, wherein Z¹ is selected from C₁₋₁₀alkylene, C₂₋₁₀alkenylene and C₂₋₁₀alkynelene, unsubstituted or substituted with one or more of halo or OH and/or are optionally interrupted by one to three heteromoieties independently selected from O, NH and S.

60. The conjugate of claim 59, wherein Z¹ is selected from C₁₋₄alkylene, C₂₋₄alkenylene and C₂₋₄alkynelene, unsubstituted or substituted with one or more of halo or OH and/or are optionally interrupted by one to three heteromoieties independently selected from O, NH and S.

61. The conjugate of claim 60, wherein Z¹ is C₃alkylene.

62. The conjugate of any one of claims 51 to 61, wherein R³ is sulfonate.

63. The conjugate of claim 51, wherein L is selected from

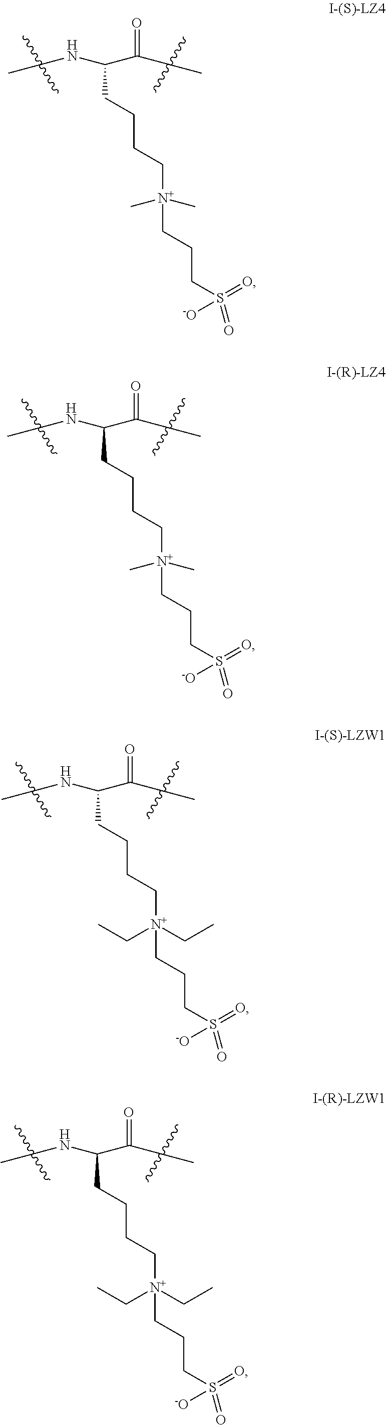

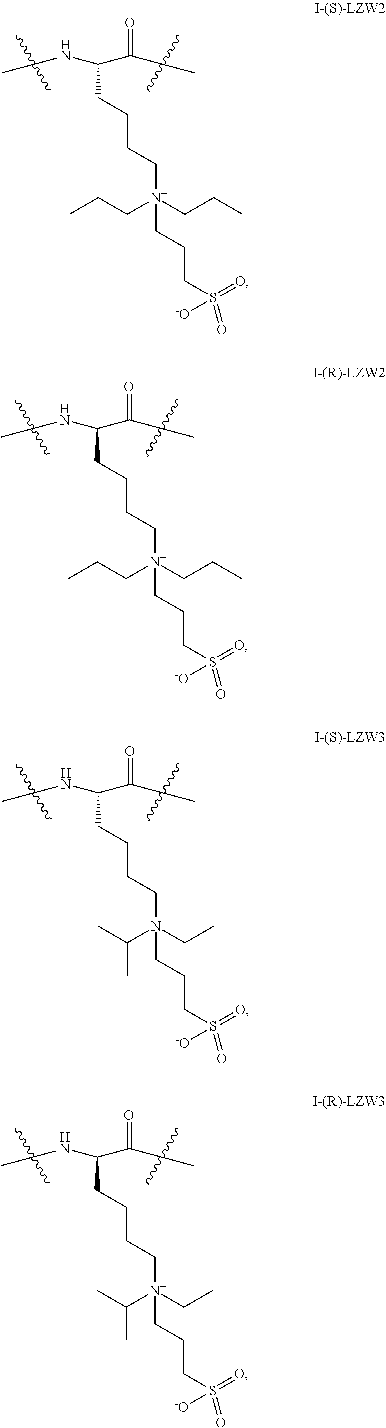

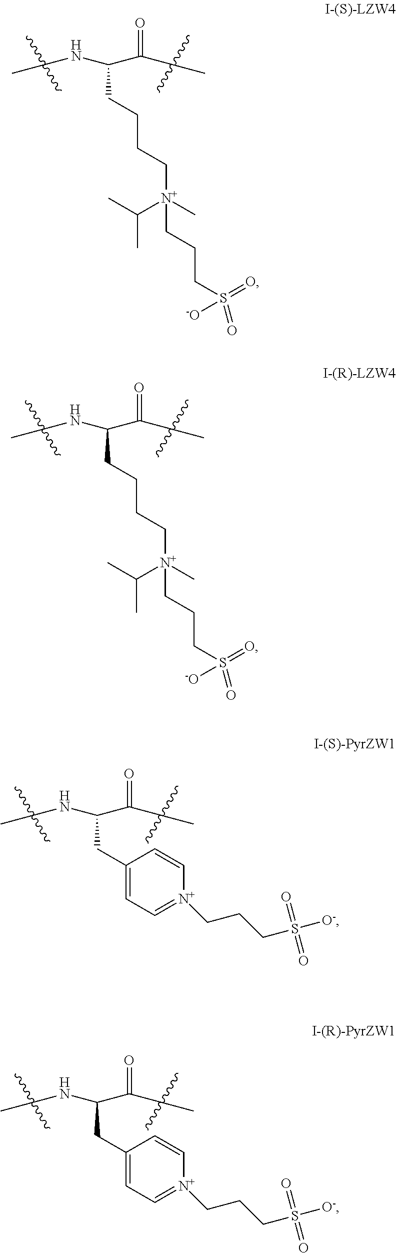

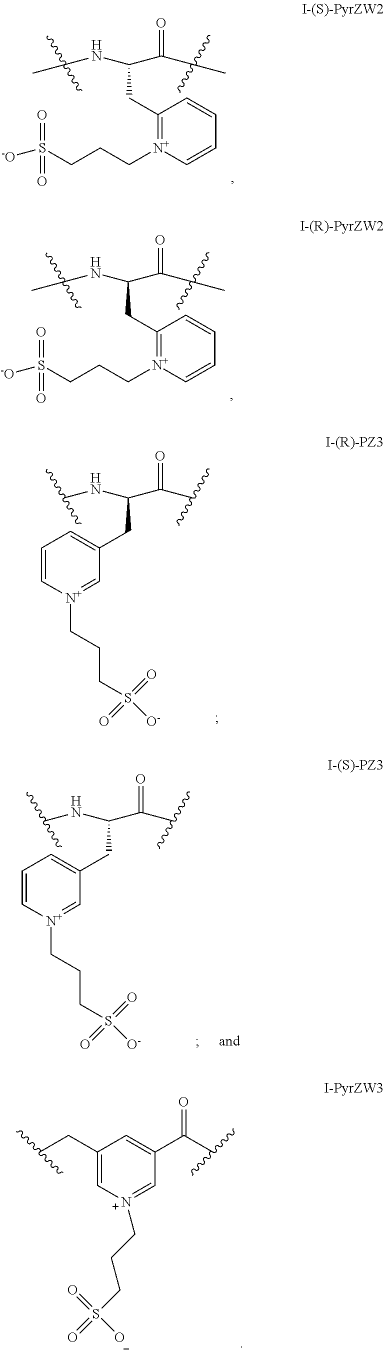

64. The conjugate of claim 63, wherein L is

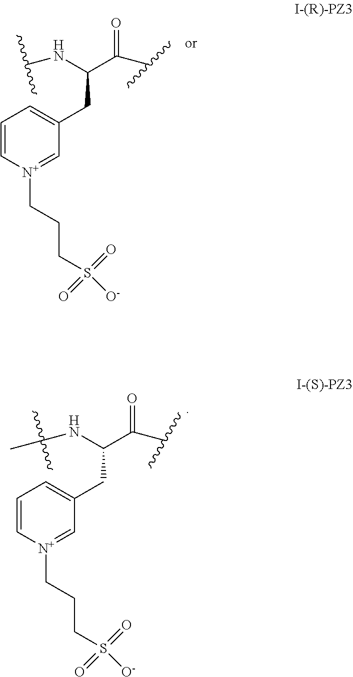 [SMILES: *N[C@H](Cc1ccc[n+](CCCS(=O)(=O)[O-])c1)C(*)=O ; "or" ; *N[C@@H](Cc1ccc[n+](CCCS(=O)(=O)[O-])c1)C(*)=O ; "."]

65. The conjugate of any one of claims 51 to 64, wherein the chelating agent is DFO.

66. The conjugate of any one of claims 51 to 64, wherein the chelating agent is a compound of Formula V, wherein at least one of R⁷, R⁶, R⁹ and R¹⁰ is C₁₋₂alkyl.

67. The conjugate of claim 66, wherein R⁷ is CH₃ and each one of R⁸, R⁹ and R¹⁰ is H.

68. The conjugate of claim 66 or 67, wherein each a, c and e is an integer independently selected from 4, 5 and 6.

69. The conjugate of claim 68, wherein a, c and e are 5.

70. The conjugate of any one of claims 66 to 69, wherein each b and d is an integer independently selected from 1, 2 and 3.

71. The conjugate of claim 70, wherein b and d are 2.

72. The conjugate of any one of claims 64 to 71, wherein f is selected from the integer 1, 2 and 3.

73. The conjugate of claim 72, wherein f is 2.

74. The conjugate of any one of claims 64 to 73, wherein when Y is 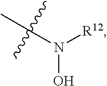
R¹² is selected from H and C₁₋₂alkyl.

75. The conjugate of claim 74, wherein R¹² is selected from H and CH₃.

76. The conjugate of any one of claims 64 to 73, wherein when Y is 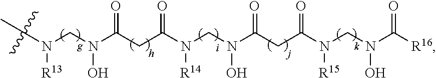
R¹³, R¹⁴, R¹⁵ and R¹⁶ are independently selected from H and C₁₋₂alkyl.

77. The conjugate of claim 76, wherein at least one of R¹³, R¹⁴, R¹⁵ and R¹⁶ is C₁₋₂alkyl.

78. The conjugate of claim 77, wherein R¹⁶ is CH₃ and each one of R¹³, R¹⁴ and R¹⁵ is H.

79. The conjugate of any one of claims 76 to 78, wherein each g, l and k is an integer independently selected from 4, 5 and 6.

80. The conjugate of claim 79, wherein g, l and k are 5.

81. The conjugate of any one of claims 76 to 80, wherein each h and j is an integer independently selected from 1, 2 and 3.

82. The conjugate of claim 81, wherein h and j are 2.

83. The conjugate of any one of claims 64 to 82, wherein a, c and e are 5 and b and d are 2 and the compound of Formula V has the following structure:

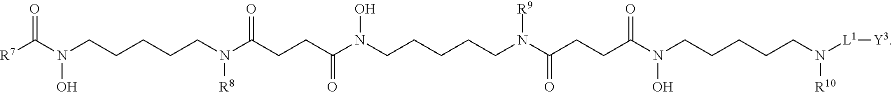

84. The conjugate of claim 83, wherein Y³ is 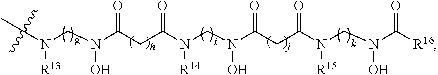
g, i and k are 5 and h and j are 2 and the compound of Formula V has the following structure:

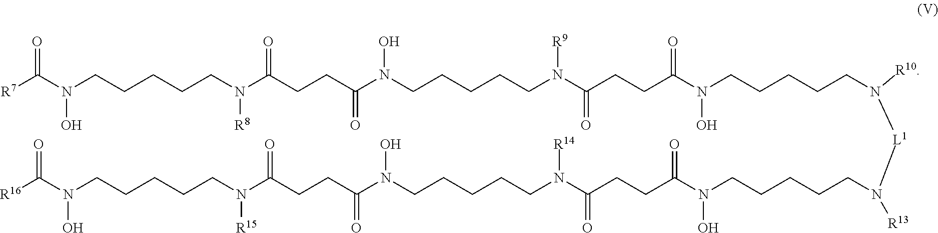

85. The conjugate of claim 64, wherein the compound of Formula V is selected from

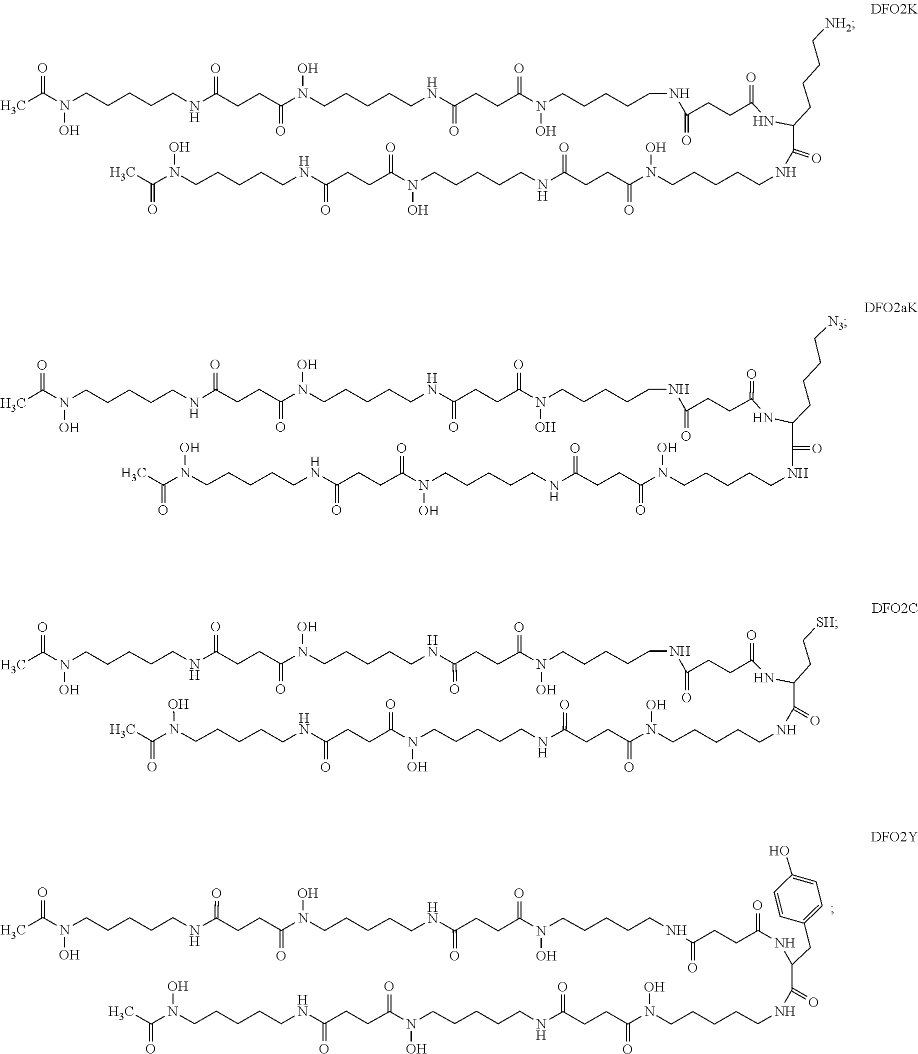

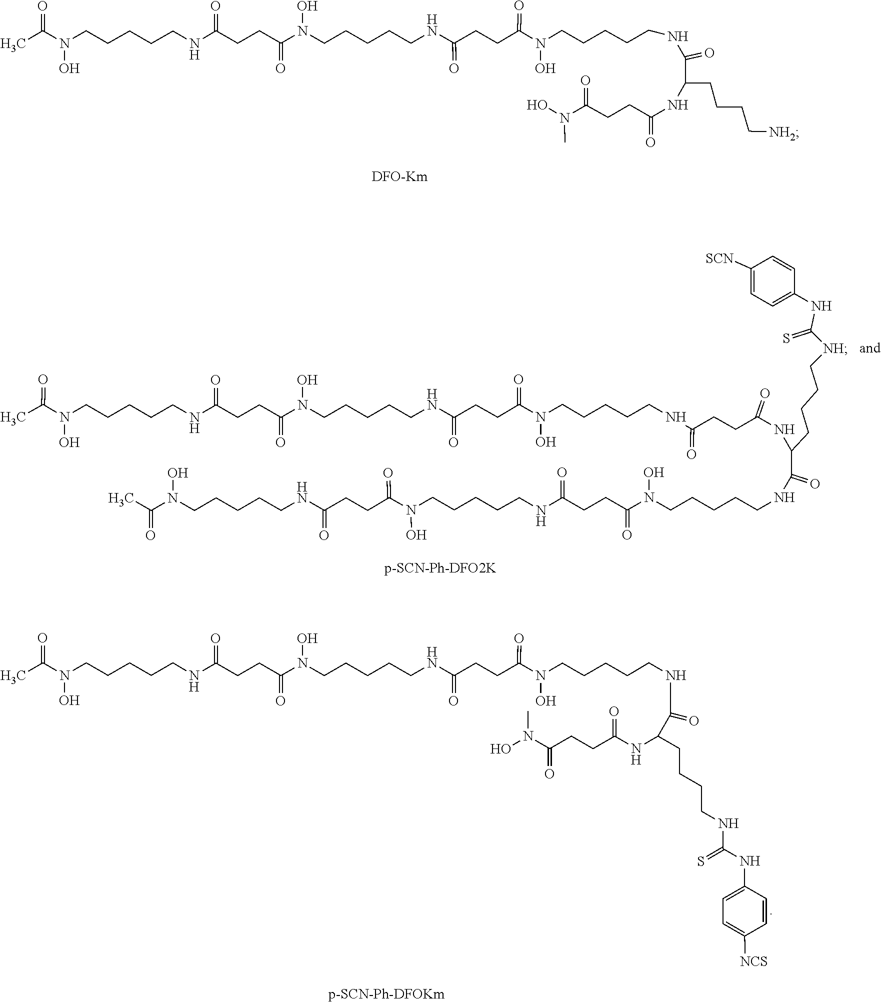

86. The conjugate of any one of claims 51 to 85, wherein n is an integer 1 to 3.

87. The conjugate of any one of claims 51 to 86, wherein the chelating agent is a compound of Formula V and Y¹ is attached to L at R¹¹ of L¹ group

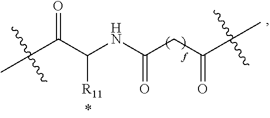

88. The conjugate of claim 87, wherein L is further optionally attached to one or two additional L groups.

89. The conjugate of any one of claims 51 to 86, wherein the chelating agent is a compound of Formula V and L is attached between the Y³ 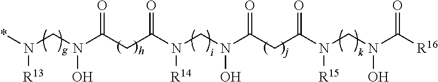
and L¹ 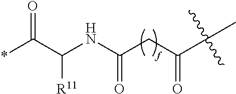
groups of Y¹.

90. The conjugate of claim 89, wherein L is further optionally attached to one or two additional L groups.

91. The conjugate of any one of claims 51 to 86, wherein the chelating agent is a compound of Formula V and one L is attached at R¹¹ of L¹ group 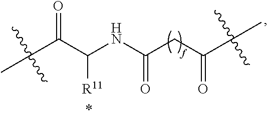
and another L is attached between the Y³ 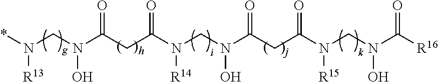
and L¹ 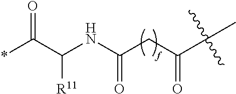
groups of Y¹.

92. The conjugate of claim 91, wherein L group attached at R¹¹ of L¹ group is further attached to one to three additional L groups.

93. The conjugate of claim 91, wherein the chelating agent is DFO, the point of attachment of L is at the amine

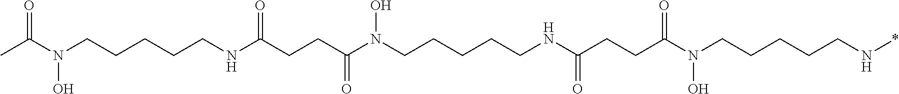 [SMILES: *NCCCCCN(O)C(=O)CCC(=O)NCCCCCN(O)C(=O)CCC(=O)NCCCCCN(O)C(C)=O]

94. The conjugate of any one of claims 51 to 92, wherein the Y¹-L is selected from

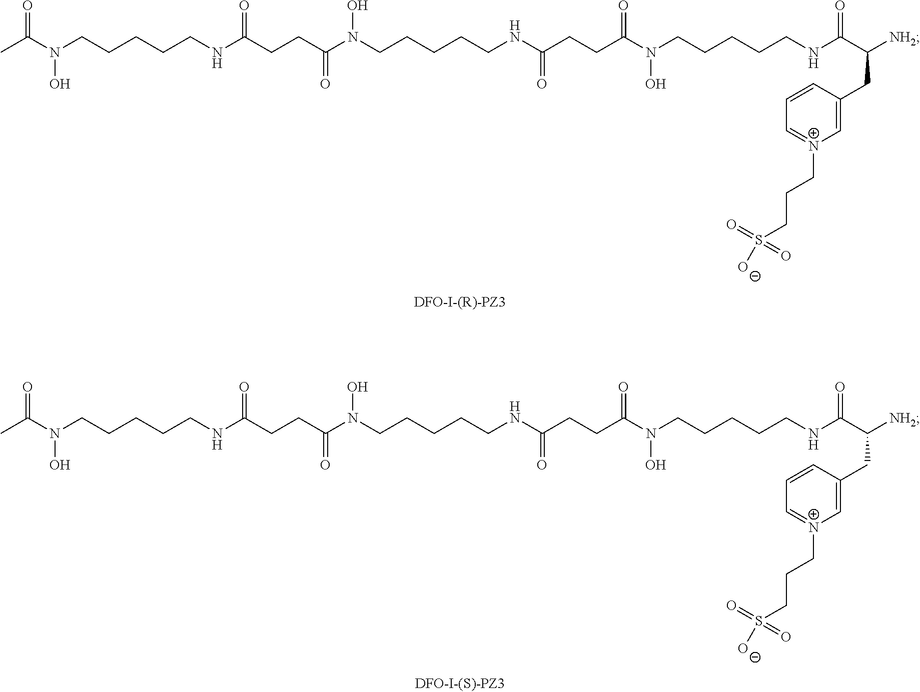 [SMILES: CC(=O)N(O)CCCCCNC(=O)CCC(=O)N(O)CCCCCNC(=O)CCC(=O)N(O)CCCCCNC(=O)[C@@H](N)Cc1ccc[n+](CCCS(=O)(=O)[O-])c1 ; CC(=O)N(O)CCCCCNC(=O)CCC(=O)N(O)CCCCCNC(=O)CCC(=O)N(O)CCCCCNC(=O)[C@H](N)Cc1ccc[n+](CCCS(=O)(=O)[O-])c1]

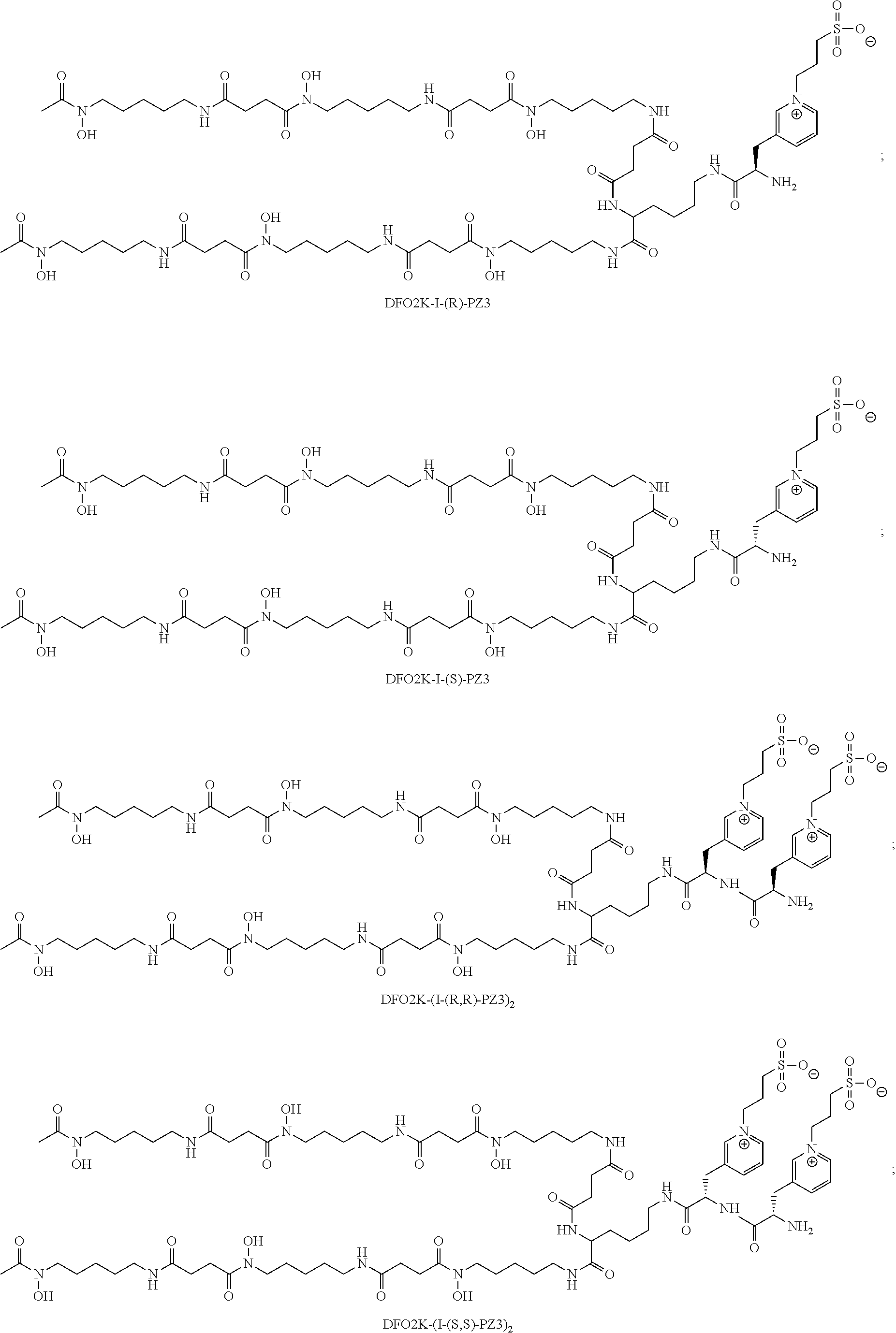

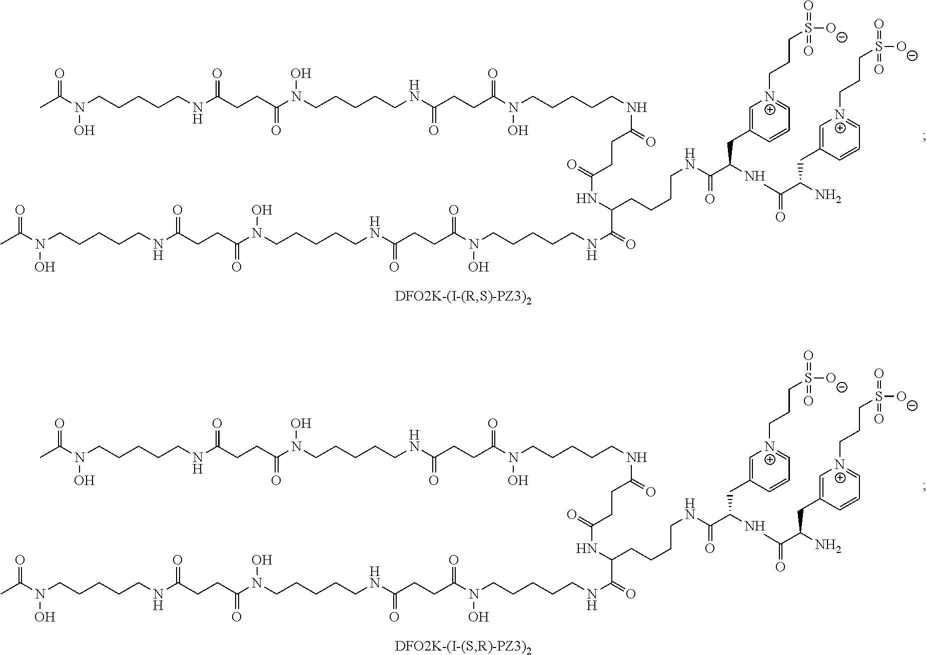 [SMILES: CC(=O)N(O)CCCCCNC(=O)CCC(=O)N(O)CCCCCNC(=O)CCC(=O)N(O)CCCCCNC(=O)CCC(=O)NC(CCCCNC(=O)[C@@H](Cc1ccc[n+](CCCS(=O)(=O)[O-])c1)NC(=O)[C@@H](N)Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O ; CC(=O)N(O)CCCCCNC(=O)CCC(=O)N(O)CCCCCNC(=O)CCC(=O)N(O)CCCCCNC(=O)CCC(=O)NC(CCCCNC(=O)[C@H](Cc1ccc[n+](CCCS(=O)(=O)[O-])c1)NC(=O)[C@H](N)Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O]

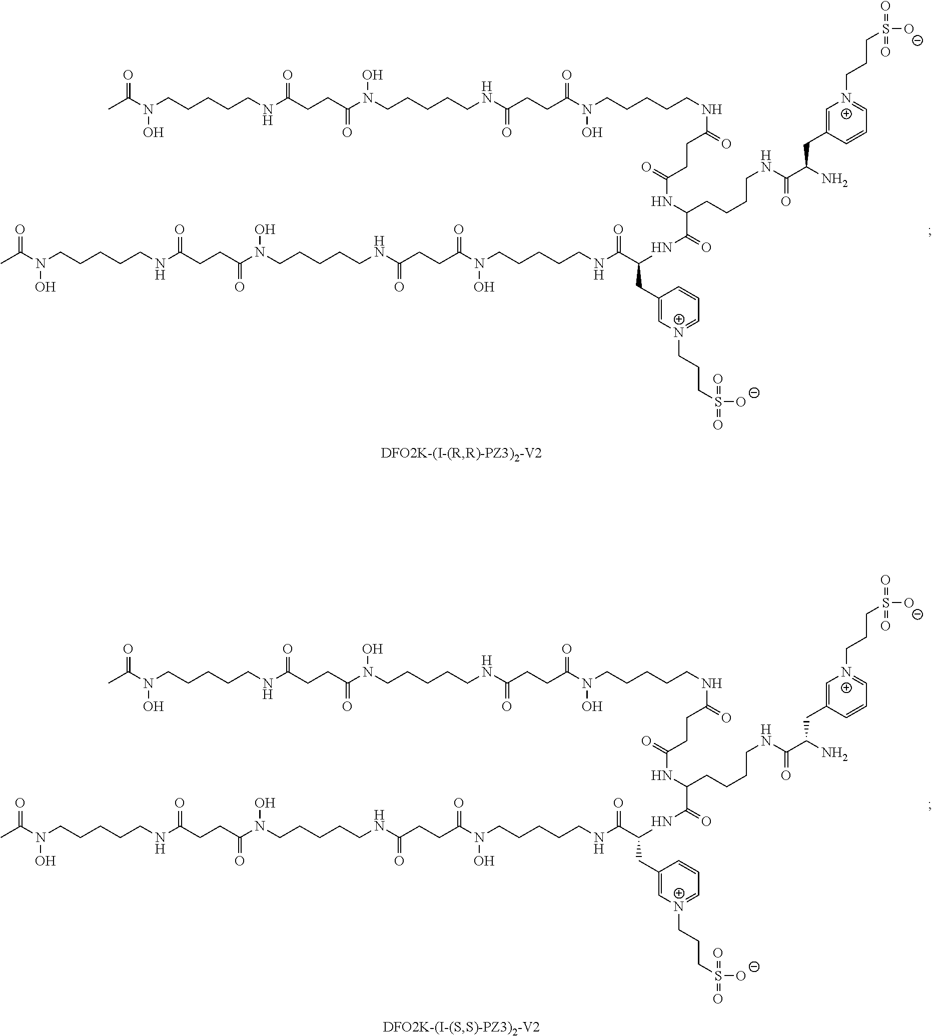 [SMILES: CC(=O)N(O)CCCCCNC(=O)CCC(=O)N(O)CCCCCNC(=O)CCC(=O)N(O)CCCCCNC(=O)CCC(=O)NC(CCCCNC(=O)[C@H](N)Cc1ccc[n+](CCCS(=O)(=O)[O-])c1)C(=O)N[C@@H](Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O ; CC(=O)N(O)CCCCCNC(=O)CCC(=O)N(O)CCCCCNC(=O)CCC(=O)N(O)CCCCCNC(=O)CCC(=O)NC(CCCCNC(=O)[C@@H](N)Cc1ccc[n+](CCCS(=O)(=O)[O-])c1)C(=O)N[C@H](Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O]

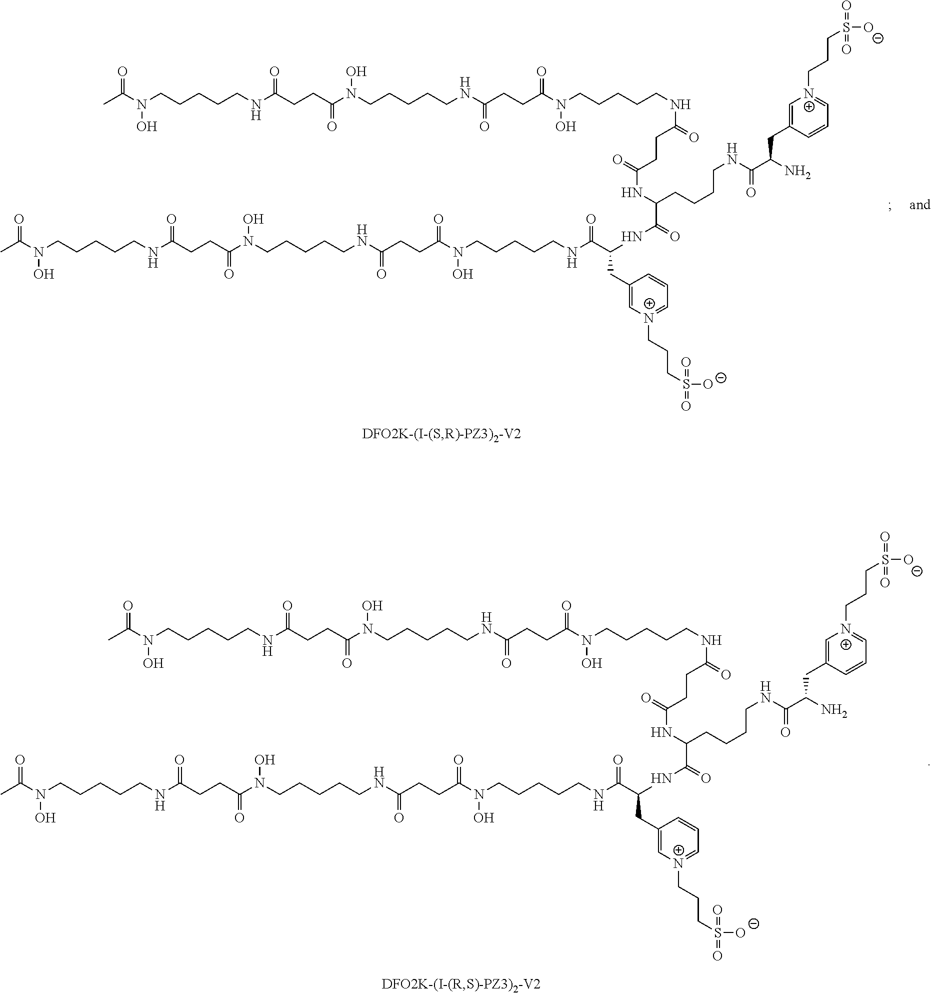 [SMILES: CC(=O)N(O)CCCCCNC(=O)CCC(=O)N(O)CCCCCNC(=O)CCC(=O)N(O)CCCCCNC(=O)CCC(=O)NC(CCCCNC(=O)[C@H](N)Cc1ccc[n+](CCCS(=O)(=O)[O-])c1)C(=O)N[C@H](Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O ; CC(=O)N(O)CCCCCNC(=O)CCC(=O)N(O)CCCCCNC(=O)CCC(=O)N(O)CCCCCNC(=O)CCC(=O)NC(CCCCNC(=O)[C@@H](N)Cc1ccc[n+](CCCS(=O)(=O)[O-])c1)C(=O)N[C@@H](Cc1ccc[n+](CCCS(=O)(=O)[O-])c1)C(=O)NCCCCCN(O)C(=O)CCC(=O)NCCCCCN(O)C(=O)CCC(=O)NCCCCCN(O)C(C)=O]

95. The conjugate of any one of claims 51 to 94, wherein S′ is a biomolecule conjugating group selected from NH₂, OH, SH, N₃, CO₂H, ONH₂, NHNH₂, C(O)C₁₋₄alkyl, C≡CH, SCN, C(O)NH₂, NHC(X)NH₂, C₁₋₄alkyleneN₃, NHC(O)C₁₋₄alkyleneN₃, NHC(O)C₁₋₄alkyleneONH₂, NHC(O)C₁₋₄alkyleneNHNH₂, NHC(O)OC₁₋₄alkyleneN₃, C(O)NHC₁₋₄alkylene-phenylene-tetrazine, 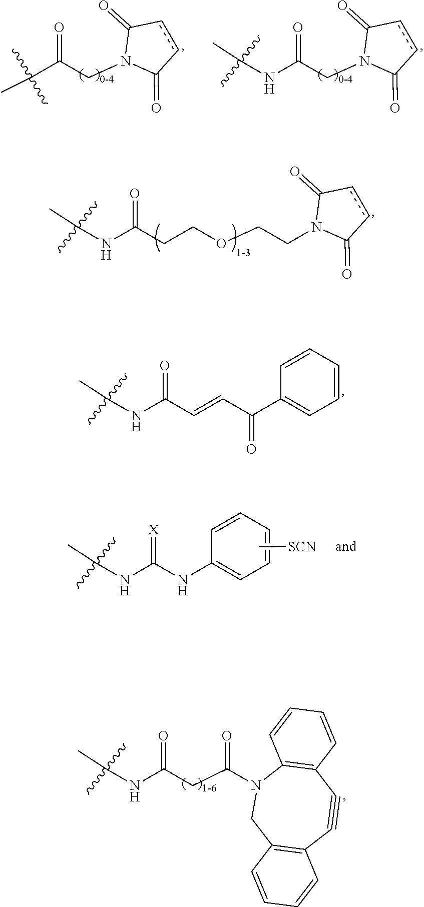
wherein [symbol] represents a single or a double bond and X is O, S or NH.

96. The conjugate of claim 95, wherein the biomolecule conjugating group is 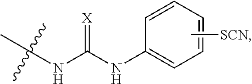
wherein X is O, S or NH.

97. The conjugate of claim 96, wherein the biomolecule conjugating group is

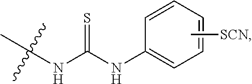

98. The conjugate of any one of claims 51 to 97, wherein the one or more radionuclides is selected from ²²⁵Ac, ²²⁶Ac, ²²⁷Ac, ²²⁸Ac, ¹⁰⁵Ag, ^{106m}Ag, ^{110m}Ag, ¹¹¹Ag, ¹¹²Ag, ¹¹³Ag, ²³⁹Am, ²⁴⁰Am, ²⁴²Am, ²⁴⁴Am, ³⁷Ar, ⁷¹As, ⁷²As, ⁷³As, ⁷⁴As ⁷⁶As, ⁷⁷As, ²⁰⁹At, ²¹⁰At, ¹⁹¹Au, ¹⁹²Au, ¹⁹³Au, ¹⁹⁴Au, ¹⁹⁵Au, ¹⁹⁶Au, ^{196m2}Au, ¹⁹⁸Au, ^{198m}Au, ¹⁹⁹Au ^{200m}Au, ¹²⁸Ba, ¹³¹Ba, ^{133m}Ba, ^{135m}Ba, ¹⁴⁰Ba, ⁷Be, ²⁰³Bi, ²⁰⁴Bi, ²⁰⁵Bi, ²⁰⁶Bi, ²¹⁰Bi, ²¹²Bi, ²⁴³Bk, ²⁴⁴Bk ²⁴⁵Bk, ²⁴⁶Bk, ^{248m}Bk, ²⁵⁰Bk, ⁷⁶Br, ⁷⁷Br, ^{80m}Br, ⁸²Br, ¹¹C, ¹⁴C, ⁴⁵Ca, ⁴⁷Ca, ¹⁰⁷Cd, ¹¹⁵Cd, ¹¹⁵mCd ^{117m}d, ¹³²Ce, ^{133m}Ce, ¹³⁴Ce, ¹³⁵Ce, ¹³⁷Ce, ^{137m}Ce, ¹³⁹Ce, ¹⁴¹Ce, ¹⁴³Ce, ¹⁴⁴Ce, ²⁴⁶Cf, ²⁴⁷Cf, ²⁴⁷Cf, ²⁵³Cf, ²⁵⁴Cf, ²⁴⁰Cm, ²⁴¹Cm, ²⁴²Cm, ²⁵²Cm, ⁵⁵Co, ⁵⁶Co, ⁵⁷Co, ⁵⁸Co, ^{58m}Co, ⁶⁰Co, ⁴⁸Cr, ⁵¹Cr, ¹²⁷Cs, ¹²⁹Cs ¹³¹Cs, ¹³²Cs, ¹³⁶Cs, 137Cs, ⁶¹Cu, ⁶²Cu, ⁶⁴Cu, ⁶⁷Cu, ¹⁵³Dy, ¹⁵⁵Dy, ¹⁵⁷Dy, ¹⁵⁹Dy, ¹⁶⁵Dy, ¹⁶⁶Dy, ¹⁶⁰Er ¹⁶¹Er, ¹⁶⁵Er, ¹⁶⁹Er, ¹⁷¹Er, ¹⁷²Er, ²⁵⁰Es, 251Es, ²⁵³Es, ²⁵⁴Es, ^{254m}Es, ²⁵⁵Es, ^{256m}Es, ¹⁴⁵Eu, ¹⁴⁶Eu, ¹⁴⁷Eu ¹⁴⁸Eu, ¹⁴⁹Eu, ^{150m}Eu, ^{152m}Eu, ¹⁵⁶Eu, ¹⁵⁷Eu, [¹⁸F]AlF, ⁵²Fe, ⁵⁹Fe, ²⁵¹Fm, ²⁵²Fm, ²⁵³Fm, ²⁵⁴Fm, ²⁵⁵Fm, ²⁵⁷Fm ⁶⁶Ga, ⁶⁷Ga, ⁶⁸Ga, ⁷²Ga, ⁷³Ga, ¹⁴⁶Gd, ¹⁴⁷Gd, ¹⁴⁹Gd, ¹⁵¹Gd, ¹⁵³Gd, ¹⁵⁹Gd, ⁶⁸Ge, ⁶⁹Ge, ⁷¹Ge, ⁷⁷Ge ¹⁷⁰Hf, ¹⁷¹Hf, ¹⁷³Hf, ¹⁷⁵Hf, ^{179m2}Hf, ^{180m}Hf, ¹⁸¹Hf, ¹⁸⁴Hf, ¹⁹²Hg, ¹⁹³Hg, ^{193m}Hg, ¹⁹⁵Hg, ^{195m}Hg, ¹⁹⁷Hg ^{197m}Hg, ²⁰³Hg, ^{160m}Ho, ¹⁶⁶Ho, ¹⁶⁷Ho, ¹²³I, ¹²⁴I, ¹²⁶I, ¹³⁰I, ¹³²I, ¹³³I, ¹³⁵I, ¹⁰⁹In, ¹¹⁰In, ¹¹¹In, ^{114m}In, ^{115m}In, ¹⁸⁴Ir, ¹⁸⁵Ir, ¹⁸⁶Ir, ¹⁸⁷Ir, ¹⁸⁸Ir, ¹⁸⁹Ir, ¹⁹⁰Ir, ^{190m2}Ir, ¹⁹²Ir, ^{193m}Ir, ¹⁹⁴Ir, ^{194m2}Ir, ^{195m}Ir, ⁴²K, ⁴³K, ⁷⁸Kr ⁷⁹Kr, ^{81m}Kr, ^{85m}Kr, ¹³²La, ¹³³La, ¹³⁵La, ¹⁴⁰La, ¹⁴¹La, ²⁶²Lr, ¹⁶⁹Lu, ¹⁷⁰Lu, ¹⁷¹Lu, ¹⁷²Lu, ^{174m}Lu, ^{176m}Lu ¹⁷⁷Lu, ^{177m}Lu, ¹⁷⁹Lu, ²⁵⁷Md, ²⁵⁸Md, ²⁶⁰Md, ²⁸Mg, ⁵²Mn, ⁹⁰Mo, ^{93m}Mo, ⁹⁹Mo, ¹⁰⁰Mo, ¹³N, ²⁴Na, ⁹⁰Nb, ^{91m}Nb ^{92m}Nb, ⁹⁵Nb, ^{95m}Nb, ⁹⁶Nb, ¹³⁸Nd, ^{139m}Nd, ¹⁴⁰Nd, ¹⁴⁷Nd, ⁵⁶Ni, ⁵⁷Ni, ⁶⁶Ni, ²³⁴Np, ²³⁶Np, ⁹⁹Mo, Np, ²³⁸Np ²³⁹Np, ¹⁵O, ¹⁸²Os, ¹⁸³Os, ^{183m}Os, ¹⁸⁵Os, ^{189m}Os, ¹⁹¹Os, ^{191m}Os, ¹⁹³Os, ³²P, ³³P, ²²⁸Pa, ²²⁹Pa, ²³⁰Pa, ²³²Pa, ²³³Pa, ²³⁴Pa, ²⁰⁰Pb, ²⁰¹Pb, ²⁰²mPb, ²⁰³Pb, ²⁰⁹Pb, ²¹²Pb, ¹⁰⁰Pd, ¹⁰¹Pd, ¹⁰³Pd, ¹⁰⁹Pd ^{111m}Pd, ¹¹²Pd, ¹⁴³Pm, ¹⁴⁸Pm, ^{148m}Pm, ¹⁴⁹Pm, ¹⁵¹Pm, ²⁰⁴Po, ²⁰⁶Po, ²⁰⁷Po, ²¹⁰Po, ¹³⁹Pr, ¹⁴²Pr, ¹⁴³Pr, ¹⁴⁵Pr, ¹⁸⁸Pt, ¹⁹¹Pt, ¹⁹¹Pt, ^{193m}Pt, ^{195m}Pt, ¹⁹⁷Pt, ²⁰⁰Pt, ²⁰²Pt, ²³⁴Pu, ²³⁷Pu, ²⁴³Pu, ²⁴⁵Pu, ²⁴⁶Pu, ²⁴⁷Pu ²²³Ra, ²²⁴Ra, ²²⁵Ra, ⁸¹Rb, ⁸²Rb, ⁸²mRb, ⁸³Rb, ⁸⁴Rb, ⁸⁶Rb, ¹⁸¹Re, ¹⁸²Re, ^{182m}Re, ¹⁸³Re, ¹⁸⁴Re, ^{184m}Re, ¹⁸⁶Re, ¹⁸⁸Re, ¹⁸⁹Re, ^{190m}Re, ⁹⁹Rh, ^{99m}Rh, ¹⁰⁰Rh, ^{101m}Rh, ¹⁰²Rh, ^{103m}Rh, ¹⁰⁵Rh, ²¹¹Rn, ²²²Rn, ⁹⁷Ru, ¹⁰³Ru, ¹⁰⁵Ru, ³⁵S, ^{118m}Sb, ¹¹⁹Sb, ¹²⁰Sb, ^{120m}Sb, ¹²²Sb, ¹²⁴Sb, ¹²⁶Sb, ¹²⁷Sb, ¹²⁸Sb, ¹²⁹Sb, ⁴³Sc, ⁴⁴Sc, ^{44m}Sc, ⁴⁶Sc, ⁴⁷Sc, ⁴⁸Sc, ⁷²Se, ⁷³Se, ⁷⁵Se, ¹⁵³Sm, ¹⁵⁶Sm, ¹¹⁰Sn, ¹¹³Sn, ^{117m}Sn ^{119m}Sn, ¹²¹Sn, ¹²³Sn, ¹²⁵Sn, ⁸²Sr, ⁸³Sr, ⁸⁵Sr, ⁸⁹Sr, ⁹¹Sr, ¹⁷³Ta, ¹⁷⁵Ta, ¹⁷⁶Ta, ¹⁷⁷Ta, ¹⁸⁰Ta, ¹⁸²Ta ¹⁸³Ta, ¹⁸⁷⁴Ta, ¹⁴⁹Tb, ¹⁵⁰Tb, ¹⁵¹Tb, ¹⁵²Tb, ¹⁵³Tb, ¹⁵⁴Tb, ^{154m}Tb, ^{154m2}Tb, ¹⁵⁵Tb, ¹⁵⁶Tb, ^{156m}Tb ^{156m2}Tb, ¹⁶⁰Tb, ¹⁶¹Tb, ⁹⁴Tc, ⁹⁵Tc, ^{95m}Tc, ⁹⁶Tc, ^{97m}Tc, ^{99m}Tc, ¹¹⁸Te, ¹¹⁹Te, ^{119m}Te, ¹²¹Te, ^{121m}Te, ^{123m}Te, ^{125m}Te, ¹²⁷Te, ^{127m}Te, ^{129m}Te, ^{131m}Te, ¹³²Te, ²²⁷Th, ²²⁹Th, ²³¹Th, ²³⁴Th, ⁴⁵Ti, ¹⁹⁸Tl, ¹⁹⁹Tl, ²⁰⁰Tl, ²⁰¹Tl, ²⁰²Tl, ²⁰⁴Tl, ¹⁶⁵Tm, ¹⁶⁶Tm, ¹⁶⁷Tm, ¹⁶⁸Tm, ¹⁷⁰Tm, ¹⁷²Tm, ¹⁷³Tm, ²³⁰U, ²³¹U, ²³⁷U, ²⁴⁰U, ⁴⁸V, ¹⁷⁸W, ¹⁸¹W, ¹⁸⁵W, ¹⁸⁷W, ¹⁸⁸W, ¹²²Xe, ¹²⁵Xe, ¹²⁷Xe, ^{129m}Xe, ^{131m}Xe, ¹³³Xe, ^{133m}Xe, ¹³⁵Xe, ^{85m}Y, ⁸⁶Y, ⁸⁷Y, ^{87m}Y, ⁸⁸Y, ⁹⁰Y, ^{90m}Y, ⁹¹Y, ⁹²Y, ⁹³Y, ₁₆₆Yb, ¹⁶⁹Yb, ¹⁷⁵Yb, ⁶²Zn, ⁶⁵Zn, ^{69m}Zn, ^{71m}Zn, ⁷²Zn, ⁸⁶Zr, ⁸⁸Zr, ⁸⁹Zr, ⁹⁵Zr, and ⁹⁷Zr.

99. The conjugate of any one of claims 51 to 98, wherein the targeting ligand is selected from an oligonucleotide, an oligopeptide, a protein, a peptide and a small molecule compound.

100. A composition comprising a conjugate of any one of claims 51 to 99 and a carrier.

101. A pharmaceutical composition comprising a conjugate of any one of claims 51 to 99 and a pharmaceutically acceptable carrier.

102. The pharmaceutical composition of claim 101, wherein the composition is a diagnostic composition.

103. The pharmaceutical composition of claim 101, wherein the composition is a theranostic composition.

104. A method of medical diagnosing comprising administering an effective amount of a conjugate of any one of claims 24 to 99 or a composition of claim 101 to a subject in need thereof.

105. The method of claim 104, wherein the medical diagnostic method is performed using positron emission tomography (PET), single-photon emission computed tomography (SPECT), radioisotope renography or scintigraphy.

106. A method of treatment of one or more diseases, disorders or conditions, comprising administering an effective amount of a conjugate of any one of claims 24 to 99 or a composition of claim 101 to a subject in need thereof.

107. A method of radionuclide therapy comprising administering an effective amount of a conjugate of any one of claims 24 to 99 or a composition of claim 101 to a subject in need thereof.

108. A method of theranostic treatment comprising administering an effective amount of a conjugate of any one of claims 24 to 99 or a composition of claim 101 to a subject in need thereof.

109. The method any one of claims 104 to 108, wherein the method is for treating one or more diseases, disorders or conditions associated with cellular proliferation.

110. The method any one of claims 104 to 108, wherein the method is for treating one or more of cancers, thyroid diseases (e.g., hyperthyroidism or thyrotoxicosis), blood disorders (e.g., Polycythemia vera, an excess of red blood cells produced in the bone marrow), and/or cellular proliferation in blood vessels following balloon angioplasty and/or stent placement (known as restenosis).

111. The method of claim 110, wherein the method is for treating cancer.

112. The method of claim 111, wherein the cancer is adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease cancer, intestinal cancer, kidney cancer, larynx cancer, leukemia, liver cancer, lymph node cancer, lymphoma, lung cancer, melanoma, mesothelioma, myeloma, nasopharynx cancer, neuroblastoma, non-Hodgkin's lymphoma, oral cancer, ovarian cancer, pancreatic cancer, penile cancer, pharynx cancer, prostate cancer, rectal cancer, sarcomcancer, seminomcancer, skin cancer, stomach cancer, teratomcancer, testicular cancer, thyroid cancer, uterine cancer, vaginal cancer, vascular tumor cancer, and/or cancer from metastases thereof.

113. Use of a conjugate of any one of claims 24 to 99 or a composition of claim 101 in the manufacture of a medicament for medical diagnosis.

114. Use of a conjugate of any one of claims 24 to 99 or a composition of claim 101 in the manufacture of a medicament for treatment of one or more diseases, disorders or conditions.

115. Use of a conjugate of any one of claims 24 to 99 or a composition of claim 101 in the manufacture of a medicament for theranostic applications.

116. Use of a conjugate of any one of claims 24 to 99 or a composition of claim 101 in the manufacture of a medicament for radionuclide therapy.

117. The use of any one of claims 113 to 116, wherein the use is for treating one or more diseases, disorders or conditions associated with cellular proliferation.

118. The use of any one of claims 113 to 116, wherein the use is for treating one or more of cancers, thyroid diseases (e.g., hyperthyroidism or thyrotoxicosis), blood disorders (e.g., Polycythemia vera, an excess of red blood cells produced in the bone marrow), and/or cellular proliferation in blood vessels following balloon angioplasty and/or stent placement (known as restenosis).

119. The use of claim 118, wherein the use for treating cancer.

120. The use of claim 119, wherein the cancer is adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease cancer, intestinal cancer, kidney cancer, larynx cancer, leukemia, liver cancer, lymph node cancer, lymphoma, lung cancer, melanoma, mesothelioma, myeloma, nasopharynx cancer, neuroblastoma, non-Hodgkin's lymphoma, oral cancer, ovarian cancer, pancreatic cancer, penile cancer, pharynx cancer, prostate cancer, rectal cancer, sarcomcancer, seminomcancer, skin cancer, stomach cancer, teratomcancer, testicular cancer, thyroid cancer, uterine cancer, vaginal cancer, vascular tumor cancer, and/or cancer from metastases thereof.

121. A kit comprising a compound of claims 1 to 23 or a conjugate of any one of claims 24 to 99 or a composition of claim 101 and a pharmaceutically acceptable excipient.

122. The kit of claim 121 for use in medical diagnosis.

123. The kit of claim 121 for use in radionuclide therapy.

124. The kit of claim 121 for use in theranostic applications.
